SEQUENCE LISTING (3 sequences)

SEQ ID NO: 1 - DNA, 20 bp, Artificial
  primer
gtgtctgcggcgttttatca

SEQ ID NO: 2 - DNA, 21 bp, Artificial
  primer
gacaaacgggcaacatacctt

SEQ ID NO: 3 - DNA, 28 bp, Artificial
  probe
cctctkcatcctgctgctatgcctcatc

(19) United States
(12) Patent Application Publication
(10) Pub. No.: US 2023/0165865 A1
(43) Pub. Date: Jun. 1, 2023

(54) HETEROARYLDIHYDROPYRIMIDINE DERIVATIVES AND METHODS OF TREATING HEPATITIS B INFECTIONS

(71) Applicant: Janssen Pharmaceutica NV, Beerse (BE)

(72) Inventors: Yanping XU, Noblesville, IN (US); Yimin JIANG, Londonberry, NH (US); Gang DENG, Shanghai (CN); Zhanling CHENG, Shanghai (CN); Chao LIANG, Shanghai (CN)

(21) Appl. No.: 17/415,712

(22) PCT Filed: Dec. 19, 2019
(86) PCT No.: PCT/CN2019/126739

§ 371 Date: Jun. 17, 2021

Related U.S. Application Data
(60) Provisional application No. 62/791,576, filed on Jan. 11, 2019.

(30) Foreign Application Priority Data
Dec. 20, 2018 (CN) PCT/CN2018/122258

(51) Int. Cl. A61K 31/52; A61P 31/20

(52) U.S. Cl. CPC A61K 31/52; A61P 31/20

(57) ABSTRACT

Provided herein are compounds useful for the treatment of HBV infection in a subject in need thereof, pharmaceutical compositions thereof, and methods of inhibiting, suppressing, or preventing HBV infection in the subject.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress. Capsid structures also respond to environmental cues to allow un-coating after viral entry. Consistently, the appropriate timing of capsid assembly and dis-assembly, the appropriate capsid stability and the function of core protein have been found to be critical for viral infectivity.

There is a need in the art for therapeutic agents that can increase the suppression of virus production and that can treat, ameliorate, or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

Background art on heteroaryldihydropyrimidines for use in the treatment of HBV includes WO 2015/132276, WO2013/102655 and WO99/54326.

### SUMMARY

Provided herein are compounds useful for the treatment of HBV infection in a subject in need thereof. Thus, in an aspect, provided herein is a compound of Formula I:

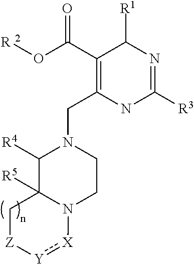

including the deuterated isomers, stereoisomers or tautomeric forms thereof, or a pharmaceutically acceptable salt thereof, wherein:

R¹ is selected from the group consisting of phenyl, thiophenyl, pyridyl, and pyridonyl, optionally substituted with one or more substituents selected from the group consisting of C₁₋₄ alkyl, halogen, and CN;

R² is C₁₋₄alkyl;

R³ is selected from the group consisting of thiazolyl, pyridyl, and oxazolyl, optionally substituted with one or more substituents selected from fluorine, and C₁₋₆alkyl;

n is an integer of 0 or 1;

R⁴ and R⁵ are independently selected from the group consisting of H and —COOH;

[symbol] (i.e., the bond between X and Y) is a single bond or a double bond;

when X and Y are linked by a single bond, X is selected from the group consisting of C(═S), C(═NR⁶), C(═CHR⁷) and CHR⁸, and Y is NR⁹;

when X and Y are linked by a double bond, X is C—SR⁹ or C—OR⁹, and Y is N atom;

Z is selected from the group consisting of CH₂ and C(═O);

R⁶ is selected from the group consisting of CN, C(═O)CH₃, and SO₂CH₃;

R⁷ is CN;

R⁸ is CF₃;

R⁹ is selected from the group consisting of H, —C₁₋₆alkyl, —C₁₋₆alkyl-R¹⁰, —C₁₋₆alkoxy-C₁₋₆ alkyl-R¹⁰, —(CH₂)ₚ—C(R¹¹R¹²)—R¹⁰ and —(CH₂)ₚ-Q-R¹⁰;

p is an integer of 0, 1, 2, or 3;

R¹¹ and R¹² together with carbon atom to which they are attached form a 3- to 7-saturated membered ring, optionally containing a heteroatom, the heteroatom being an oxygen or a nitrogen, the nitrogen being substituted with H, —C₁₋₆alkyl, —C₁₋₆alkoxy-C₁₋₆alkyl and —C₁₋₆ alkylcarbonyl;

Q is selected from the group consisting of aryl, heteroaryl, and a 3- to 7-membered saturated ring, optionally containing a heteroatom, the heteroatom being an oxygen or a nitrogen, the nitrogen being substituted with H, —C₁₋₆alkyl, —C₁₋₆alkoxy-C₁₋₆alkyl and —C₁₋₆ alkylcarbonyl;

R¹⁰ is selected from —COOH, —C(═O)NHS(═O)₂—C₁₋₆alkyl, tetrazolyl and carboxylic acid bioisosteres.

In another aspect, provided herein is a pharmaceutical composition comprising at least one compound of Formula I, or a pharmaceutically acceptable salt thereof, together with a pharmaceutically acceptable carrier.

In another aspect, provided herein is a pharmaceutical composition comprising at least one disclosed compound, together with a pharmaceutically acceptable carrier.

In another aspect, provided herein is a method of treating an HBV infection or of an HBV-induced disease in an individual in need thereof, comprising administering to the individual a therapeutically effective amount of a compound of Formula I or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula I, or a pharmaceutically acceptable salt thereof.

In an embodiment, any of the methods provided herein can further comprising administering to the individual at least one additional therapeutic agent selected from the group consisting of HBV inhibitors as herein further defined.

### DETAILED DESCRIPTION

Provided herein are compounds, e.g., the compounds of I, or pharmaceutically acceptable salts thereof, that are useful in the treatment and prevention of HBV infection in subject.

Without being bound to any particular mechanism of action, these compounds are believed to modulate or disrupt HBV assembly and other HBV core protein functions necessary for HBV replication or the generation of infectious particles and/or may disrupt HBV capsid assembly leading to empty capsids with greatly reduced infectivity or replication capacity. In other words, the compounds provided herein may act as capsid assembly modulators.

The compounds provided herein have potent antiviral activity, exhibit favorable metabolic properties, tissue distribution, safety and pharmaceutical profiles, and are suitable for use in humans. Disclosed compounds may modulate (e.g., accelerate, delay, inhibit, disrupt or reduce) normal viral capsid assembly or disassembly, bind capsid or alter metabolism of cellular polyproteins and precursors. The modulation may occur when the capsid protein is mature, or during viral infectivity. Disclosed compounds can be used in methods of modulating the activity or properties of HBV cccDNA, or the generation or release of HBV RNA particles from within an infected cell.

In an embodiment, the compounds described herein are suitable for monotherapy and are effective against natural or native HBV strains and against HBV strains resistant to currently known drugs. In another embodiment, the compounds described herein are suitable for use in combination therapy.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of 20% or ±10%, including ±5%, ±1%, and ±0.1% from the specified value, as such variations are appropriate to perform the disclosed methods.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In an embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity or is lethal to the virus.

As used herein, the term “treatment” or “treating” is defined as the application or administration of a therapeutic agent, i.e., a disclosed compound (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection, or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “alkyl,” by itself or as part of another substituent means, unless otherwise stated, a straight or branched chain hydrocarbon having the number of carbon atoms designated (i.e., C₁-C₃alkyl means an alkyl having one to three carbon atoms, C₁-C₄alkyl means an alkyl having one to four carbon) and includes straight and branched chains. Examples include methyl, ethyl, propyl, isopropyl, butyl, isobutyl, tert-butyl. Embodiments of alkyl generally include, but are not limited to, C₁-C₁₀ alkyl, such as C₁-C₆ alkyl, such as C₁-C₄ alkyl.

As used herein, the term “alkenyl,” by itself or as part of another substituent means, unless otherwise stated, a linear or branched chain of hydrocarbons comprising at least one carbon to carbon double bond, having the number of carbon atoms designated (i.e., C₂-C₄ alkenyl or C₂₋₄alkenyl means an alkenyl having two to four to eight carbon atoms. C₄-C₈ alkenyl or C₄₋₈alkenyl means an alkenyl having four carbon atoms. Embodiments of alkenyl generally include, but are not limited to, C₂-C₆ alkenyl, such as C₂-C₄ alkenyl, such as C₂-C₃ alkenyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “3-7 membered saturated ring” refers to a mono cyclic non-aromatic saturated radical, wherein each of the atoms forming the ring (i.e., skeletal atoms) is a carbon atom, unless such ring contains one or more heteroatoms if so further defined. 3-7 Membered saturated rings include groups having 3 to 7 ring atoms. Monocyclic 3-7 membered saturated rings include, but are not limited to, cyclopropyl, cyclobutyl, cyclopentyl, cyclohexyl, cycloheptyl.

As used herein, a 3-7 membered saturated ring may optionally contain a heteroatom, said heteroatom being an oxygen, or a nitrogen substituted with H, C₁₋₆alkyl, or C₁₋₆alkoxy-C₁₋₆ alkyl.

As used herein, the term “aromatic” refers to a carbocycle or heterocycle with one or more polyunsaturated rings and having aromatic character, i.e., having (4n+2) delocalized R (pi) electrons, where n is an integer.

As used herein, the term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. Preferred examples are phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆-C₁₂-aryl). In some embodiments, aryl groups have six carbon atoms (e.g., C₆-aryl).

As used herein, the term “heteroaryl” or “heteroaromatic” refers to a heterocycle having aromatic character. Heteroaryl substituents may be defined by the number of carbon atoms, e.g., C₁-C₉-heteroaryl indicates the number of carbon atoms contained in the heteroaryl group without including the number of heteroatoms. For example, a C₁-C₉-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group.

As used herein, the terminology “selected from . . . ” (e.g., “R⁴ is selected from A, B and C”) is understood to be equivalent to the terminology “selected from the group consisting of . . . ” (e.g., “R⁴ is selected from the group consisting of A, B and C”).

An embodiment relates to a compound of Formula I as defined herein wherein the carboxylic acid bioisosteres are —S(═O)₂(OH), —P(═O)(OH)₂, —C(═O)NHOH, —C(═O)NHCN, 1,2,4-oxadiazol-5(4H)-one, or 3-hydroxy-4-methylcyclobut-3-ene-1,2-dione. This refers to the following structures:

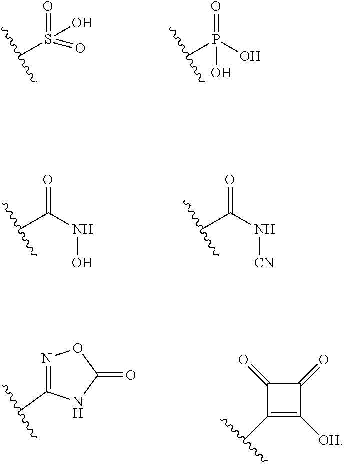 [SMILES: *S(=O)(=O)O ; *P(=O)(O)O ; *C(=O)NO ; *C(=O)NC#N ; *c1noc(=O)[nH]1 ; *c1c(O)c(=O)c1=O]

An embodiment relates to a compound of Formula I as defined herein, wherein R¹ is phenyl substituted with one or more substituents selected from halogens and C₁₋₆alkyl.

An embodiment relates to a compound of Formula I as defined herein, wherein R² is methyl or ethyl.

An embodiment relates to a compound of Formula I as defined herein, wherein R³ is thiazolyl.

An embodiment relates to a compound of Formula I as defined herein, wherein R⁴ and R⁵ are H.

An embodiment relates to a compound of Formula I as defined herein, wherein X is C(═S).

An embodiment relates to a compound of Formula I as defined herein, wherein Z is CH₂.

An embodiment relates to a compound of Formula I as defined herein, wherein R⁹ is —C₁₋₆alkyl-CO₂H, —(CH₂)ₚ—C(R¹¹R¹²)—R¹⁰ or —(CH₂)ₚ-Q-R¹⁰.

An embodiment relates to a compound of Formula I as defined herein, wherein Q is phenyl, or wherein Q is a C₃₋₆cycloalkyl, or wherein Q is a 3- to 6-saturated membered ring containing an oxygen.

An embodiment relates to a compound of Formula I as defined herein, wherein R¹¹ and R¹² together with carbon atom to which they are attached form a C₃₋₆cycloalkyl, or R¹¹ and R¹² together with carbon atom to which they are attached form a 3- to 6-saturated membered ring containing an oxygen.

An embodiment relates to a compound selected from the group consisting of compound satisfying the following formulae:

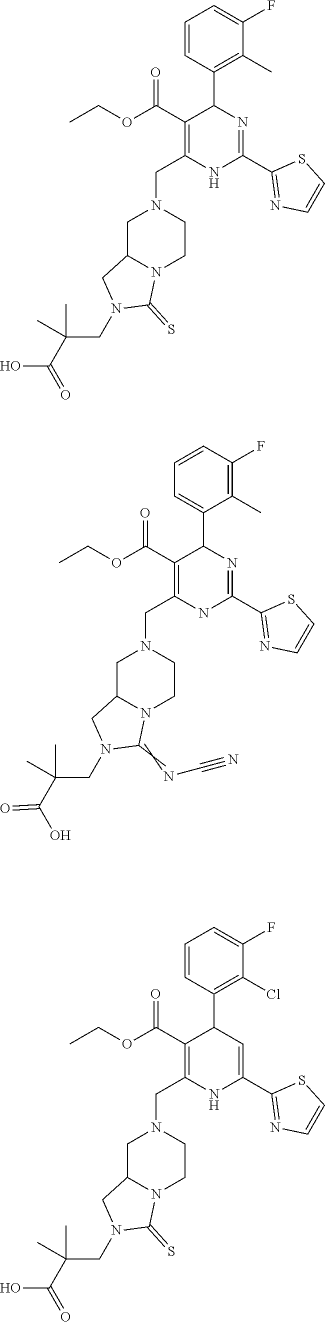 [SMILES: CCOC(=O)C1=C(CN2CCN3C(=S)N(CC(C)(C)C(=O)O)CC3C2)NC(c2nccs2)=NC1c1cccc(F)c1C ; CCOC(=O)C1=C(CN2CCN3C(=NC#N)N(CC(C)(C)C(=O)O)CC3C2)NC(c2nccs2)=NC1c1cccc(F)c1C ; CCOC(=O)C1=C(CN2CCN3C(=S)N(CC(C)(C)C(=O)O)CC3C2)NC(c2nccs2)=CC1c1cccc(F)c1Cl]

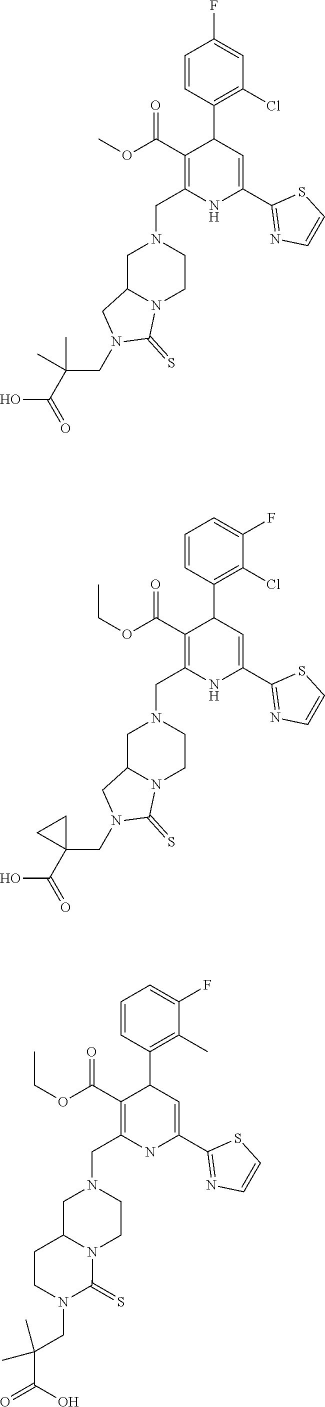 [SMILES: COC(=O)C1=C(CN2CCN3C(=S)N(CC(C)(C)C(=O)O)CC3C2)NC(c2nccs2)=CC1c1ccc(F)cc1Cl ; CCOC(=O)C1=C(CN2CCN3C(=S)N(CC4(C(=O)O)CC4)CC3C2)NC(c2nccs2)=CC1c1cccc(F)c1Cl ; CCOC(=O)C1=C(CN2CCN3C(=S)N(CC(C)(C)C(=O)O)CCC3C2)NC(c2nccs2)=CC1c1cccc(F)c1C]

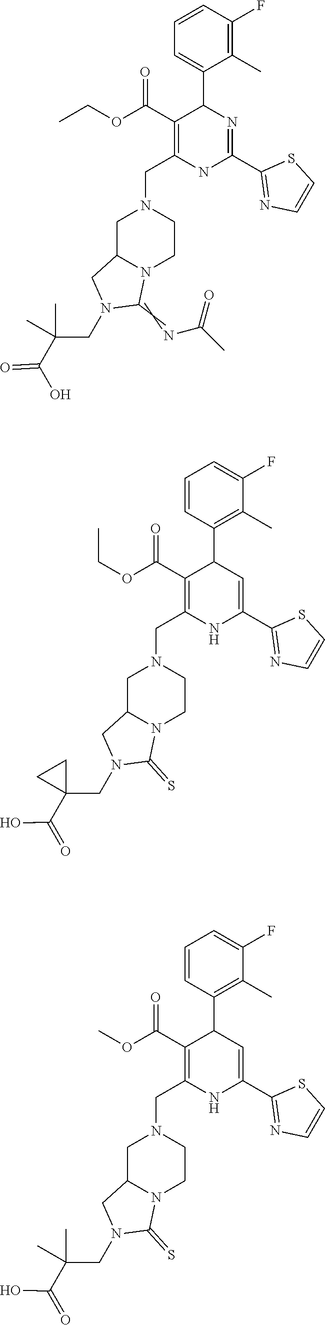 [SMILES: CCOC(=O)C1=C(CN2CCN3C(=NC(C)=O)N(CC(C)(C)C(=O)O)CC3C2)NC(c2nccs2)=NC1c1cccc(F)c1C ; CCOC(=O)C1=C(CN2CCN3C(=S)N(CC4(C(=O)O)CC4)CC3C2)NC(c2nccs2)=CC1c1cccc(F)c1C ; COC(=O)C1=C(CN2CCN3C(=S)N(CC(C)(C)C(=O)O)CC3C2)NC(c2nccs2)=CC1c1cccc(F)c1C]

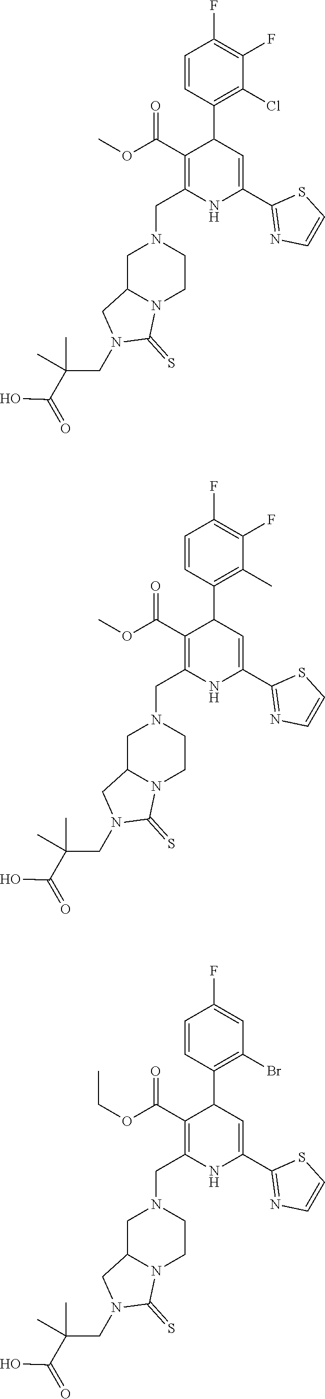 [SMILES: COC(=O)C1=C(CN2CCN3C(=S)N(CC(C)(C)C(=O)O)CC3C2)NC(c2nccs2)=CC1c1ccc(F)c(F)c1Cl ; COC(=O)C1=C(CN2CCN3C(=S)N(CC(C)(C)C(=O)O)CC3C2)NC(c2nccs2)=CC1c1ccc(F)c(F)c1C ; CCOC(=O)C1=C(CN2CCN3C(=S)N(CC(C)(C)C(=O)O)CC3C2)NC(c2nccs2)=CC1c1ccc(F)cc1Br]

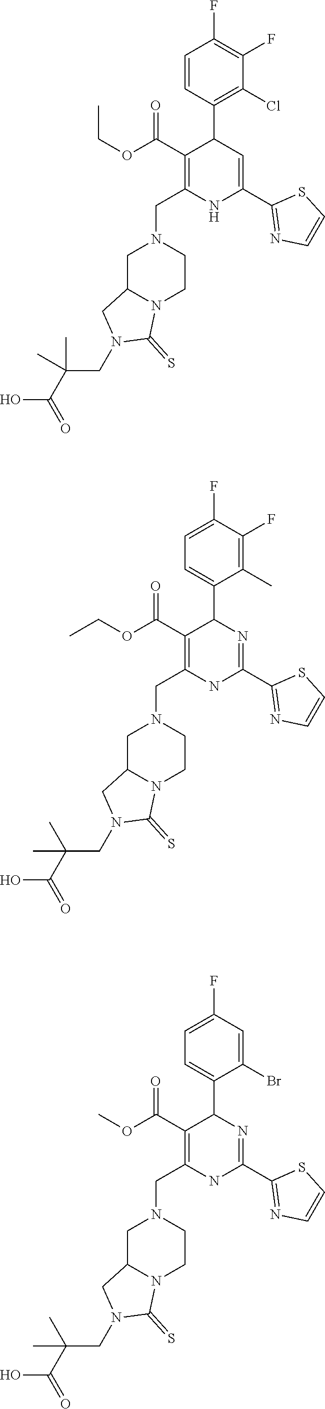 [SMILES: CCOC(=O)C1=C(CN2CCN3C(=S)N(CC(C)(C)C(=O)O)CC3C2)NC(c2nccs2)=CC1c1ccc(F)c(F)c1Cl ; CCOC(=O)C1=C(CN2CCN3C(=S)N(CC(C)(C)C(=O)O)CC3C2)NC(c2nccs2)=NC1c1ccc(F)c(F)c1C ; COC(=O)C1=C(CN2CCN3C(=S)N(CC(C)(C)C(=O)O)CC3C2)NC(c2nccs2)=NC1c1ccc(F)cc1Br]

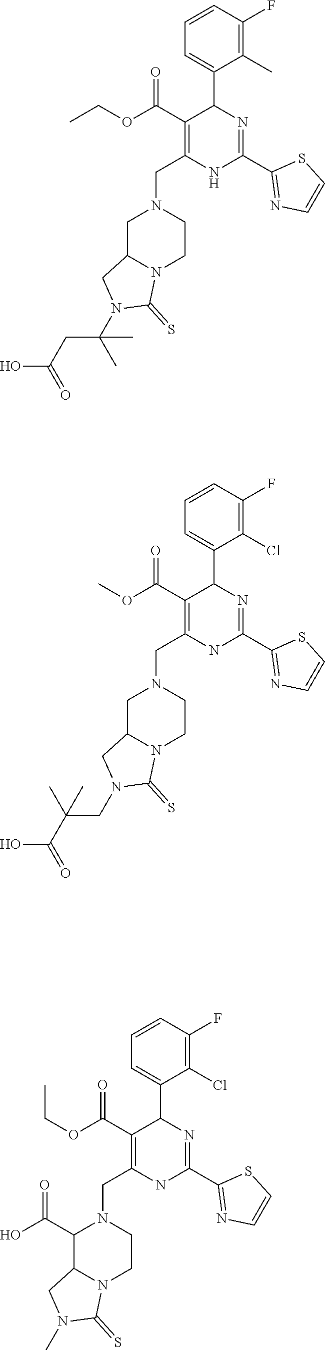 [SMILES: CCOC(=O)C1=C(CN2CCN3C(=S)N(C(C)(C)CC(=O)O)CC3C2)NC(c2nccs2)=NC1c1cccc(F)c1C ; COC(=O)C1=C(CN2CCN3C(=S)N(CC(C)(C)C(=O)O)CC3C2)NC(c2nccs2)=NC1c1cccc(F)c1Cl ; CCOC(=O)C1=C(CN2CCN3C(=S)N(C)CC3C2C(=O)O)NC(c2nccs2)=NC1c1cccc(F)c1Cl]

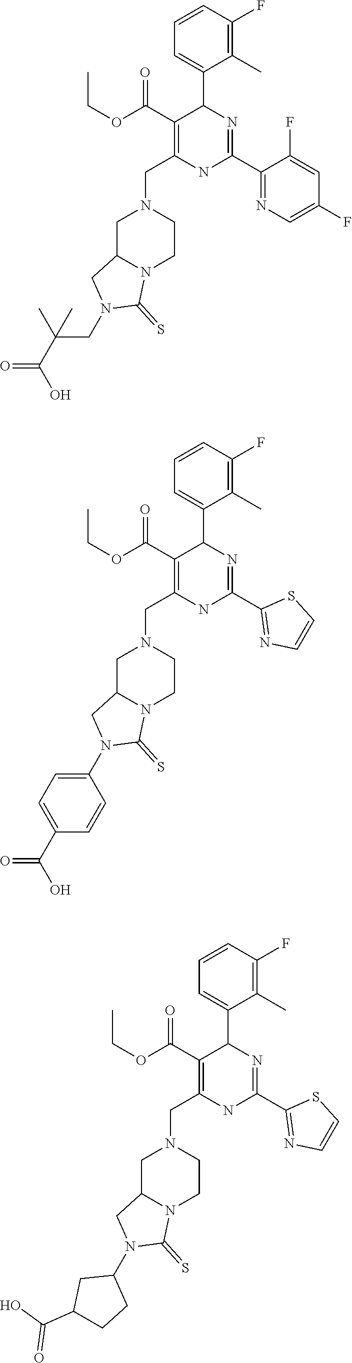 [SMILES: CCOC(=O)C1=C(CN2CCN3C(=S)N(CC(C)(C)C(=O)O)CC3C2)NC(c2ncc(F)cc2F)=NC1c1cccc(F)c1C ; CCOC(=O)C1=C(CN2CCN3C(=S)N(c4ccc(C(=O)O)cc4)CC3C2)NC(c2nccs2)=NC1c1cccc(F)c1C ; CCOC(=O)C1=C(CN2CCN3C(=S)N(C4CCC(C(=O)O)C4)CC3C2)NC(c2nccs2)=NC1c1cccc(F)c1C]

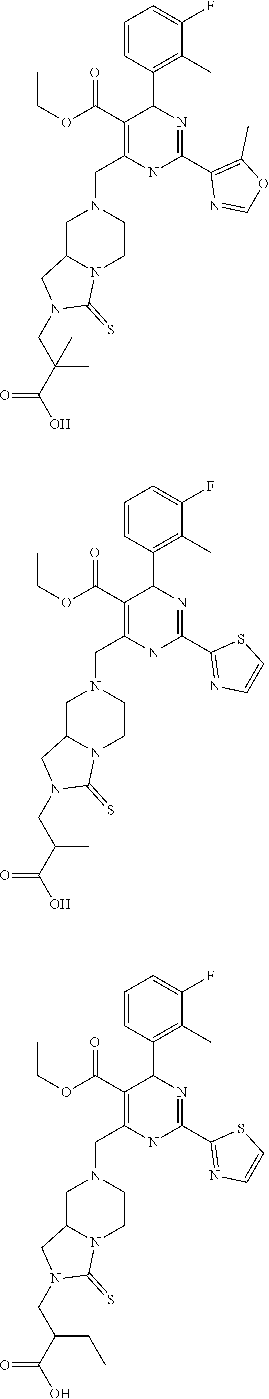 [SMILES: CCOC(=O)C1=C(CN2CCN3C(=S)N(CC(C)(C)C(=O)O)CC3C2)NC(c2ncoc2C)=NC1c1cccc(F)c1C ; CCOC(=O)C1=C(CN2CCN3C(=S)N(CC(C)C(=O)O)CC3C2)NC(c2nccs2)=NC1c1cccc(F)c1C ; CCOC(=O)C1=C(CN2CCN3C(=S)N(CC(CC)C(=O)O)CC3C2)NC(c2nccs2)=NC1c1cccc(F)c1C]

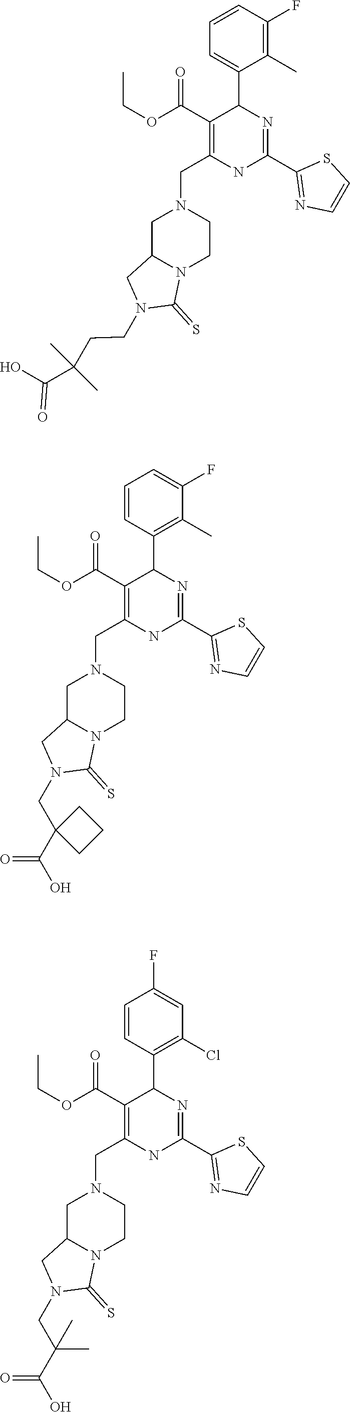 [SMILES: CCOC(=O)C1=C(CN2CCN3C(=S)N(CCC(C)(C)C(=O)O)CC3C2)NC(c2nccs2)=NC1c1cccc(F)c1C ; CCOC(=O)C1=C(CN2CCN3C(=S)N(CC4(C(=O)O)CCC4)CC3C2)NC(c2nccs2)=NC1c1cccc(F)c1C ; CCOC(=O)C1=C(CN2CCN3C(=S)N(CC(C)(C)C(=O)O)CC3C2)NC(c2nccs2)=NC1c1ccc(F)cc1Cl]

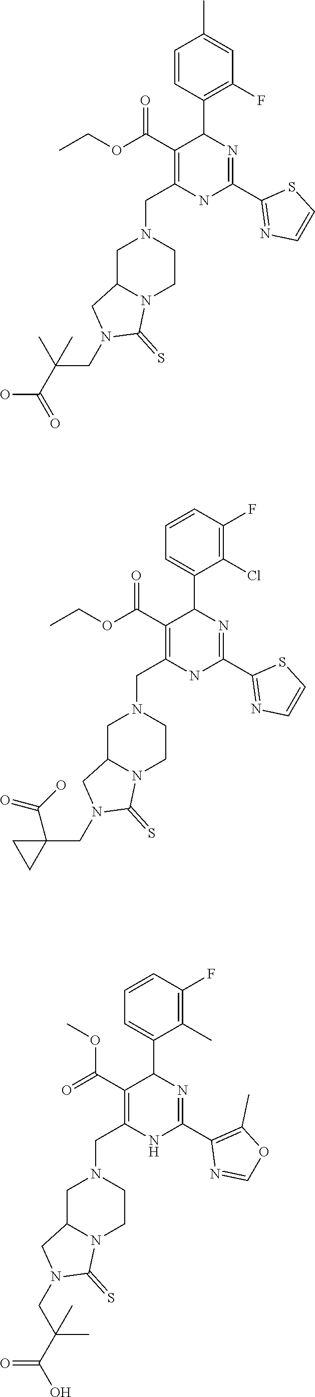 [SMILES: CCOC(=O)C1=C(CN2CCN3C(=S)N(CC(C)(C)C(=O)O)CC3C2)NC(c2nccs2)=NC1c1ccc(C)cc1F ; CCOC(=O)C1=C(CN2CCN3C(=S)N(CC4(C(=O)O)CC4)CC3C2)NC(c2nccs2)=NC1c1cccc(F)c1Cl ; COC(=O)C1=C(CN2CCN3C(=S)N(CC(C)(C)C(=O)O)CC3C2)NC(c2ncoc2C)=NC1c1cccc(F)c1C]

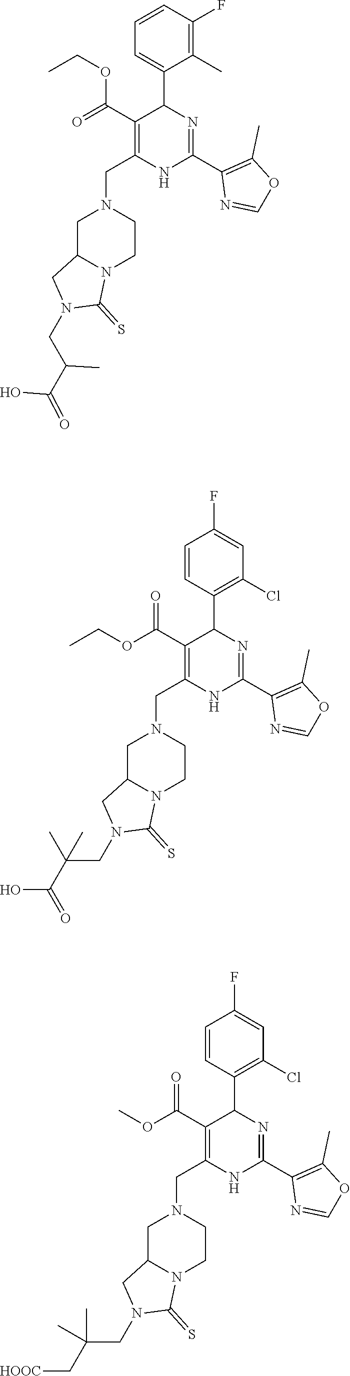 [SMILES: CCOC(=O)C1=C(CN2CCN3C(=S)N(CC(C)C(=O)O)CC3C2)NC(c2ncoc2C)=NC1c1cccc(F)c1C ; CCOC(=O)C1=C(CN2CCN3C(=S)N(CC(C)(C)C(=O)O)CC3C2)NC(c2ncoc2C)=NC1c1ccc(F)cc1Cl ; COC(=O)C1=C(CN2CCN3C(=S)N(CC(C)(C)CC(=O)O)CC3C2)NC(c2ncoc2C)=NC1c1ccc(F)cc1Cl]

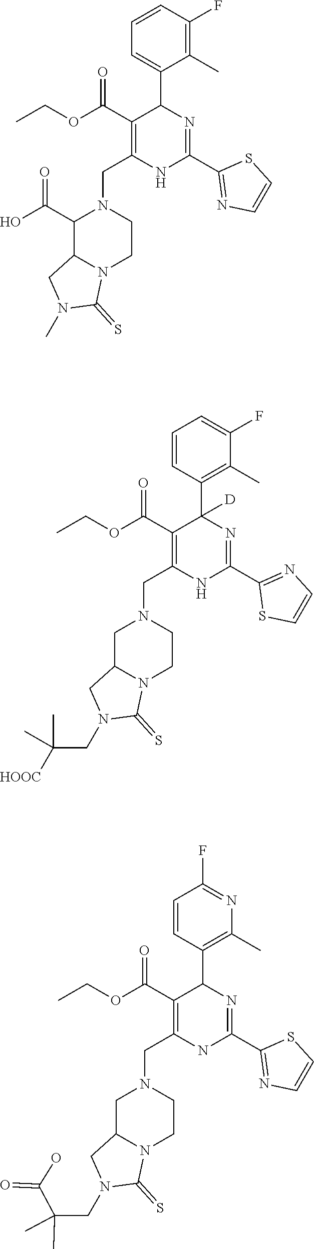 [SMILES: CCOC(=O)C1=C(CN2CCN3C(=S)N(C)CC3C2C(=O)O)NC(c2nccs2)=NC1c1cccc(F)c1C ; [2H]C1(c2cccc(F)c2C)N=C(c2nccs2)NC(CN2CCN3C(=S)N(CC(C)(C)C(=O)O)CC3C2)=C1C(=O)OCC ; CCOC(=O)C1=C(CN2CCN3C(=S)N(CC(C)(C)C(=O)O)CC3C2)NC(c2nccs2)=NC1c1ccc(F)nc1C]

An embodiment relates to a compound selected from the group consisting of compound satisfying the following formulae:

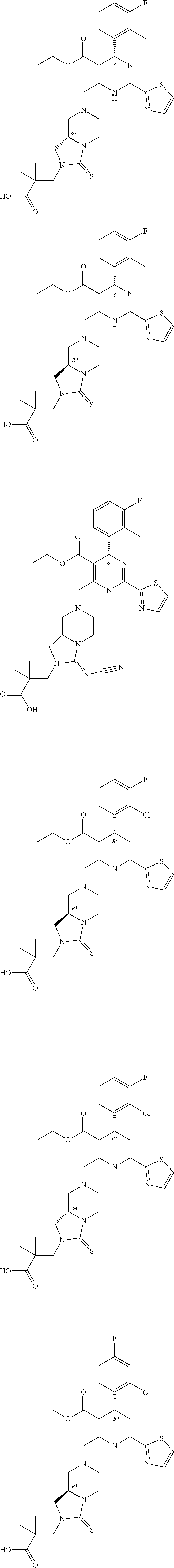

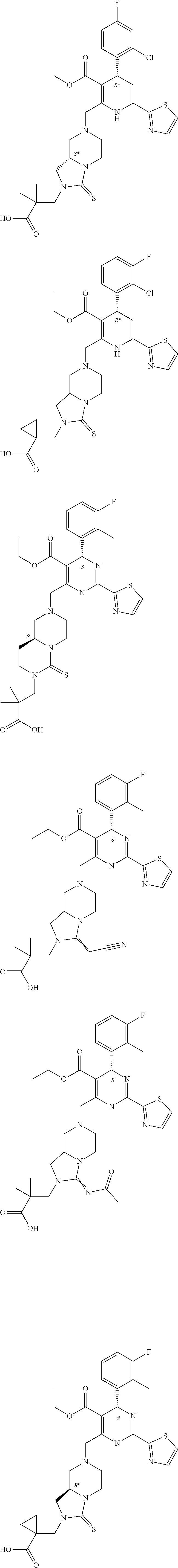

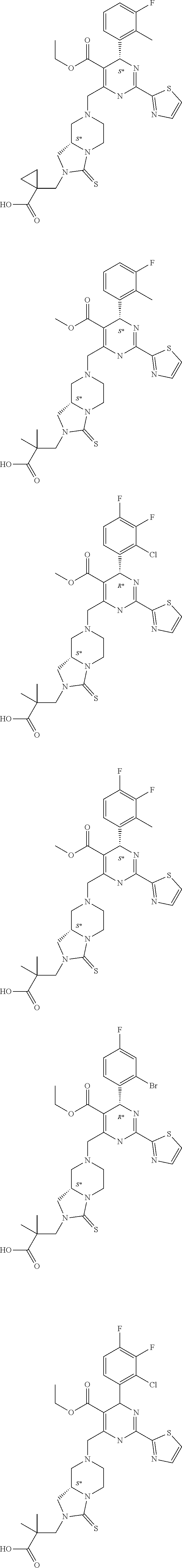

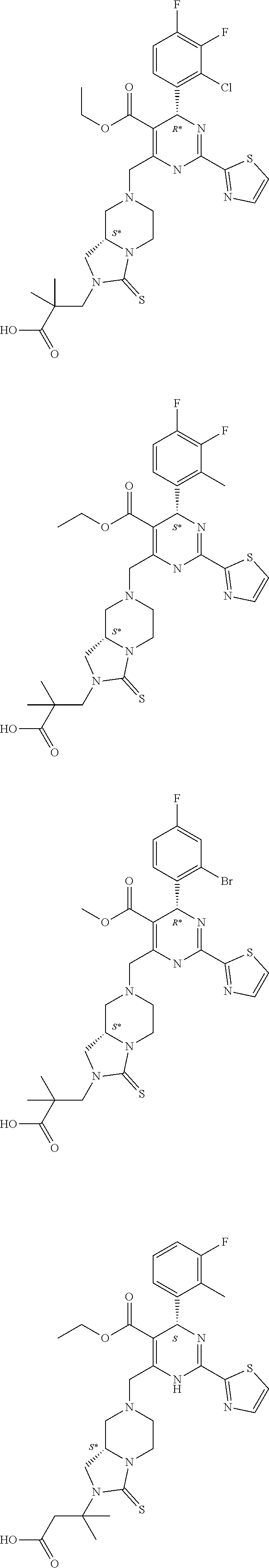

An embodiment relates to a compound selected from the group consisting of compound satisfying the following formulae:

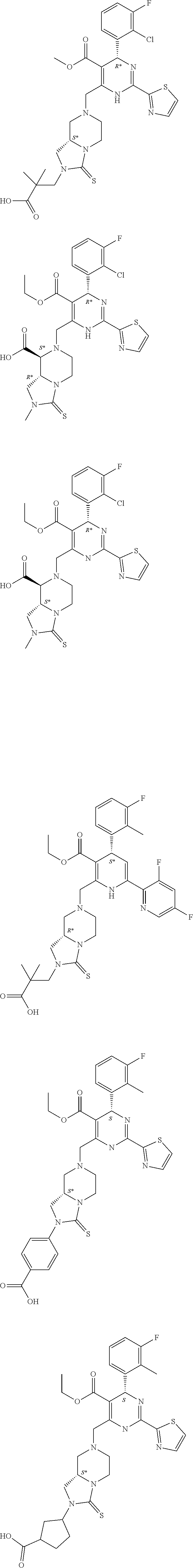

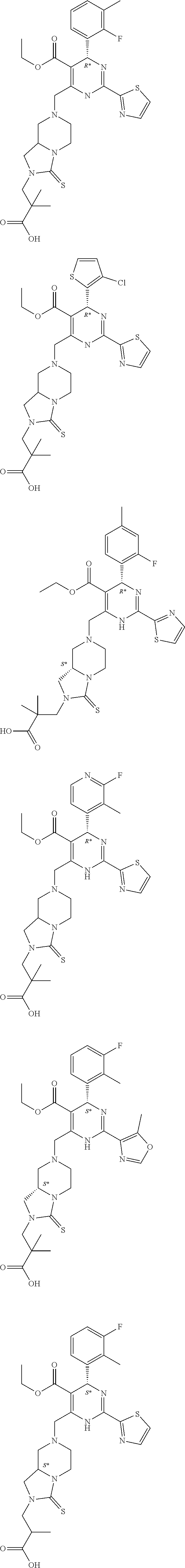

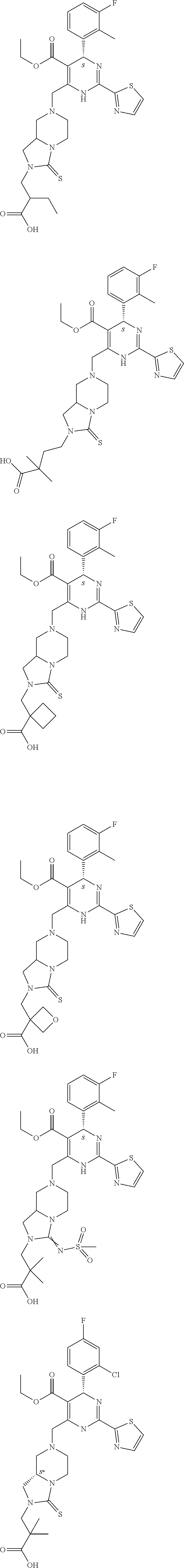

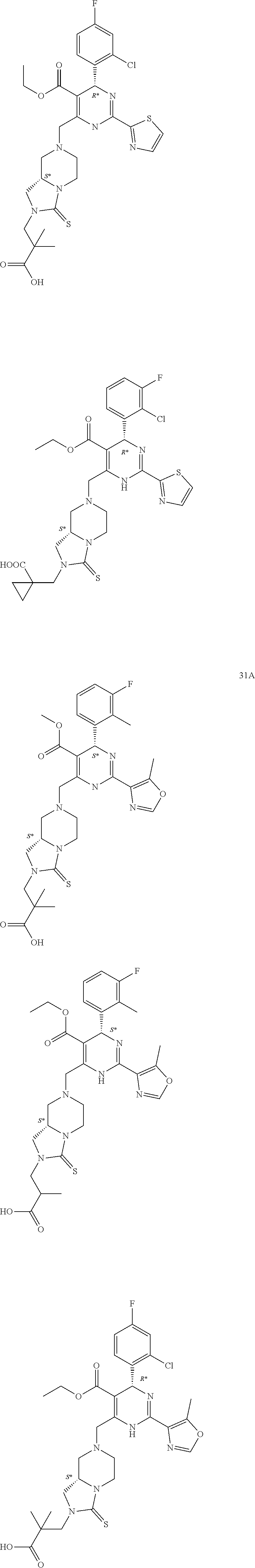 [SMILES: CCOC(=O)C1=C(CN2CCN3C(=S)N(CC(C)(C)C(=O)O)C[C@@H]3C2)NC(c2nccs2)=N[C@H]1c1ccc(F)cc1Cl ; CCOC(=O)C1=C(CN2CCN3C(=S)N(CC4(C(=O)O)CC4)C[C@@H]3C2)NC(c2nccs2)=N[C@H]1c1cccc(F)c1Cl ; COC(=O)C1=C(CN2CCN3C(=S)N(CC(C)(C)C(=O)O)C[C@@H]3C2)NC(c2ncoc2C)=N[C@H]1c1cccc(F)c1C ; CCOC(=O)C1=C(CN2CCN3C(=S)N(CC(C)C(=O)O)C[C@@H]3C2)NC(c2ncoc2C)=N[C@H]1c1cccc(F)c1C ; CCOC(=O)C1=C(CN2CCN3C(=S)N(CC(C)(C)C(=O)O)C[C@@H]3C2)NC(c2ncoc2C)=N[C@H]1c1ccc(F)cc1Cl]

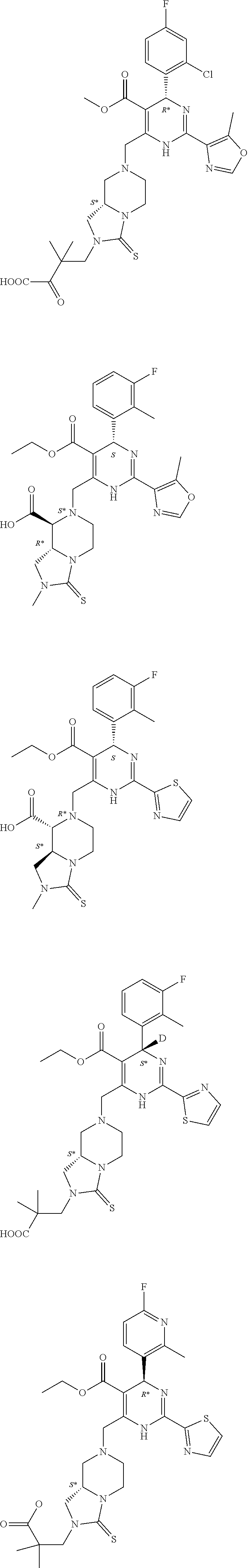

The disclosed compounds may possess one or more stereocenters, and each stereocenter may exist independently in either the R or S configuration. For some compounds, the stereochemical configuration at indicated centres has been assigned as “R*”, “S*” when the absolute stereochemistry is undetermined although the compound itself has been isolated as a single stereoisomer and is enantiomerically/diastereomerically pure. In an embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In an embodiment, a mixture of one or more isomer is utilized as the disclosed compound described herein. In another embodiment, compounds described herein contain one or more chiral centers. These compounds are prepared by any means, including stereoselective synthesis, enantioselective synthesis or separation of a mixture of enantiomers or diastereomers. Resolution of compounds and isomers thereof is achieved by any means including, by way of non-limiting example, chemical processes, enzymatic processes, fractional crystallization, distillation, and chromatography.

When the absolute R or S stereochemistry of a compound cannot be determined, it can be identified by the retention time after chromatography under particular chromatographic conditions as determined by chromatography column, eluent etc.

In an embodiment, the disclosed compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In an embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements).

In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In an embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and techniques known to a person skilled in the art. General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources or are prepared using procedures described herein.

Compounds of the application also includes intermediate compounds, including any salts thereof, such as

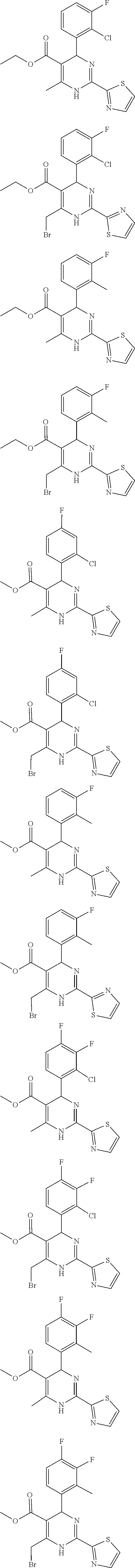

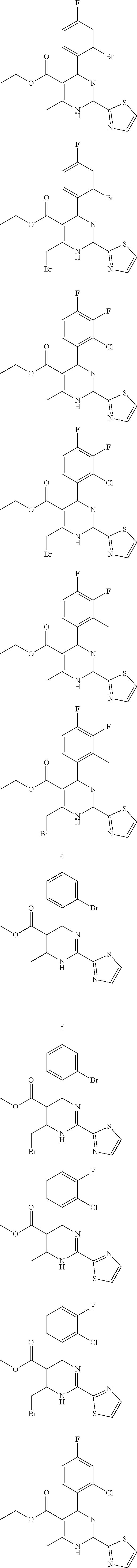

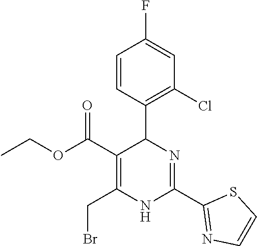 [SMILES: CCOC(=O)C1=C(CBr)NC(c2nccs2)=NC1c1ccc(F)cc1Cl]

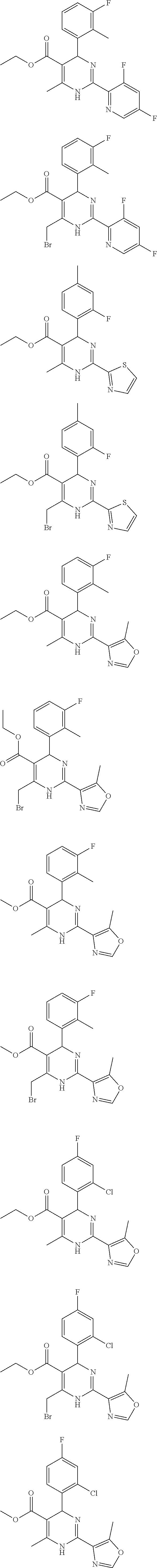

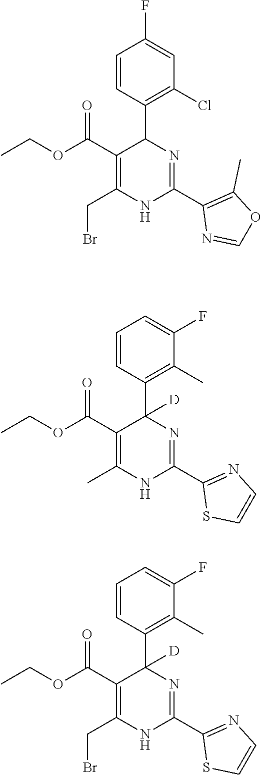 [SMILES: CCOC(=O)C1=C(CBr)NC(c2ncoc2C)=NC1c1ccc(F)cc1Cl ; [2H]C1(c2cccc(F)c2C)N=C(c2nccs2)NC(C)=C1C(=O)OCC ; [2H]C1(c2cccc(F)c2C)N=C(c2nccs2)NC(CBr)=C1C(=O)OCC]

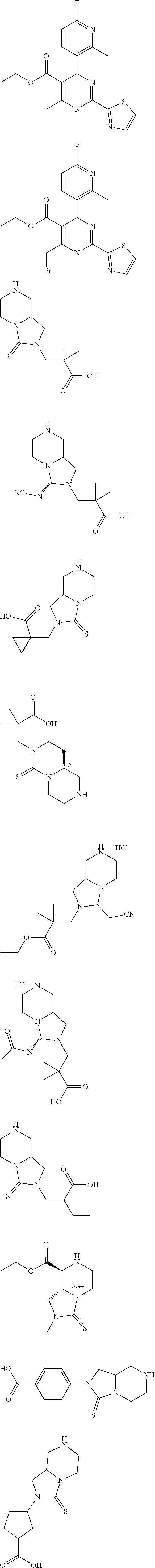

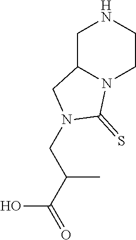 [SMILES: CC(CN1CC2CNCCN2C1=S)C(=O)O]

### Methods

Provided herein is a method of treating an HBV infection in an individual in need thereof, comprising administering to the individual a therapeutically effective amount of a disclosed compound.

Also provided herein is a method of eradicating an HBV infection in an individual in need thereof, comprising administering to the individual a therapeutically effective amount of a disclosed compound.

Provided herein is a method of reducing viral load associated with an HBV infection in an individual in need thereof, comprising administering to the individual a therapeutically effective amount of a disclosed compound.

Further, provided herein is a method of reducing reoccurrence of an HBV infection in an individual in need thereof, comprising administering to the individual a therapeutically effective amount of a disclosed compound.

Provided herein is a method of inhibiting or reducing the formation or presence of HBV DNA-containing particles or HBV RNA-containing particles in an individual in need thereof, comprising administering to the individual a therapeutically effective amount of a disclosed compound.

In certain aspects, the methods and/or compositions described herein are effective for inhibiting or reducing the formation or presence of HBV-associated particles in vitro or in vivo (e.g., in a cell, in a tissue, in an organ (e.g., in the liver), in an organism or the like). HBV-associated particles may contain HBV DNA (i.e., linear and/or covalently closed circular DNA (cccDNA)) and/or HBV RNA (i.e., pre-genomic RNA and/or sub-genomic RNA). Accordingly, HBV-associated particles include HBV DNA-containing particles or HBV RNA-containing particles.

As used herein, “HPV-associated particles” refer to both infectious HBV virions (i.e., Dane particles) and non-infectious HBV subviral particles (i.e., HBV filaments and/or HBV spheres). HBV virions comprise an outer envelope including surface proteins, a nucleocapsid comprising core proteins, at least one polymerase protein, and an HBV genome. HBV filaments and HBV spheres comprise HBV surface proteins, but lack core proteins, polymerase and an HBV genome. HBV filaments and HBV spheres are also known collectively as surface antigen (HBsAg) particles. HBV spheres comprise middle and small HBV surface proteins. HBV filaments also include middle, small and large HBV surface proteins.

HBV subviral particles can include the nonparticulate or secretory HBeAg, which serves as a marker for active replication of HBV.

Provided herein is a method of reducing an adverse physiological impact of an HBV infection in an individual in need thereof, comprising administering to the individual a therapeutically effective amount of a disclosed compound.

Also provided herein is a method of reducing, slowing, or inhibiting an HBV infection in an individual in need thereof, comprising administering to the individual a therapeutically effective amount of a disclosed compound.

Provided herein is a method of inducing reversal of hepatic injury from an HBV infection in an individual in need thereof, comprising administering to the individual a therapeutically effective amount of a disclosed compound.

Provided herein is a method of reducing the physiological impact of long-term antiviral therapy for HBV infection in an individual in need thereof, comprising administering to the individual a therapeutically effective amount of a disclosed compound.

Provided herein is a method of prophylactically treating an HBV infection in an individual in need thereof, wherein the individual is afflicted with a latent HBV infection, comprising administering to the individual a therapeutically effective amount of a disclosed compound.

In an embodiment, the individual is refractory to other therapeutic classes of HBV drugs (e.g., HBV polymerase inhibitors, interferons, viral entry inhibitors, viral maturation inhibitors, literature-described capsid assembly modulators, antiviral compounds of distinct or unknown mechanism, and the like, or combinations thereof). In another embodiment, the disclosed method reduces viral load in an individual suffering from an HBV infection to a greater extent or at a faster rate compared to the extent that other therapeutic classes of HBV drugs reduce viral load in the individual.

In an embodiment, the administering of a disclosed compound, or a pharmaceutically acceptable salt thereof, allows for administering of the at least one additional therapeutic agent at a lower dose or frequency as compared to the administering of the at least one additional therapeutic agent alone that is required to achieve similar results in prophylactically treating an HBV infection in an individual in need thereof.

In an embodiment, the administering of a disclosed compound, or a pharmaceutically acceptable salt thereof, reduces the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In an embodiment, the disclosed method reduces viral load in an individual suffering from an HBV infection, thus allowing lower doses or varying regimens of combination therapies to be used.

In an embodiment, the disclosed method causes a lower incidence of viral mutation or viral resistance compared to other classes of HBV drugs, thereby allowing for long term therapy and minimizing the need for changes in treatment regimens.

In an embodiment, the administering of a compound the invention, or a pharmaceutically acceptable salt thereof, causes a lower incidence of viral mutation or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In an embodiment, the disclosed method increases the seroconversion rate from HBV infected to non-HBV infected or from detectable HBV viral load to non-detectable HBV viral load beyond that of current treatment regimens. As used herein, “seroconversion” refers to the period of time during which HBV antibodies develop and become detectable.

In an embodiment, the disclosed method increases or normalizes or restores normal health, elicits full recovery of normal health, restores life expectancy, or resolves the viral infection in the individual in need thereof.

In an embodiment, the disclosed method eliminates or decreases the number of HBV RNA particles that are released from HBV infected cells thus enhancing, prolonging, or increasing the therapeutic benefit of the disclosed compounds.

In an embodiment, the disclosed method eradicates HBV from an individual infected with HBV, thereby obviating the need for long term or life-long treatment, or shortening the duration of treatment, or allowing for reduction in dosing of other antiviral agents.

In another embodiment, the disclosed method further comprises monitoring or detecting the HBV viral load of the subject, and wherein the method is carried out for a period of time including until such time that the HBV virus is undetectable.

Accordingly, in an embodiment, provided herein is a method of treating an HBV infection in an individual in need thereof, comprising administering to the individual a therapeutically effective amount of a compound of Formula I, or a pharmaceutically acceptable salt thereof.

Accordingly, in an embodiment, provided herein is a method of treating an HBV infection in an individual in need thereof, comprising administering to the individual a therapeutically effective amount of a compound of Formula I, or a pharmaceutically acceptable salt thereof.

In another embodiment, provided herein is a method of treating an HBV infection in an individual in need thereof, comprising administering to the individual a therapeutically effective amount of a compound of Table 1, or a pharmaceutically acceptable salt thereof.

In an embodiment of any of the methods provided herein, the method can further comprise monitoring the HBV viral load of the subject, wherein the method is carried out for a period of time such that the HBV virus is undetectable.

### Combination Therapies

The disclosed compounds may be useful in combination with one or more additional compounds useful for treating HBV infection, or a HBV-associated or -induced disease, or a liver disease. These additional compounds may comprise other disclosed compounds and/or compounds known to treat, prevent, or reduce the symptoms or effects of HBV infection, or of an HBV-associated or -induced disease, or of a liver disease.

Particularly, in an aspect a product is provided comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt of the application or the pharmaceutical composition of the application, and wherein said second compound is another HBV inhibitor which is selected from the group consisting of HBV combination drugs, HBV DNA polymerase inhibitors, immunomodulators toll-like (TLR) receptor modulators, interferon alpha receptor ligands, hyaluronidase inhibitors, hepatitis b surface antigen (HbsAg) inhibitors, cytotoxic T-lymphocyte-associated protein 4 (ipi4) inhibitors, cyclohilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, farnsoid X receptor agonists, HBV antibodies, CCR2 chemokine antagonists, thymosin agonists, cytokines, nuceloprotein modulators, retinoic acid-inducible gene 1 stimulators, NOD2 stimulators, phosphatidylinositol 3-kinase (PI3K) inhibitors, indoleamine 2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and (other) anti-HBV drugs.

The one or more additional compounds may e.g., be selected from interferon (for example, interferon-alpha-2a is pegylated interferon-alpha-2a (PEGASYS)), nucleoside or nucleotide or non-nucleos(t)ide polymerase inhibitors, immunomodulatory agents (e.g., IL-12, IL-18, IFN-alpha, -beta, and -gamma and TNF-alpha among others), TLR agonists, siRNAs and antisense oligonucleotides.

In another embodiment, the disclosed compound and the at least one additional therapeutic agent are co-formulated. In yet another embodiment, the disclosed compound and the at least one additional therapeutic agent are co-administered.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In an embodiment of any of the methods of administering combination therapies provided herein, the method can further comprise monitoring or detecting the HBV viral load of the subject, wherein the method is carried out for a period of time including until such time that the HBV virus is undetectable.

### Administration/Dosage/Formulations

In another aspect, provided herein is a pharmaceutical composition comprising at least one disclosed compound, or a pharmaceutically acceptable salt thereof, together with a pharmaceutically acceptable carrier.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

In particular embodiments, the compound is formulated in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of HBV infection in a patient.

In an embodiment, the compositions of the invention are formulated using one or more pharmaceutically acceptable excipients or carriers. In an embodiment, the pharmaceutical compositions of the invention comprise a therapeutically effective amount of a disclosed compound and a pharmaceutically acceptable carrier.

In some embodiments, the dose of a disclosed compound is from about 1 mg to about 2,500 mg. In some embodiments, a dose of a disclosed compound used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In an embodiment, the present invention is directed to a packaged pharmaceutical composition comprising a container holding a therapeutically effective amount of a disclosed compound, alone or in combination with a second pharmaceutical agent; and instructions for using the compound to treat, prevent, or reduce one or more symptoms of HBV infection in a patient.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the invention.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

### EXAMPLES

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

### PREPARATIVE EXAMPLES

Unless otherwise indicated, LCMS and NMR was conducted by using one of the following general methods.

General Methods of LCMS and NMR

General Procedure A

The LCMS measurement was performed using an Agilent system comprising a binary pump with degasser, an autosampler, a column oven (set at 40° C., unless otherwise indicated) and a column as specified in the respective methods below. Flow from the column was split to a MS and UV spectrometer. The MS detector was configured with an electrospray ionization source. Mass spectra were acquired by scanning from 100 to 1000 in 1.06 sec/cycle. The capillary voltage was 3 kV for positive ionization mode and 2.5 kV for negative ionization mode and the source temperature was maintained at 100° C. Nitrogen was used as the nebulizer gas. Data acquisition was performed with an Agilent ChemStation data system.

Method 1

In addition to the general procedure A: reversed phase LCMS for quality control was performed by Agilent 1200 with a diode-array detector (DAD) and carried out on a Sunfire C18 column (5 m, 4.6×50 mm) with a flow rate of 1.5 ml/min. Two mobile phases (mobile phase A1:0.02% ammoniumacetate in water; mobile phase A2:0.1% TFA in water; mobile phase B1: acetonitrile) were employed to run a gradient condition from 95% A1 or A2 and 5% B to 5% A1 or A2 and 95% B in 4.0 minutes. An injection volume of 1˜10 μl was used.

Method 2

In addition to the general procedure A: reversed phase LCMS for monitoring the reactions was performed by Agilent 1260 with a variable wavelength detector (VWD) and carried out on a Dikma Diamonsil plus C18 column (5 μm, 4.6×30 mm) with a flow rate of 2.0 ml/min. Two mobile phases (mobile phase A1: H₂O+0.02% ammoniumacetate+5% ACN; mobile phase A2: H₂O+0.1% TFA+5% ACN; mobile phase B: acetonitrile) were employed to run a gradient condition from 95% A1 or A2 and 5% B to 5% A1 or A2 and 95% B in 1.4 minutes. An injection volume of 1-5 μl was used.

Method 3

In addition to the general procedure A: reversed phase LCMS for monitoring the reactions was performed by Agilent 6120 (stationary phase Sunfire C18 2.5 μm, 3.0×30 mm. Mobile phase: 0.01% FA solution in water, and ACN, Gradient from 5% ACN to 95% in 2.5 min and stay in 95% for 1 min.

General Procedure B

The LCMS measurement was performed using a UPLC (Ultra Performance Liquid Chromatography) Acquity (Waters) system comprising a quaternary pump with degasser, an autosampler, a photo-diode array detector (PDA) and a column as specified in the respective methods below, the column is hold at a temperature of 40° C. Flow from the column was brought to MS detector. The MS detector was configured with an electrospray ionization source. Mass spectra were acquired by scanning from 100 to 1000 in 0.25 sec/cycle. The capillary needle voltage was 3 kV and the source temperature was maintained at 120° C. Cone voltage was 30 V for positive ionization mode and 30 V for negative ionization mode. Nitrogen was used as the nebulizer gas. Data acquisition was performed with a Waters-Micromass MassLynx-Openlynx data system.

Reversed phase UPLC was carried out on a Waters Acquity BEH (bridged ethylsiloxane/silica hybrid) C18 column (1.7 μm, 2.1×50 mm) with a flow rate of 0.5 ml/min. Two mobile phases (mobile phase A: 95% (H₂O+0.02% ammoniumacetate+5% ACN); mobile phase B: acetonitrile; mobile phase C: 95% (H₂O+0.1% TFA+5% ACN) were employed to run a gradient condition from 95% A or C and 5% B to 5% A or C and 95% B in 1 minute. An injection volume of 0.5 μl was used.

General Procedure C

The reversed phase preparation was performed using a system comprising two unit pumps without degasser, a UV/Vis detector and a column as specified in the respective methods below. Flow from the column was split to a UV spectrometer.

Method 1

In addition to the general procedure C: Prep-reversed phase LC was carried out on a Gilson with an autosampler, an Xbridge prep C18 OBD column (5 m, 19×150 mm) with a flow rate of 15-20 ml/min. Two mobile phases (mobile phase A1: H₂O (0.1% Ammonium bicarbonate); mobile phase A2: H₂O (Ammonium hydroxide); mobile phase A3: H₂O (0.1% TFA); mobile phase B: acetonitrile) were employed to run a gradient condition from 95% A1 or A2 or A3 and 5% B to 20% A1 or A2 or A3 and 80% B. Data acquisition was performed with a Trilution LC data system.

Method 2

In addition to the general procedure C: reversed phase preparation was carried out on a automatic medium pressure flash separation—Compact Purifier from Lisure Science Ltd. with reversed phase SW-5231 C18 column (40-60 m, 12011, 18 g, 40 g, 130 g) with a flow rate of 30-100 ml/min. Two mobile phases (mobile phase A1: H₂O (0.1% Ammonium bicarbonate); mobile phase A2: H₂O (Ammonium hydroxide); mobile phase A3: H₂O (0.1% Hydrochloric acid); mobile phase A4: H₂O; mobile phase B: acetonitrile) were employed to run a gradient condition from 95% A1 or A2 or A3 or A4 and 5% B to 5% A1 or A2 or A3 or A4 and 95% B. Data acquisition was performed with a Compact data system.

Method 3

In addition to the general procedure C: Prep-reversed phase LC was carried out on a Waters with an autosampler, a Xbridge prep C18 OBD column (Sum, 19*150 mm) with a flow rate of 20 ml/min. Two mobile phases (mobile phase A: H₂O (0.1% Ammonium bicarbonate); mobile phase B: acetonitrile) were employed to run a gradient condition from 95% A and 5% B to 50% A and 50% B. Data acquisition was performed with a Waters MassLynx data system.

General Procedure D

The chiral measurement was performed using a system comprising an autosampler, a column oven (set at ambient, unless otherwise indicated), a diode-array detector (DAD) and a column as specified in the respective methods below. Flow from the column was split to a UV spectrometer. LC spectra were acquired by scanning from 190 nm to 400 nm with deuterium lamp and from 401 nm to 800 nm with tungsten lamp using a slit width of 1.2 nm. The chiral chiralpak or chiralcel columns from Daicel Chiral technologies (China) Ltd. are divided into two types according to the different stuffings: Type 1: IA, IB, IC, ID, IE, IF, IG, IH; Type 2: AD-H, AS-H, OD-H, OJ-H.

Method 1:

In addition to the general procedure D: Chiral HPLC was carried out on an Agilent 1200 or Shimadzu LC-20A with a quaternary pump with degasser, a chiral column (5 μm, 4.6*250 mm) with a flow rate of 1.0 ml/min for chiral analysis or a chiral column (5 um, 20*250 mm) with a flow rate of 10-20 ml/min for chiral preparation. The mobile phases are the different ratios among MeOH, EtOH, Hex, IPA etc. Data acquisition was performed with an Agilent ChemStation or Shimadzu LabSolutions data system.

Method 2:

In addition to the general procedure D: chiral analysis was carried out on a Waters-TharSFC with a column oven (40° C.) with a flow rate of 2-3 ml/min and data acquisition was performed with TharSFC Chrom Scope data system. Chiral-preparation was carried out on a Waters-SFC-80 with a flow rate of 45-60 ml/min and data acquisition was performed with Waters-TharSFC SuperChrom data system. The mobile phase is CO₂ and MeOH, EtOH can be used as co-solvents.

General Procedure E

The below NMR experiments were carried out using a NMR spectrometers at ambient temperature, using internal deuterium lock and equipped with BBO 400 MHz S1 5 mm with Z-gradient; PLUS(²H, ¹H, BBF) probe head for the 400 MHz and DUL 300 MHz S1 5 mm Z-gradient (²H, ¹H, ¹³C) probe head for the 300 MHz. Chemical shifts (δ) are reported in parts per million (ppm).

Method 1:

In addition to the general procedure E: A Bruker Avance III 400 MHz spectrometer was used to measure the NMR experiment.

Method 2:

In addition to the general procedure E: A Bruker Avance Neo 400 MHz spectrometer was used to measure the NMR experiment.

Method 3:

In addition to the general procedure E: A ZKNJ BIXI-1 300 MHz spectrometer was used to measure the NMR experiment.

Method 4:

In addition to the general procedure E: A Bruker Ascend 400 MHz spectrometer was used to measure the NMR experiment.

Exemplary compounds useful in methods of the invention will now be described by reference to the illustrative synthetic schemes for their general preparation below and the specific examples to follow.

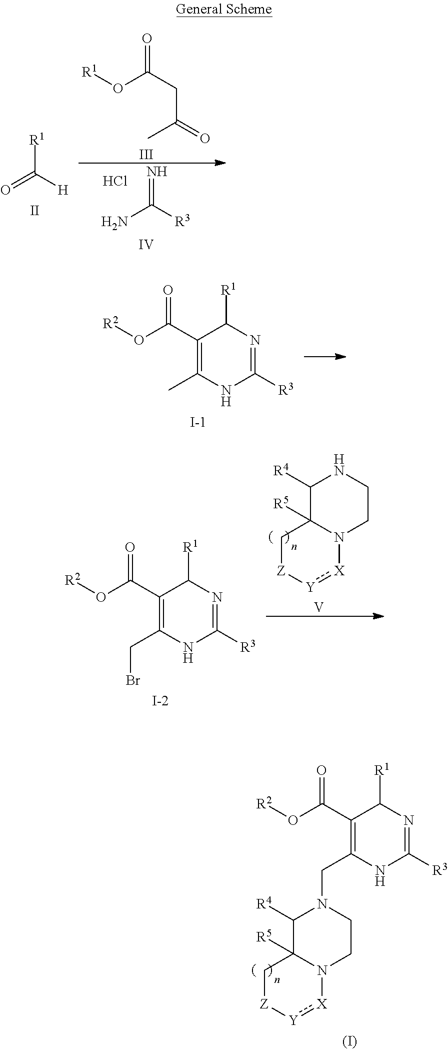

The preparation of compound I is shown in the above general scheme.

Compound I-1 can be prepared by the condensation of aldehyde II, acetoacetate III and amidine IV in the presence of a base such as NaOAc. Compound I-2 was prepared from compound I-1 using brominating reagent such as N-Bromosuccinimide. Coupling of compound I-2 and compound V in the presence of a base such as triethylamine affords compound I.

### Preparation of ethyl 4-(2-chloro-3-fluorophenyl)-6-methyl-2-(thiazol-2-yl)-1,4-dihydropyrimidine-5-carboxylate (H1)

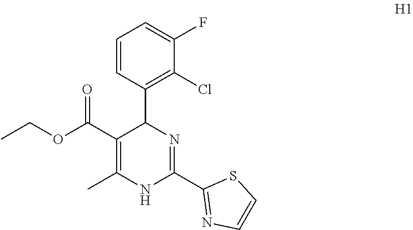 [SMILES: CCOC(=O)C1=C(C)NC(c2nccs2)=NC1c1cccc(F)c1Cl]

To a solution of 2-chloro-3-fluorobenzaldehyde (8.8 g, 55.7 mmol), ethyl 3-oxobutanoate (7.24 g, 55.7 mmol) in isopropanol (40 mL) was added piperidine (473 mg, 5.57 mmol) and AcOH (334 mg, 5.57 mmol). After stirred at room temperature for 4 hours, the mixture was added thiazole-2-carboximidamide (6.4 g, 39 mmol) and triethylamine (5.62 g, 55.7 mmol) at room temperature over 15 minutes. The reaction mixture was stirred at 75° C. for 12 hours. It was cooled to room temperature, extracted with ethyl acetate, washed with brine, dried over Na₂SO₄ and purified by silica gel column chromatography (petroleum ether:ethyl acetate=20:1) to give the title compound 11 (5.45 g, 95% purity from 1H NMR, 26% yield) as yellow solids. LC-MS (ESI): R_{T}=1.74 min, mass calcd. for C₁₇H₁₅ClFN₃O₂S 379.1, m/z found 380.1 [M+H]+. ¹H NMR (400 MHz, CDCl₃) δ 7.84-7.80 (m, 1.7H), 7.50 (d, J=3.6 Hz, 0.3H), 7.47 (s, 0.3H), 7.44 (d, J=3.2 Hz, 0.7H), 7.23-7.14 (m, 2H), 7.09-7.01 (m, 1H), 6.27 (s, 0.7H), 6.14 (d, J=2.4 Hz, 0.3H), 4.13-3.98 (m, 2H), 2.57 (s, 0.7H), 2.52 (s, 2.3H), 1.13-1.10 (m, 3H).

Chiral separation of ethyl 4-(2-chloro-3-fluorophenyl)-6-methyl-2-(thiazol-2-yl)-1,4-dihydropyrimidine-5-carboxylate (H1)

The racemic mixture ethyl 4-(2-chloro-3-fluorophenyl)-6-methyl-2-(thiazol-2-yl)-1,4-dihydropyrimidine-5-carboxylate H1 (5.45 g, 13.7 mmol) was separated by chiral separation (separation condition: column: Chiralpak IC 5 μm 20*250 mm; Mobile Phase: Hex:EtOH:DEA=95:5:0.3 at 28 mL/min, Temp: 30° C., Wavelength: 254 nm) to give H1-A (2.5 g, 90% purity from ¹HNMR, 46% yield, 100% ee) and H1-B (2.48 g, 90% purity from ¹HNMR, 46% yield, 92.1% ee) as yellow solids.

H1-A: LC-MS (ESI): R_{T}=3.886 min, mass calcd. for C₁₇H₁₅ClFN₃O₂S 379.06, m/z found 380.1 [M+H]+. Chiral analysis (Column: Chiralpak IA 5 μm 4.6*250 mm; Mobile Phase: Hex:EtOH:DEA=90:10:0.2 at 1.0 mL/min; Temp: 30° C.; Wavelength: 254 nm, R_{T}=7.438 min). ¹H NMR (400 MHz, CDCl₃) δ 7.84-7.80 (m, 1.7H), 7.51-7.44 (m, 1.3H), 7.22-7.14 (m, 2H), 7.09-7.01 (m, 1H), 6.27 (s, 0.7H), 6.14 (s, 0.3H), 4.05-4.00 (m, 2H), 2.57 (s, 0.7H), 2.52 (s, 2.3H), 1.13-1.10 (m, 3H).

H1-B: LC-MS (ESI): R_{T}=3.887 min, mass calcd. for C₁₇H₁₅ClFN₃O₂S 379.06, m/z found 380.1 [M+H]+. Chiral analysis (Column: Chiralpak IA 5 μm 4.6*250 mm; Mobile Phase: Hex:EtOH:DEA=90:10:0.2 at 1.0 mL/min; Temp: 30° C.; Wavelength: 254 nm, R_{T}=6.903 min). ¹H NMR (400 MHz, CDCl₃) δ 7.84-7.80 (m, 1.7H), 7.51-7.43 (m, 1.3H), 7.22-7.14 (m, 2H), 7.09-7.01 (m, 1H), 6.27 (s, 0.7H), 6.14 (s, 0.3H), 4.10-3.98 (m, 2H), 2.57 (s, 0.7H), 2.51 (s, 2.3H), 1.13-1.10 (m, 3H).

### Preparation of ethyl 6-(bromomethyl)-4-(2-chloro-3-fluorophenyl)-2-(thiazol-2-yl)-1,4-dihydropyrimidine-5-carboxylate (H1-1A) (Single Enantiomer)

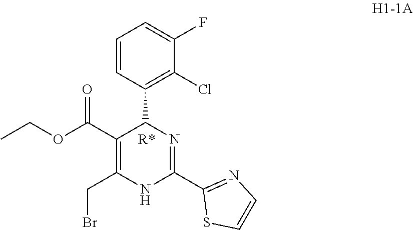

To a solution of ethyl 4-(2-chloro-3-fluorophenyl)-6-methyl-2-(thiazol-2-yl)-1,4-dihydropyrimidine-5-carboxylate H1-A (300 mg, 90% purity, 0.711 mmol) in carbon tetrachloride (5 mL) was added N-bromosuccinimide (120 mg, 0.674 mmol). After stirred at 60° C. for 1 hour, the reaction mixture was concentrated to give a residue, which was purified by gel column chromatography (petroleum ether:ethyl acetate=20:1 to 10:1) to give the title compound (H1-1A) (240 mg, 90% purity from HNMR, 66% yield) as yellow solids. LC-MS (ESI): R_{T}=1.852 min, mass calcd. for C₁₇H₁₄BrClFN₃O₂S 456.9, m/z found 457.9 [M+H]⁺. ¹H NMR (400 MHUz, CDCl₃) δ 8.26 (s, 0.31H), 7.84 (d, J=2.8 Hz, 1H), 7.53-7.46 (in, 1.71H), 7.24-7.14 (m, 2H), 7.09-7.01 (m, 1H), 6.26 (s, 0.31H), 6.17 (s, 0.71H), 4.92 (d, J=8.0 Hz, 1H), 4.76 (d, J=11.2 Hz, 0.31H), 4.60 (d, J=8.0 Hz, 0.71H), 4.12 (q, J=7.2 Hz, 2H), 1.14 (t, J=11.2 Hz, 3H).

Using the same procedure, the following intermediates were prepared.

| | | | | Brominated |
|---|---|---|---|---|
| Aldehyde | Ketoester | Amidine | Intermediate | Intermediate |
| 2-methyl-3- fluoro- benzaldehyde | Ethyl 3- oxobutanoate | thiazole-2- carbox- imidamide | 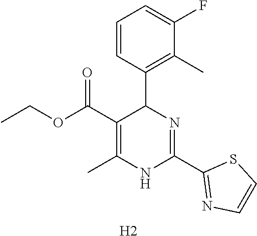 [SMILES: CCOC(=O)C1=C(C)NC(c2nccs2)=NC1c1cccc(F)c1C ; "H2"] | 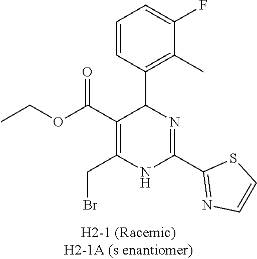 [SMILES: CCOC(=O)C1=C(CBr)NC(c2nccs2)=NC1c1cccc(F)c1C] |
| 2-chloro-4- fluoro- benzaldehyde | Methyl 3- oxobutanoate | thiazole-2- carbox- imidamide | 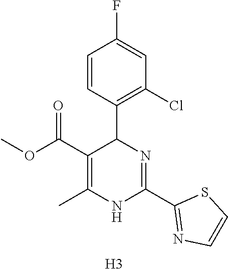 [SMILES: COC(=O)C1=C(C)NC(c2nccs2)=NC1c1ccc(F)cc1Cl ; "H3"] | 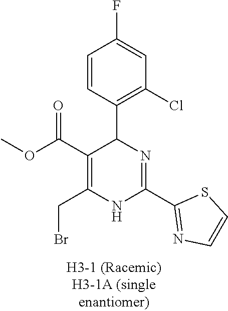 [SMILES: COC(=O)C1=C(CBr)NC(c2nccs2)=NC1c1ccc(F)cc1Cl] |
| 2-methyl-3- fluoro- benzaldehyde | Methyl 3- oxobutanoate | thiazole-2- carbox- imidamide | 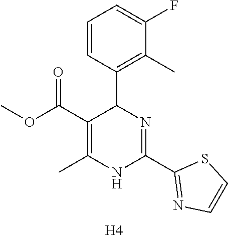 [SMILES: COC(=O)C1=C(C)NC(c2nccs2)=NC1c1cccc(F)c1C ; "H4"] | 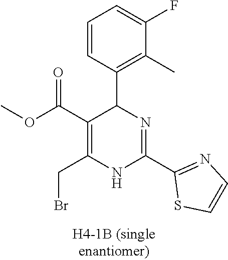 [SMILES: COC(=O)C1=C(CBr)NC(c2nccs2)=NC1c1cccc(F)c1C] |
| 2-chloro-3,4- difluoro- benzaldehyde | Methyl 3- oxobutanoate | thiazole-2- carbox- imidamide | 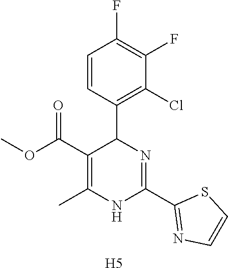 [SMILES: COC(=O)C1=C(C)NC(c2nccs2)=NC1c1ccc(F)c(F)c1Cl ; "H5"] | 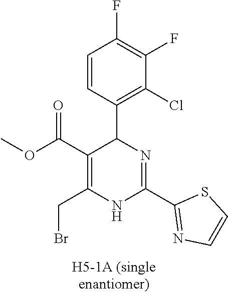 [SMILES: COC(=O)C1=C(CBr)NC(c2nccs2)=NC1c1ccc(F)c(F)c1Cl] |
| 3,4-difluoro-2- methyl- benzaldehyde | Methyl 3- oxobutanoate | thiazole-2- carbox- imidamide | 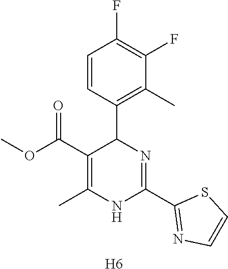 [SMILES: COC(=O)C1=C(C)NC(c2nccs2)=NC1c1ccc(F)c(F)c1C ; "H6"] | 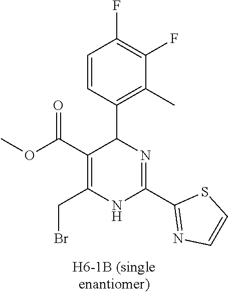 [SMILES: COC(=O)C1=C(CBr)NC(c2nccs2)=NC1c1ccc(F)c(F)c1C] |
| 2-bromo-4- fluoro- benzaldehyde | Ethyl 3- oxobutanoate | thiazole-2- carbox- imidamide | 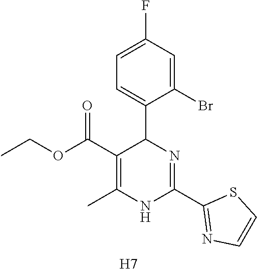 [SMILES: CCOC(=O)C1=C(C)NC(c2nccs2)=NC1c1ccc(F)cc1Br ; "H7"] | 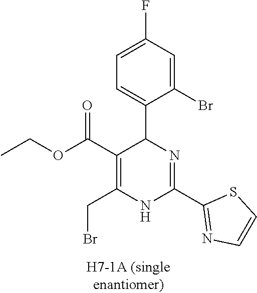 [SMILES: CCOC(=O)C1=C(CBr)NC(c2nccs2)=NC1c1ccc(F)cc1Br] |
| 2-chloro-3,4- difluoro- benzaldehyde | Ethyl 3- oxobutanoate | thiazole-2- carbox- imidamide | 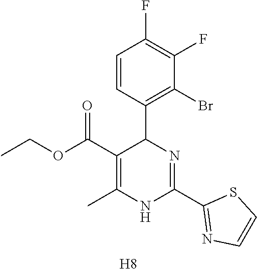 [SMILES: CCOC(=O)C1=C(C)NC(c2nccs2)=NC1c1ccc(F)c(F)c1Br ; "H8"] | 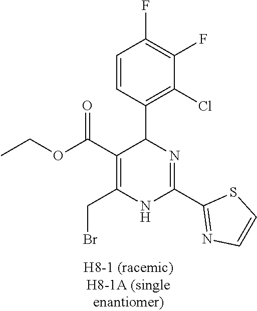 [SMILES: CCOC(=O)C1=C(CBr)NC(c2nccs2)=NC1c1ccc(F)c(F)c1Cl] |
| 3,4-difluoro-2- methyl- benzaldehyde | Ethyl 3- oxobutanoate | thiazole-2- carbox- imidamide | 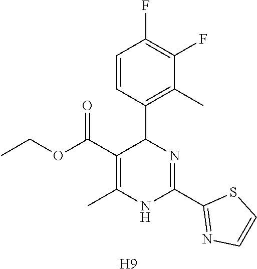 [SMILES: CCOC(=O)C1=C(C)NC(c2nccs2)=NC1c1ccc(F)c(F)c1C ; "H9"] | 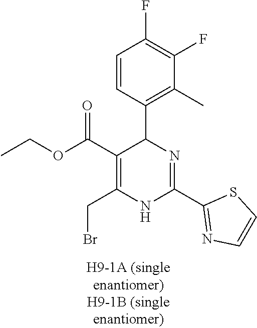 [SMILES: CCOC(=O)C1=C(CBr)NC(c2nccs2)=NC1c1ccc(F)c(F)c1C] |
| 2-bromo-4- fluoro- benzaldehyde | Methyl 3- oxobutanoate | thiazole-2- carbox- imidamide | 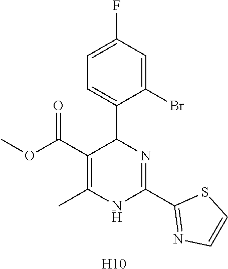 [SMILES: COC(=O)C1=C(C)NC(c2nccs2)=NC1c1ccc(F)cc1Br ; "H10"] | 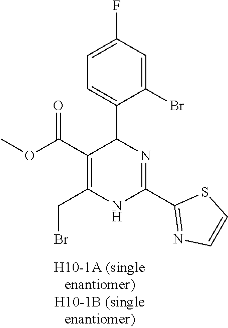 [SMILES: COC(=O)C1=C(CBr)NC(c2nccs2)=NC1c1ccc(F)cc1Br] |
| 2-chloro-3- fluoro- benzaldehyde | Methyl 3- oxobutanoate | thiazole-2- carbox- imidamide | 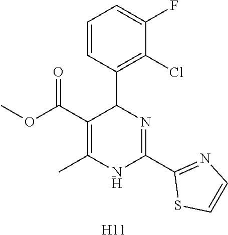 [SMILES: COC(=O)C1=C(C)NC(c2nccs2)=NC1c1cccc(F)c1Cl ; "H11"] | 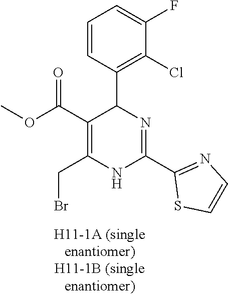 [SMILES: COC(=O)C1=C(CBr)NC(c2nccs2)=NC1c1cccc(F)c1Cl] |
| 2-chloro-4- fluoro- benzaldehyde | Ethyl 3- oxobutanoate | thiazole-2- carbox- imidamide | 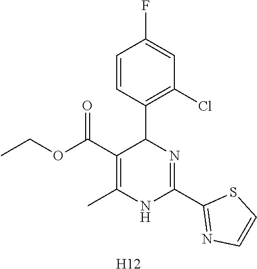 [SMILES: CCOC(=O)C1=C(C)NC(c2nccs2)=NC1c1ccc(F)cc1Cl ; "H12"] | 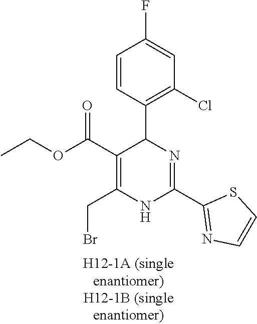 [SMILES: CCOC(=O)C1=C(CBr)NC(c2nccs2)=NC1c1ccc(F)cc1Cl] |
| 3-fluoro-2- methyl- benzaldehyde | Ethyl 3- oxobutanoate | 3,5-difluoro- picolin imidamide hydrochloride | 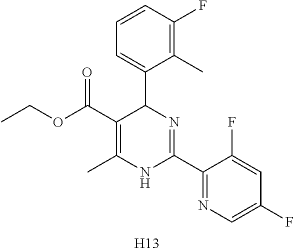 [SMILES: CCOC(=O)C1=C(C)NC(c2ncc(F)cc2F)=NC1c1cccc(F)c1C ; "H13"] | 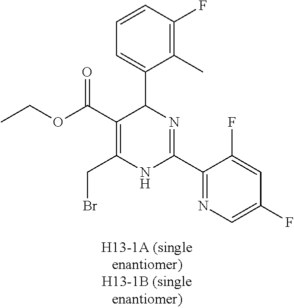 [SMILES: CCOC(=O)C1=C(CBr)NC(c2ncc(F)cc2F)=NC1c1cccc(F)c1C] |
| 2-fluoro-4- methyl- benzaldehyde | Ethyl 3- oxobutanoate | thiazole-2- carbox- imidamide | 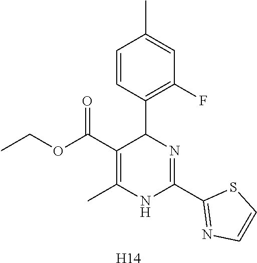 [SMILES: CCOC(=O)C1=C(C)NC(c2nccs2)=NC1c1ccc(C)cc1F ; "H14"] | 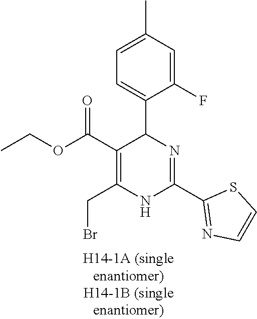 [SMILES: CCOC(=O)C1=C(CBr)NC(c2nccs2)=NC1c1ccc(C)cc1F] |
| 3-fluoro-2- methyl- benzaldehyde | Ethyl 3- oxobutanoate | 5-Methyl- oxazole- 4-carbox- imidamide hydrochloride | 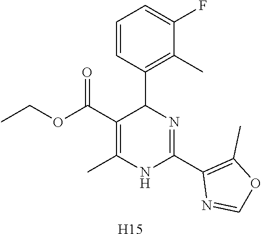 [SMILES: CCOC(=O)C1=C(C)NC(c2ncoc2C)=NC1c1cccc(F)c1C ; "H15"] | 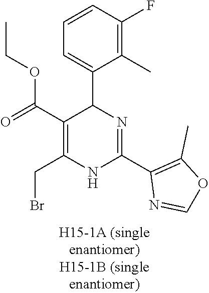 [SMILES: CCOC(=O)C1=C(CBr)NC(c2ncoc2C)=NC1c1cccc(F)c1C] |
| 3-fluoro-2- methyl- benzaldehyde | methyl 3- oxobutanoate | 5-methyl oxazole- 4-carbox imidamide | 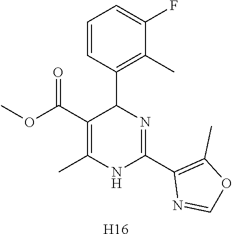 [SMILES: COC(=O)C1=C(C)NC(c2ncoc2C)=NC1c1cccc(F)c1C ; "H16"] | 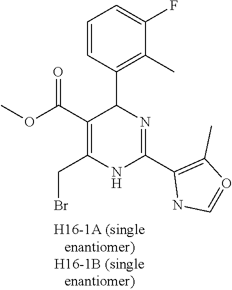 |
| 2-chloro-4- fluoro- benzaldehyde | Ethyl 3- oxobutanoate | 5-methyl oxazole- 4-carbox imidamide | 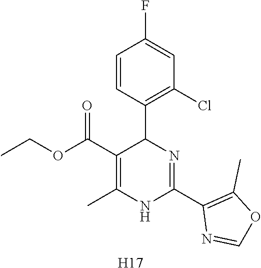 [SMILES: CCOC(=O)C1=C(C)NC(c2ncoc2C)=NC1c1ccc(F)cc1Cl ; "H17"] | 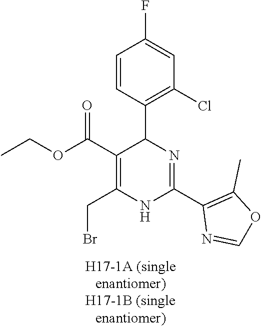 [SMILES: CCOC(=O)C1=C(CBr)NC(c2ncoc2C)=NC1c1ccc(F)cc1Cl] |
| 2-chloro-4- fluoro- benzaldehyde | methyl 3- oxobutanoate | 5-methyl oxazole- 4-carbox imidamide | 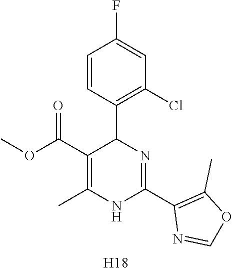 [SMILES: COC(=O)C1=C(C)NC(c2ncoc2C)=NC1c1ccc(F)cc1Cl ; "H18"] | 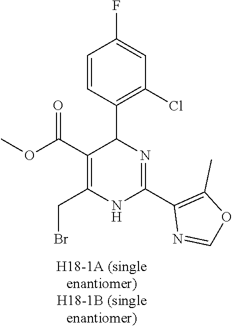 [SMILES: COC(=O)C1=C(CBr)NC(c2ncoc2C)=NC1c1ccc(F)cc1Cl] |
| Deuteride-3- fluoro-2- methyl- benzaldehyde | Ethyl 3- oxobutanoate | thiazole-2- carbox- imidamide | 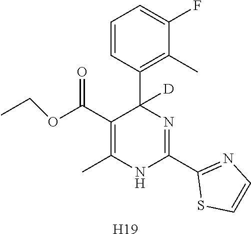 [SMILES: [2H]C1(c2cccc(F)c2C)N=C(c2nccs2)NC(C)=C1C(=O)OCC ; "H19"] | 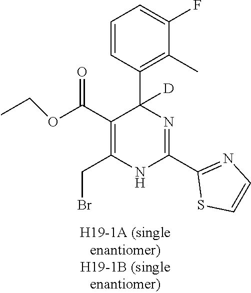 [SMILES: [2H]C1(c2cccc(F)c2C)N=C(c2nccs2)NC(CBr)=C1C(=O)OCC] |
| ethyl6- (bromomethyl)- 2-(thiazol-2- yl)-1,4- dihydro- pyrimidine-5- carboxylate 6- fluoro-2- methylnicotinate | Ethyl 3- oxobutanoate | thiazole-2- carbox- imidamide | 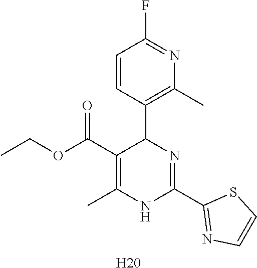 [SMILES: CCOC(=O)C1=C(C)NC(c2nccs2)=NC1c1ccc(F)nc1C ; "H20"] | 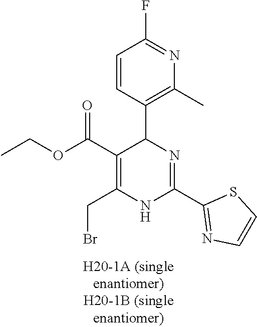 [SMILES: CCOC(=O)C1=C(CBr)NC(c2nccs2)=NC1c1ccc(F)nc1C] |

### Intermediate H2 Ethyl 4-(3-fluoro-2-methylphenyl)-6-methyl-2-(thiazol-2-yl)-1,4-dihydropyrimidine-5-carboxylate

Intermediate H2 was prepared using same condition as for H1.

¹H NMR (400 MHz, DMSO-d₆) δ 9.86 (s, 0.81H), 9.52 (d, J=2.8 Hz, 0.21H), 8.00-7.98 (in, 0.4H), 7.96 (d, J=3.2 Hz, 0.8H), 7.88 (d, J=2.8 Hz, 0.8H), 7.20-7.15 (m, 1.2H), 7.06-6.99 (m, 1.8H), 5.83 (s, 0.8H), 5.73 (d, J=3.2 Hz, 0.2H), 3.99-3.93 (m, 2H), 2.48 (s, 2.4H), 2.45 (s, 1.2H), 2.44 (s, 1.2H), 2.41 (s, 0.3H), 2.40 (s, 0.3H), 2.37 (s. 0.6H), 1.08-1.02 (m, 3H).

Intermediate H2 was separated by chiral Prep-HPLC (separation condition: Column: Chiralpak OJ-H 5 μm 20*250 mm; Mobile Phase: Hex:EtOH:DEA=90:10:0.3 at 15 mL/min; Temp: 30° C.; Wavelength: 214 nm) to afford 112-A and 112-B as yellow solids.

Intermediate H2-A: Chiral analysis (Column: Chiralpak OJ-H 5 μm 4.6*250 mm; Mobile Phase: Hex:EtOH:DEA=85:15:0.2 at 1.0 mL/min; Temp: 30° C.; Wavelength: 230 nm, R_{T}=7.251 min). 112-A was certificated to absolute S stereochemistry by the following chemical resolution which is consistent with reported data (J. Med. Chem., 2017, 60 (8), pp 3352-3371). Optical rotation: [a]_{D}²⁰-24° (c 0.10, MeOH).

Intermediate H2-B: Chiral analysis (Column: Chiralpak OJ-H 5 μm 4.6*250 mm; Mobile Phase: Hex:EtOH:DEA=85:15:0.2 at 1.0 mL/min; Temp: 30° C.; Wavelength: 230 nm, R_{T}=9.072 min). Optical rotation: [a]_{D}²⁰+35° (c 0.10, MeOH).

### Intermediate H2-1A: (S)-Ethyl 6-(bromomethyl)-4-(3-fluoro-2-methylphenyl)-2-(thiazol-2-yl)-1,4-dihydropyrimidine-5-carboxylate

Intermediate H2-1A was prepared from 112-A using same condition as for H1-1A. LC-MS (ESI): R_{T}=1.84 min, mass calcd. for C₁₈H₁₇BrFN₃O₂S 437.0, m/z found 440.0 [M+H]⁺. ¹H NMR (400 MHz, CDCl₃) δ 8.22 (s, 0.5H), 7.82 (d, J=3.2 Hz, 1H), 7.53 (s, 0.4H), 7.44 (s, 0.6H), 7.25-7.08 (m, 2.5H), 6.96-6.92 (s, 1H), 5.99 (s, 0.6H), 5.93 (s, 0.4H), 4.92-4.77 (m, 1.6H), 4.67-4.65 (m, 0.4H), 4.13-4.07 (m, 2H), 2.53 (s, 1.7H), 2.41 (s, 1.3H), 1.14 (t, J=7.2 Hz, 3H). Optical rotation: [a]_{D}2+0.093° (c 0.10, MeOH).

### Intermediate H3: Methyl 4-(2-chloro-4-fluorophenyl)-6-methyl-2-(thiazol-2-yl)-1,4-dihydropyrimidine-5-carboxylate (racemic)

Intermediate H3 was prepared using same condition as for 111.

LC-MS (ESI): R_{T}=1.70 min, mass calcd. for C₁₆H₁₃ClFN₃O₂S 365.04, m/z found 366.1 [M+H]⁺. ¹H NMR (400 MHz, CDCl₃) δ 7.84-7.83 (m, 0.9H), 7.81-7.80 (m, 0.8H), 7.55-7.50 (m, 0.6H), 7.44-7.43 (m, 0.7H), 7.33-7.26 (m, 1H), 7.13-7.11 (m, 1H), 6.95-6.88 (m, 1H), 6.18 (s, 0.7H), 6.05 (s, 0.3H), 3.63 (s, 0.8H), 3.60 (s, 2.2H), 2.57 (s, 0.8H), 2.51 (s, 2.2H). Racemic H3 (20 g, 95% purity, 51.9 mmol) was separated by chiral Prep-HPLC (Column: Chiralpak IG 5 μm 30*250 mm; Mobile Phase: CO₂:MeOH=70:30 at 55 g/min; Col. Temp: 40° C.; Wavelength: 230 nm, Back pressure: 100 bar) to afford the title compounds H3-A (9.46 g, 95% purity from NMR, 47% yield, 100% ee) and H3-B (9.5 g, 95% purity from NMR, 48% yield, 98.0% ee) as yellow solids.

Intermediate H3-A: LC-MS (ESI): R_{T}=1.69 min, mass calcd. for C₁₆H₁₃ClFN₃O₂S 365.0, m/z found 366.0. Chiral analysis (Column: Chiralpak IA 5 μm 4.6*250 mm; Mobile Phase: Hex:EtOH=80:20 at 1.0 mL/min; Temp: 30° C.; Wavelength: 254 nm, R_{T}=5.593 min). ¹H NMR (400 MHz, CDCl₃) δ 7.84-7.83 (m, 1H), 7.80 (d, J=2.8 Hz, 0.7H), 7.52-7.50 (m, 0.5H), 7.44 (d, J=2.8 Hz, 0.7H), 7.34-7.30 (m, 1H), 7.15-7.11 (m, 1H), 6.96-6.88 (m, 1H), 6.19 (s, 0.7H), 6.06 (d, J=2.4 Hz, 0.3H), 3.63 (s, 0.8H), 3.60 (s, 2.2H), 2.57 (s, 0.8H), 2.51 (s, 2.2H).

Intermediate H3-B: LC-MS (ESI): R_{T}=1.68 min, mass calcd. for C₁₆H₁₃ClFN₃O₂S 365.0, m/z found 366.0. Chiral HPLC (Column: Chiralpak IA 5 μm 4.6*250 mm; Mobile Phase: Hex:EtOH=80:20 at 1.0 mL/min; Temp: 30° C.; Wavelength: 254 nm, R_{T}=6.827 min). ¹H NMR (400 MHz, CDCl₃) 7.85-7.82 (m, 1H), 7.80 (d, J=3.2 Hz, 0.7H), 7.54-7.50 (m, 0.5H), 7.43 (d, J=3.2 Hz, 0.7H), 7.34-7.30 (m, 1H), 7.14-7.11 (m, 1H), 6.96-6.88 (m, 1H), 6.18 (s, 0.7H), 6.06 (d, J=2.4 Hz, 0.3H), 3.62 (s, 0.8H), 3.60 (s, 2.2H), 2.57 (s, 0.8H), 2.50 (s, 2.2H).

### Intermediate H3-1A: methyl 6-(bromomethyl)-4-(2-chloro-4-fluorophenyl)-2-(thiazol-2-yl)-1,4-dihydropyrimidine-5-carboxylate

Intermediate H3-1A was prepared from H3-A using same condition as for H1-1A.

LC-MS (ESI): R_{T}=1.802 min, mass calcd. for C₁₆H₁₂BrClFN₃O₂S 442.9, m/z found 443.9 [M+H]⁺. ¹H NMR (400 MHz, CDCl₃) δ 8.29 (br s, 0.3H), 7.84 (d, J=3.2 Hz, 1H), 7.59-7.53 (m, 1.4H), 7.47 (br s, 0.3H), 7.41-7.31 (m, 1H), 7.14 (d, J=8.4 Hz, 1H), 6.99-6.90 (m, 1H), 6.18 (s, 0.3H), 6.09 (d, J=2.0 Hz, 0.7H), 4.93 (d, J=8.4 Hz, 1H), 4.74 (d, J=11.2 Hz, 0.3H), 4.58 (d, J=8.4 Hz, 0.7H), 3.67 (s, 2.1H), 3.65 (s, 0.9H).

### Intermediate H4: Methyl 4-(3-fluoro-2-methylphenyl)-6-methyl-2-(thiazol-2-yl)-1,4-dihydropyrimidine-5-carboxylate (racemic)

Intermediate H4 was prepared using same condition as for H1.

¹H NMR (400 MHz, CDCl₃) δ 7.93 (d, J=3.2 Hz, 0.1H), 7.80-7.77 (m, 1.8H), 7.52-7.50 (m, 0.1H), 7.41 (d, J=3.2 Hz, 0.9H), 7.20 (br s, 0.1H), 7.16-7.00 (m, 2H), 6.94-6.87 (m, 1H), 6.00 (s, 0.9H), 5.90 (s, 0.1H), 3.60 (s, 3H), 2.55-2.49 (m, 5.8H), 2.40 (br s, 0.2H).

A racemic mixture of methyl 4-(3-fluoro-2-methylphenyl)-6-methyl-2-(thiazol-2-yl)-1,4-dihydropyrimidine-5-carboxylate H4 (1.30 g, 95% purity, 3.58 mmol) was separated by chiral Prep-HPLC (separation condition: Column: Chiralpak AS-H 5 μm 30*250 mm; Mobile Phase: Hex:EtOH=75:25 at 15 mL/min; Temp: 30° C.; Wavelength: 214 nm) to afford the title compounds (H4-A)(610 mg, 95% purity from ¹H NMR, 44% yield, 100% stereopure) and (H4-B) (520 mg, 95% purity from ¹H NMR, 40% yield, 97.7% stereopure) as yellow oil.

Intermediate H4-A: Chiral analysis (Column: Chiralpak AS 5 μm 4.6*250 mm; Mobile Phase: Hex:EtOH=80:20 at 1 mL/min; Temp: 30° C.; Wavelength: 254 nm, R_{T}=5.247 min). ¹H NMR (400 MHz, CDCl₃) δ 7.93 (d, J=2.8 Hz, 0.1H), 7.80 (br s, 0.9H), 7.78 (d, J=2.8 Hz, 1H), 7.52-7.50 (m, 0.1H), 7.41 (d, J=3.2 Hz, 0.9H), 7.10-7.02 (m, 2H), 6.92-6.87 (m, 1H), 6.00 (s, 0.9H), 5.91 (s, 0.1H), 3.61 (s, 3H), 2.55 (s, 3H), 2.53 (s, 3H).

Intermediate H4-B: Chiral analysis (Column: Chiralpak AS 5 μm 4.6*250 mm; Mobile Phase: Hex:EtOH=80:20 at 1 mL/min; Temp: 30° C.; Wavelength: 254 nm, R_{T}=9.049 min). H NMR (400 MHz, CDCl₃) δ 7.78 (d, J=3.2 Hz, 2H), 7.42 (d, J=2.4 Hz, 1H), 7.10-7.05 (m, 2H), 6.92-6.89 (m, 1H), 5.99 (s, 1H), 3.61 (s, 3H), 2.54 (s, 3H), 2.53 (m, 3H).

### Intermediate H4-1B: Methyl 6-(bromomethyl)-4-(3-fluoro-2-methylphenyl)-2-(thiazol-2-yl)-1,4-dihydropyrimidine-5-carboxylate

Intermediate H4-1B was prepared from H4-B using same condition as for H1-1A.

¹H NMR (400 MHz, CDCl₃) δ 8.23 (s, 1H), 7.82 (d, J=3.2 Hz, 1H), 7.53-7.44 (m, 1H), 7.12-7.07 (m, 2H), 6.93 (s, 1H), 5.98-5.94 (m, 1H), 4.89-4.66 (m, 2H), 3.65 (s, 3H), 2.53-2.41 (m, 3H).

### Intermediate H5: Methyl 4-(2-chloro-3,4-difluorophenyl)-6-methyl-2-(thiazol-2-yl)-1,4-dihydropyrimidine-5-carboxylate

Intermediate H5 was prepared using same condition as for H1.

¹H NMR (400 MHz, CD₃OD) δ 8.08 (d, J=2.8 Hz, 0.1H), 7.98 (d, J=2.8 Hz, 0.1H), 7.93 (d, J=2.8 Hz, 0.9H), 7.72 (d, J=2.8 Hz, 0.9H), 7.26-7.18 (m, 2H), 6.13 (s, 0.9H), 6.09 (s, 0.1H), 3.61 (s, 3H), 2.53 (s, 3H).

Racemic H5 (1.10 g, 2.90 mmol) was separated by chiral Prep-HPLC (separation condition: Column: Chiralpak IC 5 μm 20*250 mm; Mobile Phase: Hex:EtOH=90:10 at 18 mL/min; Temp: 30° C.; Wavelength: 214 nm) to afford the title compounds 115-A (450 mg, 41% yield, 100% stereopure) and 115-B (450 μm g, 41% yield, 99.8% stereopure) as yellow solids.

Intermediate H5-A: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=6.457 min).

Intermediate H5-B: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=7.641 min).

### Intermediate H5-1A: Methyl 6-(bromomethyl)-4-(2-chloro-3,4-difluorophenyl)-2-(thiazol-2-yl)-1,4-dihydropyrimidine-5-carboxylate

Intermediate H5-1A was prepared from H5-A using same condition as for H1-1A.

¹H NMR (400 MHz, CD₃OD) δ 7.92 (d, J=3.2 Hz, 1H), 7.80 (d, J=3.2 Hz, 0.5H), 7.70 (d, J=3.2 Hz, 0.5H), 7.32-7.17 (m, 2H), 6.11 (s, 0.5H), 6.09 (s, 0.5H), 4.91 (d, J=10.0 Hz, 0.5H), 4.81 (d, J=10.0 Hz, 1H), 4.57 (d, J=8.4 Hz, 0.5H), 3.64 (s, 1.5H), 3.62 (s, 1.5H).

### Intermediate H6: Methyl 4-(3,4-difluoro-2-methylphenyl)-6-methyl-2-(thiazol-2-yl)-1,4-dihydropyrimidine-5-carboxylate

Intermediate H6 was prepared using same condition as for H1.

LC-MS (ESI): R_{T}=1.58 min, mass calcd. for C₁₇H₁₅F₂N₃O₂S 363.3, m/z found 364.0 [M+H]⁺. ¹H NMR (400 MHz, CDCl₃) δ 7.80-7.78 (m, 2H), 7.42 (d, J=3.2 Hz, 1H), 7.00-6.85 (m, 2H), 5.93 (s, 1H), 3.61 (s, 3H), 2.58 (s, 1.5H), 2.57 (s, 1.5H), 2.53 (s, 1.5H), 2.51 (s, 1.5H).

Racemic H6 (1.00 g, 90% purity, 2.48 mmol) was separated by chiral Prep-HPLC (separation condition: Column: Chiralpak IH 5 μm 30*250 mm; Mobile Phase: Hex:EtOH=90:10 at 18 mL/min; Temp: 30° C.; Wavelength: 214 nm) to afford the desired products 116-A (400 mg, 90% purity from H NMR, 40% yield, 100% stereopure) and 116-B (400 mg, 95% purity from ¹H NMR, 42% yield, 99.9% stereopure) as yellow solids.

Intermediate H6-A: Chiral analysis (Column: Chiralpak IH 5 μm 4.6*150 mm; Mobile Phase: Hex:EtOH=90:10 at 1 mL/min; Temp: 30° C.; Wavelength: 230 nm, R_{T}=4.809 min). ¹H NMR (400 MHz, CDCl₃) δ 7.84 (br s, 1H), 7.78 (d, J=3.2 Hz, 1H), 7.42 (d, J=3.2 Hz, 1H), 6.96-6.86 (m, 2H), 5.93 (s, 1H), 3.61 (s, 3H), 2.57 (d, J=1.6 Hz, 3H), 2.52 (s, 3H).

Intermediate H6-B: Chiral analysis (Column: Chiralpak IH 5 μm 4.6*150 mm; Mobile Phase: Hex:EtOH=90:10 at 1 mL/min; Temp: 30° C.; Wavelength: 230 nm, R_{T}=7.018 min). ¹H NMR (400 MHz, CDCl₃) δ 7.82 (br s, 1H), 7.79 (d, J=3.2 Hz, 1H), 7.42 (d, J=3.2 Hz, 1H), 6.97-6.88 (m, 2H), 5.93 (s, 1H), 3.61 (s, 3H), 2.58 (d, J=2.0 Hz, 3H), 2.52 (s, 3H).

### Intermediate H6-1B: Methyl 6-(bromomethyl)-4-(3,4-difluoro-2-methylphenyl)-2-(thiazol-2-yl)-1,4-dihydropyrimidine-5-carboxylate

Intermediate H6-1B was prepared from 116-B using same condition as for H1-1A.

¹H NMR (400 MHz, CDCl₃) δ 8.24 (s, 1H), 7.83 (d, J=3.6 Hz, 1H), 7.54-7.45 (m, 1H), 7.00-6.93 (m, 2H), 5.91 (s, 1H), 4.94-4.80 (s, 21H), 3.66 (s, 3H), 2.56-2.45 (m, 3H).

### Intermediate H7: ethyl 4-(2-bromo-4-fluorophenyl)-6-methyl-2-(thiazol-2-yl)-1,4-dihydropyrimidine-5-carboxylate

Intermediate H7 was prepared using same condition as for H1.

LC-MS (ESI): R_{T}=3.63 min, mass calcd. for C₁₇H₁₅BrFN₃O₂S 423.0, m/z found 423.9 [M+H]⁺. ¹H NMR (400 MHz, DMSO-d₆) δ 9.95 (s, 1H), 7.97 (d, J=2.8 Hz, 1H), 7.90 (d, J=3.2 Hz, 1H), 7.57-7.54 (m, 1H), 7.37-7.33 (m, 1H), 7.26-7.23 (m, 1H), 5.96 (s, 0.9H), 5.89 (s, 0.1H), 3.93 (q, J=7.2 Hz, 2H), 2.47 (s, 2.7H), 2.39 (s, 0.3H), 1.03 (t, J=7.2 Hz, 3H).

Racemic H7 (55.7 g, 127 mmol) was separated by chiral Prep-HPLC (separation condition: Column: OZ-H 5 μm 30*250 nm; Mobile Phase: CO₂:MeOH (0.1% NH₃—H₂O)=70:30 at 60 mL/min; Temp: 38° C.; Wavelength: 254 nm) to give the title compounds 117-A (30.0 g, 100% purity, 99.2% ee, 56% yield) as yellow solids and 117-B (27.0 g, 100% purity, 99.5% ee, 50% yield) as light brown oil.

Intermediate H7-A: LC-MS (ESI): R_{T}=1.66 min, mass calcd. for C₁₇H₁₅BrFN₃O₂S 423.0, m/z found 424.0 [M+H]⁺. Chiral analysis (Column: Chiralpak IE 5 μm 4.6*250 mm; Mobile Phase: Hex:EtOH=90:10 at 1 mL/min; Temp: 30° C.; Wavelength: 230 nm, R_{T}=8.259 min). ¹H NMR (400 MHz, CDCl₃) δ 7.83-7.80 (m, 1.7H), 7.51-7.43 (m, 1.3H), 7.35-7.30 (m, 2H), 6.99-6.94 (m, 1H), 6.17 (s, 0.7H), 6.05 (s, 0.3H), 4.08-4.01 (m, 2H), 2.57 (s, 0.8H), 2.52 (s, 2.2H), 1.13 (t, J=7.2 Hz, 3H). Optical rotation: [a]_{D}²⁵−36° (c 0.30, MeOH).

Intermediate H7-B: LC-MS (ESI): R_{T}=1.65 min, mass calcd. for C₁₇H₁₅BrFN₃O₂S 423.0, m/z found 424.0 [M+H]⁺. Chiral analysis (Column: Chiralpak IE 5 μm 4.6*250 mm; Mobile Phase: Hex:EtOH=90:10 at 1 mL/min; Temp: 30° C.; Wavelength: 230 nm, R_{T}=10.485 min). 1H NMR (400 MHz, CDCl₃) 7.85-7.79 (m, 1.7H), 7.57-7.43 (m, 1.3H), 7.35-7.30 (m, 2H), 6.99-6.94 (m, 1H), 6.17 (s, 0.7H), 6.05 (s, 0.3H), 4.11-4.02 (m, 2H), 2.57 (s, 0.8H), 2.51 (s, 2.2H), 1.13 (t, J=7.2 Hz, 3H).

### Intermediate H7-1A: Ethyl 4-(2-bromo-4-fluorophenyl)-6-(bromomethyl)-2-(thiazol-2-yl)-1,4-dihydropyrimidine-5-carboxylate

Intermediate H7-1A was prepared from H7-A using same condition as for H1-1A.

¹H NMR (400 MHz, CDCl₃) δ 8.23 (br s, 0.3H), 7.87-7.82 (m, 1H), 7.54-7.53 (m, 1H), 7.51 (br s, 0.7H), 7.45-7.39 (m, 1H), 7.34-7.31 (m, 1H), 7.05-6.98 (m, 1H), 6.18 (s, 0.3H), 6.08 (d, J=2.4 Hz, 0.7H), 5.00-4.92 (m, 1H), 4.75 (d, J=10.8 Hz, 0.3H), 4.59 (d, J=8.4 Hz, 0.7H), 4.14-4.09 (m, 2H), 1.16 (t, J=6.8 Hz, 3H).

### Intermediate H8: Ethyl 4-(2-chloro-3,4-difluorophenyl)-6-methyl-2-(thiazol-2-yl)-1,4-dihydropyrimidine-5-carboxylate

Intermediate H8 was prepared using same condition as for H1.

¹H NMR (400 MHz, CDCl₃) δ7.83-7.81 (m, 1.8H), 7.52-7.44 (m, 1.2H), 7.13-7.10 (m, 1H), 7.08-7.00 (m, 1H), 6.20 (s, 0.8H), 6.08 (s, 0.2H), 4.11-4.00 (m, 2H), 2.57 (s, 0.5H), 2.51 (s, 2.5H), 1.13 (t, J=7.2 Hz, 3H).

Racemic H8 (1.00 g, 2.51 mmol) was separated by chiral Prep-HPLC (Column: Chiralpak IC 5 μm 20*250 mm; Mobile Phase: Hex:EtOH=90:10 at 18 mL/min; Temp: 30° C.; Wavelength: 214 nm) to give the desired compound 118-A (353 mg, 35% yield, 98.1% stereopure) and 118-B (321 mg, 32% yield, 99.8% stereopure) as yellow solids.

Intermediate H8-A: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=5.901 min).

Intermediate H8-B: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=6.914 min).

### Intermediate H8-1: Ethyl 6-(bromomethyl)-4-(2-chloro-3,4-difluorophenyl)-2-(thiazol-2-yl)-1,4-dihydropyrimidine-5-carboxylate

Intermediate H8-1 was prepared from H8 using same condition as for H1-1A.

¹H NMR (400 MHz, CDCl₃) δ 8.25 (s, 0.3H), 7.85 (d, J=2.8 Hz, 1H), 7.54-7.44 (m, 1.5H), 7.20-7.04 (m, 2.2H), 6.19-6.11 (m, 1H), 4.98-4.95 (m, 1H), 4.74-4.72 (m, 0.4H), 4.58-4.56 (m, 0.6H), 4.13-4.11 (m, 2H), 1.19-1.15 (m, 3H).

### Intermediate H8-1A: Ethyl 6-(bromomethyl)-4-(2-chloro-3,4-difluorophenyl)-2-(thiazol-2-yl)-1,4-dihydropyrimidine-5-carboxylate

Intermediate H8-1A was prepared from 118-A using same condition as for H1-1A.

¹H NMR (400 MHz, CDCl₃) δ 8.25 (s, 0.3H), 7.85 (d, J=3.2 Hz, 1H), 7.54 (d, J=3.2 Hz, 0.6H), 7.47-7.45 (m, 0.9H), 7.22-7.00 (m, 2.2H), 6.19 (s, 0.4H), 6.11 (d, J=2.4 Hz, 0.6H), 4.97 (d, J=11.2 Hz, 0.4H), 4.94 (d, J=8.8 Hz, 0.6H), 4.73 (d, J=11.2 Hz, 0.4H), 4.56 (d, J=8.4 Hz, 0.6H), 4.16-4.04 (m, 2H), 1.19-1.13 (m, 3H).

### Intermediate H9: Ethyl 4-(3,4-difluoro-2-methylphenyl)-6-methyl-2-(thiazol-2-yl)-1,4-dihydropyrimidine-5-carboxylate

Intermediate H9 was prepared using same condition as for H1.

LC-MS (ESI): R_{T}=1.78 min, mass calcd. for C₁₈H₁₇F₂N₃O₂S 377.4, m/z found 378.1 [M+H]⁺. ¹H NMR (400 MHz, CDCl₃) δ 7.81-7.76 (m, 2H), 7.42 (d, J=3.2 Hz, 1H), 6.98-6.86 (m, 2H), 5.94 (s, 1H), 4.11-4.00 (m, 2H), 2.58 (s, 1.5H), 2.57 (s, 1.5H), 2.52 (s, 3H), 1.14 (t, J=7.2 Hz, 3H).

Racemic H9 (1.20 g, 90% purity, 2.86 mmol) was separated by chiral Prep-HPLC (separation condition: Column: Chiralpak IC 5 μm 30*250 mm; Mobile Phase: Hex:IPA=95:5 at 18 mL/min; Temp: 30° C.; Wavelength: 214 nm) to afford the desired compounds 119-A (580 mg, 90% purity, 48% yield, 97.8% ee) as yellow solids and 119-B (500 mg, 90% purity, 42% yield, 99.4% ee) as yellow solids.

Intermediate H9-A: Chiral analysis (Column: Chiralpak IC 5 μm 4.6*250 mm; Mobile Phase: Hex:IPA=95:5 at 1 mL/min; Temp: 30° C.; Wavelength: 230 nm, R_{T}=7.550 min).

¹H NMR (400 MHz, CDCl₃) δ 7.79-7.77 (m, 2H), 7.42 (d, J=3.6 Hz, 1H), 7.00-6.88 (m, 2H), 5.94 (s, 1H), 4.08-4.01 (m, 2H), 2.58 (s, 2.5H), 2.55 (s, 0.5H), 2.52 (s, 3H), 1.14 (t, J=7.2 Hz, 3H).

Intermediate H9-B: Chiral analysis (Column: Chiralpak IC 5 μm 4.6*250 mm; Mobile Phase: Hex:IPA=95:5 at 1 mL/min; Temp: 30° C.; Wavelength: 230 nm, R_{T}=8.495 min).

¹H NMR (400 MHz, CDCl₃) δ 7.79-7.75 (m, 2H), 7.42 (d, J=2.8 Hz, 1H), 6.98-6.86 (m, 2H), 5.94 (s, 1H), 4.08-4.00 (m, 2H), 2.58 (d, J=2.0 Hz, 3H), 2.52 (s, 3H), 1.14 (t, J=7.2 Hz, 3H).

Intermediate H9-1A: Ethyl 6-(bromomethyl)-4-(3,4-difluoro-2-methylphenyl)-2-(thiazol-2-yl)-1,4-dihydropyrimidine-5-carboxylate Intermediate H9-1A was prepared from 119-A using same condition as for H1-1A.

LC-MS (ESI): R_{T}=1.85 min, mass calcd. for C₁₈H₁₆BrF₂N₃O₂S 455.0, m/z found 456.0 [M+H]⁺. ¹H NMR (400 MHz, CDCl₃) δ 7.83 (d, J=2.8 Hz, 1H), 7.54 (d, J=2.8 Hz, 0.4H), 7.44 (d, J=2.8 Hz, 0.6H), 7.21-7.06 (m, 1H), 7.02-6.89 (m, 2H), 5.93 (s, 0.6H), 5.87 (d, J=2.0 Hz, 0.4H), 4.93 (d, J=11.6 Hz, 0.6H), 4.81-4.78 (m, 1H), 4.61 (d, J=8.4 Hz, 0.4H), 4.11-4.06 (m, 2H), 2.56 (d, J=2.0 Hz, 2H), 2.45 (d, J=2.0 Hz, 1H), 1.19-1.13 (m, 3H).

### Intermediate H9-1B: Ethyl 6-(bromomethyl)-4-(3,4-difluoro-2-methylphenyl)-2-(thiazol-2-yl)-1,4-dihydropyrimidine-5-carboxylate

Intermediate H9-1B was prepared from 119-B using same condition as for H1-1A.

LC-MS (ESI): R_{T}=1.85 min, mass calcd. for C₁₈H₁₆BrF₂N₃O₂S 455.0, m/z found 456.0 [M+H]⁺. ¹H NMR (400 MHz, CDCl₃) δ 7.83 (d, J=3.2 Hz, 1H), 7.54-7.44 (m, 1H), 7.20-7.10 (m, 1H), 7.00-6.89 (m, 2H), 5.92-5.88 (m, 1H), 4.91-4.63 (m, 2H), 4.11-4.08 (m, 2H), 2.56 (s, 2H), 2.45 (s, 1H), 1.17-1.14 (m, 3H).

### Intermediate H10: Methyl 4-(2-bromo-4-fluorophenyl)-6-methyl-2-(thiazol-2-yl)-1,4-dihydropyrimidine-5-carboxylate

Intermediate H10 was prepared using same condition as for H1.

¹H NMR (400 MHz, CDCl₃) δ 7.89-7.75 (m, 1.7H), 7.62-7.55 (m, 0.3H), 7.49-7.40 (m, 1H), 7.33-7.29 (m, 2H), 7.00-6.94 (m, 1H), 6.15 (s, 0.7H), 6.03 (s, 0.3H), 3.61 (s, 3H), 2.52 (s, 3H).

Racemic H10 (1.80 g, 90% purity, 3.95 mmol) was separated by chiral Prep-HPLC (Column: Chiralpak IG 5 μm 20 mm*250 mm; Mobile Phase: CO₂:MeOH=75:25 at 50 g/min; Col. Temp: 40° C.; Wavelength: 230 nm, Back pressure: 100 bar) to afford the title compounds H10-A (850 mg, 90% purity from ¹H NMR, 47% yield, 99.6% ee) and H10-B (850 mg, 90% purity from ¹H NMR, 47% yield, 99.4% ee) as yellow solids.

Intermediate H10-A: LC-MS (ESI): R_{T}=1.717 min, mass calcd. for C₁₆H₁₃BrFN₃O₂S 409.0, m/z found 410.0 [M+H]⁺. Chiral analysis (Column: Chiralpak IG 5 μm 4.6*250 mm; Mobile Phase: CO₂:MeOH=75:25 at 3 g/min; Temp: 40° C.; Wavelength: 230 nm; Back pressure: 100 bar, R_{T}=3.92 min). ¹H NMR (400 MHz, CDCl₃) δ 7.87-7.84 (m, 1H), 7.80 (d, J=3.2 Hz, 0.7H), 7.57 (br s, 0.3H), 7.51 (d, J=3.2 Hz, 0.3H), 7.44 (d, J=3.2 Hz, 0.7H), 7.34-7.29 (m, 2H), 7.01-6.93 (m, 1H), 6.16 (s, 0.7H), 6.02 (d, J=2.4 Hz, 0.3H), 3.62 (s, 1H), 3.60 (s, 2H), 2.57 (s, 1H), 2.51 (s, 2H).

Intermediate H10-B: LC-MS (ESI): R_{T}=1.713 min, mass calcd. for C₁₆H₁₃BrFN₃O₂S 409.0, m/z found 410.0 [M+H]⁺. Chiral analysis (Column: Chiralpak IG 5 μm 4.6*250 mm; Mobile Phase: CO₂:MeOH=75:25 at 3 g/min; Temp: 40° C.; Wavelength: 230 nm; Back pressure: 100 bar, R_{T}=4.92 min). H NMR (400 MHz, CDCl₃) δ 7.88-7.83 (m, 1H), 7.80 (d, J=3.2 Hz, 0.7H), 7.58 (br s, 0.3H), 7.50 (d, J=3.2 Hz, 0.3H), 7.44 (d, J=3.2 Hz, 0.7H), 7.34-7.29 (m, 2H), 7.01-6.93 (m, 1H), 6.16 (s, 0.7H), 6.02 (d, J=2.0 Hz, 0.3H), 3.62 (s, 1H), 3.60 (s, 2H), 2.57 (s, 1H), 2.51 (s, 2H).

### Intermediate H10-1A: Methyl 4-(2-bromo-4-fluorophenyl)-6-(bromomethyl)-2-(thiazol-2-yl)-1,4-dihydropyrimidine-5-carboxylate

Intermediate H10-1A was prepared from H10-A using same condition as for H1-1A.

¹H NMR (400 MHz, CDCl₃) δ 7.85 (d, J=3.2 Hz, 1H), 7.52 (d, J=2.8 Hz, 1H), 7.40-7.36 (m, 1H), 7.34-7.32 (m, 1H), 7.04-6.99 (m, 1H), 6.09 (s, 1H), 4.95 (d, J=9.2 Hz, 1H), 4.63 (d, J=8.4 Hz, 1H), 3.67 (s, 3H).

### Intermediate H10-1B: Methyl 4-(2-bromo-4-fluorophenyl)-6-(bromomethyl)-2-(thiazol-2-yl)-1,4-dihydropyrimidine-5-carboxylate

Intermediate H10-1B was prepared from H10-B using same condition as for H1-1A.

¹H NMR (400 MHz, CDCl₃) δ 7.85 (d, J=3.2 Hz, 1H), 7.60 (br s, 1H), 7.56-7.47 (m, 1H), 7.40-7.37 (m, 1H), 7.34-7.31 (m, 1H), 7.03-6.99 (m, 1H), 6.08 (s, 1H), 4.94 (d, J=9.2 Hz, 1H), 4.64 (br s, 1H), 3.67 (s, 3H).

### Intermediate H11: methyl 4-(2-chloro-3-fluorophenyl)-6-methyl-2-(thiazol-2-yl)-1,4-dihydropyrimidine-5-carboxylate

Intermediate H11 was prepared using same condition as for H1.

¹H NMR (400 MHz, CDCl₃) δ 7.86 (s, 0.8H), 7.83 (d, J=2.8 Hz, 0.3H), 7.80 (d, J=2.8 Hz, 0.7H), 7.55 (s, 0.2H), 7.50 (d, J=2.8 Hz, 0.2H), 7.44 (d, J=2.8 Hz, 0.8H), 7.23-7.13 (m, 2H), 7.11-7.00 (m, 1H), 6.25 (s, 0.8H), 6.11 (d, J=1.6 Hz, 0.2H), 3.62 (s, 0.6H), 3.60 (s, 2.4H), 2.58 (s, 0.6H), 2.51 (s, 2.4H).

Racemic H11 (3.00 g, 95% purity, 7.79 mmol) was separated by chiral Prep. HPLC (Column: Chiralpak IC 5 μm 20*250 mm, Mobile Phase: Hex:IPA:DEA=90:10:0.3 at 18 mL/min, Temp: 30° C., Wavelength: 230 nm) to afford the title compounds H11-A (820 mg, 96% purity, 28% yield, 100% stereopure) and H11-B (800 mg, 97% purity, 27% yield, 99.2% stereopure) as yellow solids.

Intermediate H11-A: LC-MS (ESI): R_{T}=1.587 min, mass calcd. for C₁₆H₁₃ClFN₃O₂S 365.0, m/z found 366.0 [M+H]⁺. Chiral analysis (Column: Chiralpak IC 5 μm 4.6*250 mm; Mobile Phase: Hex:IPA:DEA=90:10:0.2 at 1 mL/min; Col. Temp: 30° C.; Wavelength: 254 nm, R_{T}=10.808 min). ¹H NMR (400 MHz, CDCl₃) δ 7.86 (s, 0.7H), 7.83 (d, J=3.2 Hz, 0.2H), 7.80 (d, J=2.8 Hz, 0.8H), 7.55 (s, 0.3H), 7.50 (d, J=3.2 Hz, 0.2H), 7.44 (d, J=3.2 Hz, 0.8H), 7.22-7.13 (m, 2H), 7.08-6.99 (m, 1H), 6.25 (s, 0.8H), 6.12 (d, J=2.4 Hz, 0.2H), 3.62 (s, 1H), 3.60 (s, 2H), 2.58 (s, 1H), 2.51 (s, 2H).

Compound H11-B: LC-MS (ESI): R_{T}=1.584 min, mass calcd. for C₁₆H₁₃ClFN₃O₂S 365.0 m/z found 366.0 [M+H]⁺. Chiral analysis (Column: Chiralpak IC 5 μm 4.6*250 mm; Mobile Phase: Hex:IPA:DEA=90:10:0.2 at 1 Ll/min; Col. Temp: 30° C.; Wavelength: 254 nm, R_{T}=12.482 min). ¹H NMR (400 MHz, CDCl₃) δ 7.86 (s, 0.7H), 7.83 (d, J=3.2 Hz, 0.3H), 7.80 (d, J=3.2 Hz, 0.7H), 7.56 (s, 0.3H), 7.50 (d, J=2.8 Hz, 0.3H), 7.43 (d, J=3.2 Hz, 0.7H), 7.23-7.13 (m, 2H), 7.09-7.00 (m, 1H), 6.25 (s, 0.8H), 6.11 (d, J=2.0 Hz, 0.2H), 3.60 (s, 3H), 2.57 (s, 0.6H), 2.52 (s, 2.4H).

### H11-1A: methyl 6-(bromomethyl)-4-(2-chloro-3-fluorophenyl)-2-(thiazol-2-yl)-1,4-dihydropyrimidine-5-carboxylate

H11-1A was prepared from H11-A using same condition as for H1-1A.

LC-MS (ESI): R_{T}=1.695 min, mass calcd. for C₁₆H₁₂BrClFN₃O₂S 442.9 m/z found 444.0 [M+H]⁺. ¹H NMR (400 MHz, DMSO-d₆) δ 8.15-7.91 (m, 2H), 7.41-7.31 (m, 2H), 7.26-7.24 (m, 1H), 6.03 (s, 1H), 4.99-4.68 (m, 2H), 3.56 (s, 3H).

### H11-1B: methyl 6-(bromomethyl)-4-(2-chloro-3-fluorophenyl)-2-(thiazol-2-yl)-1,4-dihydropyrimidine-5-carboxylate

H11-1B was prepared from H11-B using same condition as for H1-1A.

LC-MS (ESI): R_{T}=1.77 min, mass calcd. for C₁₆H₁₂BrClFN₃O₂S 442.9 m/z found 445.9 [M+H]⁺. ¹H NMR (400 MHz, DMSO-d₆) δ 8.02-7.93 (m, 2H), 7.41-7.31 (m, 2H), 7.26-7.24 (m, 1H), 6.03 (s, 1H), 4.97-4.58 (m, 2H), 3.56 (s, 3H).

### H12: ethyl 4-(2-chloro-4-fluorophenyl)-6-methyl-2-(thiazol-2-yl)-1,4-dihydropyrimidine-5-carboxylate

Intermediate H12 was prepared using same condition as for H1.

LC-MS (ESI): R_{T}=1.75 min, mass calcd. for C₁₇H₁₅ClFN₃O₂S 379.1, m/z found 380.0 [M+H]⁺. ¹H NMR (400 MHz, CDCl₃) δ 7.83 (d, J=3.2 Hz, 0.3H), 7.81-7.80 (m, 1.4H), 7.50 (d, J=3.6 Hz, 0.3H), 7.46 (br s, 0.3H), 7.43 (d, J=3.2 Hz, 0.7H), 7.36-7.32 (m, 1H), 7.14-7.11 (m, 1H), 6.94-6.89 (m, 1H), 6.20 (s, 0.7H), 6.08 (s, 0.3H), 4.10-4.01 (m, 2H), 2.57 (s, 0.7H), 2.51 (s, 2.3H), 1.15-1.11 (t, J=7.2 Hz, 3H).

Racemic H12 (1.00 g, 90% purity, 2.37 mmol) was separated by chiral Prep. HPLC (separation condition: Column: Chiralpak IE 5 μm 20*250 mm; Mobile Phase: Hex:EtOH=90:10 at 10 mL/min; Temp: 30° C.; Wavelength: 254 nm) to give the title compounds 1112-A (400 mg, 98.1% purity, 44% yield, 100% ee) and H12-B (405 mg, 98.6% purity, 40% yield, 99.7% ee) as yellow solids.

Intermediate H12-A: LC-MS (ESI): R_{T}=4.295 min, mass calcd. for C₁₇H₁₅ClFN₃O₂S 379.1, m/z found 380.1 [M+H]⁺. Chiral analysis (Column: Chiralpak IE 5 μm 4.6*250 mm; Mobile Phase: Hex:EtOH=90:10 at 1.0 mL/min; Temp: 30° C.; Wavelength: 254 nm; R_{T}=7.663 min). ¹H NMR (400 MHz, CDCl₃) δ 7.83 (d, J=3.2 Hz, 0.3H), 7.80 (d, J=2.8 Hz, 1H), 7.50 (d, J=3.2 Hz, 0.3H), 7.43 (d, J=3.2 Hz, 1H), 7.36-7.32 (m, 1H), 7.14-7.11 (m, 1H), 6.94-6.89 (m, 1H), 6.20 (s, 0.7H), 6.08 (s, 0.3H), 4.08-4.01 (m, 2H), 2.57 (s, 0.8H), 2.51 (s, 2.2H), 1.13 (t, J=7.2 Hz, 3H).

Intermediate H12-B: LC-MS (ESI): R_{T}=3.578 min, mass calcd. for C₁₇H₁₅ClFN₃O₂S 379.1, m/z found 380.1 [M+H]⁺. Chiral analysis (Column: Chiralpak IE 5 μm 4.6*250 mm; Mobile Phase: Hex:EtOH=90:10 at 1.0 mL/min; Temp: 30° C.; Wavelength: 254 nm; R_{T}=9.471 min). ¹H NMR (400 MHz, CDCl₃) δ 7.83 (d, J=3.2 Hz, 0.3H), 7.80 (d, J=2.8 Hz, 1H), 7.50 (d, J=3.2 Hz, 0.3H), 7.43 (d, J=3.2 Hz, 1H), 7.36-7.32 (m, 1H), 7.14-7.11 (m, 1H), 6.94-6.89 (m, 1H), 6.20 (s, 0.7H), 6.08 (s, 0.3H), 4.08-4.00 (m, 2H), 2.57 (s, 0.8H), 2.51 (s, 2.2H), 1.13 (t, J=7.2 Hz, 3H).

### H12-1A: ethyl 6-(bromomethyl)-4-(2-chloro-4-fluorophenyl)-2-(thiazol-2-yl)-1,4-dihydropyrimidine-5-carboxylate

H12-1A was prepared from H12-A using same condition as for H1-1A.

LC-MS (ESI): R_{T}=1.80 min, mass calcd. for C₁₇H₁₄BrClFN₃O₂S 457.0, m/z found 458.0 [M+H]⁺.

### H12-1B: ethyl 6-(bromomethyl)-4-(2-chloro-4-fluorophenyl)-2-(thiazol-2-yl)-1,4-dihydropyrimidine-5-carboxylate

H12-1B was prepared from H12-B using same condition as for H1-1A.

¹H NMR (400 MHz, CDCl₃) δ 8.24 (s, 0.2H), 7.85-7.84 (d, J=2.8 Hz, 1H), 7.53-7.39 (m, 2.6H), 7.16-7.13 (m, 1H), 6.97-6.96 (m, 1H), 6.20 (s, 0.3H), 6.12 (s, 0.7H), 4.94-4.92 (m, 1H), 4.76-4.73 (m, 0.3H), 4.60-4.58 (m, 0.7H), 4.15-4.09 (m, 2H), 1.17-1.14 (t, J=6.8 Hz, 3H).

### H13: ethyl 2-(3,5-difluoropyridin-2-yl)-4-(3-fluoro-2-methylphenyl)-6-methyl-1,4-dihydropyrimidine-5-carboxylate

Intermediate H13 was prepared using same condition as for H1.

¹H NMR (400 MHz, CDCl₃) δ 8.42 (s, 1H), 7.47-7.43 (m, 1H), 7.21-7.13 (m, 2H), 7.05-7.00 (m, 1H), 6.22 (s, 1H), 4.13 (q, J=7.2 Hz, 2H), 2.65 (s, 3H), 2.48 (s, 3H), 1.17 (t, J=7.2 Hz, 3H).

Racemic H13 (500 mg, 90% purity, 1.16 mmol) was separated by chiral Prep. HPLC (separation condition: Column: Chiralpak IG 5 μm 20*250 mm; Mobile Phase: CO₂:MeOH=75:25 at 55 g/min; Temp: 30° C.; Wavelength: 230 nm, Back pressure: 100 bar) to give the title compounds H13-A (140 mg, 90% purity from ¹H NMR, 28% yield, 100% stereopure) and H13-B (200 mg, 90% purity from H NMR, 40% yield, 96.2% stereopure) as yellow solids.

Intermediate H13-A: LC-MS (ESI): R_{T}=1.75 min, mass calcd. for C₂₀H₁₈F₃N₃O₂ 389.1, m/z found 390.2 [M+H]⁺. Chiral analysis (Column: Chiralpak IG 5 um 4.6*250 mm; Mobile Phase: CO₂:MeOH=85:15 at 3 g/min; Col. Temp: 40° C.; Wavelength: 230 nm, Back pressure: 100 bar, R_{T}=3.15 min). ¹H NMR (400 MHz, CDCl₃) δ 8.28 (s, 1H), 7.33-7.29 (m, 1H), 7.11-7.04 (m, 2H), 6.94-6.89 (m, 1H), 6.14 (s, 1H), 4.06 (q, J=7.2 Hz, 2H), 2.57 (s, 3H), 2.56 (d, J=2 Hz, 3H), 1.14 (t, J=7.2 Hz, 3H).

Intermediate H13-B: LC-MS (ESI): R_{T}=1.75 min, mass calcd. for C₂₀H₁₈F₃N₃O₂ 389.1, m/z found 390.1 [M+H]⁺. Chiral analysis (Column: Chiralpak IG 5 um 4.6*250 mm; Mobile Phase: CO₂:MeOH=85:15 at 3 g/min; Col. Temp: 40° C.; Wavelength: 230 nm, Back pressure: 100 bar, R_{T}=3.74 min). ¹H NMR (400 MHz, CDCl₃) δ 8.36 (s, 1H), 7.41-7.37 (m, 1H), 7.17-7.12 (m, 2H), 7.02-6.98 (m, 1H), 6.19 (s, 1H), 4.11 (q, J=7.2 Hz, 2H), 2.62 (s, 3H), 2.48 (s, 3H), 1.16 (t, J=7.2 Hz, 3H).

### H13-1A: ethyl 6-(bromomethyl)-4-(2-chloro-4-fluorophenyl)-2-(thiazol-2-yl)-1,4-dihydropyrimidine-5-carboxylate

H13-1A was prepared from H13-A using same condition as for H1-1A.

LC-MS (ESI): R_{T}=1.80 min, mass calcd. for C₂₀H₁₇BrF₃N₃O₂ 467.1, m/z found 468.0 [M+H]⁺. ¹H NMR (400 MHz, CDCl₃) δ 8.28 (s, 1H), 7.13-6.86 (m, 4H), 6.07 (br s, 1H), 4.95-4.79 (m, 2H), 4.14-4.04 (m, 2H), 2.57-2.54 (m, 3H), 1.15 (t, J=7.2 Hz, 3H).

### H13-1B: ethyl 6-(bromomethyl)-4-(2-chloro-4-fluorophenyl)-2-(thiazol-2-yl)-1,4-dihydropyrimidine-5-carboxylate

H13-1B was prepared from H13-B using same condition as for H1-1A.

LC-MS (ESI): R_{T}=1.79 min, mass calcd. for C₂₀H₁₇BrF₃N₃O₂ 467.1, m/z found 469.9 [M+H]⁺. ¹H NMR (400 MHz, CDCl₃) δ 8.72-8.54 (m, 1H), 8.29 (s, 1H), 7.34-7.29 (m, 1H), 7.17-7.00 (m, 2H), 6.94-6.87 (m, 1H), 6.11 (br s, 0.8H), 5.98-5.90 (m, 0.2H), 5.02-4.76 (m, 2H), 4.13-4.04 (m, 2H), 2.62-2.38 (m, 3H), 1.15 (t, J=7.2 Hz, 3H).

### H14: Ethyl 4-(2-fluoro-4-methylphenyl)-6-methyl-2-(thiazol-2-yl)-1,4-dihydropyrimidine-5-carboxylate

Intermediate H14 was prepared using same condition as for H1.

¹H NMR (400 MHz, CD₃OD) δ 7.88 (d, J=2.8 Hz, 1H), 7.67 (d, J=2.8 Hz, 1H), 7.19 (t, J=8.0 Hz, 1H), 6.90-6.85 (m, 2H), 5.92 (s, 1H), 4.02 (q, J=7.2 Hz, 2H), 2.44 (s, 3H), 2.27 (s, 3H), 1.13 (t, J=7.2 Hz, 3H).

Racemic H14 (1.00 g, 2.78 mmol) was separated by chiral Prep. HPLC (separation condition: Column: Chiralpak IC 5 μm 20*250 mm; Mobile Phase: Hex:EtOH=85:15 at 18 mL/min; Temp: 30° C.; Wavelength: 230 nm) to afford the title compounds H14-A (450 mg, 45% yield, 100% stereopure) and H14-B (420 μm g, 42% yield, 99.8% stereopure) as yellow solids.

Intermediate H14-A: Chiral analysis (Column: Chiralpak IC 5 μm 4.6*250 mm; Mobile Phase: Hex:EtOH=85:15 at 1.0 mL/min; Temp: 30° C.; Wavelength: 254 nm, R_{T}=7.009 min).

Intermediate H14-B: Chiral analysis (Column: Chiralpak IC 5 μm 4.6*250 mm; Mobile Phase: Hex:EtOH=85:15 at 1.0 mL/min; Temp: 30° C.; Wavelength: 254 nm, R_{T}=8.255 min).

### H14-1A: Ethyl 6-(bromomethyl)-4-(2-fluoro-4-methylphenyl)-2-(thiazol-2-yl)-1,4-dihydropyrimidine-5-carboxylate

H14-1A was prepared from H14-A using same condition as for H1-1A.

¹H NMR (400 MHz, CD₃OD) δ 7.92 (s, 1H), 7.80-7.70 (m, 1H), 7.26 (t, J=7.6 Hz, 1H), 6.94-6.89 (m, 2H), 5.92 (d, J=2.0 Hz, 1H), 4.76-4.66 (m, 1.5H), 4.11-4.06 (m, 2H), 2.30 (d, J=1.6 Hz, 3H), 2.00 (d, J=2.8 Hz, 0.5H), 1.28-1.15 (m, 3H).

### H14-1B: Ethyl 6-(bromomethyl)-4-(2-fluoro-4-methylphenyl)-2-(thiazol-2-yl)-1,4-dihydropyrimidine-5-carboxylate

H14-1B was prepared from H14-B using same condition as for H1-1A.

¹H NMR (400 MHz, CD₃OD) δ 7.92 (s, 1H), 7.78-7.73 (m, 1H), 7.26 (t, J=8.0 Hz, 1H), 6.94-6.90 (m, 2H), 5.93 (s, 1H), 4.74-4.67 (m, 1.4H), 4.09 (q, J=7.2 Hz, 2H), 2.31 (s, 3H), 2.01 (s, 0.6H), 1.25-1.16 (m, 3H).

### H15: Ethyl 4-(3-fluoro-2-methylphenyl)-6-methyl-2-(5-methyloxazol-4-yl)-1,4-dihydropyrimidine-5-carboxylate

Intermediate H15 was prepared using same condition as for H1.

¹H NMR (400 MHz, DMSO-d₆) δ 9.21 (s, 0.8H), 8.94 (s, 0.2H), 8.35 (s, 1H), 7.16-7.06 (m, 1H), 6.99-6.94 (m, 2H), 5.80 (s, 0.8H), 5.67 (s, 0.2H), 3.97-3.94 (m, 2H), 2.46-2.40 (m, 7H), 2.38-2.30 (m, 2H), 1.04 (t, J=7.2 Hz, 3H).

Racemic H15 (1.0 g, 90% purity, 2.460 mmol) was separated by chiral Prep. HPLC (Column: Chiralpak IF 5 μm 20*250 mm, Mobile Phase: Hex:EtOH=98:2 at 18 mL/min, Temp: 30° C., Wavelength: 254 nm) to afford the title compounds H15-A (461 mg, 95% purity from H NMR, 46% yield, 100% stereopure) as yellow solids and H15-B (466 mg, 95% purity from NMR, 47% yield, 99.0% stereopure) as yellow solids.

Intermediate H15-A: LC-MS (ESI): RT=1.666 min, mass calcd. for C₁₉H₂₀FN₃O₃ 357.1, m/z found 358.1 [M+H]⁺. Chiral analysis (Column: Chiralpak IF 5 μm 4.6*250 mm; Mobile Phase: Hex:EtOH=98:2 at 1 mL/min; Col. Temp: 30° C.; Wavelength: 254 nm, R_{T}=10.686 min). ¹H NMR (400 MHz, CDCl₃) δ 7.66 (s, 1H), 7.51 (s, 1H), 7.09-7.04 (m, 1H), 7.00-6.93 (m, 1H), 6.88 (t, J=8.8 Hz, 1H), 5.98 (s, 1H), 4.07-3.98 (m, 2H), 2.54 (s, 5H), 2.51 (s, 4H), 1.11 (t, J=7.2 Hz, 3H).

Intermediate H15-B: LC-MS (ESI): RT=1.666 min, mass calcd. for C₁₉H₂₀FN₃O₃ 357.1 m/z found 358.1 [M+H]⁺. Chiral analysis (Column: Chiralpak IF 5 μm 4.6*250 mm; Mobile Phase: Hex:EtOH=98:2 at 1 ml/min; Col. Temp: 30° C.; Wavelength: 254 nm, R_{T}=13.222 min). ¹H NMR (400 MHz, CDCl₃) δ 7.66 (s, 1H), 7.51 (s, 1H), 7.09-7.04 (m, 1H), 7.00-6.98 (m, 1H), 6.88 (t, J=8.4 Hz, 1H), 5.98 (s, 1H), 4.08-4.01 (m, 2H), 2.55 (s, 5H), 2.51 (s, 4H), 1.11 (t, J=6.8 Hz, 3H).

### H15-1A: Ethyl 4-(3-fluoro-2-methylphenyl)-6-methyl-2-(5-methyloxazol-4-yl)-1,4-dihydropyrimidine-5-carboxylate

H15-1A was prepared from H15-A using same condition as for H1-1A.

LC-MS (ESI): R_{T}=1.757 min, mass calcd. for C₁₉H₁₉BrFN₃O₃ 435.0 m/z found 438.1 [M+H]⁺. ¹H NMR (400 MHz, CDCl₃) δ 7.67 (s, 1H), 7.17-7.06 (m, 1H), 7.00-6.85 (m, 2H), 5.89 (br s, 1H), 4.75 (br s, 2H), 4.08 (q, J=6.8 Hz, 2H), 2.85-2.70 (m, 2H), 2.64-2.04 (m, 4H), 1.13 (t, J=7.2 Hz, 3H).

### H15-1B: Ethyl 4-(3-fluoro-2-methylphenyl)-6-methyl-2-(5-methyloxazol-4-yl)-1,4-dihydropyrimidine-5-carboxylate

H15-1B was prepared from H15-B using same condition as for H1-1A.

¹H NMR (400 MHz, DMSO-d₆) δ 9.13 (br s, 1H), 8.40 (s, 1H), 7.21-7.19 (m, 2H), 7.10-7.03 (m, 1H), 5.74 (br s, 1H), 4.72 (br s, 2H), 4.01 (q, J=7.2 Hz, 2H), 2.75-2.65 (m, 2H), 2.46-2.37 (m, 4H), 1.07 (t, J=7.2 Hz, 3H).

### H16: Methyl 4-(3-fluoro-2-methylphenyl)-6-methyl-2-(5-methyloxazol-4-yl)-1,4-dihydropyrimidine-5-carboxylate

Intermediate H16 was prepared using same condition as for H1.

¹H NMR (400 MHz, CDCl₃) δ 7.68 (s, 1H), 7.57 (s, 1H), 7.15-7.05 (m, 1H), 7.00-6.88 (m, 2H), 5.99 (s, 0.9H), 5.87 (s, 0.1H), 3.62 (s, 3H), 2.57 (s, 6H), 2.54 (s, 3H).

Racemic H16 (270 mg, 95% purity, 0.747 mmol) was separated by chiral prep. HPLC (separation condition: Column: Chiralpak IF 5 μm 20*250 mm; Mobile Phase: Hex:IPA:DEA=98:2:0.3 at 13 mL/min; Temp: 30° C.; Wavelength: 214 nm) to give the title compounds H16-A (55 mg, 99.9% purity, 21% yield, 100% stereopure) and H16-B (50 mg, 99.9% purity, 19% yield, 96.1% stereopure) as yellow solids.

H16-A: LC-MS (ESI): R_{T}=4.185 min, mass calcd. for C₁₈H₁₈FN₃O₃ 343.1, m/z found 344.1 [M+H]⁺. Chiral analysis (Column: Chiralpak IF 5 μm 4.6*250 mm; Mobile Phase: Hex: IPA:DEA=98:2:0.2 at 1 mL/min; Temp: 30° C.; Wavelength: 254 nm; R_{T}=14.108 min).

¹H NMR (400 MHz, CDCl₃) δ 7.66 (s, 1H), 7.54 (s, 1H), 7.09-7.03 (m, 1H), 6.98-6.96 (m, 1H), 6.91-6.86 (m, 1H), 5.97 (s, 0.9H), 5.85 (s, 0.1H), 3.60 (s, 3H), 2.55 (s, 6H), 2.52 (s, 3H).

H16-B: LC-MS (ESI): R_{T}=4.186 min, mass calcd. for C₁₈H₁₈FN₃O₃ 343.1, m/z found 344.1 [M+H]⁺. Chiral analysis (Column: Chiralpak IF 5 μm 4.6*250 mm; Mobile Phase: Hex: IPA:DEA=98:2:0.2 at 1 mL/min; Temp: 30° C.; Wavelength: 254 nm; R_{T}=17.983 min).

¹H NMR (400 MHz, CDCl₃) δ 7.66 (s, 1H), 7.60-7.46 (br s, 1H), 7.09-7.04 (m, 1H), 7.00-6.94 (m, 1H), 6.91-6.87 (m, 1H), 5.97 (s, 1H), 3.60 (s, 3H), 2.55 (s, 5.2H), 2.52 (s, 3.8H).

### H16-1A: Methyl 6-(bromomethyl)-4-(3-fluoro-2-methylphenyl)-2-(5-methyloxazol-4-yl)-1,4-dihydropyrimidine-5-carboxylate

H16-1A was prepared from H16-1A using same condition as for H1-1A.

LC-MS (ESI): R_{T}=1.74 min, mass calcd. for C₁₈H₁₇BrFN₃O₃ 422.3, m/z found 424.1 [M+H]⁺. ¹H NMR (400 MHz, CDCl₃) δ 7.69 (s, 0.4H), 7.66 (s, 0.6H), 7.22-7.12 (m, 1H), 7.09-6.91 (m, 2H), 5.97 (s, 0.4H), 5.86 (s, 0.6H), 4.90 (d, J=11.2 Hz, 0.4H), 4.84 (d, J=11.2 Hz, 0.4H), 4.73 (d, J=8.4 Hz, 0.6H), 4.69 (d, J=8.4 Hz, 0.6H), 3.63 (s, 3H), 2.78 (s, 1H), 2.55-2.54 (m, 2H), 2.39 (s, 2H), 2.19 (d, J=2.4 Hz, 1H).

### H17: Ethyl 4-(2-chloro-4-fluorophenyl)-6-methyl-2-(5-methyloxazol-4-yl)-1,4-dihydropyrimidine-5-carboxylate

Intermediate H17 was prepared using same condition as for H1.

LC-MS (ESI): R_{T}=1.779 min, mass calcd. for C₁₈H₁₇ClFN₃O₃ 377.0 m/z found 378.1 [M+H]⁺. ¹H NMR (400 MHz, DMSO-d₆) δ 9.31 (s, 0.8H), 8.80 (s, 0.2H), 8.36 (s, 1H), 7.40 (dd, J=8.4, 2.4 Hz, 1H), 7.28 (t, J=7.6 Hz, 1H), 7.17 (t, J=7.6 Hz, 1H), 5.95 (s, 0.8H), 5.87 (s, 0.2H), 3.95 (q, J=7.2 Hz, 2H), 2.68 (s, 0.5H), 2.46-2.30 (m, 5.5H), 1.04 (t, J=7.2 Hz, 3H).

Racemic H17 (12.7 g, 99% purity, 32.9 mmol) was separated by chiral Prep. HPLC (Column: Chiralpak IF 5 μm 20*250 mm, Mobile Phase: Hex:IPA=95:5 at 20 mLl/min, Temp: 30° C., Wavelength: 214 nm) to afford the title compounds H17-A (5.28 g, 95% purity from ¹H NMR, 40% yield, 100% stereopure) and H17-B (5.37 g, 95% purity from ¹H NMR, 41% yield, 97.5% stereopure) as yellow solids.

Intermediate H17-A: LC-MS (ESI): R_{T}=3.541 min, mass calcd. for C₁₈H₁₇ClFN₃O₃ 377.1 m/z found 378.1 [M+H]⁺. Chiral analysis (Column: Chiralpak IF 5 μm 4.6*250 mm; Mobile Phase: Hex:IPA=95:5 at 1 mL/min; Temp: 30° C.; Wavelength: 254 nm, R_{T}=8.593 min).

¹H NMR (400 MHz, DMSO-d₆) δ 9.28 (s, 0.8H), 8.77 (d, J=3.6 Hz, 0.2H), 8.35 (s, 0.8H), 8.34 (s, 0.2H), 7.39 (dd, J=8.8, 2.8 Hz, 1H), 7.27 (dd, J=8.8, 6.4 Hz, 1H), 7.19-7.14 (m, 1H), 5.95 (s, 0.8H), 5.86 (d, J=3.2 Hz, 0.2H), 3.94 (q, J=6.8 Hz, 2H), 2.68 (s, 0.5H), 2.46 (s, 5H), 2.37 (s, 0.5H), 1.04 (t, J=7.2 Hz, 3H).

Intermediate H17-B: LC-MS (ESI): R_{T}=3.538 min, mass calcd. for C₁₈H₁₇ClFN₃O₃ 377.1 m/z found 378.1 [M+H]⁺. Chiral analysis (Column: Chiralpak IF 5 μm 4.6*250 mm; Mobile Phase: Hex:IPA=95:5 at 1 mL/min; Temp: 30° C.; Wavelength: 254 nm, R_{T}=10.412 min).

¹H NMR (400 MHz, DMSO-d₆) δ 9.28 (s, 0.8H), 8.77 (d, J=3.6 Hz, 0.2H), 8.35 (s, 0.8H), 8.34 (s, 0.2H), 7.39 (dd, J=8.8, 2.8 Hz, 1H), 7.27 (dd, J=8.8, 6.4 Hz, 1H), 7.19-7.16 (m, 1H), 5.95 (s, 0.8H), 5.85 (d, J=3.6 Hz, 0.2H), 3.94 (q, J=6.8 Hz, 2H), 2.68 (s, 0.5H), 2.46 (s, 5H), 2.37 (s, 0.5H), 1.04 (t, J=7.2 Hz, 3H).

### H17-1A: Ethyl 6-(bromomethyl)-4-(2-chloro-4-fluorophenyl)-2-(5-methyloxazol-4-yl)-1,4-dihydropyrimidine-5-carboxylate

H17-1A was prepared from H17-A using same condition as for H1-1A.

LC-MS (ESI): R_{T}=1.887 min, mass calcd. for C₁₈H₁₆BrClFN₃O₃ 455.0, 457.0 m/z found 458.0 [M+H]⁺. ¹H NMR (400 MHz, DMSO-d₆) δ 9.05 (br s, 1H), 8.39 (s, 1H), 7.45-7.35 (m, 2H), 7.24-7.20 (m, 1H), 5.92 (s, 1H), 4.73 (s, 2H), 4.06-3.99 (m, 2H), 2.79-2.62 (m, 3H), 1.08 (t, J=7.2 Hz, 3H).

### H18: Methyl 4-(2-chloro-4-fluorophenyl)-6-methyl-2-(5-methyloxazol-4-yl)-1,4-dihydropyrimidine-5-carboxylate

Intermediate H18 was prepared using same condition as for H1.

LC-MS (ESI): R_{T}=1.52 min, mass calcd. for C₁₇H₁₅ClFN₃O₃ 363.1, m/z found 364.0 [M+H]⁺. ¹H NMR (400 MHz, CDCl₃) δ 7.67 (s, 1H), 7.59-7.50 (m, 0.6H), 7.34-7.31 (m, 0.8H), 7.23-7.14 (m, 0.6H), 7.13 (dd, J=8.4, 2.0 Hz, 1H), 6.96-6.86 (m, 1H), 6.14 (s, 0.6H), 5.99 (s, 0.4H), 3.60 (s, 3H), 2.72 (s, 1.2H), 2.62-2.51 (m, 4.8H).

### H18-2:1-tert-Butyl 5-methyl 6-(2-chloro-4-fluorophenyl)-4-methyl-2-(5-methyloxazol-4-yl)pyrimidine-1,5(6H)-dicarboxylate

H18-2 was prepared from H18 by treating with Boc2O and DIPEA at 50° C.

LC-MS (ESI): R_{T}=1.853 min, mass calcd. for C₂₂H₂₃ClFN₃O₅ 463.1, m/z found 464.1 [M+H]⁺. ¹H NMR (400 MHz, CDCl₃) δ 7.68 (s, 1H), 7.17-7.10 (m, 2H), 6.79 (td, J=8.4, 2.4 Hz, 1H), 6.70 (s, 1H), 3.71 (s, 3H), 2.58 (s, 3H), 2.44 (s, 3H), 1.34 (s, 9H).

Racemic H18-2 (15.2 g, 90% purity, 29.5 mmol) was separated by prep. chiral HPLC (Chiral Column: Chiralpak IC 5 μm 30*250 mm; Mobile Phase: Hex:EtOH=98:2 at 30 mL/min; Temp: 30° C.; Wavelength: 254 nm) to give H118-2A (6.58 g, 95% purity from ¹H NMR, 99.5% ee, 46% yield) as yellow solids and 1118-2B (5.76 g, 95% purity from 1H NMR, 97.9% ee, 40% yield) as yellow solids.

Intermediate H18-2A: LC-MS (ESI): R_{T}=1.72 min, mass calcd. for C₂₂H₂₃ClFN₃O₅ 463.1, m/z found 464.0 [M+H]⁺. Chiral analysis (Chiral Column: Chiralpak IC 5 μm 4.6*250 mm; Mobile Phase: Hex:EtOH=98:2 at 1 mL/min; Temp: 30° C.; Wavelength: 254 nm; R_{T}=10.327 min). ¹H NMR (400 MHz, CDCl₃) δ 7.68 (s, 1H), 7.17-7.10 (m, 2H), 6.79 (td, J=8.0, 2.4 Hz, 1H), 6.70 (s, 1H), 3.71 (s, 3H), 2.58 (s, 3H), 2.44 (s, 3H), 1.34 (s, 9H).

Intermediate H18-2B: LC-MS (ESI): R_{T}=1.72 min, mass calcd. for C₂₂H₂₃ClFN₃O₅ 463.1, m/z found 464.0 [M+H]⁺. Chiral analysis (Column: Chiralpak IC 5 μm 4.6*250 mm; Mobile Phase: Hex:EtOH=98:2, at 1 mL/min; Temp: 30° C.; Wavelength: 254 nm; R_{T}=11.793 min). ¹H NMR (400 MHz, CDCl₃) δ 7.68 (s, 1H), 7.17-7.10 (m, 2H), 6.79 (td, J=8.0, 2.4 Hz, 1H), 6.70 (s, 1H), 3.71 (s, 3H), 2.57 (s, 3H), 2.43 (s, 3H), 1.34 (s, 9H).

### H18-A: Methyl 4-(2-chloro-4-fluorophenyl)-6-methyl-2-(5-methyloxazol-4-yl)-1,4-dihydropyrimidine-5-carboxylate

H18-A was prepared from H18-2Aby treating with TFA.

LC-MS (ESI): R_{T}=1.717 min, mass calcd. for C₁₇H₁₅ClFN₃O₃ 363.1, m/z found 364.1 [M+H]⁺. ¹H NMR (400 MHz, CDCl₃) δ 7.67 (s, 1H), 7.61-7.52 (s, 0.7H), 7.38-7.28 (m, 0.6H), 7.26-7.22 (m, 0.7H), 7.13 (dd, J=8.8, 2.8 Hz, 1H), 6.91-6.85 (m, 1H), 6.14 (s, 0.7H), 5.99 (s, 0.3H), 3.60 (s, 3H), 2.72 (s, 0.9H), 2.64-2.51 (m, 5.1H).

### H18-1A: Methyl 6-(bromomethyl)-4-(2-chloro-4-fluorophenyl)-2-(5-methyloxazol-4-yl)-1,4-dihydropyrimidine-5-carboxylate

H18-1A was prepared from H18-A using same condition as for H1-1A.

LC-MS (ESI): R_{T}=1.76 min, mass calcd. for C₁₇H₁₄BrClFN₃O₃ 441.0, m/z found 442.0 [M+H]⁺. ¹H NMR (400 MHz, CDCl₃) δ 7.68 (s, 1H), 7.40-7.34 (m, 1H), 7.14 (dd, J=8.4, 2.4 Hz, 1H), 6.98-6.94 (m, 1H), 6.02 (s, 1H), 4.89 (d, J=8.4 Hz, 1H), 4.64 (d, J=8.4 Hz, 1H), 3.65 (s, 3H), 2.76 (s, 3H).

### H19: Ethyl 4-(3-fluoro-2-methylphenyl)-6-methyl-2-(thiazol-2-yl)-1-hydro-4-deuteropyrimidine-5-carboxylate

Intermediate H19 was prepared using same condition as for H1.

¹H NMR (300 MHz, CDCl₃) δ 7.88 (d, J=3.0 Hz, 0.3H), 7.82 (d, J=3.0 Hz, 1.7H), 7.56 (d, J=3.0 Hz, 0.3H), 7.45 (d, J=3.3 Hz, 0.7H), 7.13-7.08 (m, 2H), 6.97-6.91 (m, 1H), 4.10 (q, J=6.9 Hz, 2H), 2.59 (s, 3H), 2.57 (s, 3H), 1.17 (t, J=7.2 Hz, 3H).

Racemic H19 (48.0 g, 90% purity, 120 mmol) was separated by chiral Prep. HPLC (separation condition: Column: Chiralpak AS 5 μm 20*250 mm; Mobile Phase: Hex:EtOH:DEA=80:20:0.3 at 18 mL/min; Temp: 30° C.; Wavelength: 254 nm) to afford the desired compounds H19-A (19.0 g, 90% purity from H NMR, 40% yield, 100% stereopure) and H19-B (17.8 g, 90% purity from ¹H NMR, 37% yield, 99.9% stereopure) as yellow solids.

Intermediate H19-A: Chiral analysis (Column: Chiralpak OJ-H 5 μm 4.6*250 mm; Mobile Phase: Hex:EtOH:DEA=85:15:0.2 at 1 mL/min; Temp: 30° C.; Wavelength: 254 nm, R_{T}=9.457 min). ¹H NMR (400 MHz, CDCl₃) δ 7.78 (d, J=3.6 Hz, 2H), 7.43 (s, 1H), 7.09-7.06 (m, 2H), 6.92-6.88 (m, 1H), 4.06 (q, J=7.2 Hz, 2H), 2.53 (s, 6H), 1.13 (t, J=7.2 Hz, 3H).

Intermediate H19-B: Chiral analysis (Column: Chiralpak OJ-H 5 μm 4.6*250 mm; Mobile Phase: Hex:EtOH:DEA=85:15:0.2 at 1 mL/min; Temp: 30° C.; Wavelength: 254 nm, R_{T}=7.311 min). ¹H NMR (400 MHz, CDCl₃) δ 7.78 (d, J=3.2 Hz, 2H), 7.42 (s, 1H), 7.10-7.06 (m, 2H), 6.92-6.88 (m, 1H), 4.06 (q, J=7.2 Hz, 2H), 2.53 (s, 6H), 1.13 (t, J=7.2 Hz, 3H).

### H19-1B: Ethyl 6-(bromomethyl)-4-deutero-4-(3-fluoro-2-methylphenyl)-2-(thiazol-2-yl)-1,4-dihydropyrimidine-5-carboxylate

H19-1B was prepared from H19-B using same condition as for H1-1A.

¹H NMR (400 MHz, CDCl₃) δ 8.20 (s, 0.6H), 7.82 (d, J=2.4 Hz, 1H), 7.53 (d, J=2.8 Hz, 0.5H), 7.44 (d, J=2.8 Hz, 0.5H), 7.23 (s, 0.4H), 7.15-7.08 (m, 2H), 6.97-6.89 (m, 1H), 4.92-4.76 (m, 1.6H), 4.66 (d, J=8.4 Hz, 0.4H), 4.10 (q, J=7.2 Hz, 2H), 2.53 (s, 1.6H), 2.41 (s, 1.4H), 1.14 (t, J=6.8 Hz, 3H)

### H20: ethyl 4-(6-fluoro-2-methylpyridin-3-yl)-6-methyl-2-(thiazol-2-yl)-1,4-dihydropyrimidine-5-carboxylate

Intermediate H20 was prepared using same condition as for H1.

LC-MS (ESI): R_{T}=1.42 min, mass calcd. for C₁₇H₁₇FN₄O₂S 360.1, m/z found 361.3 [M+H]⁺.

¹H NMR (400 MHz, CDCl₃) δ 7.83 (s, 1H), 7.81 (d, J=3.2 Hz, 1H), 7.60 (t, J=8.0 Hz, 1H), 7.45 (d, J=3.2 Hz, 1H), 6.68 (dd, J=8.4, 3.2 Hz, 1H), 5.98 (s, 1H), 4.11-4.03 (m, 2H), 2.80 (s, 3H), 2.53 (s, 3H), 1.15 (t, J=7.2 Hz, 3H).

Racemic H20 was chiral separated to give H20-A and 1120-B.

H20-A: LC-MS (ESI): R_{T}=1.40 min, mass calcd. for C₁₇H₁₇FN₄O₂S 360.11, m/z found 361.3 [M+H]⁺. ¹H NMR (400 MHz, CDCl₃) δ 7.83 (s, 1H), 7.80 (d, J=3.2 Hz, 1H), 7.60 (t, J=8.0 Hz, 1H), 7.45 (d, J=3.2 Hz, 1H), 6.68 (dd, J=8.4, 3.2 Hz, 1H), 5.98 (s, 1H), 4.11-4.03 (m, 2H), 2.80 (s, 3H), 2.53 (s, 3H), 1.15 (t, J=7.2 Hz, 3H). Chiral analysis (100% stereopure, Chiralpak IE 5 μm 4.6*250 mm; Mobile Phase: Hex:EtOH=70:30 at 1 mL/min; Temp: 30° C., Wavelength: 254 nm, R_{T}=5.773 min).

H20-B: LC-MS (ESI): R_{T}=1.41 min, mass calcd. for C₁₇H₁₇FN₄O₂S 360.11, m/z found 361.3 [M+H]⁺. ¹H NMR (400 MHz, CDCl₃) δ 7.83 (s, 1H), 7.80 (d, J=3.2 Hz, 1H), 7.60 (t, J=8.0 Hz, 1H), 7.45 (d, J=3.2 Hz, 1H), 6.68 (dd, J=8.0, 3.2 Hz, 1H), 5.98 (s, 1H), 4.11-4.03 (m, 2H), 2.80 (s, 3H), 2.53 (s, 3H), 1.15 (t, J=6.8 Hz, 3H). Chiral analysis (99.9% stereopure, Chiralpak IE 5 μm 4.6*250 mm; Mobile Phase: Hex:EtOH=70:30 at 1 mL/min; Temp: 30° C., Wavelength: 254 nm, R_{T}=6.724 min).

### H20-1A: ethyl 6-(bromomethyl)-4-(6-fluoro-2-methylpyridin-3-yl)-2-(thiazol-2-yl)-1,4-dihydropyrimidine-5-carboxylate

H20-1A was prepared from H20-A using same condition as for H1-1A.

LC-MS (ESI): R_{T}=1.70 min, mass calcd. for C₁₇H₁₆BrFN₄O₂S 438.0, m/z found 441.2 [M+H]⁺. ¹H NMR (400 MHz, CDCl₃) δ 8.32-8.18 (m, 0.4H), 7.84 (d, J=3.2 Hz, 1H), 7.73-7.59 (m, 0.7H), 7.54-7.44 (m, 1H), 6.76-6.69 (m, 1H), 5.02-4.85 (m, 1H), 4.79-4.61 (m, 0.3H), 4.16-4.05 (m, 2H), 2.83-2.65 (s, 3H), 1.17 (t, J=7.2 Hz, 3H).

### Preparation of Deuteride-3-fluoro-2-methylbenzaldehyde

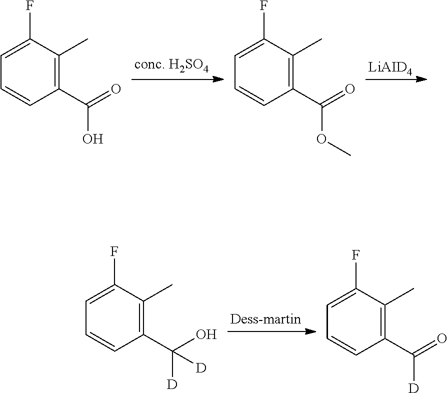

### Methyl 3-fluoro-2-methylbenzoate

To a solution of 3-fluoro-2-methylbenzoic acid (50.0 g, 324 mmol) in methanol (500 mL) was added concentrated sulfuric acid (25 mL) at room temperature. After stirred at 80° C. overnight, the mixture was cooled down to room temperature and diluted with ethyl acetate (400 mL). The mixture was washed with water (200 mL), dried over Na₂SO₄₍ₛ₎ and filtered. The filtrate was concentrated under reduced pressure to give a residue, which was purified by silica gel column chromatography (petroleum ether:ethyl acetate=100:1) to give the title compound (52.1 g, 90% purity from H NMR, 86% yield) as colorless oil. ¹H NMR (400 MHz, CDCl₃) δ 7.66-7.64 (m, 1H), 7.18-7.13 (m, 2H), 3.89 (s, 3H), 2.48 (d, J=2.4 Hz, 3H).

### Dideutero(3-fluoro-2-methylphenyl)methanol

To a solution of methyl 3-fluoro-2-methylbenzoate (52.1 g, 90% purity, 279 mmol) in tetrahydrofuran (500 mL) was slowly added lithium aluminum deuteride (13.6 g, 324 mmol) at 0° C. After stirred at room temperature overnight, the mixture was quenched with water (25 mL), 15% sodium hydroxide aqueous solution (40 mL) and dry magnesium sulphate (55 g) at 0° C. After stirred for 30 minutes, the mixture was filtered and filtrate was concentrated under reduced pressure to give the title compound (49.2 g, 80% purity from H NMR, 99% yield) as colorless oil. ¹H NMR (400 MHz, CDCl₃) δ 7.17-7.12 (m, 2H), 6.98-6.93 (m, 1H), 2.24 (d, J=2.4 Hz, 3H).

### Deuteride-3-fluoro-2-methylbenzaldehyde

To a solution of dideutero(3-fluoro-2-methylphenyl)methanol (49.2 g, 80% purity, 277 mmol) in dichloromethane (500 mL) was added 1,1-dihydro-1,1,1-triacetoxy-1,2-benzoiodoxol-3(1 h)-one (264 g, 622 mmol) slowly at room temperature. After stirred at room temperature for 1 hour, the mixture was quenched with sodium thiosulfate and saturated sodium carbonate aqueous solution. The mixture was filtered and filtrate was concentrated under reduced pressure to give a residue, which was dissolved in ethyl acetate (50 mL) and washed with water (50 mL), dried over Na₂SO₄₍ₛ₎ and filtered. The filtrate was concentrated under reduced pressure to give a residue, which was purified by silica gel column chromatography (petroleum ether:ethyl acetate=100:1) to give the title compound (44.1 g, 85% purity from ¹H NMR, 97% yield) as colorless oil. ¹H NMR (400 MHz, CDCl₃) δ 7.61 (d, J=7.6 Hz, 1H), 7.35-7.24 (m, 2H), 2.58 (d, J=1.6 Hz, 3H).

### Compound 1A: 3-(7-(((S)-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

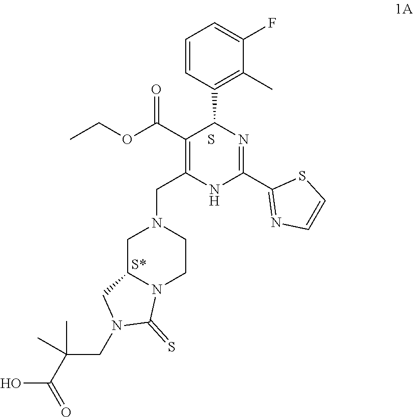

### Preparation of 2,2-dimethyl-3-(3-thioxohexahydroimidazo[1,5-a]pyrazin-2(3H)-yl)propanoic acid (Intermediate S1)

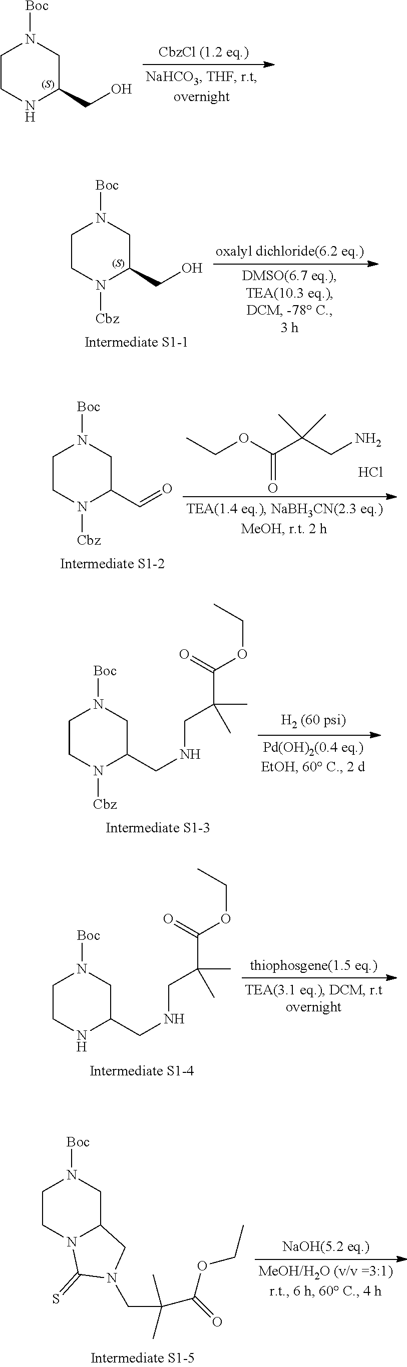

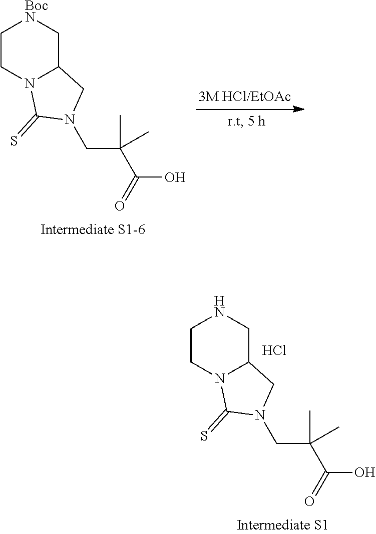 [SMILES: CC(C)(C)OC(=O)N1CCN2C(=S)N(CC(C)(C)C(=O)O)CC2C1 ; CC(C)(CN1CC2CNCCN2C1=S)C(=O)O ; Cl]

### Intermediate S1-1:1-benzyl 4-(tert-butyl) (S)-2-(hydroxymethyl)piperazine-1,4-dicarboxylate

To the solution of (S)-tert-butyl 3-(hydroxymethyl)piperazine-1-carboxylate (10.0 g, 46.2 mmol) and saturated sodium bicarbonate aqueous solution (64 mL) in tetrahydrofuran (106 mL) was added dropwise benzyl chloroformate (9.16 g, 53.7 mmol) at 0° C. under nitrogen atmosphere. After stirred at room temperature overnight, the mixture was concentrated under reduced pressure to remove tetrahydrofuran, added water (50 mL) and extracted with ethyl acetate (50 mL) for three times. The combined organic layers were washed with brine (100 mL), dried over Na₂SO₄₍ₛ₎ and filtered. The filtrate was concentrated and purified by silica gel column chromatography (petroleum ether:ethyl acetate=4:1 to 1:1) to give the title compound S1-1 (14.8 g, 82% yield) as colorless oil. LC-MS (ESI): R_{T}=2.056 min, mass calcd. for C₁₈H₂₆N₂O₅ 350.2, m/z found 373.1[M+Na]⁺. ¹H NMR (400 MHz, CDCl₃) δ 7.43-7.30 (m, 5H), 5.17 (d, J=12.4 Hz, 1H), 5.12 (d, J=12.4 Hz, 1H), 4.31-4.12 (m, 2H), 4.07-3.84 (m, 2H), 3.73-3.50 (m, 2H), 3.15-2.79 (m, 3H), 1.47 (s, 9H).

### Intermediate S1-2:1-Benzyl 4-tert-butyl 2-formylpiperazine-1,4-dicarboxylate (mixture of 2 enantiomers)

To a solution of anhydrous dimethyl sulfoxide (38.5 g, 493 mmol) in anhydrous dichloromethane (300 mL) was added dropwise oxalyl dichloride (57.8 g, 455 mmol) at −78° C. After stirred at −78° C. under nitrogen atmosphere for 1.5 hours, a solution of (S)-1-benzyl 4-tert-butyl 2-(hydroxymethyl)piperazine-1,4-dicarboxylate S1-1 (28.8 g, 90% purity, 73.9 mmol) in anhydrous dichloromethane (50 mL) was added dropwise. The mixture was stirred at −78° C. for 1.5 hours and triethylamine (60.9 g, 602 mmol) was then added. After stirred at room temperature for 0.5 hour, the reaction mixture was diluted with ice water (100 mL) and neutralized with 1 M hydrochloride aqueous solution to pH 6˜7, extracted with dichloromethane (150 mL) for three times. The combined organic phases were washed with saturated sodium bicarbonate (100 mL) and brine (100 mL) for three times, dried over Na₂SO₄₍ₛ₎, filtered and evaporated to give the title compound S1-2 (28.8 g, 89% yield) as light yellow oil. LC-MS (ESI): R_{T}=1.68 min, mass calcd. for C₁₈H₂₄N₂O₅ 348.2, m/z found 293.1[M+H−56]⁺. ¹H NMR (400 MHz, CDCl₃) δ 9.60 (d, J=7.2 Hz, 1H), 7.37-7.29 (m, 5H), 5.18 (s, 1H), 5.14 (s, 1H), 4.91-4.51 (m, 2H), 4.07-3.82 (m, 2H), 3.29-3.07 (m, 2H), 3.00-2.79 (m, 1H), 1.44 (s, 9H).

### Intermediate S1-3: 1-Benzyl 4-tert-butyl 2-(((3-ethoxy-2,2-dimethyl-3-oxopropyl)amino)methyl)piperazine-1,4-dicarboxylate (mixture of 2 enantiomers)

To a solution of ethyl 3-amino-2,2-dimethylpropanoate hydrochloride (17.7 g, 97.4 mmol) in methanol (200 mL) was added triethylamine (9.86 g, 97.4 mmol) at room temperature. After stirred at room temperature under nitrogen atmosphere for 0.5 hour, a solution of 1-benzyl 4-tert-butyl 2-formylpiperazine-1,4-dicarboxylate S1-2 (29.5 g, 80% purity, 67.7 mmol) in methanol (100 mL) was added and stirred at room temperature for 1 hour. Then sodium cyanoborohydride (9.84 g, 157 mmol) was added at 0° C. and the mixture was stirred at room temperature for 2 hours, quenched with ice water (100 mL), removed methanol under vacuo and extracted with ethyl acetate (100 mL) for three times. The combined organic layers were dried over Na₂SO₄₍ₛ₎ and filtered. The filtrate was concentrated and purified by silica gel chromatography (petroleum ether:ethyl acetate=8:1 to 2:1) to give the title compound S1-3 (29.6 g, 82% yield) as light yellow oil. LC-MS (ESI): R_{T}=2.533 min, mass calcd. for C₂₅H₃₉N₃O₆ 477.3, m/z found 478.3 [M+H]⁺. ¹H NMR (400 MHz, CDCl₃) δ 7.39-7.31 (m, 5H), 7.27 (s, 1H), 5.83-5.77 (m, 0.7H), 5.67-5.62 (m, 0.3H), 5.16 (s, 2H), 4.30 (t, J=6.4 Hz, 2H), 3.68 (s, 3H), 2.89 (t, J=7.2 Hz, 0.5H), 2.86 (t, J=6.4 Hz, 1.5H), 2.71-2.62 (m, 1H), 2.46-2.42 (m, 2H), 2.40-2.33 (m, 2H), 2.31 (s, 1H), 2.30 (s, 2H), 2.17-2.12 (m, 1H), 1.92-1.79 (m, 1H).

### Intermediate S1-4: tert-Butyl 3-(((3-ethoxy-2,2-dimethyl-3-oxopropyl)amino)methyl)piperazine-1-carboxylate (mixture of 2 enantiomers)

To a solution of 1-benzyl 4-tert-butyl 2-(((3-ethoxy-2,2-dimethyl-3-oxopropyl)amino) methyl)piperazine-1,4-dicarboxylate S1-3 (17.6 g, 33.2 mmol) in ethanol (300 mL) was added 20% wt. palladium hydroxide on carbon (8.0 g, 11.4 mmol) and then the mixture was stirred at 60° C. under 60 psi hydrogen atmosphere overnight. Another 20% palladium hydroxide on carbon (500 mg, 0.712 mmol) was added and stirring continued at 60° C. under 60 psi hydrogen atmosphere overnight. Then the reaction mixture was filtered and the filtrate was concentrated under reduced pressure to give the title compound S1-4 (11.7 g, 82% yield) as colorless oil. LC-MS (ESI): R_{T}=1.362 min, mass calcd. for C₁₇H₃₃N₃O₄ 343.2, m/z found 344.11 [M+H]⁺. ¹H NMR (400 MHz, CDCl₃) δ 4.12 (q, J=7.2 Hz, 2H), 4.02-3.80 (m, 2H), 2.99-2.96 (m, 1H), 2.94-2.81 (m, 1H), 2.74-2.63 (m, 4H), 2.60-2.47 (m, 3H), 1.46 (s, 9H), 1.25 (t, J=7.2 Hz, 3H), 1.19 (s, 3H), 1.17 (s, 3H).

### Intermediate S1-5: tert-Butyl 2-(3-ethoxy-2,2-dimethyl-3-oxopropyl)-3-thioxohexahydroimidazo[1,5-a]pyrazine-7(1H)-carboxylate

To a solution of tert-butyl 3-(((3-ethoxy-2,2-dimethyl-3-oxopropyl)amino)methyl)pip erazine-1-carboxylate S1-4 (3.70 g, 8.62 mmol) and triethylamine (2.72 g, 26.9 mmol) in dichloromethane (25 mL) was added a solution of thiophosgene (1.48 g, 12.9 mmol) in dichloromethane (5 mL) at 0° C. under nitrogen atmosphere. After stirred at room temperature overnight, the mixture was diluted with ice water (20 mL) and extracted with dichloromethane (15 mL) for three times. The combined organic layers were washed with brine (30 mL), dried over Na₂SO₄₍ₛ₎ and filtered. The filtrate was concentrated and purified by C18 column (acetonitrile:water=5% to 100%) to give the title compound S1-5 (2.1 g, 57% yield) as white solids. LC-MS (ESI): R_{T}=2.380 min, mass calcd. for C₁₈H₃₁N₃O₄S 385.2, m/z found 386.2 [M+H]⁺. ¹H NMR (400 MHz, CDCl₃) 4.49-4.45 (m, 1H), 4.16 (q, J=7.2 Hz, 2H), 4.11-4.10 (m, 1H), 4.08-4.00 (m, 1H), 3.94 (d, J=14.4 Hz, 1H), 3.87 (d, J=14.0 Hz, 1H), 3.78-3.69 (m, 1H), 3.60 (t, J=9.6 Hz, 1H), 3.11-3.07 (m, 1H), 3.03-2.99 (m, 1H), 2.92-2.78 (m, 1H), 2.67-2.51 (m, 1H), 1.46 (s, 9H), 1.28 (t, J=7.2 Hz, 3H), 1.25 (s, 3H), 1.24 (s, 3H).

A racemic mixture of tert-butyl 2-(3-ethoxy-2,2-dimethyl-3-oxopropyl)-3-thioxohexa hydroimidazo[1,5-a]pyrazine-7(1H)-carboxylate S1-5 (7.3 g, 90% purity, 17.0 mmol) was separated by chiral Prep-HPLC (separation condition: Column: Chiralpak IF 5 μm 20*250 mm; Mobile Phase: Hex:EtOH:DEA=80:20:0.3 at 15 mL/min; Temp: 30° C.; Wavelength: 230 nm) to afford the title compound S1-5A (4.38 g) as white solids and S1-5B (1.89 g) as white solids.

S1-5A: LC-MS (ESI): R_{T}=1.74 min, mass calcd. for C₁₈H₃₁N₃O₄S 385.2, m/z found 386.3 [M+H]⁺. Chiral analysis (Column: Chiralpak IF 5 μm 4.6*250 mm; Mobile Phase: Hex: EtOH:DEA=80:20:0.2 at 1 mL/min; Temp: 30° C.; Wavelength: 254 nm, R_{T}=9.710 min). H NMR (400 MHz, CDCl₃) δ 4.48-4.46 (m, 1H), 4.28-4.18 (m, 1H), 4.16 (q, J=7.2 Hz, 2H), 4.11-4.00 (m, 1H), 3.94 (d, J=14.0 Hz, 1H), 3.87 (d, J=14.40 Hz, 1H), 3.78-3.679 (m, 1H), 3.610 (t, J=9.6 Hz, 1H), 3.11-3.07 (m, 1H), 3.03-2.97 (m, 1H), 2.92-2.75 (m, 1H), 2.69-2.51 (m, 1H), 1.47 (s, 9H), 1.28 (t, J=7.2 Hz, 3H), 1.25 (s, 3H), 1.24 (s, 3H).

S1-5B: LC-MS (ESI): R_{T}=1.74 min, mass calcd. for C₁₈H₃₁N₃O₄S 385.2, m/z found 386.3 [M+H]⁺. Chiral analysis: (Column: Chiralpak IF 5 μm 4.6*250 mm; Mobile Phase: Hex: EtOH:DEA=80:20:0.2 at 1 mL/min; Temp: 30° C.; Wavelength: 254 nm, R_{T}=7.397 min). ¹H NMR (400 MHz, CDCl₃) δ 4.49-4.46 (m, 1H), 4.33-4.18 (m, 1H), 4.16 (q, J=7.2 Hz, 2H), 4.11-3.99 (m, 1H), 3.94 (d, J=14.4 Hz, 1H), 3.87 (d, J=14.0 Hz, 1H), 3.79-3.69 (m, 1H), 3.60 (t, J=9.6 Hz, 1H), 3.11-3.07 (m, 1H), 3.03-2.97 (m, 1H), 2.92-2.75 (m, 1H), 2.68-2.50 (m, 1H), 1.47 (s, 9H), 1.28 (t, J=7.2 Hz, 3H), 1.25 (s, 3H), 1.24 (s, 3H).

### Intermediate S1-6A: 3-(7-(tert-butoxycarbonyl)-3-thioxohexahydroimidazo[1,5-a]pyrazin-2(3H)-yl)-2,2-dimethylpropanoic acid

To a solution of tert-butyl 2-(3-ethoxy-2,2-dimethyl-3-oxopropyl)-3-thioxohexahydroimidazo[1,5-a]pyrazine-7(1H)-carboxylate S1-5A (4.38 g, 10.2 mmol) in methanol (30 mL) and water (10 mL) was added sodium hydroxide (1.43 g, 35.8 mmol) under nitrogen atmosphere at 0° C. After stirred at room temperature for 6 hours, the mixture was added sodium hydroxide (700 mg, 17.5 mmol) and stirred at 60° C. for 4 hours. Then the reaction was diluted with water (20 mL), removed methanol under vacuo and extracted with ethyl acetate (20 mL) twice. The combined aqueous phase was acidified with saturated citric acid aqueous solution to pH 3˜4, extracted with ethyl acetate (20 mL) for three times. The combined organic layers were washed with brine (30 mL), dried over Na₂SO₄₍ₛ₎ and filtered. The filtrate was concentrated to give the title compound S1-6A (3.6 g, 90% purity from ¹H NMR, 89% yield) as white solids. LC-MS (ESI): R_{T}=1.612 min, mass calcd. For C₁₆H₂₇N₃O₄S 357.2, m/z found 358.2 [M+H]⁺. ¹H NMR (400 MHz, DMSO-d₆) δ 12.47 (br s, 1H), 4.25-4.21 (m, 1H), 4.06-4.02 (m, 1H), 3.95-3.92 (m, 1H), 3.81 (d, J=14.0 Hz, 1H), 3.79-3.74 (m, 1H), 3.73 (d, J=13.6 Hz, 1H), 3.65 (t, J=9.6 Hz, 1H), 3.18-3.13 (m, 1H), 2.99-2.92 (m, 1H), 2.80-2.54 (m, 2H), 1.41 (s, 9H), 1.12 (s, 3H), 1.11 (s, 3H).

### Intermediate S1-6B: 3-(7-(tert-Butoxycarbonyl)-3-thioxohexahydroimidazo[1,5-a]pyrazin-2(3H)-yl)-2,2-dimethylpropanoic acid

To a solution of tert-butyl 2-(3-ethoxy-2,2-dimethyl-3-oxopropyl)-3-thioxohexa hydroimidazo[1,5-a]pyrazine-7(1H)-carboxylate S1-5B (810 mg, 1.89 mmol) in methanol (15 mL) and water (5 mL) was added sodium hydroxide (263 mg, 6.58 mmol) under nitrogen atmosphere at 0° C. After stirred at room temperature for 6 hours, the mixture was added sodium hydroxide (130 mg, 3.25 mmol) and stirred at 60° C. for 4 hours. Then the reaction was diluted with water (10 mL), removed methanol under vacuo and extracted with ethyl acetate (20 mL) twice. The combined aqueous phase were acidified with saturated citric acid aqueous solution to pH 3˜4, extracted with ethyl acetate (20 mL) for three times. The combined organic layers were washed with brine (20 mL), dried over Na₂SO₄₍ₛ₎ and filtered. The filtrate was concentrated to give the title compound S1-6B (650 mg, 87% yield) as white solids. LC-MS (ESI): R_{T}=1.654 min, mass calcd. For C₁₆H₂₇N₃O₄S 357.2, m/z found 358.2 [M+H]⁺. ¹H NMR (400 MHz, DMSO-d₆) δ 12.46 (br s, 1H), 4.25-4.21 (m, 1H), 4.10-4.00 (m, 1H), 3.95-3.92 (m, 1H), 3.81 (d, J=13.6 Hz, 1H), 3.79-3.74 (m, 1H), 3.73 (d, J=14.0 Hz, 1H), 3.65 (t, J=10.0 Hz, 1H), 3.18-3.14 (m, 1H), 2.99-2.92 (m, 1H), 2.80-2.55 (m, 2H), 1.41 (s, 9H), 1.12 (s, 3H), 1.11 (s, 3H).

### Intermediate S1-A: 2,2-Dimethyl-3-(3-thioxohexahydroimidazo[1,5-a]pyrazin-2(3H)-yl)propanoic acid hydrochloride

3-(7-(tert-butoxycarbonyl)-3-thioxohexahydroimidazo[1,5-a]pyrazin-2(3H)-yl)-2,2-dimethylpropanoic acid S1-6A (3.6 g, 9.06 mmol) was added into 3 M hydrochloride in ethyl acetate (50 mL, 150 mmol). The reaction was stirred at room temperature under nitrogen atmosphere for 5 hours, the completed reaction was concentrated under reduced pressure to give the title compound (2.9 g, 98% yield) as white solids. LC-MS (ESI): R_{T}=0.513 min, mass calcd. for C₁₁H₂₀ClN₃O₂S 293.1, m/z found 258.1 [M+H—HCl]⁺. ¹H NMR (400 MHz, DMSO-d₆) δ 12.41 (br s, 1H), 9.62 (br s, 2H), 4.39-4.35 (m, 1H), 4.23-4.13 (m, 1H), 3.82 (d, J=13.6 Hz, 1H), 3.74-3.69 (m, 2H), 3.54-3.39 (m, 2H), 3.33-3.24 (m, 2H), 2.88-2.73 (m, 2H), 1.40 (s, 3H), 1.12 (s, 3H).

### Intermediate S1-B: 2,2-Dimethyl-3-(3-thioxohexahydroimidazo[1,5-a]pyrazin-2(3H)-yl)propanoic acid hydrochloride

(R)-3-(7-(tert-butoxycarbonyl)-3-thioxohexahydroimidazo[1,5-a]pyrazin-2(3H)-yl)-2,2-dimethylpropanoic acid S1-6B (650 mg, 1.64 mmol) was added into 3 M hydrochloride in ethyl acetate (20 mL, 60 mmol). The reaction was stirred at room temperature under nitrogen atmosphere for 4 hours, the completed reaction was concentrated under reduced pressure to give the title compound Intermediate S1-B (530 mg, 90% purity from ¹HNMR, 99% yield) as white solids. LC-MS (ESI): R_{T}=0.82 min, mass calcd. for C₁₁H₂₀ClN₃O₂S 293.1, m/z found 258.1 [M+H—HCl]⁺. ¹H NMR (400 MHz, DMSO-d₆) δ 12.52 (br s, 1H), 9.41 (br s, 2H), 4.40-4.36 (m, 1H), 4.21-4.10 (m, 1H), 3.83 (d, J=14.0 Hz, 1H), 3.74-3.69 (m, 2H), 3.39-3.35 (m, 2H), 3.28-3.24 (m, 2H), 2.91-2.77 (m, 2H), 1.41 (s, 3H), 1.12 (s, 3H).

### Preparation of Compounds

### Compound 1A: 3-(7-(((S)-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid

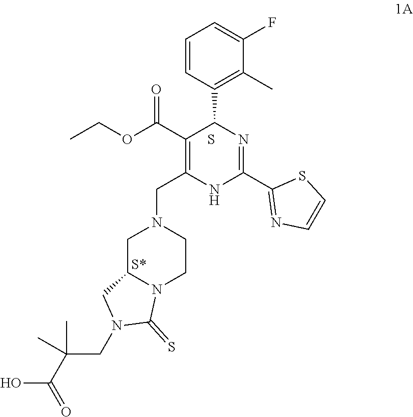

To the solution of 2,2-dimethyl-3-(3-thioxohexahydroimidazo[1,5-a]pyrazin-2 (3H)-yl)propanoic acid hydrochloride Intermediate S1-A (1.87 g, 5.73 mmol) in tetrahydrofuran (160 mL) was added triethylamine (3.4 mL, 24.5 mmol). The mixture was stirred at room temperature for 10 minutes before (S)-ethyl 6-(bromomethyl)-4-(3-fluoro-2-methylphenyl)-2-(thiazol-2-yl)-1,4-dihydropyrimidine-5-carboxylate (112-1A) (2.5 g, 5.14 mmol) was added. After stirred at 40° C. under nitrogen atmosphere for 2.5 hours and then stirred at room temperature overnight, the mixture was filtered and the filtrate was concentrated and purified by C18 column (acetonitrile:water (+0.05% hydrochloride)=45% 50%) to give the desired compound (1.69 g, 48% yield) as light yellow solids. LC-MS (ESI): R_{T}=8.325 min, mass calcd. for C₂₉H₃₅FN₆O₄S₂ 614.8, m/z found 615.2 [M+H]⁺. Chiral analysis: (Column: Chiralpak IE 5 μm 4.6*250 mm; Mobile Phase: Hex:IPA:TFA=50:50:0.2 at 1 mL/min; Temp: 30° C.; Wavelength: 254 nm, R_{T}=11.063 min). H NMR (400 MHz, DMSO-d₆) δ 12.45 (s, 1H), 9.58 (s, 0.9H), 9.53 (d, J=3.2 Hz, 0.1H), 8.01-7.92 (m, 2H), 7.21-7.16 (m, 1H), 7.06-7.01 (m, 2H), 5.88 (s, 0.9H), 5.77 (d, J=3.2 Hz, 0.1H), 4.35 (d, J=11.6 Hz, 0.9H), 4.22 (d, J=14 Hz, 0.1H), 4.02-3.88 (m, 5H), 3.81-3.73 (m, 2H), 3.66-3.61 (m, 1H), 3.18-3.12 (m, 2H), 3.06-3.03 (m, 0.1H), 2.95-2.89 (m, 1.9H), 2.45 (d, J=1.6 Hz, 2.8H), 2.39 (d, J=1.6 Hz, 0.2H), 2.27 (dt, J=11.6, 3.2 Hz, 1H), 2.07 (t, J=10.8 Hz, 1H), 1.13-1.04 (m, 9H).

### Compound IB: 3-(7-(((S)-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

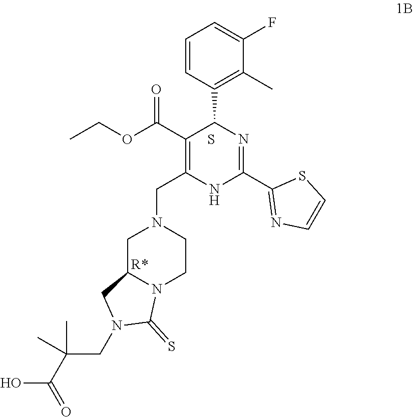

Compound 1B was prepared using H2-1A and S1-B under condition for compound 1A, purified by Prep-HPLC (Column: Xbridge C18 (5 μm 19*150 mm), Mobile Phase A: water (0.1% ammonium bicarbonate), Mobile Phase B: acetonitrile, UV: 214 nm, Flow rate: 15 mL/min, Gradient: 30-75% (% B)), LC-MS (ESI): R_{T}=3.915 min, mass calcd. for C₂₉H₃₅FN₆O₄S₂ 614.2, m/z found 615.2 [M+H]⁺. Chiral analysis: (Column: Chiralpak IE 5 μm 4.6*250 mm; Mobile Phase: Hex:EtOH:TFA=70:30:0.2 at 1 mL/min; Temp: 30° C.; Wavelength: 254 nm, R_{T}=19.029 min). ¹H NMR (400 MHz, DMSO-d₆) δ 12.22 (br s, 1H), 9.62 (s, 1H), 8.01-7.99 (m, 1H), 7.94 (d, J=2.8 Hz, 1H), 7.21-7.15 (m, 1H), 7.07-7.02 (m, 2H), 5.89 (s, 0.9H), 5.76 (s, 0.1H), 4.30-4.27 (m, 1H), 4.04-3.89 (m, 5H), 3.82-3.74 (m, 2H), 3.72-3.67 (m, 1H), 3.22-3.17 (m, 1H), 3.14-3.04 (m, 2H), 2.78-2.75 (m, 1H), 2.45 (s, 3H), 2.22-2.12 (m, 2H), 1.14 (s, 3H), 1.13 (s, 3H), 1.05 (t, J=7.2 Hz, 3H).

### Compound 2: 3-(3-(cyanoimino)-7-(((S)-5-(ethoxycarbonyl)-6-(3-fluoro-2-methylphenyl)-2-(thiazol-2-yl)-3,6-dihydropyrimidin-4-yl)methyl)hexahydroimidazo[1,5-a]pyrazin-2(3H)-yl)-2,2-dimethylpropanoic acid (Mixture of 2 diastereomers)

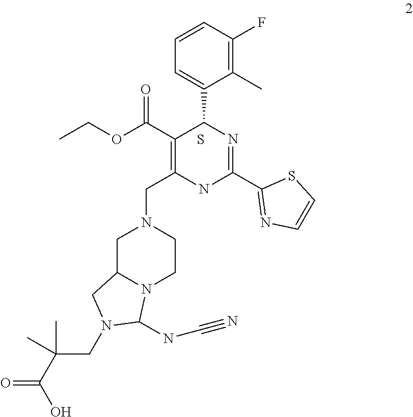

### Preparation of Intermediate S2:3-(3-(cyanoimino)hexahydroimidazo[1,5-a]pyrazin-2(3H)-yl)-2,2-dimethylpropanoic acid

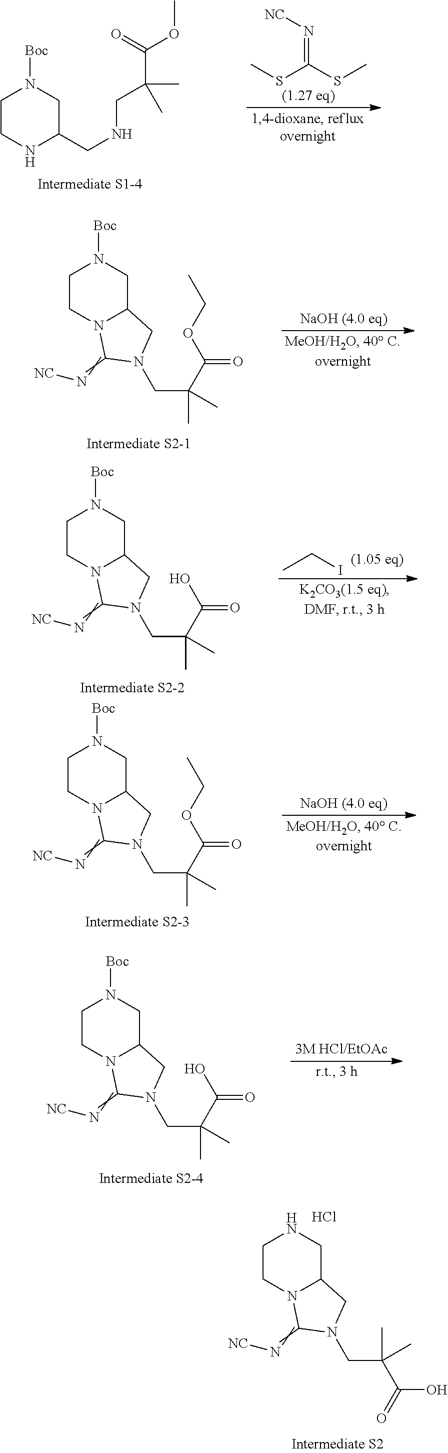

### Intermediate S2-1: tert-Butyl 3-(cyanoimino)-2-(3-ethoxy-2,2-dimethyl-3-oxopropyl)hexahydroimidazo[1,5-a]pyrazine-7(1H)-carboxylate

To a solution of tert-butyl 3-(((3-ethoxy-2,2-dimethyl-3-oxopropyl)amino)methyl)piperazine-1-carboxylate S1-4 (3.00 g, 6.99 mmol) in 1,4-dioxane (30 mL) was added dimethyl cyanocarbonimidodithioate (1.30 g, 8.89 mmol). After heated to reflux and stirred overnight, the reaction mixture was cooled down to room temperature and diluted with water (150 mL). The mixture was extracted with ethyl acetate (50 mL) twice. The combined organic layers were washed with brine (50 mL), dried over Na₂SO₄₍ₛ₎, filtered and concentrated to afford a crude product (4.00 g, 83% yield) as yellow oil. LC-MS (ESI): R_{T}=1.62 min, mass calcd. for C₁₉H₃₁N₅O₄ 393.2, m/z found 394.2 [M+H]⁺.

### Intermediate S2-2:3-(7-(tert-Butoxycarbonyl)-3-(cyanoimino)hexahydroimidazo-[1,5-a]pyrazin-2(3H)-yl)-2,2-dimethylpropanoic acid

To a solution of tert-butyl 3-(cyanoimino)-2-(3-ethoxy-2,2-dimethyl-3-oxopropyl)hexahydroimidazo[1,5-a]pyrazine-7(1H)-carboxylate S2-1 (4.68 g, 6.66 mmol) in methanol (50 mL) was added a solution of sodium hydroxide (1.10 g, 27.5 mmol) in water (20 mL) at 0° C. After stirred at 40° C. overnight, method was removed and the remained aqueous phase was extracted with ethyl acetate (50 mL). The aqueous layer was separated and acidified by 2 M hydrochloride solution to pH ˜3, then extracted with ethyl acetate (50 mL) twice. The combined organic layers were dried over Na₂SO₄₍ₛ₎, filtered and concentrated to give the crude product (2.60 g, 86% purity, 91% yield) as white solids. LC-MS (ESI): R_{T}=1.46 min, mass calcd. for C₁₇H₂₇N₅O₄ 365.2, m/z found 366.2 [M+H]⁺.

### Intermediate S2-3: tert-Butyl 3-(cyanoimino)-2-(3-ethoxy-2,2-dimethyl-3-oxopropyl)hexahydroimidazo[1,5-a]pyrazine-7(1H)-carboxylate

To a mixture of 3-(7-(tert-butoxycarbonyl)-3-(cyanoimino)hexahydroimidazo[1,5-a]pyrazin-2(3H)-yl)-2,2-dimethylpropanoic acid S2-2 (2.60 g, 6.12 mmol) and potassium carbonate (1.30 g, 9.41 mmol) in N,N-dimethylformamide (30 mL) at 0° C. was added iodoethane (1.00 g, 6.41 mmol) by dropwise. After stirred at room temperature for 3 hours, the mixture was diluted with water (150 mL), extracted with ethyl acetate (150 mL) twice. The combined extracts were washed with brine (150 ml) twice, dried over Na₂SO₄₍ₛ₎, filtered and concentrated to give the crude product, which was purified by C₁₈ (acetonitrile:water=5% to 45%) to give the title compound (2.40 g, 89% yield) as white solids. LC-MS (ESI): R_{T}=1.60 min, mass calcd. for C₁₉H₃₁N₅O₄ 393.2, m/z found 394.3 [M+H]⁺. ¹H NMR (400 MHz, CDCl₃) δ 4.64 (d, J=11.6 Hz, 1H), 4.31-3.96 (m, 4H), 3.65-3.52 (m, 4H), 3.11-3.03 (m, 2H), 2.89-2.56 (m, 2H), 1.47 (s, 9H), 1.28 (t, J=7.2 Hz, 3H), 1.22 (s, 6H).

### Intermediate S2-4:3-(7-(tert-Butoxycarbonyl)-3-(cyanoimino)hexahydroimidazo-[1,5-a]pyrazin-2(3H)-yl)-2,2-dimethylpropanoic acid

To a solution of tert-butyl 3-(cyanoimino)-2-(3-ethoxy-2,2-dimethyl-3-oxopropyl)hexahydroimidazo[1,5-a]pyrazine-7(1H)-carboxylate S2-3 (500 mg, 1.14 mmol) in methanol (20 mL) at 0° C. was added a solution of sodium hydroxide (180 mg, 4.50 mmol) in water (10 mL). After stirred at 40° C. overnight, methanol was removed and the remained aqueous phase was extracted with ethyl acetate (30 mL). The aqueous phase was separated and acidified by 2 M hydrochloride aqueous solution to pH ˜3, extracted with ethyl acetate (50 mL) twice. The combined extracts were dried over Na₂SO₄₍ₛ₎, filtered and concentrated to give the crude product (400 mg, 92% yield) as white solids. LC-MS (ESI): R_{T}=1.21 min, mass calcd. for C₁₇H₂₇N₅O₄ 365.2, m/z found 364.2 [M−H]⁺.

### Intermediate S2:3-(3-(Cyanoimino)hexahydroimidazo[1,5-a]pyrazin-2(3H)-yl)-2,2-dimethylpropanoic acid hydrochloride

A mixture of 3-(7-(tert-butoxycarbonyl)-3-(cyanoimino)hexahydroimidazo[1,5-a]pyrazin-2(3H)-yl)-2,2-dimethylpropanoic acid S2-4 (150 mg, 0.398 mmol) in 3 M hydrochloride in ethyl acetate (6 mL, 18.0 mmol) was stirred at room temperature for 3 hours. Then the mixture was concentrated to give the desired product (120 mg, 99% yield) as white solids. The crude product was used for next step directly. LC-MS (ESI): R_{T}=0.87 min, mass calcd. for C₁₂H₂₀ClN₅O₂ 301.1, m/z found 266.2 [M+H—HCl]⁺.

### Compound 2: 3-(3-(Cyanoimino)-7-((5-(ethoxycarbonyl)-6-(3-fluoro-2-methylphenyl)-2-(thiazol-2-yl)-3,6-dihydropyrimidin-4-yl)methyl)-hexahydroimidazo[1,5-a]pyrazin-2(3H)-yl)-2,2-dimethylpropanoic acid (mixture of 2 diastereomers)

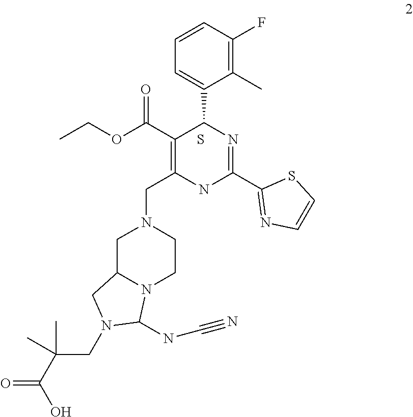

To a mixture of 3-(3-(cyanoimino)hexahydroimidazo[1,5-a]pyrazin-2(3H)-yl)-2,2-dimethylpropanoic acid hydrochloride S2 (120 mg, 0.398 mmol) in dichloromethane (10 mL) was added triethanolamine (300 mg, 2.01 mmol). After stirred for 0.5 hour at room temperature, (S)-ethyl 6-(bromomethyl)-4-(3-fluoro-2-methylphenyl)-2-(thiazol-2-yl)-1,4-dihydropyrimidine-5-carboxylate (112-1A) (150 mg, 0.308 mmol) was added. After stirred at room temperature overnight, the reaction mixture was diluted by dichloromethane (50 mL), washed by brine (50 mL) twice, dried over Na₂SO₄₍ₛ₎, filtered and concentrated to give a residue, which was purified was by C18 column (acetonitrile:water=5% to 45%) to give the title compound (48 mg, 97.4% purity, 18% yield) as yellow solids. LC-MS (ESI): R_{T}=3.677 min, mass calcd. for C₃₀H₃₅FN₈O₄S 622.3, m/z found 623.3 [M+H]⁺. ¹H NMR (400 MHz, DMSO-d₆) δ 9.60-9.52 (m, 1H), 8.01-8.00 (m, 1H), 7.93-7.92 (m, 1H), 7.21-7.15 (m, 1H), 7.06-7.01 (m, 2H), 5.89-5.88 (m, 1H), 4.47-4.36 (m, 1H), 4.04-3.91 (m, 4H), 3.85-3.76 (m, 1H), 3.64-3.43 (m, 3H), 3.22-2.91 (m, 4H), 2.45 (s, 3H), 2.39-2.13 (m, 2H), 1.13-1.04 (m, 9H).

### Compound 3A: 3-(7-((6-(2-chloro-3-fluorophenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

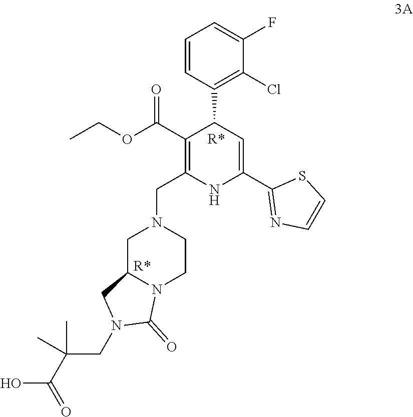 [SMILES: CCOC(=O)C1=C(CN2CCN3C(=O)N(CC(C)(C)C(=O)O)C[C@H]3C2)NC(c2nccs2)=C[C@H]1c1cccc(F)c1Cl]

Compound 3A was prepared from H1-1A and Intermediate S1-B using same condition as for Compound 3B.

Compound 3A: purified by Prep-HPLC (Column: gilson Xbrige C18 (5 μm 19*150 mm), Mobile phase A: water (+0.1% ammonium bicarbonate), Mobile phase B: acetonitrile, UV: 214 nm, Flow rate: 15 mL/min, Gradient: 10-70% (% B)) to give the title compound (30 mg, 99.6% purity, 31% yield) as yellow solids. LC-MS (ESI): R_{T}=3.262 min, mass calcd. for C₂₈H₃₂ClFN₆O₄S₂ 634.2, m/z found 635.2. ¹H NMR (400 MHz, DMSO-d₆) δ 9.67 (s, 1H), 8.03 (d, J=3.2 Hz, 1H), 7.95 (d, J=3.2 Hz, 1H), 7.38-7.25 (m, 3H), 6.11 (s, 0.97H), 6.00 (s, 0.03H), 4.31-4.28 (m, 1H), 4.02-3.89 (m, 5H), 3.82-3.74 (m, 2H), 3.72-3.67 (m, 1H), 3.22-3.18 (m, 1H), 3.15-3.04 (m, 2H), 2.81-2.78 (m, 1H), 2.21-2.14 (m, 2H), 1.14 (s, 6H), 1.03 (t, J=7.2 Hz, 3H).

### Compound 3B: 3-(7-((6-(2-chloro-3-fluorophenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

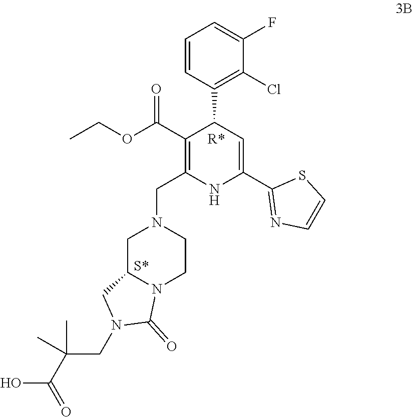 [SMILES: CCOC(=O)C1=C(CN2CCN3C(=O)N(CC(C)(C)C(=O)O)C[C@@H]3C2)NC(c2nccs2)=C[C@H]1c1cccc(F)c1Cl]

To a solution of 2,2-dimethyl-3-(3-thioxohexahydroimidazo[1,5-a]pyrazin-2(3H)-yl)propanoic acid hydrochloride Intermediate S1-A (100 mg, 0.31 mmol) in dichloromethane (3 mL) was added triethanolamine (230 mg, 1.54 mmol). After stirred at 40° C. for 30 minutes, a solution of ethyl 6-(bromomethyl)-4-(2-chloro-3-fluorophenyl)-2-(thiazol-2-yl)-1,4-dihydropyrimidine-5-carboxylate (H1-1A) (157 mg, 90% purity, 0.279 mmol) in dichloromethane (2 mL) was added dropwise. After stirred at 40° C. for 16 hours, the reaction mixture was concentrated to give a residue, which was purified by Prep-HPLC (Column: Waters Xbridge C18 (5 μm 19*150 mm), Mobile Phase A: Water (0.1% ammonium bicarbonate), Mobile Phase B: acetonitrile, UV: 214 nm, Flow rate: 15 mL/min, Gradient: 20-60% (% B)) give the title compound 3B (34.8 mg, 17.8% yield) as yellow solids. LC-MS (ESI): R_{T}=3.542 min, mass calcd. for C₂₈H₃₂ClFN₆O₄S₂ 634.2, m/z found 635.2 [M+H]⁺. ¹H NMR (400 MHz, DMSO-d₆) δ 9.67 (br s, 1H), 8.02 (d, J=3.2 Hz, 1H), 7.94 (d, J=3.2 Hz, 1H), 7.39-7.29 (m, 2H), 7.29-7.24 (m, 1H), 6.10 (s, 1H), 4.35 (d, J=11.6 Hz, 1H), 4.00-3.87 (m, 5H), 3.78 (d, J=14.0 Hz, 1H), 3.74 (d, J=14.0 Hz, 1H), 3.64 (t, J=9.6 Hz, 1H), 3.19-3.12 (m, 2H), 2.95-2.92 (m, 2H), 2.32-2.21 (m, 1H), 2.08 (t, J=10.8 Hz, 1H), 1.12 (s, 6H), 1.05 (t, J=7.2 Hz, 3H).

### Compound 4A: 3-(7-((6-(2-chloro-4-fluorophenyl)-5-(methoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

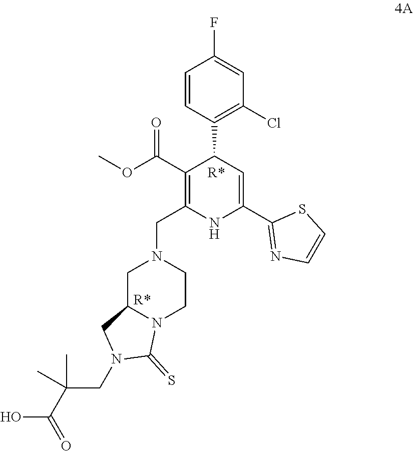 [SMILES: COC(=O)C1=C(CN2CCN3C(=S)N(CC(C)(C)C(=O)O)C[C@H]3C2)NC(c2nccs2)=C[C@H]1c1ccc(F)cc1Cl]

Compound 4A was prepared from Intermediate S1-B and intermediate H3-1A using same condition as for Compound 4B and purified by Prep-HPLC (Column: gilson Xbrige C18 (5 μm 19*150 mm), Mobile phase A: water (+0.1% ammonium bicarbonate), Mobile phase B: acetonitrile, UV: 214 nm, Flow rate: 15 mL/min, Gradient: 05-95% (% B)). LC-MS (ESI): R_{T}=3.658 min, mass calcd. for C₂₇H₃₀ClFN₆O₄S₂ 620.1, m/z found 621.1. ¹H NMR (400 MHz, DMSO-d₆) δ 9.71 (br s, 0.9H), 8.03 (d, J=3.2 Hz, 1H), 8.01 (s, 0.1H), 7.95 (d, J=3.2 Hz, 1H), 7.45-7.39 (m, 2H), 7.17 (td, J=8.4, 2.4 Hz, 1H), 6.05 (s, 0.97H), 5.93 (s, 0.03H), 4.30-4.27 (m, 1H), 4.02-3.89 (m, 3H), 3.81-3.67 (m, 3H), 3.52 (s, 3H), 3.22-3.18 (m, 1H), 3.15-3.04 (m, 2H), 2.79-2.76 (m, 1H), 2.22-2.14 (m, 2H), 1.13 (s, 6H).

### Compound 4B: 3-(7-((6-(2-chloro-4-fluorophenyl)-5-(methoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

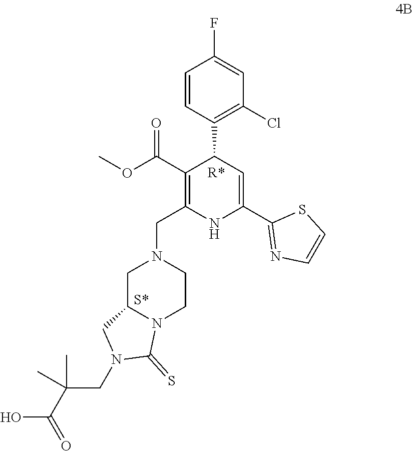 [SMILES: COC(=O)C1=C(CN2CCN3C(=S)N(CC(C)(C)C(=O)O)C[C@@H]3C2)NC(c2nccs2)=C[C@H]1c1ccc(F)cc1Cl]

To a solution of 2,2-dimethyl-3-(3-thioxohexahydroimidazo[1,5-a]pyrazin-2(3H)-yl)propanoic acid hydrochloride Intermediate S1-A (80 mg, 0.250 mmol) in dichloromethane (3 mL) was added triethanolamine (184 mg, 1.23 mmol) at room temperature and the resulting mixture was stirred at 40° C. for 30 minutes. Then a solution of (R)-methyl 6-(bromomethyl)-4-(2-chloro-4-fluorophenyl)-2-(thiazol-2-yl)-1,4-dihydropyrimidine-5-carboxylate (113-A) (122 mg, 0.250 mmol) in dichloromethane (2 mL) was added dropwise. After stirred at 40° C. for 16 hours, the reaction mixture was concentrated to give a residue, which was purified by Prep-HPLC (Column: Waters Xbridge C18 (5 μm 19*150 mm), Mobile Phase A: water (0.1% ammonium bicarbonate), Mobile Phase B: acetonitrile, UV: 214 nm, Flow rate: 15 mL/min, Gradient: 20-55% (% B)) give the title compound (6.1 mg, 99.3% purity, 4% yield) as yellow solids. LC-MS (ESI): R_{T}=3.754 min, mass calcd. for C₂₇H₃₀ClFN₆O₄S₂ 620.1, m/z found 621.2 [M+H]⁺. ¹H NMR (400 MHz, CD₃OD) δ7.84 (d, J=2.8 Hz, 1H), 7.64 (d, J=3.6 Hz, 1H), 7.31 (dd, J=8.8, 6.0 Hz, 1H), 7.12 (dd, J=8.8, 2.8 Hz, 1H), 6.97-6.92 (m, 1H), 6.05 (s, 1H), 4.43-4.39 (m, 1H), 4.01-3.93 (m, 2H), 3.84-3.73 (m, 3H), 3.60-3.56 (m, 1H), 3.49 (s, 3H), 3.18-3.11 (m, 2H), 2.87-2.77 (m, 2H), 2.35-2.30 (m, 1H), 2.10-2.04 (m, 1H), 1.13 (s, 3H), 1.12 (s, 3H).

### Compound 5: 1-((7-((6-(2-Chloro-3-fluorophenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)methyl)cyclopropanecarboxylic acid (mixture of two diasteromers)

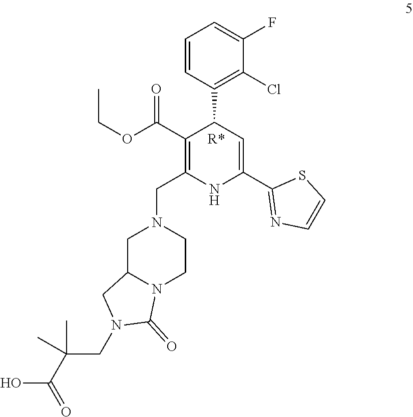 [SMILES: CCOC(=O)C1=C(CN2CCN3C(=O)N(CC(C)(C)C(=O)O)CC3C2)NC(c2nccs2)=C[C@H]1c1cccc(F)c1Cl]

### Preparation of Intermediate S3

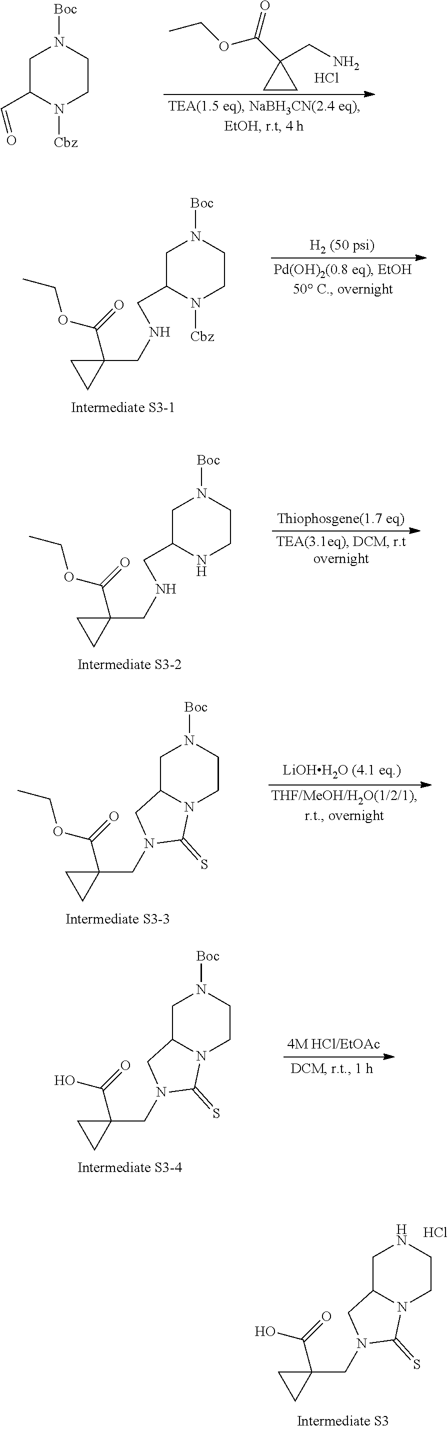

### Intermediate S3-1:1-Benzyl 4-tert-butyl 2-((((1-(ethoxycarbonyl)cyclopropyl)methyl)amino)methyl) piperazine-1,4-dicarboxylate

To a solution of ethyl 1-(aminomethyl)cyclopropanecarboxylate hydrochloride (2.04 g, 11.4 mmol) in ethanol (50 mL) was added triethylamine (1.15 g, 11.4 mmol) at room temperature. After stirred at room temperature under nitrogen atmosphere for 0.5 hour, a solution of 1-benzyl 4-tert-butyl 2-formylpiperazine-1,4-dicarboxylate S1-2 (3.10 g, 7.56 mmol) in ethanol (10 mL) was added and stirred at room temperature for 1.5 hours. Then sodium cyanoborohydride (1.12 g, 17.8 mmol) was added at 0° C. After stirred at room temperature for 2 hours, the mixture was quenched with ice water (15 mL), then removed ethanol under vacuo. The residue was diluted with water (40 mL) and extracted with ethyl acetate (20 mL) for three times. The combined organic layers were dried over Na2SO4(s) and filtered. The filtrate was concentrated to give a residue, which was purified by C18 column (acetonitrile:water=65% to 95%) to give the title compound (2.00 g, 50% yield) as yellow oil. LC-MS (ESI): RT=1.767 min, mass calcd. for C₂₅H₃₇N₃O₆ 475.3, m/z found 476.3 [M+H]⁺. 1H NMR (400 MHz, CDCl3) δ 7.36-7.32 (m, 5H), 5.14 (s, 1H), 4.27-3.93 (m, 6H), 3.05-2.66 (m, 7H), 1.71 (br s, 1H), 1.46 (s, 9H), 1.23-1.19 (m, 5H), 0.81-0.68 (m, 2H).

### Intermediate S3-2: tert-Butyl 3-((((1-(ethoxycarbonyl)cyclopropyl)-methyl)amino)-methyl)piperazine-1-carboxylate

To a solution of 1-benzyl 4-tert-butyl 2-((((1-(ethoxycarbonyl)cyclopropyl)-methyl)amino)methyl)piperazine-1,4-dicarboxylate (S3-1) (1.80 g, 3.41 mmol) in ethanol (80 mL) was added 20% palladium hydroxide on carbon (2.00 g, 2.85 mmol) under nitrogen atmosphere. After stirred at 50° C. under hydrogen atmosphere (50 psi) overnight, the mixture was cooled to room temperature. Then the catalyst was filtered, and the filtrate was concentrated to give the desired compound (1.10 g, 85% yield) as yellow oil. LC-MS (ESI): RT=1.374 min, mass calcd. for C₁₇H₃₁N₃O₄ 341.2, m/z found 342.2 [M+H]⁺. ¹H NMR (300 MHz, CDCl³) δ 4.13 (q, J=7.2 Hz, 2H), 3.93-3.90 (m, 1H), 3.00-2.43 (m, 8H), 2.25 (br s, 2H), 1.46 (s, 9H), 1.26-1.21 (m, 4.6H), 0.81-0.77 (m, 1.4H).

### Intermediate S3-3: tert-Butyl 2-((1-(ethoxycarbonyl)cyclopropyl)methyl)-3-thioxohexahydroimidazo[1,5-a]pyrazine-7(1H)-carboxylate

To a solution of tert-butyl 3-((((1-(ethoxycarbonyl)cyclopropyl)methyl)amino)-methyl)piperazine-1-carboxylate (S3-2) (1.10 g, 2.90 mmol) and triethylamine (900 mg, 8.89 mmol) in dichloromethane (25 mL) was added a solution of thiophosgene (550 mg, 4.78 mmol) in dichloromethane (5 mL) at 0° C. under nitrogen atmosphere. After stirred at room temperature overnight, the mixture was diluted with ice water (40 mL) and extracted with dichloromethane (10 mL) for three times. The combined organic layers were washed with brine (20 mL), dried over Na2SO4(s) and filtered. The filtrate was concentrated to give a residue, which was purified by silica gel column chromatography (petroleum ether:ethyl acetate=8:1 to 2:1) to give the crude compound, which was further purified by C18 column (acetonitrile:water=45% to 95%) to give the title compound (650 mg, 53% yield) as yellow solids. LC-MS (ESI): RT=1.701 min, mass calcd. for C₁₈H₂₉N₃O₄S 383.2, m/z found 384.2 [M+H]⁺. 1H NMR (400 MHz, CDCl₃) δ 4.44 (d, J=11.6 Hz, 1H), 4.15-4.10 (m, 4H), 3.97 (s, 2H), 3.87-3.82 (m, 1H), 3.78-3.71 (m, 1H), 3.30-3.26 (m, 1H), 3.04-2.98 (m, 1H), 2.86-2.81 (m, 1H), 2.65-2.58 (m, 1H), 1.47 (s, 9H), 1.31 (s, 2H), 1.26-1.19 (m, 5H).

### Intermediate S3-3A and S3-3B

A racemic mixture of tert-butyl 2-((1-(ethoxycarbonyl)cyclopropyl)methyl)-3-thioxohexahydroimidazo[1,5-a]pyrazine-7(1H)-carboxylate S3-3 (400 mg, 0.939 mmol) was separated by chiral Prep-HPLC (separation condition: Column: Chiralpak ID 5 μm 20*250 mm; Mobile Phase: Hex:EtOH:DEA=85:15:0.3 at 18 mL/min; Temp: 35° C.; Wavelength: 214 nm) to give the title compounds S3-3A (90 mg, 90% purity from 1H NMR, 23% yield, 100% stereopure) and S3-3B (204 mg, 90% purity from 1H NMR, 51% yield, 99.2% stereopure).

Intermediate S3-3A: LC-MS (ESI): RT=1.71 min, mass calcd. for C₁₈H₂₉N₃O₄S 383.2, m/z found 384.1 [M+H]⁺. Chiral analysis (Column: Chiralpak IE 5 um 4.6*250 mm; Mobile Phase: Hex:EtOH:DEA=85:15:0.2 at 1 mL/min; Temp: 30° C.; Wavelength: 254 nm, RT=15.778 min). 1H NMR (400 MHz, CDCl3) δ 4.46-4.43 (m, 1H), 4.26-4.03 (m, 4H), 3.97 (s, 2H), 3.87-3.82 (m, 1H), 3.80-3.68 (m, 1H), 3.31-3.26 (m, 1H), 3.05-2.98 (m, 1H), 2.89-2.78 (m, 1H), 2.69-2.54 (m, 1H), 1.47 (s, 9H), 1.32-1.19 (m, 7H).

Intermediate S3-3B: LC-MS (ESI): RT=1.71 min, mass calcd. for C₁₈H₂₉N₃O₄S 383.2, m/z found 384.1 [M+H]⁺. Chiral analysis (Column: Chiralpak IE 5 um 4.6*250 mm; Mobile Phase: Hex:EtOH:DEA=85:15:0.2 at 1 mL/min; Temp: 30° C.; Wavelength: 254 nm, RT=18.449 min). 1H NMR (400 MHz, CDCl3) δ 4.46-4.43 (m, 1H), 4.27-4.02 (m, 4H), 3.97 (s, 2H), 3.85-3.82 (m, 1H), 3.78-3.68 (m, 1H), 3.31-3.26 (m, 1H), 3.05-2.98 (m, 1H), 2.92-2.77 (m, 1H), 2.70-2.55 (m, 1H), 1.47 (s, 9H), 1.32-1.19 (m, 7H).

### Intermediate S3-4:1-((7-(tert-Butoxycarbonyl)-3-thioxohexahydroimidazo[1,5-a]pyrazin-2(3H)-yl)methyl)cyclopropanecarboxylic acid

To a solution of tert-butyl 2-((1-(ethoxycarbonyl)cyclopropyl)methyl)-3-thioxohexahydroimidazo[1,5-a]pyrazine-7(1H)-carboxylate (S3-3) (100 mg, 0.235 mmol) in tetrahydrofuran (1 mL), methanol (2 mL) and water (1 mL) was added lithium hydroxide monohydrate (40 mg, 0.953 mmol) under nitrogen atmosphere. After stirred at room temperature overnight, the reaction was concentrated at 35° C. to give a residue, which was purified by C18 column (acetonitrile:water=30% to 90%) to give the desired compound (88 mg) as light yellow solids. LC-MS (ESI): RT=1.24 min, mass calcd. for C₁₆H₂₅N₃O₄S 355.2, m/z found 356.2 [M+H]⁺.

Intermediate S3-4A was prepared from S3-3A using same condition as for S3-4. LC-MS (ESI): R_{T}=1.21 min, mass calcd. for C₁₆H₂₅N₃O₄S 355.2, m/z found 356.1 [M+H]⁺.

Intermediate S3-4B was prepared from S3-3B using same condition as for S3-4. LC-MS (ESI): R_{T}=1.24 min, mass calcd. for C₁₆H₂₅N₃O₄S 355.2, m/z found 356.1 [M+H]⁺.

### Intermediate S3: 1-((3-Thioxohexahydroimidazo[1,5-a]pyrazin-2(3H)-yl)methyl)cyclopropanecarboxylic acid hydrochloride

To a solution of 1-((7-(tert-butoxycarbonyl)-3-thioxohexahydroimidazo[1,5-a]pyrazin-2(3H)-yl)methyl)cyclopropanecarboxylic acid (S3-4) (88 mg, 0.235 mmol) in dichloromethane (3 mL) was added 4 M hydrochloride in ethyl acetate (2 mL, 8 mmol) under nitrogen atmosphere. After stirred at room temperature for 1 hour, the reaction mixture was concentrated to give the title compound (63 mg, 78% yield) as white solids. ¹H NMR (400 MHz, CD₃OD) δ 4.67-4.63 (m, 0.5H), 4.62-4.60 (m, 0.5H), 4.21-4.12 (m, 1H), 3.99-3.88 (m, 3H), 3.59-3.34 (m, 4H), 3.06-2.85 (m, 2H), 1.31-1.26 (m, 2H), 1.18-1.13 (m, 2H).

Intermediate S3A was prepared from S3-4A using same condition as for intermediate S3. ¹H NMR (400 MHz, DMSO-d₆) δ 12.61-12.13 (m, 1H), 10.14-9.27 (m, 2H), 4.37-4.33 (m, 1H), 4.25-4.11 (m, 1H), 3.85-3.78 (m, 2.4H), 3.73-3.65 (m, 0.6H), 3.40-3.29 (m, 4H), 2.87-2.69 (m, 2H), 1.18-1.10 (m, 2H), 1.09-1.02 (m, 2H).

Intermediate S3B was prepared from S3-4B using same condition as for intermediate S3. H NMR (400 MHz, DMSO-d₆) δ 12.77-12.05 (m, 1H), 10.16-9.64 (m, 2H), 4.39-4.32 (m, 1H), 4.26-4.15 (m, 1H), 3.85-3.77 (m, 2.4H), 3.72-3.65 (m, 0.6H), 3.47-3.29 (m, 4H), 2.85-2.70 (m, 2H), 1.16-1.14 (m, 2H), 1.07-1.01 (m, 2H).

### Compound 5: 1-((7-((6-(2-Chloro-3-fluorophenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)methyl)cyclopropanecarboxylic acid (mixture of two diasteromers)

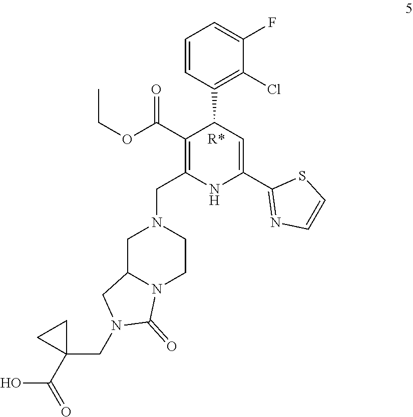 [SMILES: CCOC(=O)C1=C(CN2CCN3C(=O)N(CC4(C(=O)O)CC4)CC3C2)NC(c2nccs2)=C[C@H]1c1cccc(F)c1Cl]

To a solution of ethyl 6-(bromomethyl)-4-(2-chloro-3-fluorophenyl)-2-(thiazol-2-yl)-1,4-dihydropyrimidine-5-carboxylate (H1-1A) (110 mg, 0.216 mmol) in tetrahydrofuran (3 ml) was added 1-((3-thioxohexahydroimidazo[1,5-a]pyrazin-2(3H)-yl)methyl)cyclopropanecarboxylic acid hydrochloride (S3) (63 mg, 0.194 mmol) and triethylamine (110 mg, 1.09 mmol) under nitrogen atmosphere. After stirred at 40° C. under nitrogen atmosphere for 2.5 hours and then stirred at room temperature overnight, the reaction mixture was diluted with water (10 mL) and extracted with ethyl acetate (10 mL) twice. The combined organic layers were washed with brine (10 mL), dried over Na₂SO₄₍ₛ₎, filtered and concentrated to give a residue, which was purified by C18 column (acetonitrile:water=40% to 70%) to give the title compound (24.2 mg, 17% yield) as yellow solids. LC-MS (ESI): RT=3.723 min, mass calcd. for C₂₈H₃₀ClFN₆O₄S₂ 632.1, m/z found 633.2 [M+H]⁺. H NMR (400 MHz, CD₃OD) δ 7.85 (d, J=3.2 Hz, 1H), 7.64 (d, J=3.2 Hz, 1H), 7.22-7.14 (m, 2H), 7.06-7.02 (m, 1H), 6.13 (s, 0.4H), 6.12 (s, 0.6H), 4.41-4.37 (m, 0.6H), 4.34-4.30 (m, 0.4H), 4.05-3.90 (m, 4H), 3.85-3.70 (m, 4H), 3.34-3.25 (m, 1.2H), 3.18-3.13 (m, 0.8H), 2.99-2.94 (m, 0.4H), 2.87-2.81 (m, 1H), 2.79-2.66 (m, 0.6H), 2.36-2.30 (m, 0.5H), 2.24-2.05 (m, 1.5H), 1.21-1.15 (m, 2H), 1.07-0.99 (m, 5H).

### Compound 6: 3-((S)-2-(((S)-5-(ethoxycarbonyl)-6-(3-fluoro-2-methylphenyl)-2-(thiazol-2-yl)-3,6-dihydropyrimidin-4-yl)methyl)-6-thioxohexahydro-2H-pyrazino[1,2-c]pyrimidin-7(6H)-yl)-2,2-dimethylpropanoic acid

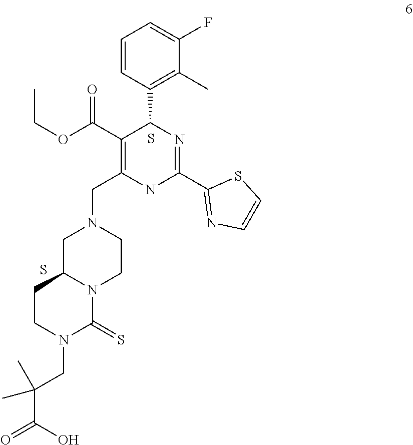

### Preparation of Intermediate S4: (S)-2,2-Dimethyl-3-(6-thioxotetrahydro-1H-pyrazino[1,2-c]pyrimidin-7(2H,6H,8H)-yl)propanoic acid hydrochloride

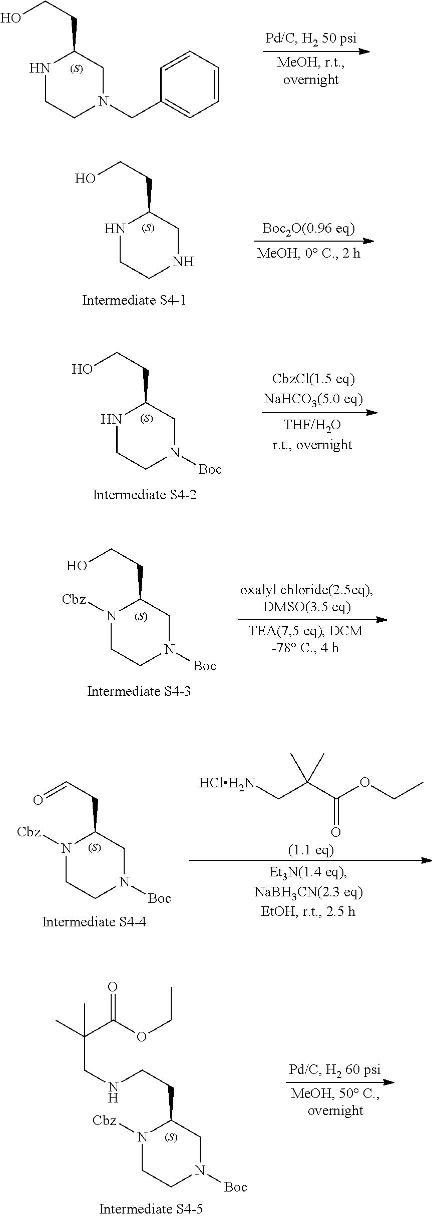

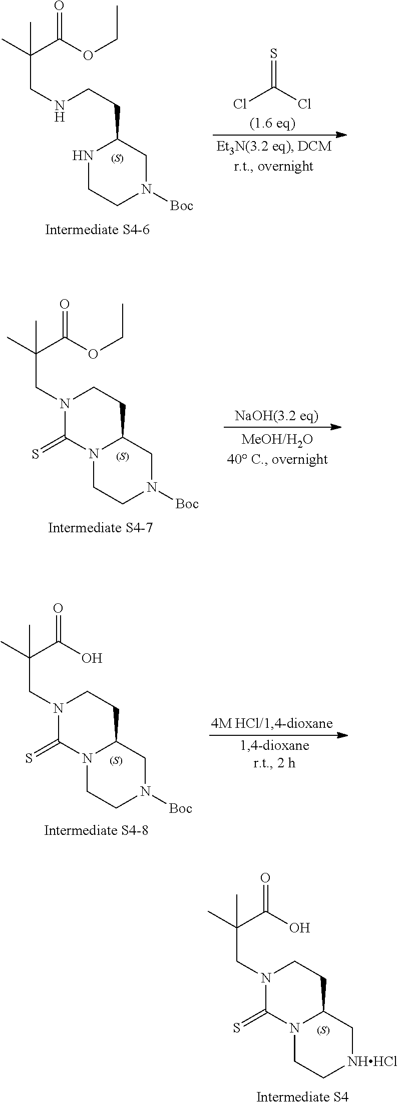

### Intermediate S4-1: (S)-2-(Piperazin-2-yl)ethanol

To a solution of (S)-2-(4-benzylpiperazin-2-yl)ethanol (1.50 g, 6.82 mmol, cas #477220-33-0) in methanol (30 mL) was added 10% palladium on charcoal wt. (500 mg). The reaction mixture was stirred at room temperature under hydrogen atmosphere (50 psi) overnight. Then it was filtered and concentrated to give the title compound (900 mg, 92% yield) as white colorless oil. LC-MS (ESI): R_{T}=0.31 min, mass calcd. for C₆H₁₄N₂O 130.1, m/z found 131.0 [M+H]⁺. ¹H NMR (400 MHz, CDCl₃) δ 3.84-3.74 (m, 1H), 3.69-3.66 (m, 1H), 2.99-2.91 (m, 2.3H), 2.84-2.61 (m, 4.1H), 2.55-2.49 (m, 0.6H), 1.70-1.67 (m, 1H), 1.60-1.56 (m, 1H).

### Intermediate S4-2: (S)-tert-Butyl 3-(2-hydroxyethyl)piperazine-1-carboxylate

To a solution of (S)-2-(Piperazin-2-yl)ethanol dihydrochloride S4-1 (750 mg, 3.33 mmol) in methanol (15 mL) was added triethylamine (660 mg, 6.53 mmol) and di-tert-butyl dicarbonate (654 mg, 3.00 mmol) at −10° C. Then the mixture was warmed to 0° C. and stirred overnight. The mixture was evaporated to give a residue, which was diluted with dichloromethane (20 mL) and washed with brine (20 mL), dried over Na₂SO₄₍ₛ₎, filtered and concentrated to give the title compound (800 mg, 83% yield) as yellow oil. LC-MS (ESI): R_{T}=1.19 min, mass calcd. for C₁₁H₂₂N₂O₃ 230.2, m/z found 231.1 [M+H]⁺. ¹H NMR (400 MHz, CD₃OD) δ 3.86-3.83 (m, 2H), 3.80-3.77 (m, 2H), 3.58-3.55 (m, 2H), 2.84-2.81 (m, 1H), 2.76-2.73 (m, 1H), 2.65-2.54 (m, 3H), 1.52-1.47 (m, 2H), 1.36 (s, 9H).

### Intermediate S4-3: (S)-1-Benzyl 4-tert-butyl 2-(2-hydroxyethyl)piperazine-1,4-dicarboxylate

To a solution of (S)-tert-butyl 3-(2-hydroxyethyl)piperazine-1-carboxylate S4-2 (800 mg, 2.78 mmol) and sodium bicarbonate (2.60 g, 13.9 mmol) in tetrahydrofuran (10 mL) and water (5 mL) was added benzyl chloroformate (709 mg, 4.17 mmol) at 0° C. After stirred at room temperature overnight, the mixture was diluted with water (50 mL) and extracted with ethyl acetate (30 mL) for three times. The combined organic layers were washed with brine (50 mL), dried over Na₂SO₄₍ₛ₎, filtered and concentrated to give a residue, which was purified by silica gel column chromatography (petroleum ether:ethyl acetate=2:1) to give the title compound (760 mg, 75% yield) as colorless oil. LC-MS (ESI): R_{T}=1.58 min, mass calcd. for C₁₉H₂₈N₂O₅ 364.2, m/z found 365.2 [M+H]⁺. ¹H NMR (400 MHz, CDCl₃) δ 7.39-7.32 (m, 5H), 5.16 (s, 2H), 4.36 (s, 1H), 3.95-3.93 (m, 2H), 3.65-3.59 (m, 1H), 3.37-2.83 (m, 5H), 1.87-1.81 (m, 2H), 1.48 (s, 9H).

### Intermediate S4-4: (S)-1-Benzyl 4-tert-butyl 2-(2-oxoethyl)piperazine-1,4-dicarboxylate

To a solution of oxalyl chloride (619 mg, 4.88 mmol) in dichloromethane (15 mL) was added a solution of dimethyl sulfoxide (533 mg, 6.83 mmol) in dichloromethane (50 mL) at −78° C. After stirred at −78° C. for 1 hour, a solution of (S)-1-benzyl 4-tert-butyl 2-(2-hydroxyethyl)piperazine-1,4-dicarboxylate S4-3 (750 mg, 1.95 mmol) was added at −78° C. After stirring at −78° C. for 3 hours, triethylamine (1.50 g, 14.6 mmol) was added dropwise to quench the reaction. The reaction mixture was allowed to warm to room temperature and extracted with dichloromethane (30 mL) for three times. The combined organic layers were dried over anhydrous Na₂SO₄₍ₛ₎, filtered and concentrated to give the title compound (750 mg, 95% yield) as light yellow oil. LC-MS (ESI): R_{T}=1.59 min, mass calcd. for C₁₉H₂₆N₂O₅ 362.2, m/z found 363.2 [M+H]⁺. ¹H NMR (400 MHz, CDCl₃) δ 9.74 (s, 1H), 7.39-7.30 (m, 5H), 5.14 (s, 2H), 4.74-4.71 (m, 1H), 4.11-3.97 (m, 3H), 3.05-2.84 (m, 2H), 2.83-2.74 (m, 2H), 2.61-2.57 (m, 1H), 1.46 (s, 9H).

### Intermediate S4-5: (S)-1-Benzyl 4-tert-butyl 2-(2-((3-ethoxy-2,2-dimethyl-3-oxopropyl)amino)-ethyl)piperazine-1,4-dicarboxylate

To a solution of ethyl 3-amino-2,2-dimethylpropanoate hydrochloride (378 mg, 2.08 mmol) in ethanol (5 mL) was added triethylamine (263 mg, 2.60 mmol) at room temperature. After stirred at room temperature under nitrogen atmosphere for 0.5 hour, a solution of (S)-1-benzyl 4-tert-butyl 2-(2-oxoethyl)piperazine-1,4-dicarboxylate S4-4 (750 mg, 1.86 mmol) in ethanol (5 mL) was added. The mixture was stirred at room temperature for 1 hours, then sodium cyanoborohydride (269 mg, 4.28 mmol) was added at 0° C. After stirred at room temperature for 2 hours, the mixture was quenched with ice water (5 mL), concentrated under vacuo. The residue was diluted with water (15 mL) and extracted with ethyl acetate (20 mL) for three times. The combined organic layers were dried over Na₂SO₄₍ₛ₎, filtered and concentrated to give a residue, which was purified by silica gel column chromatography (dichloromethane:methanol=30:1) to give the title compound (600 mg, 66% yield) as colorless oil. LC-MS (ESI): R_{T}=1.89 min, mass calcd. for C₂₆H₄₁N₃O₆ 491.3, m/z found 492.3 [M+H]⁺. H NMR (400 MHz, CDCl₃) δ 7.39-7.29 (m, 5H), 5.14 (s, 2H), 4.27-4.13 (m, 1H), 4.12-4.09 (q, J=7.2 Hz, 2H), 4.08-3.94 (m, 2H), 3.06-3.00 (m, 2H), 2.95-2.79 (m, 2H), 2.62-2.57 (m, 4H), 1.75-1.69 (m, 2H), 1.45 (s, 9H), 1.23 (t, J=7.2 Hz, 3H), 1.15 (s, 6H).

### Intermediate S4-6: (S)-tert-Butyl 3-(2-((3-ethoxy-2,2-dimethyl-3-oxopropyl)amino)ethyl)piperazine-1-carboxylate

To a solution of (S)-1-benzyl 4-tert-butyl 2-(2-((3-ethoxy-2,2-dimethyl-3-oxopropyl)amino)ethyl)piperazine-1,4-dicarboxylate S4-5 (600 mg, 1.16 mmol) in ethanol (10 mL) was added 20% palladium hydroxide on carbon (300 mg) under nitrogen atmosphere. After stirred at 50° C. under hydrogen atmosphere (60 psi) overnight, the mixture was cooled to room temperature. Then the catalyst was filtered, and the filtrate was concentrated to give the title compound (430 mg, 93% yield) as yellow oil. LC-MS (ESI): R_{T}=1.66 min, mass calcd. for C₁₈H₃₅N₃O₄ 357.3, m/z found 358.4 [M+H]⁺. ¹H NMR (400 MHz, CDCl₃) δ 4.12 (q, J=7.2 Hz, 2H), 3.92 (br s, 2H), 2.96-2.94 (m, 1H), 2.85-2.78 (m, 2H), 2.75-2.61 (m, 6H), 2.28 (br s, 2H), 1.57-1.51 (m, 2H), 1.46 (s, 9H), 1.25 (t, J=7.2 Hz, 3H), 1.19 (s, 6H).

### Intermediate S4-7: (S)-tert-Butyl 7-(3-ethoxy-2,2-dimethyl-3-oxopropyl)-6-thioxohexahydro-1H-pyrazino[1,2-c]pyrimidine-2(6H)-carboxylate

To a solution of (S)-tert-butyl 3-(2-((3-ethoxy-2,2-dimethyl-3-oxopropyl)amino)ethyl)piperazine-1-carboxylate S4-6 (330 mg, 90% purity, 0.83 mmol) and triethylamine (268 mg, 2.66 mmol) in dichloromethane (25 mL) was added a solution of thiophosgene (153 mg, 1.33 mmol) in dichloromethane (10 mL) at 0° C. under nitrogen atmosphere. After stirred at room temperature overnight, the mixture was diluted with ice water (10 mL) and extracted with dichloromethane (20 mL) for three times. The combined organic layers were washed with brine (20 mL), dried over Na₂SO₄₍ₛ₎ and filtered. The filtrate was concentrated to give a residue, which was purified by silica gel column chromatography (petroleum ether:ethyl acetate=4:1) to give the title compound (135 mg, 41% yield) as yellow oil. LC-MS (ESI): R_{T}=1.73 min, mass calcd. for C₁₉H₃₃N₃O₄S 399.2, m/z found 400.3 [M+H]⁺. ¹H NMR (400 MHz, CDCl₃) δ 5.42-5.39 (m, 1H), 4.37-4.29 (m, 2H), 4.14 (q, J=6.8 Hz, 2H), 3.97-3.93 (m, 2H), 3.46-3.38 (m, 1H), 3.28-3.25 (m, 2H), 3.07-2.99 (m, 2H), 2.63-2.60 (m, 1H), 2.14-2.09 (m, 1H), 1.75-1.66 (m, 1H), 1.47 (s, 9H), 1.29-1.26 (m, 9H).

### Intermediate S4-8: (S)-3-(2-(tert-Butoxycarbonyl)-6-thioxotetrahydro-1H-pyrazino[1,2-c]pyrimidin-7(2H,6H,8H)-yl)-2,2-dimethylpropanoic acid

To a solution of (S)-tert-butyl 7-(3-ethoxy-2,2-dimethyl-3-oxopropyl)-6-thioxohexahydro-1H-pyrazino[1,2-c]pyrimidine-2(6H)-carboxylate S4-7 (170 mg, 0.405 mmol) in methanol (3 mL) and water (1 mL) was added sodium hydroxide (51 mg, 1.28 mmol) under nitrogen atmosphere. After stirred at 40° C. overnight, the reaction was concentrated to give a residue, which was diluted with water (5 mL) and acidified with 1 N hydrochloride aqueous solution to pH ˜3. The aqueous phase was extracted with ethyl acetate (20 mL) for three times. The combined organic layers were dried over Na₂SO₄₍ₛ₎, filtered and concentrated to give the desired compound (130 mg, 78% yield) as yellow solids. LC-MS (ESI): R_{T}=1.17 min, mass calcd. for C₁₇H₂₉N₃O₄S 371.2, m/z found 370.3 [M−H]⁻. ¹H NMR (400 MHz, CDCl₃) δ 5.40-5.37 (m, 1H), 4.40-3.96 (m, 2H), 4.02-3.96 (m, 2H), 3.45-3.40 (m, 1H), 3.37-3.34 (m, 2H), 3.07-3.02 (m, 2H), 2.60 (br s, 1H), 2.18-2.11 (m, 1H), 1.76-1.72 (m, 1H), 1.47 (s, 9H), 1.31 (m, 6H).

### Intermediate S4: (S)-2,2-Dimethyl-3-(6-thioxotetrahydro-1H-pyrazino[1,2-c]pyrimidin-7(2H,6H,8H)-yl)propanoic acid hydrochloride

To a solution of (S)-3-(2-(tert-butoxycarbonyl)-6-thioxotetrahydro-1H-pyrazino[1,2-c]pyrimidin-7(2H,6H,8H)-yl)-2,2-dimethylpropanoic acid S4-8 (130 mg, 0.315 mmol) in 1,4-dioxane (2 mL) was added 4 M hydrochloride in 1,4-dioxane (2 mL) under nitrogen atmosphere. After stirred at room temperature under nitrogen atmosphere for 2 hour, the reaction mixture was concentrated to give the title compound (102 mg, 95% yield) as yellow solids. ¹H NMR (400 MHz, CD₃OD) δ 5.56-5.52 (m, 1H), 4.26 (d, J=14.0 Hz, 1H), 4.16 (d, J=14.0 Hz, 1H), 3.78-3.72 (m, 1H), 3.40-3.36 (m, 1H), 3.34-3.27 (m, 3H), 3.17-3.13 (m, 1H), 3.06-2.99 (m, 1H), 2.82-2.76 (t, J=12.4 Hz, 1H), 2.20-2.14 (m, 1H), 1.74-1.65 (m, 1H), 1.16 (s, 3H), 1.15 (s, 3H).

### Compound 6: 3-((S)-2-(((S)-5-(Ethoxycarbonyl)-6-(3-fluoro-2-methylphenyl)-2-(thiazol-2-yl)-3,6-dihydropyrimidin-4-yl)methyl)-6-thioxotetrahydro-1H-pyrazino[1,2-c]pyrimidin-7(2H,6H,8H)-yl)-2,2-dimethylpropanoic acid

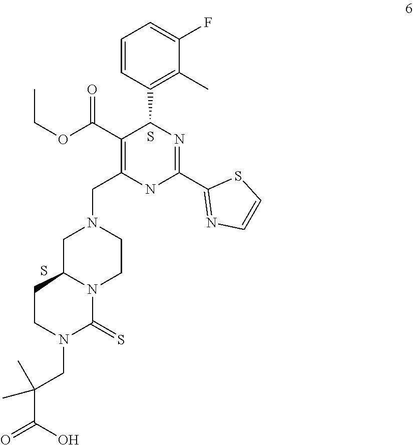

To a solution of (S)-ethyl 6-(bromomethyl)-4-(3-fluoro-2-methylphenyl)-2-(thiazol-2-yl)-1,4-dihydropyrimidine-5-carboxylate (H2-1A) (106 mg, 0.205 mmol) in tetrahydrofuran (3 ml) was added (S)-2,2-dimethyl-3-(6-thioxotetrahydro-1H-pyrazino[1,2-c]pyrimidin-7(2H,6H,8H)-yl)propanoic acid hydrochloride S4 (70 mg, 0.205 mmol) and triethylamine (80 mg, 0.792 mmol) under nitrogen atmosphere. After stirred at 40° C. under nitrogen atmosphere for 2 hours, the reaction mixture was diluted with water (10 mL) and extracted with ethyl acetate (10 mL) twice. The combined organic layers were washed with brine (10 mL), dried over Na₂SO₄₍ₛ₎, filtered and concentrated to give a residue, which was purified by pre-HPLC (Column: Waters Xbrige C18 (5 μm 19*150 mm), Mobile phase A: water (0.1% ammonium bicarbonate), Mobile phase B: acetonitrile, UV: 214 nm, Flow rate: 15 mL/min, Gradient: 20-50% (% B)) to give the title compound (30 mg, 98.5% purity, 23% yield, 99.6% stereopure) as yellow solids. LC-MS (ESI): R_{T}=3.764 min, mass calcd. for C₃₀H₃₇FN₆O₄S₂ 628.2, m/z found 629.3 [M+H]⁺. Chiral HPLC (Column: Chiralpak IE, 5 μm 4.6*250 mm; Mobile Phase: Hex:EtOH:TFA=60:40:0.2 at 1 mL/min; Temp: 30° C.; Wavelength: 254 nm; R_{T}=8.668 min). ¹H NMR (400 MHz, CDCl₃) δ 9.54 (s, 1H), 7.81 (d, J=3.2 Hz, 1H), 7.41 (d, J=3.2 Hz, 1H), 7.08-7.02 (m, 1H), 6.99-6.97 (m, 1H), 6.90 (t, J=8.4 Hz, 1H), 6.02 (s, 1H), 5.50-5.47 (m, 1H), 4.38-4.35 (m, 2H), 4.09-4.02 (m, 3H), 3.89 (d, J=16.8 Hz, 1H), 3.72-3.65 (m, 1H), 3.41-3.38 (m, 2H), 3.26-3.21 (m, 1H), 2.91-2.88 (m, 1H), 2.80-2.77 (m, 1H), 2.55 (s, 3H), 2.41 (t, J=9.2 Hz, 1H), 2.29 (t, J=10.8 Hz, 1H), 2.18-2.13 (m, 1H), 1.82-1.78 (m, 1H), 1.33 (s, 6H), 1.12 (t, J=7.2 Hz, 3H).

### Compound 6A: 3-((R)-2-(((S)-5-(ethoxycarbonyl)-6-(3-fluoro-2-methylphenyl)-2-(thiazol-2-yl)-3,6-dihydropyrimidin-4-yl)methyl)-6-thioxohexahydro-2H-pyrazino[1,2-c]pyrimidin-7(6H)-yl)-2,2-dimethylpropanoic acid

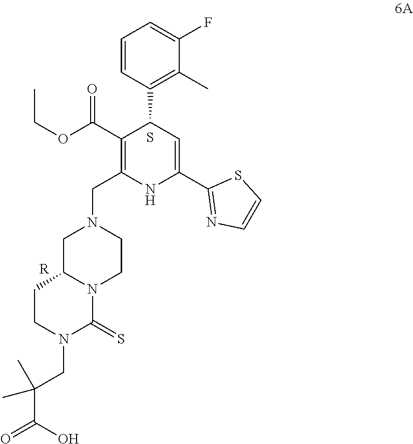

### Preparation of Intermediate S4A

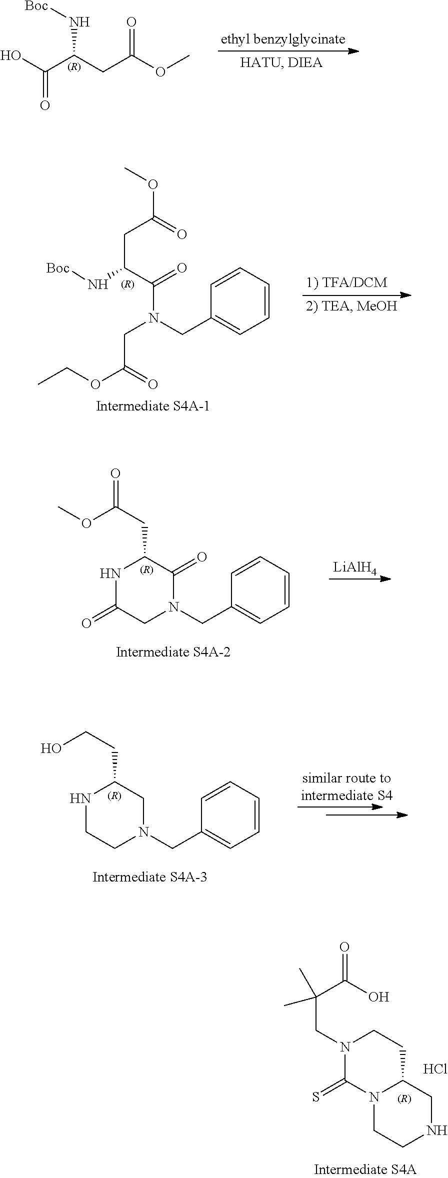

### Intermediate S4A-1: (R)-methyl 4-(benzyl(2-ethoxy-2-oxoethyl)amino)-3-((tert-butoxycarbonyl)amino)-4-oxobutanoate

To a solution of (R)-2-((tert-butoxycarbonyl)amino)-4-methoxy-4-oxobutanoic acid (10 g, 40.4 mmol) in N,N-dimethylformamide (80 mL) was added 2-(7-azabenzotriazol-1-yl)-N,N,N′,N′-tetramethyluronium hexafluorophosphate (16.9 g, 44.4 mmol), N,N-diisopropylethylamine (7.84 g, 60.7 mmol) and ethyl 2-(benzylamino)acetate (8.6 g, 44.5 mmol) at 0° C. After stirred at room temperature overnight, the mixture was poured into water (200 mL) and extracted with ethyl acetate (100 mL) twice. The combined organic layers were washed with water (100 mL) and brine (100 mL), dried over Na₂SO₄₍ₛ₎ and filtered. The filtrated was concentrated and purified by silica gel column chromatography (petroleum ether ethyl acetate=20:1 to 5:1) to give the title compound (17.3 g, 95% purity from ¹H NMR, 96% yield) as colorless oil. LC-MS (ESI): R_{T}=1.66 min, mass calcd. for C₂₁H₃₀N₂O₇ 422.2, m/z found 423.1 [M+H]⁺. ¹H NMR (400 MHz, CDCl₃) δ 7.37-7.25 (m, 4.4H), 7.20-7.16 (m, 0.6H), 5.51 (d, J=9.2 Hz, 0.6H), 5.43 (d, J=10.0 Hz, 0.4H), 5.20-5.12 (m, 0.6H), 4.87-4.83 (m, 1H), 4.74 (d, J=14.8 Hz, 0.4H), 4.63 (d, J=16.4 Hz, 0.6H), 4.57 (d, J=14.8 Hz, 0.4H), 4.33-4.28 (m, 0.4H), 4.20-4.05 (m, 3H), 3.86-3.82 (m, 0.6H), 3.70 (s, 1.8H), 3.69 (s, 1.2H), 2.87-2.80 (m, 1H), 2.73-2.66 (m, 1H), 1.43 (s, 3.6H), 1.34 (s, 5.4H), 1.28-1.22 (m, 3H).

### Intermediate S4A-2: (R)-methyl 2-(4-benzyl-3,6-dioxopiperazin-2-yl)acetate

To a solution of (R)-methyl 4-(benzyl(2-ethoxy-2-oxoethyl)amino)-3-((tert-butoxycarbonyl)amino)-4-oxobutanoate Intermediate S4A-1 (17 g, 95% purity, 38.2 mmol) in dichloromethane (169 mL) was added trifluoroacetic acid (34 mL) dropwise at 0° C. After stirred at room temperature for 1 hour, the mixture was concentrated under reduced pressure to give a residue, which was dissolved in methanol (169 mL). The resulting solution was basified with triethylamine to pH ˜8. After stirred at room temperature overnight, the reaction mixture was concentrated under reduced pressure to remove the volatile. The residue was dissolved in dichloromethane (200 mL) and washed with water (100 mL) for three times. The organic layer was dried over Na₂SO₄₍ₛ₎ and filtered. The filtrate was concentrated and purified by silica gel column chromatography (dichloromethane:methanol=50:1 to 10:1) to give the title compound (10 g, 95% purity from ¹H NMR, 90% yield) as yellow oil. LC-MS (ESI): R_{T}=1.26 min, mass calcd. for C₁₄H₁₆N₂O₄ 276.1, m/z found 277.0 [M+H]⁺. ¹H NMR (400 MHz, CDCl₃) δ 7.37-7.23 (m, 5H), 4.60 (s, 2H), 4.39 (d, J=6.0 Hz, 1H), 3.91 (d, J=17.6 Hz, 1H), 3.82 (d, J=17.6 Hz, 1H), 3.68 (s, 3H), 3.06 (d, J=3.6 Hz, 0.4H), 3.02 (d, J=3.6 Hz, 0.6H), 2.90 (d, J=7.6 Hz, 0.6H), 2.86 (d, J=7.6 Hz, 0.4H),

### Intermediate S4A-3: (R)-2-(4-benzylpiperazin-2-yl)ethanol

To a solution of (R)-methyl 2-(4-benzyl-3,6-dioxopiperazin-2-yl)acetate Intermediate S4A-2 (9 g, 95% purity, 30.9 mmol) in tetrahydrofuran (250 mL) was added lithium aluminum hydride (9.8 g, 258 mmol) at 0° C. in portions over 1 hour. After stirred at 80° C. overnight, the reaction mixture was cooled to 0° C. and quenched with 10% ammonium chloride aqueous solution (9 mL). The resulting mixture was filtered and the filtrate was concentrated under reduced pressure to give a residue. The residue was dissolved in dichloromethane (200 mL), dried over Na₂SO₄₍ₛ₎ and filtered. The filtrate was concentrated under reduced pressure to give the title compound (5.4 g, 90% purity from H NMR, 71% yield) as yellow oil. LC-MS (ESI): R_{T}=1.12 min, mass calcd. for C₁₃H₂₀N₂O 220.2, m/z found 221.1 [M+H]⁺. ¹H NMR (400 MHz, CD₃OD) δ 7.32-7.23 (m, 5H), 3.60-3.59 (m, 2H), 3.55-3.48 (m, 2H), 2.93-2.73 (m, 5H), 2.07 (dt, J=11.2 Hz, 3.6 Hz, 1H), 1.80 (t, J=10.8 Hz, 1H), 1.63-1.48 (m, 2H).

### Intermediate S4A was Prepared Analogous to Intermediate S4

¹H NMR (400 MHz, DMSO-d₆) δ 12.43 (br s, 1H), 9.08 (s, 2H), 5.41-5.38 (m, 1H), 4.27 (d, J=13.6 Hz, 1H), 4.16 (d, J=14.0 Hz, 1H), 3.80-3.72 (m, 1H), 3.27-3.16 (m, 5H), 2.93-2.82 (m, 2H), 2.18-2.08 (m, 1H), 1.74-1.66 (m, 1H), 1.16 (s, 6H).

Compound 6A was prepared analogous to compound 6. LC-MS (ESI): R_{T}=3.601 min, mass calcd. for C₃₀H₃₇FN₆O₄S₂ 628.2, m/z found 629.3 [M+H]⁺. Chiral analysis (Column: Chiralpak IE 5 μm 4.6*250 mm; Mobile Phase: Hex:EtOH:TFA=60:40:0.2 at 1.0 mL/min; Temp: 30° C.; Wavelength: 254 nm, R_{T}=10.549 min). H NMR (400 MHz, CDCl₃) δ 9.51 (s, 1H), 7.81 (d, J=3.2 Hz, 1H), 7.42 (d, J=3.2 Hz, 1H), 7.10-7.05 (m, 1H), 7.00-6.98 (m, 1H), 6.91 (t, J=8.8 Hz, 1H), 6.01 (s, 1H), 5.57-5.53 (m, 1H), 4.37 (s, 2H), 4.10-3.98 (m, 3H), 3.91 (d, J=17.2 Hz, 1H), 3.69-3.62 (m, 1H), 3.37-3.35 (m, 2H), 3.29-3.22 (m, 1H), 2.96-2.93 (m, 1H), 2.78-2.75 (m, 1H), 2.58-2.52 (m, 4H), 2.18-2.05 (m, 2H), 1.76-1.68 (m, 1H), 1.33 (s, 6H), 1.12 (t, J=7.2 Hz, 3H).

### Compound 7: 3-(3-(Cyanomethylene)-7-(((S)-5-(ethoxycarbonyl)-6-(3-fluoro-2-methylphenyl)-2-(thiazol-2-yl)-3,6-dihydropyrimidin-4-yl)methyl)hexahydroimidazo[1,5-a]pyrazin-2(3H)-yl)-2,2-dimethylpropanoic acid (Mixture of 2 diastereomers)

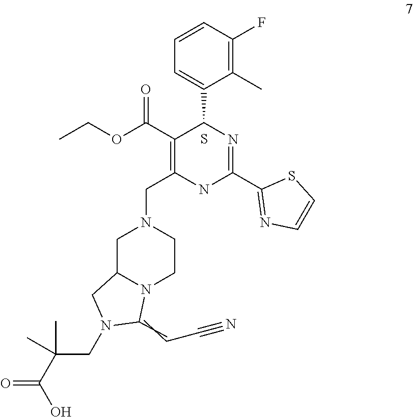

### Preparation of Intermediate S5

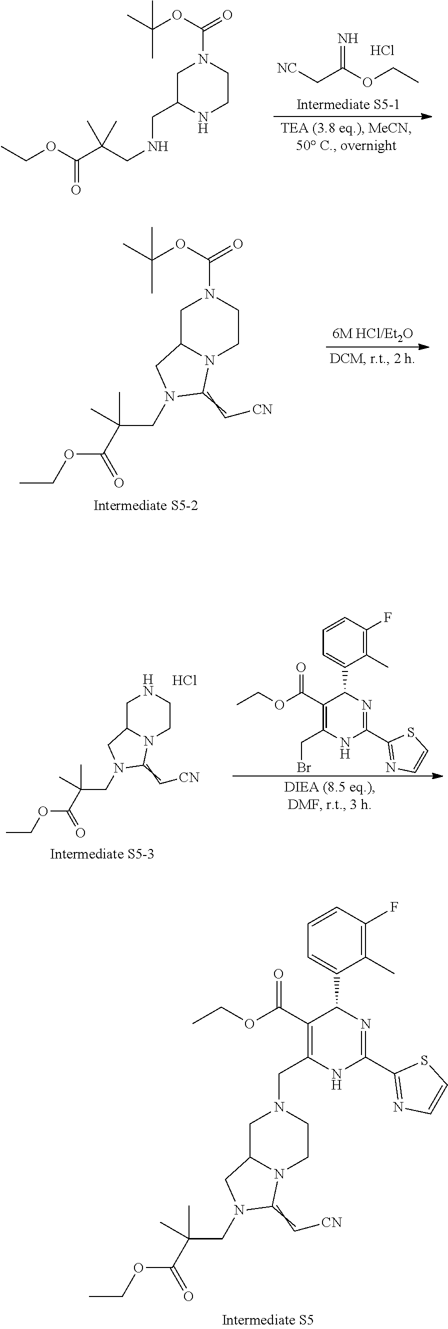

### Intermediate S5-1: Ethyl 2-cyanoacetimidate hydrochloride

To a solution of malononitrile (3.00 g, 45.4 mmol) and ethanol (2.09 g, 45.4 mmol) in diethyl ether (15 mL) was added 6 M hydrochloride in diethyl ether (10 mL, 60 mmol) at 0° C. After stirred at 0° C. for 0.5 hour, the mixture was warmed up to room temperature and stirred at room temperature overnight. It was filtered and the cake was washed with cooled diethyl ether (20 mL) twice, then suspended in diethyl ether (20 mL), and filtered again, then dried to give the title compound (6.13 g, 70% purity from H NMR, 64% yield) as white solids which was used in the next step without further purification. ¹H NMR (400 MHz, DMSO-d₆) δ 4.20-4.14 (m, 2H), 4.03 (s, 2H), 1.24-1.20 (m, 3H).

### Intermediate S5-2: tert-Butyl 3-(cyanomethylene)-2-(3-ethoxy-2,2-dimethyl-3-oxopropyl)hexahydroimidazo[1,5-a]pyrazine-7(1H)-carboxylate

To a solution of ethyl 2-cyanoacetimidate hydrochloride S5-1 (710 mg, 3.345 mmol) and triethylamine (450 mg, 4.447 mmol) in acetonitrile (20 mL) was added tert-butyl 3-(((2,2-dimethyl-3-oxo-3-propoxypropyl)amino)methyl)piperazine-1-carboxylate S1-4 (500 mg, 1.17 mmol). After stirred at 50° C. overnight, the mixture was concentrated to give a residue, which was diluted with ethyl acetate (15 mL), washed with brine (100 mL), dried over Na₂SO₄ (s) and filtered. The filtrate was concentrated in vacuo to give a residue, which was purified by by silica gel column chromatography (petroleum ether:ethyl acetate=2:1) to give the title compound (244 mg, 48% yield) as yellow oil. LC-MS (ESI): R_{T}=1.52 min, mass calcd. for C₂₀H₃₂N₄O₄ 392.2, m/z found 396.3 [M+H]⁺. ¹H NMR (400 MHz, CDCl₃) 4.57 (d, J=12.0 Hz, 0.6H), 4.29-4.22 (m, 0.4H), 4.18-4.00 (m, 3.4H), 3.90-3.87 (m, 0.6H), 3.46-3.34 (m, 2.4H), 3.21-2.81 (m, 5.6H), 2.71-2.58 (m, 1H), 1.47 (s, 9H), 1.31-1.26 (m, 6H), 1.21 (s, 3H).

### Intermediate S5-3: Ethyl 3-(3-(cyanomethylene)hexahydroimidazo[1,5-a]pyrazin-2(3H)-yl)-2,2-dimethylpropanoate hydrochloride

To a solution of tert-butyl 3-(cyanomethylene)-2-(3-ethoxy-2,2-dimethyl-3-oxoprop yl)hexahydroimidazo[1,5-a]pyrazine-7(1H)-carboxylate S5-2 (123 mg, 0.282 mmol) in dichloromethane (1 mL) was added 6 M hydrochloride in diethyl ether (3 mL, 18 mmol) at 0° C. After stirred at room temperature for 2 hours, the reaction mixture was concentrated to give the title compound (96 mg, 99% yield) as yellow solids which was used in the next step without further purification. LC-MS (ESI): R_{T}=0.95 min, mass calcd. for C₁₅H₂₅ClN₄O₂ 328.2, m/z found 293.4 [M−HCl+H]⁺.

### Intermediate S5: (4S)-Ethyl 6-((3-(cyanomethylene)-2-(3-ethoxy-2,2-dimethyl-3-oxopropyl)hexahydroimidazo[1,5-a]pyrazin-7(1H)-yl)methyl)-4-(3-fluoro-2-methylphenyl)-2-(thiazol-2-yl)-1,4-dihydropyrimidine-5-carboxylate

To a solution of ethyl 3-(3-(cyanomethylene)hexahydroimidazo[1,5-a]pyrazin-2(3H)-yl)-2,2-dimethylpropanoate hydrochloride S5-3 (96 mg, 0.280 mmol) in N,N-dimethylformamide (1 mL) was added (S)-ethyl 6-(bromomethyl)-4-(3-fluoro-2-methylphenyl)-2-(thiazol-2-yl)-1,4-dihydropyrimidine-5-carboxylate (112-1A) (100 mg, 0.205 mmol), N-ethyl-N-isopropylpropan-2-amine (225 mg, 1.74 mmol) at room temperature. After stirred at room temperature for 3 hours, the mixture poured into water (20 mL), extracted with ethyl acetate (20 mL) twice. The combined organic layers were washed with water (10 mL), brine (10 mL), dried over Na₂SO₄₍ₛ₎ and filtered. The filtrate was concentrated to give a residue, which was purified by silica gel column chromatography (petroleum ether:acetone=10:1 to 5:1) to give the title compound (71 mg, 48% yield) as yellow solids. LC-MS (ESI): R_{T}=1.84 min, mass calcd. for C₃₃H₄₀FN₇O₄S 649.3, m/z found 650.2 [M+H]⁺. ¹H NMR (400 MHz, CDCl₃) 9.48-9.43 (m, 1H), 7.82 (d, J=2.8 Hz, 1H), 7.43 (d, J=3.2 Hz, 1H), 7.12-7.06 (m, 1H), 6.99-6.97 (m, 1H), 6.93-6.88 (m, 1H), 6.05-6.01 (m, 1H), 4.72-4.60 (m, 0.6H), 4.18-3.87 (m, 6.4H), 3.76-3.58 (m, 1H), 3.46-3.30 (m, 2H), 3.21-3.02 (m, 2.6H), 2.97-2.70 (m, 3H), 2.59-2.50 (m, 3.4H), 2.48-2.31 (m, 1H), 2.22-2.15 (m, 1H), 1.33-1.21 (m, 9H), 1.12 (t, J=7.2 Hz, 3H).

### Compound 7: 3-(3-(Cyanomethylene)-7-(((S)-5-(ethoxycarbonyl)-6-(3-fluoro-2-methylphenyl)-2-(thiazol-2-yl)-3,6-dihydropyrimidin-4-yl)methyl)-hexahydroimidazo[1,5-a]pyrazin-2(3H)-yl)-2,2-dimethylpropanoic acid (Mixture of 2 diastereomers)

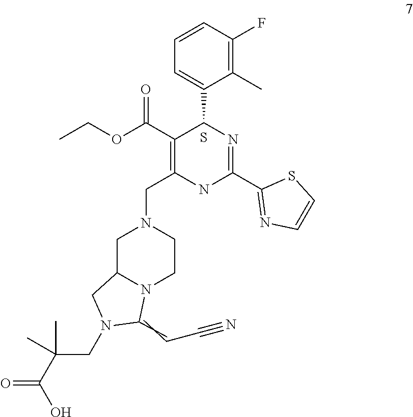

To a solution of (4S)-ethyl 6-((3-(cyanomethylene)-2-(3-ethoxy-2,2-dimethyl-3-oxopropyl)hexahydroimidazo[1,5-a]pyrazin-7(1H)-yl)methyl)-4-(3-fluoro-2-methylphenyl)-2-(thiazol-2-yl)-1,4-dihydropyrimidine-5-carboxylate S5 (71 mg, 0.079 mmol) in ethanol (0.9 mL) and water (0.3 mL) was added lithium hydroxide monohydrate (19 mg, 0.453 mmol). After stirred at room temperature overnight, the mixture was concentrated and diluted with waster (15 mL), adjusted with 0.1 M hydrochloric aqueous solution to pH 5˜6, extracted with ethyl acetate (20 mL) twice. The combined organic layers were concentrated to give a residue, which was purified by Prep-HPLC (Column: waters Xbridge C18 (5 μm 19*150 mm), Mobile Phase A: water (0.1% ammonium hydroxide), Mobile Phase B: acetonitrile, UV: 214 nm, Flow rate: 15 mL/min, Gradient: 15-45% (% B)) to give the title compound (4.9 mg, 93.6% purity, 8% yield) as yellow solids. LC-MS (ESI): R_{T}=3.554 min, mass calcd. for C₃₁H₃₆FN₇O₄S 621.3, m/z found 621.9 [M+H]⁺. ¹H NMR (400 MHz, DMSO-d₆) 12.26 (br s, 1H), 9.59-9.51 (m, 1H), 8.04-7.92 (m, 2H), 7.22-7.15 (m, 1H), 7.06-7.01 (m, 2H), 5.88 (s, 1H), 4.47 (d, J=12.8 Hz, 0.6H), 4.04-3.91 (m, 4.4H), 3.75 (s, 0.6H), 3.62-3.50 (m, 1.4H), 3.43-3.37 (m, 2H), 3.24-3.17 (m, 2H), 2.99-2.90 (m, 3H), 2.45 (s, 3H), 2.39-2.33 (m, 1H), 2.14-2.04 (m, 1H), 1.18-1.04 (m, 9H).

### Compound 8: (S)-3-(3-(Acetylimino)-7-((5-(ethoxycarbonyl)-6-(3-fluoro-2-methylphenyl)-2-(thiazol-2-yl)-3,6-dihydropyrimidin-4-yl)methyl)hexahydroimidazo[1,5-a]pyrazin-2(3H)-yl)-2,2-dimethylpropanoic acid (Mixture of 2 diastereomers)

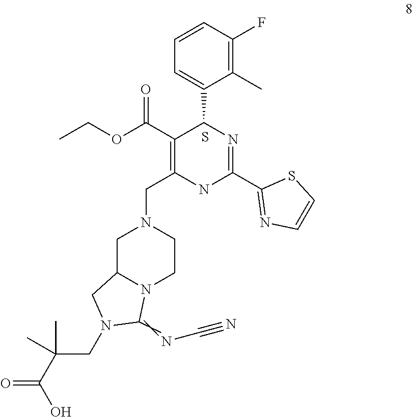

### Preparation of Intermediate S6

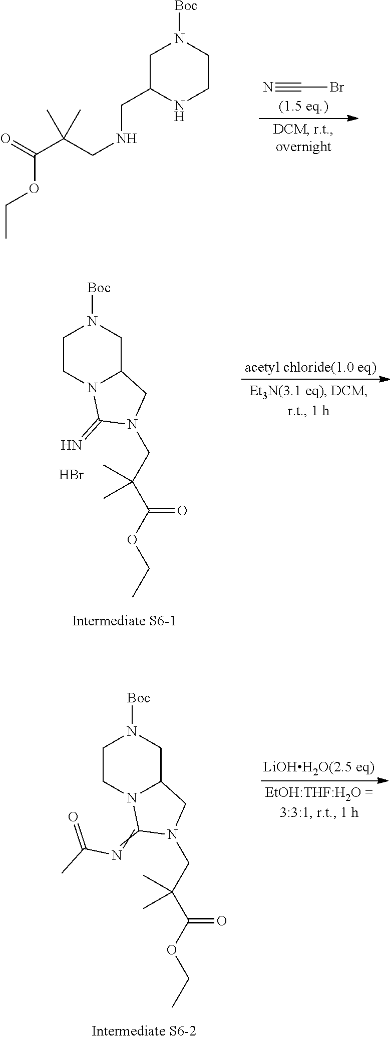

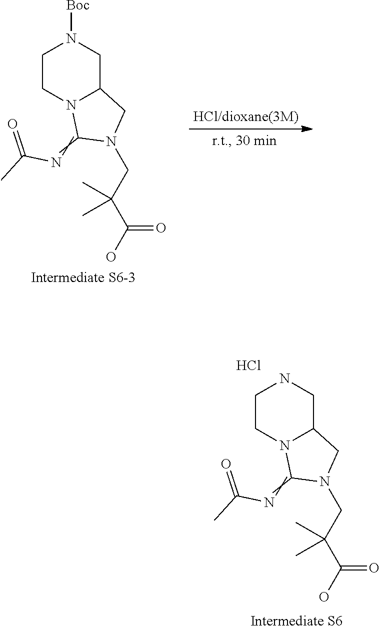 [SMILES: CC(=O)N=C1N(CC(C)(C)C(=O)O)CC2CN(C(=O)OC(C)(C)C)CCN12 ; Cl ; CC(=O)N=C1N(CC(C)(C)C(=O)O)CC2CNCCN12]

### Intermediate S6-1: tert-Butyl 2-(3-ethoxy-2,2-dimethyl-3-oxopropyl)-3-iminohexahydroimidazo[1,5-a]pyrazine-7(1H)-carboxylate hydrobromide

To a mixture of tert-butyl 3-(((3-ethoxy-2,2-dimethyl-3-oxopropyl)amino)methyl)pip erazine-1-carboxylate S1-4 (1.6 g, 4.19 mmol) in dichloromethane (2 mL) at room temperature was added the solution of cyanic bromide (666 mg, 6.29 mmol) in dichloromethane (2 mL) dropwise. After stirred at room temperature overnight, the mixture was filtered and the residue was washed with petroleum ether. The filter cake was concentrated under reduced pressure to give the title compound (1.61 g, 77% yield) as white solids. LC-MS (ESI): R_{T}=1.732 min, mass calcd. for C₁₈H₃₂N₄O₄ 368.2, m/z found 369.2 [M+H]⁺. ¹H NMR (300 MHz, DMSO-d₆) δ 8.37 (s, 2H), 4.14 (q, J=6.9 Hz, 3H), 4.00-3.90 (m, 2H), 3.87-3.71 (m, 1H), 3.64 (t, J=9.6 Hz, 1H), 3.56-3.45 (m, 2H), 3.20-3.05 (m, 2H), 2.95-2.67 (m, 1H), 1.44 (s, 9H), 1.25 (t, J=6.9 Hz, 3H), 1.20 (s, 6H).

### Intermediate S6-2: tert-Butyl 3-(acetylimino)-2-(3-ethoxy-2,2-dimethyl-3-oxopropyl)hexahydroimidazo[1,5-a]pyrazine-7(1H)-carboxylate

To a solution of tert-butyl 2-(3-ethoxy-2,2-dimethyl-3-oxopropyl)-3-iminohexahydroimidazo[1,5-a]pyrazine-7(1H)-carboxylate hydrobromide S6-1 (245 mg, 0.436 mmol) in dichloromethane (10 mL) was added triethylamine (140 mg, 1.38 mmol) and acetyl chloride (35 mg, 0.446 mmol) at room temperature. After stirred at room temperature for 1 hour, the mixture was poured into water (30 mL) and extracted with dichloromethane (30 mL) twice. The combined organic layers were washed with brine (50 mL), dried over anhydrous Na₂SO₄₍ₛ₎, filtered and concentrated in vacuo to give the title compound (190 mg, 96% yield) as brown oil. LC-MS (ESI): RT=1.460 min, mass calcd. for C₂₀H₃₄N₄O₅ 410.3, m/z found 411.2 [M+H]⁺. ¹H NMR (400 MHz, CDCl₃) δ 4.42-4.34 (m, 1H), 4.18-4.13 (m, 4H), 3.99-3.92 (m, 2H), 3.65-3.55 (m, 2H), 3.31-3.19 (m, 2H), 2.99-2.75 (m, 2H), 2.35 (s, 3H), 1.46 (s, 9H), 1.30-1.24 (m, 9H).

### Intermediate S6-3: tert-Butyl 3-(acetylimino)-2-(3-ethoxy-2,2-dimethyl-3-oxopropyl)hexahydroimidazo[1,5-a]pyrazine-7(1H)-carboxylate

A mixture of tert-butyl 3-(acetylimino)-2-(3-ethoxy-2,2-dimethyl-3-oxopropyl)hexahydroimidazo[1,5-a]pyrazine-7(1H)-carboxylate S6-2 (190 mg, 0.417 mmol) in tetrahydrofuran (3 mL) and methanol (3 mL) was added a solution of lithium hydroxide monohydrate (40 mg, 0.953 mmol) in water (1 mL). The reaction mixture was stirred at room temperature under nitrogen atmosphere for 1 hour. Then the reaction mixture was acidified to pH=5 with 0.5 M hydrochloride aqueous solution. The mixture was extracted with ethyl acetate (15 mL) for three times and the combined organic layers were concentrated in vacuo to give the title compound (120 mg, 56% yield) as yellow oil. LC-MS (ESI): R_{T}=1.068 min, mass calcd. for C₁₈H₃₀N₄O₅ 382.2, m/z found 383.2 [M+H]⁺.

### Intermediate S6: 3-(3-(Acetylimino)hexahydroimidazo[1,5-a]pyrazin-2(3H)-yl)-2,2-dimethylpropanoic acid hydrochloride

To a solution of tert-butyl 3-(acetylimino)-2-(3-ethoxy-2,2-dimethyl-3-oxopropyl)hexahydroimidazo[1,5-a]pyrazine-7(1H)-carboxylate S6-3 (120 mg, 0.235 mmol) in 3 M hydrochloride in 1,4-dioxane (7 mL, 21 mmol) was stirred at room temperature for 30 minutes. The mixture was concentrated in vacuo to give the title compound (80 mg, 90% yield) as brown solids. LC-MS (ESI): R_{T}=0.226 min, mass calcd. for C₁₃H₂₂N₄O₃ 282.2, m/z found 283.2 [M+H]⁺.

### Compound 8: (S)-3-(3-(Acetylimino)-7-((5-(ethoxycarbonyl)-6-(3-fluoro-2-methylphenyl)-2-(thiazol-2-yl)-3,6-dihydropyrimidin-4-yl)methyl)hexahydroimidazo[1,5-a]pyrazin-2(3H)-yl)-2,2-dimethylpropanoic acid (Mixture of 2 diastereomers)

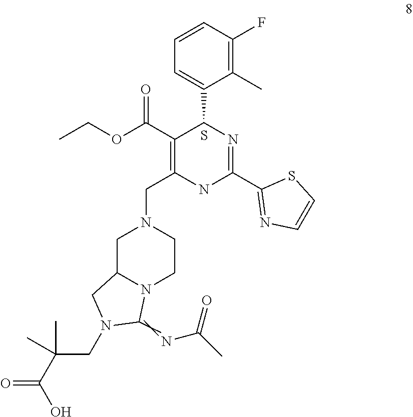

To a solution of 3-(3-(acetylimino)hexahydroimidazo[1,5-a]pyrazin-2(3H)-yl)-2,2-dimethylpropanoic acid hydrochloride S6 (60 mg, 0.159 mmol) in N,N-dimethylformamide (3 mL) was added N,N-diisopropylethylamine (150 mg, 1.16 mmol) and (S)-ethyl 6-(bromomethyl)-4-(3-fluoro-2-methylphenyl)-2-(thiazol-2-yl)-1,4-dihydropyrimidine-5-carboxylate (H2-1A) (65 mg, 0.141 mmol) at room temperature. After stirred at room temperature overnight, the mixture was concentrated and purified by C18 column (acetonitrile:water=5% to 95%) to afford the title compound (6.7 mg, 95.1% purity, 7% yield) as yellow solids. LC-MS (ESI): R_{T}=3.522 min, mass calcd. for C₃₁H₃₈FN₇O₅S 639.3, m/z found 640.3 [M+H]⁺. ¹H NMR (400 MHz, CD₃OD) δ 7.82-7.81 (m, 1H), 7.62 (d, J=3.2 Hz, 1H), 7.04-6.98 (m, 2H), 6.85-6.80 (m, 1H), 5.87 (s, 0.3H), 5.86 (s, 0.7H), 4.15-4.03 (m, 2H), 3.95 (q, J=7.2 Hz, 2H), 3.87-3.71 (m, 2H), 3.55-3.42 (m, 2H), 3.36-3.28 (m, 3H), 3.10-3.03 (m, 0.5H), 2.95-2.88 (m, 1.5H), 2.45-2.36 (m, 4H), 2.26-2.20 (m, 1H), 1.99 (s, 2H), 1.97 (s, 1H), 2.11-2.11 (m, 2H), 1.09 (s, 4H), 1.02 (t, J=7.2 Hz, 3H).

### Compound 9A: 1-((7-(((S)-5-(Ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)methyl)cyclopropanecarboxylic acid (Single Enantiomer)

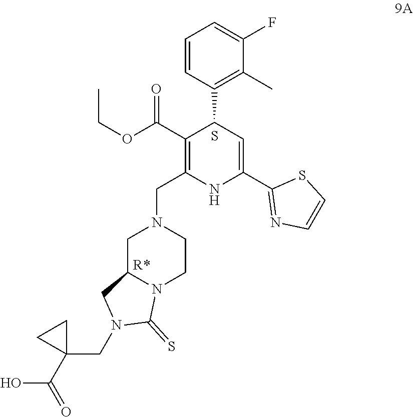

To a solution of (S)-ethyl 6-(bromomethyl)-4-(3-fluoro-2-methylphenyl)-2-(thiazol-2-yl)-1,4-dihydropyrimidine-5-carboxylate (H2-1A) (90 mg, 0.185 mmol) in tetrahydrofuran (4 ml) was added 1-((3-thioxohexahydroimidazo[1,5-a]pyrazin-2(3H)-yl)methyl)cyclopropanecarboxylic acid hydrochloride S3-A (56 mg, 0.173 mmol) and triethylamine (133 mg, 0.891 mmol) under nitrogen atmosphere. After stirred at 40° C. under nitrogen atmosphere for 2.5 hours and then stirred at room temperature overnight, the reaction mixture was diluted with water (10 mL) and extracted with ethyl acetate (10 mL) twice. The combined organic layers were washed with brine (10 mL), dried over Na₂SO₄₍ₛ₎, filtered and concentrated to give a residue, which was purified by C18 column (acetonitrile:water=40% to 70%) to give the title compound (19 mg, 16% yield) as yellow solids. LC-MS (ESI): R_{T}=3.924 min, mass calcd. for C₂₉H₃₃FN₆O₄S₂ 612.2, m/z found 613.2 [M+H]⁺. ¹H NMR (400 MHz, CD₃OD) δ 7.81 (d, J=3.2 Hz, 1H), 7.61 (d, J=3.2 Hz, 1H), 7.05-6.98 (m, 2H), 6.84-6.80 (m, 1H), 5.87 (s, 1H), 4.32-4.29 (m, 1H), 4.03-3.92 (m, 4H), 3.89-3.79 (m, 4H), 3.37-3.31 (m, 1H), 3.16-3.13 (m, 1H), 2.96-2.93 (m, 1H), 2.67-2.64 (m, 1H), 2.41 (s, 3H), 2.22-2.13 (m, 2H), 1.08-1.07 (m, 2H), 1.02 (t, J=6.8 Hz, 3H), 0.87-0.86 (m, 2H).

### Compound 9B: 1-((7-(((S)-5-(Ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)methyl)cyclopropanecarboxylic acid (Single Enantiomer)

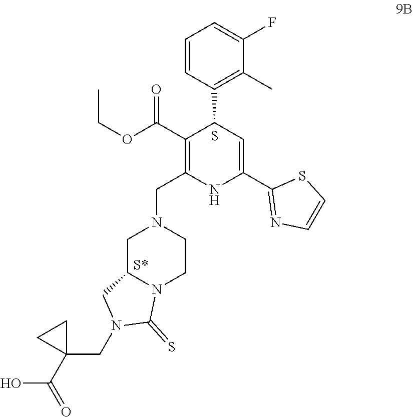

This compound was prepared from intermediate H2-1A and S3-B under same condition as for 9A. LC-MS (ESI): R_{T}=3.890 min, mass calcd. for C₂₉H₃₃FN₆O₄S₂ 612.2, m/z found 613.2 [M+H]⁺. ¹H NMR (400 MHz, CD₃OD) δ 7.94 (d, J=3.2 Hz, 1H), 7.73 (d, J=3.2 Hz, 1H), 7.18-7.10 (m, 2H), 6.96-6.92 (m, 1H), 5.99 (s, 1H), 4.52-4.49 (m, 1H), 4.15-4.03 (m, 4H), 3.97-3.89 (m, 3H), 3.88-3.81 (m, 1H), 3.41-3.36 (m, 1H), 3.32-3.27 (m, 1H), 3.01-2.88 (m, 2H), 2.52 (s, 3H), 2.48-2.41 (m, 1H), 2.19-2.13 (m, 1H), 1.22-1.19 (m, 2H), 1.13 (t, J=7.2 Hz, 3H), 1.03-0.94 (m, 2H).

### Compound 10B: 3-(-7-((6-(3-Fluoro-2-methylphenyl)-5-(methoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

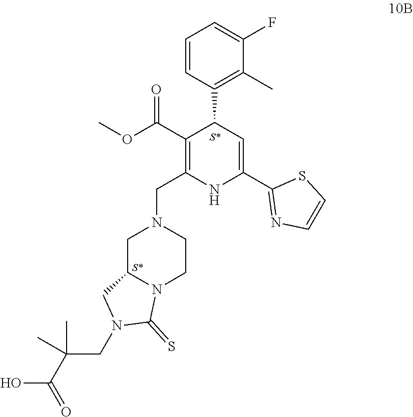 [SMILES: COC(=O)C1=C(CN2CCN3C(=S)N(CC(C)(C)C(=O)O)C[C@@H]3C2)NC(c2nccs2)=C[C@H]1c1cccc(F)c1C]

To a solution of methyl 6-(bromomethyl)-4-(3-fluoro-2-methylphenyl)-2-(thiazol-2-yl)-1,4-dihydropyrimidine-5-carboxylate (H4-1B) (100 mg, 90% purity, 0.212 mmol) in dichloromethane (6 mL) was added 2,2-dimethyl-3-(3-thioxohexahydroimidazo[1,5-a]pyrazin-2(3H)-yl)propanoic acid hydrochloride salt S1-A (69 mg, 90% purity, 0.211 mmol) and triethanolamine (348 mg, 2.33 mmol) at room temperature. After stirred at room temperature overnight, the mixture was diluted with ethyl acetate (30 mL) and washed with brine (30 mL), dried over Na₂SO₄₍ₛ₎ and filtered. The filtrate was concentrated under reduced pressure to give a residue, which was purified by Prep-HPLC (Column: Xtimate C18 (10 μm 50*250 mm); Mobile phase A: water (0.1% ammonium bicarbonate), Mobile phase B: acetonitrile; UV: 254 nm, Flow rate: 15 mL/min, Gradient: 20-60% (% B)) to afford the desired product (42 mg, 98.7% purity, 33% yield) as yellow solids. LC-MS (ESI): R_{T}=3.762 min, mass calcd. for C₂₈H₃₃FN₆O₄S₂ 600.7, m/z found 601.2 [M+H]⁺. ¹H NMR (400 MHz, CD₃OD) δ 7.95 (d, J=3.2 Hz, 1H), 7.74 (d, J=3.2 Hz, 1H), 7.18-7.08 (m, 2H), 6.96-6.92 (m, 1H), 5.98 (s, 1H), 4.54-4.51 (m, 1H), 4.13-4.04 (m, 2H), 3.96-3.84 (m, 3H), 3.70 (t, J=10.4 Hz, 1H), 3.62 (s, 3H), 3.30-3.25 (m, 2H), 2.98-2.96 (m, 1H), 2.90-2.87 (m, 1H), 2.53 (d, J=2.0 Hz, 3H), 2.49-2.42 (m, 1H), 2.18 (t, J=10.8 Hz, 1H), 1.22 (s, 3H), 1.21 (s, 3H).

### Compound 11A: 3-(7-((6-(2-Chloro-3,4-difluorophenyl)-5-(methoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

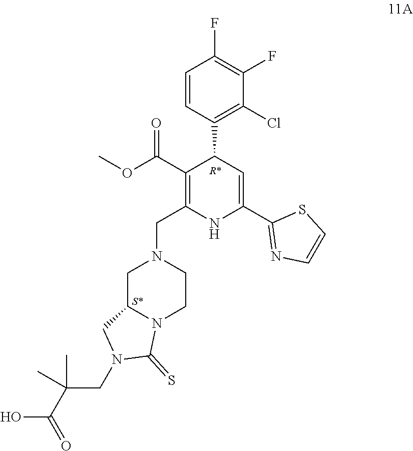 [SMILES: COC(=O)C1=C(CN2CCN3C(=S)N(CC(C)(C)C(=O)O)C[C@@H]3C2)NC(c2nccs2)=C[C@H]1c1ccc(F)c(F)c1Cl]

To a solution of 2,2-dimethyl-3-(3-thioxohexahydroimidazo[1,5-a]pyrazin-2(3H)-yl)propanoic acid hydrochloride S1-A (50 mg, 0.153 mmol) in tetrahydrofuran (5 mL) was added triethylamine (60 mg, 0.593 mmol). After stirred for 5 minutes, methyl 6-(bromomethyl)-4-(2-chloro-3,4-difluorophenyl)-2-(thiazol-2-yl)-1,4-dihydropyrimidine-5-carboxylate (H5-1A) (82 mg, 0.161 mmol) was added. The mixture was stirred at 40° C. for 2.5 hours, then acidified to pH=3 with 1 M hydrochloride aqueous solution and extracted with ethyl acetate (10 mL) for three times. The combined organic layers were washed with brine (10 mL), dried over Na₂SO₄₍ₛ₎, filtered and concentrated to give a residue, which was purified by Prep-HPLC (Column: Gilson Xbridge C18 (5 μm 19*150 mm), Mobile phase A: water (+0.1% ammonium bicarbonate), Mobile phase B: acetonitrile, UV: 214 nm, Flow rate: 15 mL/min, Gradient: 20-60% (% B)) to give the title compound (32.0 mg, 28% yield) as yellow solids. LC-MS (ESI): R_{T}=3.512 min, mass calcd. for C₂₇H₂₉ClF₂N₆O₄S₂ 638.1, m/z found 639.1[M+H]⁺. ¹H NMR (400 MHz, CD₃OD) δ 7.94 (d, J=3.2 Hz, 1H), 7.74 (d, J=3.2 Hz, 1H), 7.23-7.20 (m, 2H), 6.15 (s, 1H), 4.52-4.49 (m, 1H), 4.11-3.83 (m, 5H), 3.68 (t, J=9.6 Hz, 1H), 3.59 (s, 3H), 3.30-3.21 (m, 2H), 2.96-2.87 (m, 2H), 2.43 (td, J=11.6, 3.2 Hz, 1H), 2.17 (t, J=11.2 Hz, 1H), 1.22 (s, 3H), 1.21 (s, 3H).

### Compound 12B: 3-(7-((6-(3,4-Difluoro-2-methylphenyl)-5-(methoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

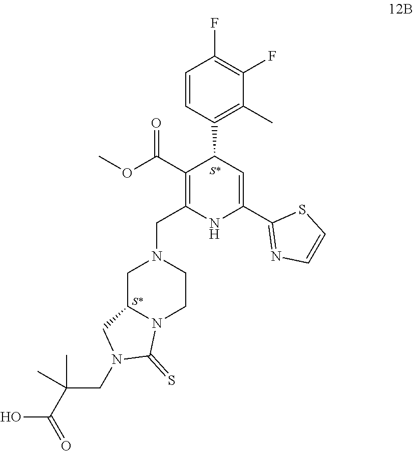 [SMILES: COC(=O)C1=C(CN2CCN3C(=S)N(CC(C)(C)C(=O)O)C[C@@H]3C2)NC(c2nccs2)=C[C@H]1c1ccc(F)c(F)c1C]

To a solution of methyl 6-(bromomethyl)-4-(3,4-difluoro-2-methylphenyl)-2-(thiazol-2-yl)-1,4-dihydropyrimidine-5-carboxylate (116-1B) (100 mg, 90% purity, 0.203 mmol) in dichloromethane (6 mL) was added 2,2-dimethyl-3-(3-thioxohexahydroimidazo[1,5-a]pyrazin-2(3H)-yl)propanoic acid hydrochloride Intermediate S1-A (66 mg, 90% purity, 0.203 mmol) and triethanolamine (334 mg, 2.24 mmol) at room temperature. After stirred at room temperature overnight, the mixture was dissolved in ethyl acetate (30 mL) and washed with brine (30 mL), dried over Na₂SO₄₍ₛ₎ and filtered. The filtrate was concentrated under reduced pressure to give a residue, which was purified by Prep-HPLC (Column: Xtimate C18 (10 μm 50*250 mm); Mobile phase A: water (0.1% ammonium bicarbonate), Mobile phase B: acetonitrile; UV: 254 nm, Flow rate: 15 mL/min, Gradient: 30-80% (% B)) to afford the desired product (18 mg, 14% yield) as yellow solids. LC-MS (ESI): R_{T}=3.474 min, mass calcd. for C₂₈H₃₂F₂N₆O₄S₂ 618.7, m/z found 619.2 [M+H]⁺. ¹H NMR (400 MHz, CD₃OD) δ 7.95 (d, J=3.2 Hz, 1H), 7.74 (d, J=3.2 Hz, 1H), 7.05-7.02 (m, 2H), 5.93 (s, 1H), 4.54-4.51 (m, 1H), 4.14-4.03 (m, 2H), 3.96 (s, 0.6H), 3.91 (s, 0.4H), 3.88-3.83 (m, 3H), 3.70 (t, J=10.0 Hz, 1H), 3.62 (s, 3H), 3.29-3.25 (m, 2H), 2.98-2.95 (m, 1H), 2.88-2.86 (m, 1H), 2.57 (d, J=2.4 Hz, 3H), 2.49-2.42 (m, 1H), 2.18 (t, J=10.8 Hz, 1H), 1.21 (s, 3H), 1.19 (s, 3H).

### Compound 13A: 3-(7-((6-(2-Bromo-4-fluorophenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

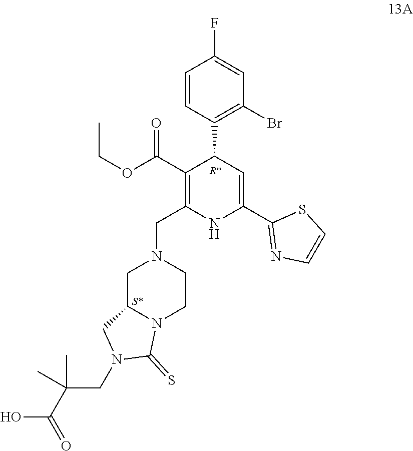 [SMILES: CCOC(=O)C1=C(CN2CCN3C(=S)N(CC(C)(C)C(=O)O)C[C@@H]3C2)NC(c2nccs2)=C[C@H]1c1ccc(F)cc1Br]

To a solution of 2,2-dimethyl-3-(3-thioxohexahydroimidazo[1,5-a]pyrazin-2(3H)-yl)propanoic acid hydrochloride Intermediate S1-A (100 mg, 0.306 mmol) in tetrahydrofuran (10 mL) were added triethylamine (149 mg, 1.48 mmol) and ethyl 4-(2-bromo-4-fluorophenyl)-6-(bromomethyl)-2-(thiazol-2-yl)-1,4-dihydro pyrimidine-5-carboxylate (117-A) (200 mg, 0.358 mmol) at room temperature. After heated at room temperature overnight under nitrogen atmosphere, the reaction mixture was quenched with water (20 mL) slowly and extracted with ethyl acetate (20 mL) for three times. The separated organic layer was washed with brine (20 mL), dried over Na₂SO₄₍ₛ₎, filtered and concentrated under reduced pressure to give a residue, which was purified by Prep-HPLC (Column: waters Xbridge C18 (5 μm 19*150 mm), Mobile Phase A: water (0.1% ammonium bicarbonate), Mobile Phase B: acetonitrile, UV: 214 nm, Flow rate: 15 mL/min, Gradient: 20-60% (% B)) to afford desired product (70 mg, 29% yield) as yellow solids. LC-MS (ESI): R_{T}=3.865 min, mass calcd. For C₂₈H₃₂BrFN₆O₄S₂ 678.1, m/z found 679.1 [M+H]⁺. ¹H NMR (400 MHz, DMSO-d₆) δ 9.63 (br s, 1H), 8.03 (d, J=2.8 Hz, 1H), 7.95 (d, J=2.8 Hz, 1H), 7.59-7.56 (m, 1H), 7.42-7.39 (m, 1H), 7.26-7.22 (m, 1H), 6.03 (s, 1H), 4.36 (d, J=14.4 Hz, 1H), 4.00-3.93 (m, 5H), 3.77 (d, J=2.8 Hz, 2H), 3.64 (t, J=10.0 Hz, 1H), 3.18-3.13 (m, 2H), 2.96-2.91 (m, 2H), 2.29-2.24 (m, 1.6H), 2.10-2.05 (m, 1.4H), 1.13 (s, 6H), 1.05 (t, J=6.8 Hz, 3H).

### Compound 14: 3-(7-((6-(2-Chloro-3,4-difluorophenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Mixture of 2 diastereomers)

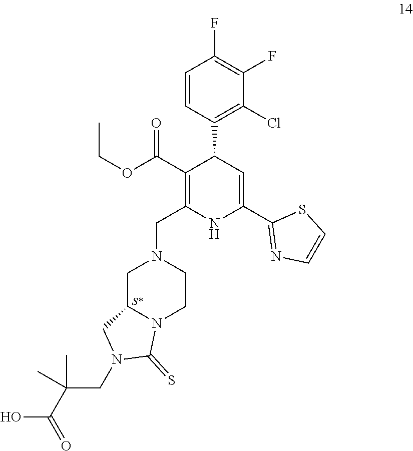 [SMILES: CCOC(=O)C1=C(CN2CCN3C(=S)N(CC(C)(C)C(=O)O)C[C@@H]3C2)NC(c2nccs2)=C[C@H]1c1ccc(F)c(F)c1Cl]

To a solution of ethyl 6-(bromomethyl)-4-(2-chloro-3,4-difluorophenyl)-2-(thiazol-2-yl)-1,4-dihydropyrimidine-5-carboxylate H8-1 (95 mg, 0.199 mmol) in dichloromethane (10 mL) was added 2,2-dimethyl-3-(3-thioxohexahydroimidazo [1,5-a]pyrazin-2(3H)-yl)propanoic acid hydrochloride S1-A (58 mg, 0.197 mmol), triethanolamine (90 mg, 0.604 mmol) at room temperature. After stirred at 40° C. overnight, the mixture was concentrated under reduced pressure to give a residue, which was purified by C18 column (acetonitrile:water=20% to 40%) to give the desired compound (34.6 mg, 98.8% purity, 26% yield) as yellow solids. LC-MS (ESI): R_{T}=3.747 min, mass calcd. for C₂₈H₃₁ClF₂N₆O₄S₂ 652.2, m/z found 653.2 [M+H]⁺. ¹H NMR (400 MHz, CD₃OD) δ 7.94 (d, J=2.8 Hz, 1H), 7.74 (d, J=3.2 Hz, 1H), 7.24-7.20 (m, 2H), 6.17 (s, 0.5H), 6.16 (s, 0.5H), 4.52-4.42 (m, 1H), 4.13-4.00 (m, 4H), 3.94-3.86 (m, 3H), 3.77-3.66 (m, 1H), 3.30-3.22 (m, 2H), 3.05-2.78 (m, 2H), 2.45-2.40 (m, 0.5H), 2.32-2.27 (m, 1H), 2.19-2.14 (m, 0.5H), 1.23-1.21 (m, 6H), 1.12 (t, J=7.2 Hz, 3H).

### Compound 14A: 3-(7-((6-(2-Chloro-3,4-difluorophenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

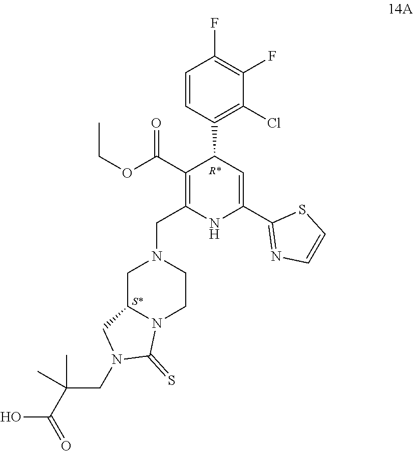 [SMILES: CCOC(=O)C1=C(CN2CCN3C(=S)N(CC(C)(C)C(=O)O)C[C@@H]3C2)NC(c2nccs2)=C[C@H]1c1ccc(F)c(F)c1Cl]

This compound was prepared from H8-1A and S1-A under same condition as for compound 14 and purified by C18 column (acetonitrile:water=20% to 40%) to give the desired compound (19.9 mg, 97.1% purity, 17% yield) as yellow solids. LC-MS (ESI): R_{T}=3.481 min, mass calcd. for C₂₈H₃₁ClF₂N₆O₄S₂ 652.2, m/z found 653.2 [M+H]⁺. ¹H NMR (400 MHz, CD₃OD) δ 7.94 (d, J=3.2 Hz, 1H), 7.74 (d, J=2.8 Hz, 1H), 7.24-7.21 (m, 2H), 6.16 (s, 1H), 4.52-4.48 (m, 1H), 4.11-4.01 (m, 4H), 3.94-3.80 (m, 3H), 3.71-3.66 (m, 1H), 3.30-3.22 (m, 2H), 2.96-2.87 (m, 2H), 2.45-2.39 (m, 1H), 2.19-2.14 (m, 1H), 1.21 (s, 6H), 1.12 (t, J=7.2 Hz, 3H).

### Compound 15A: 3-(7-((6-(3,4-Difluoro-2-methylphenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

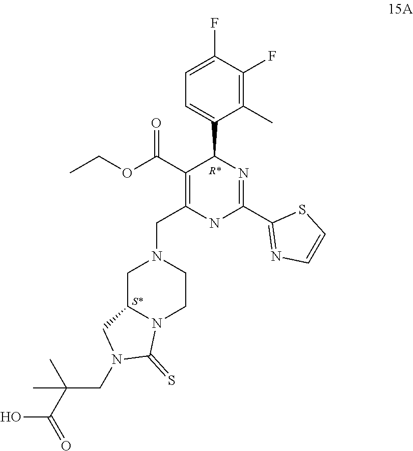 [SMILES: CCOC(=O)C1=C(CN2CCN3C(=S)N(CC(C)(C)C(=O)O)C[C@@H]3C2)NC(c2nccs2)=N[C@@H]1c1ccc(F)c(F)c1C]

To a solution of ethyl 6-(bromomethyl)-4-(3,4-difluoro-2-methylphenyl)-2-(thiazol-2-yl)-1,4-dihydropyrimidine-5-carboxylate H9-1A (100 mg, 0.197 mmol) in tetrahydrofuran (4 mL) was added 2,2-dimethyl-3-(3-thioxohexahydroimidazo[1,5-a]pyrazin-2(3H)-yl)propanoic acid hydrochloride S1-A (74 mg, 0.227 mmol) and triethylamine (0.14 mL, 0.97 mmol) at 40° C. for 2 hours. Then stirred at room temperature overnight, the mixture was dissolved in ethyl acetate (10 mL) and washed with brine (10 mL), dried over Na₂SO₄₍ₛ₎ and filtered. The filtrate was concentrated under reduced pressure to give a residue, which was purified by Prep-HPLC (separation condition: Column: Xtimate C18, 10 μm 50 mm*250 mm; Mobile Phase: acetonitrile:water (0.1% ammonium bicarbonate)=30%-80% at 15 mL/min; Temp: 35° C.; Wavelength: 254 nm) to afford the desired product (31 mg, 97.9% purity, 24% yield) as yellow solids. LC-MS (ESI): R_{T}=3.710 min, mass calcd. for C₂₉H₃₄F₂N₆O₄S₂ 632.7, m/z found 633.7 [M+H]⁺. ¹H NMR (400 MHz, CD₃OD) δ 7.95 (d, J=2.8 Hz, 1H), 7.74 (d, J=3.2 Hz, 1H), 7.10-7.00 (m, 2H), 5.94 (s, 1H), 4.53 (d, J=14.8 Hz, 1H), 4.14-4.02 (m, 4H), 3.97-3.87 (m, 3H), 3.70 (t, J=10.0 Hz, 1H), 3.30-3.23 (m, 2H), 2.98 (d, J=11.2 Hz, 1H), 2.89 (d, J=6.8 Hz, 1H), 2.58 (s, 1.5H), 2.57 (s, 1.5H), 2.45 (td, J=11.2, 3.6 Hz, 1H), 2.18 (t, J=10.0 Hz, 1H), 1.24 (s, 3H), 1.23 (s, 3H), 1.15 (t, J=6.8 Hz, 3H).

### Compound 15B: 3-(7-((6-(3,4-Difluoro-2-methylphenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

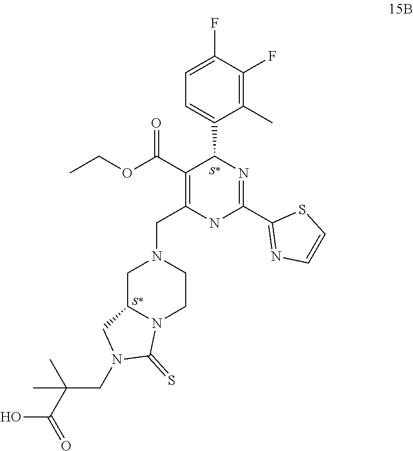 [SMILES: CCOC(=O)C1=C(CN2CCN3C(=S)N(CC(C)(C)C(=O)O)C[C@@H]3C2)NC(c2nccs2)=N[C@H]1c1ccc(F)c(F)c1C]

This compound was prepared from H9-1B and S1-A using same condition as for compound 15A and purified by Prep-HPLC (separation condition: Column: Xtimate C18, 10 μm 50 mm*250 mm; Mobile Phase: acetonitrile:water (0.1% ammonium bicarbonate)=30%-80% at 15 mL/min; Temp: 35° C.; Wavelength: 254 nm) to afford the desired product (30 mg, 98.2% purity, 24% yield) as yellow solids. LC-MS (ESI): R_{T}=3.539 min, mass calcd. for C₂₉H₃₄F₂N₆O₄S₂ 632.7, m/z found 633.7 [M+H]⁺. ¹H NMR (400 MHz, CD₃OD) δ 7.93 (d, J=3.2 Hz, 1H), 7.73 (d, J=2.8 Hz, 1H), 7.07-6.97 (m, 2H), 5.92 (s, 1H), 4.53 (d, J=14.4 Hz, 1H), 4.14-4.03 (m, 4H), 3.93-3.88 (m, 3H), 3.75 (t, J=9.6 Hz, 1H), 3.29-3.25 (m, 2H), 3.04 (d, J=10.0 Hz, 1H), 2.78 (d, J=11.2 Hz, 1H), 2.55 (s, 3H), 2.34-2.24 (m, 2H), 1.24 (s, 3H), 1.23 (s, 3H), 1.13 (t, J=7.2 Hz, 3H).

### Compound 16A: 3-(7-((6-(2-Bromo-4-fluorophenyl)-5-(methoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

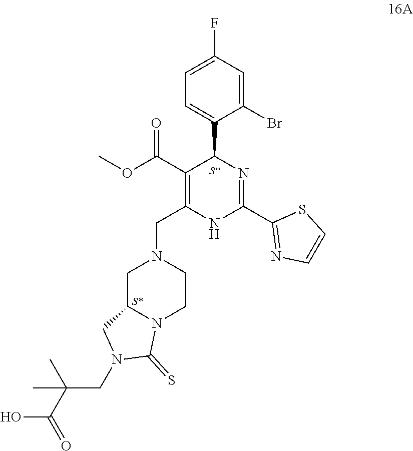 [SMILES: COC(=O)C1=C(CN2CCN3C(=S)N(CC(C)(C)C(=O)O)C[C@@H]3C2)NC(c2nccs2)=N[C@@H]1c1ccc(F)cc1Br]

To a solution of 2,2-dimethyl-3-(3-thioxohexahydroimidazo[1,5-a]pyrazin-2(3H)-yl)propanoic acid hydrochloride S1-A (126 mg, 0.386 mmol) in tetrahydrofuran (5 mL) were added triethylamine (195 mg, 1.93 mmol) and methyl 4-(2-bromo-4-fluorophenyl)-6-(bromomethyl)-2-(thiazol-2-yl)-1,4-dihydropyrimidine-5-carboxylate H10-1A (210 mg, 0.386 mmol) at room temperature. After stirred at room temperature overnight under nitrogen atmosphere, the reaction mixture was quenched with water (20 mL) slowly and extracted with ethyl acetate (20 mL) for three times. The separated organic layer was washed with brine (20 mL), dried over Na₂SO₄₍ₛ₎, filtered and concentrated under reduced pressure to give a residue, which was purified by C18 column (acetonitrile:water (0.1% ammonium bicarbonate)=05% to 95%) to give the title compound (33 mg, 99.7% purity, 13% yield) as yellow solids. LC-MS (ESI): R_{T}=3.095 min, mass calcd. for C₂₇H₃₀BrFN₆O₄S₂ 664.1, m/z found 665.1 [M+H]⁺.

¹H NMR (400 MHz, CD₃OD) δ 7.98-7.90 (m, 1H), 7.78-7.70 (m, 1H), 7.46-7.36 (m, 2H), 7.14-7.04 (m, 1H), 6.14 (s, 1H), 4.53-4.48 (m, 1H), 4.11-4.02 (m, 2H), 3.94-3.85 (m, 3H), 3.68 (t, J=9.6 Hz, 1H), 3.59 (s, 3H), 3.24-3.15 (m, 2H), 2.98-2.86 (m, 2H), 2.48-2.41 (m, 1H), 2.23-2.15 (m, 1H), 1.23 (s, 6H).

### Compound 16B: 3-(7-((6-(2-Bromo-4-fluorophenyl)-5-(methoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

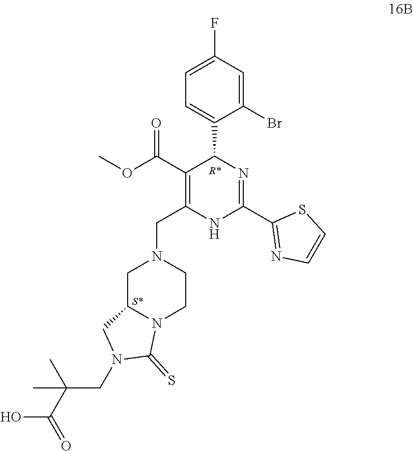 [SMILES: COC(=O)C1=C(CN2CCN3C(=S)N(CC(C)(C)C(=O)O)C[C@@H]3C2)NC(c2nccs2)=N[C@H]1c1ccc(F)cc1Br]

This compound was prepared from H10-1B and S1-A under same condition as for compound 16A and purified by Prep-HPLC (Column: sunfire C18 (5 μm 19*150 mm), Mobile Phase A: water (0.1% trifluoroacetic acid), Mobile Phase B: acetonitrile, UV: 214 nm, Flow rate: 15 mL/min, Gradient: 35-45% (% B)) to afford the product, which was further purified by C18 column (acetonitrile:water (0.1% ammonium bicarbonate)=05% to 95%) to give the title compound (47 mg, 99.4% purity, 18% yield) as yellow solids. LC-MS (ESI): R_{T}=3.096 min, mass calcd. for C₂₇H₃₀BrFN₆O₄S₂ 664.1, m/z found 665.1 [M+H]⁺. H NMR (400 MHz, CD₃OD) δ 7.94 (d, J=3.6 Hz, 1H), 7.73 (d, J=3.2 Hz, 1H), 7.42-7.38 (m, 2H), 7.10-7.06 (m, 1H), 6.14 (s, 1H), 4.46-4.42 (m, 1H), 4.12-4.03 (m, 2H), 3.92-3.85 (m, 3H), 3.75 (t, J=10.0 Hz, 1H), 3.59 (s, 3H), 3.28-3.25 (m, 2H), 3.06-3.03 (m, 1H), 2.81-2.74 (m, 1H), 2.33-2.26 (m, 2H), 1.23 (s, 3H), 1.22 (s, 3H).

### Compound 17: 2-((7-(((S)-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)methyl)butanoic acid(Mixture of Diastereomers)

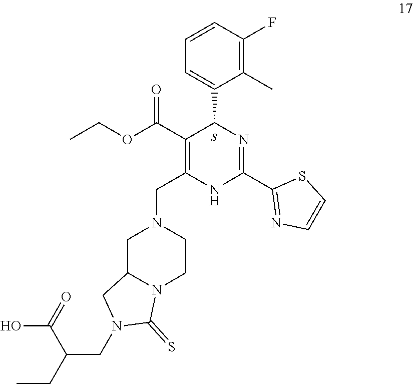

### Preparation of Intermediate S7

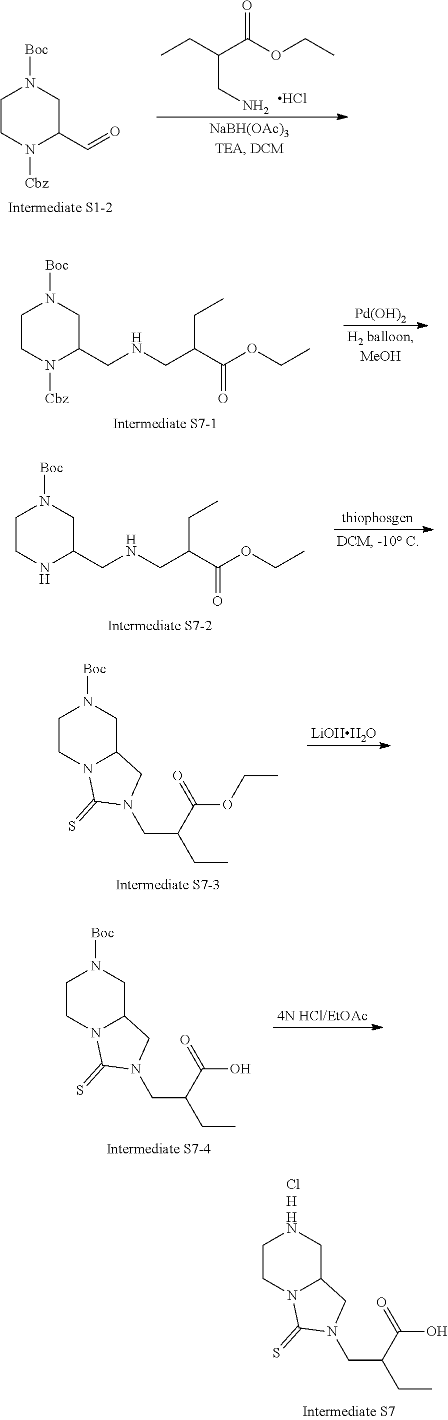

### Intermediate S7-1:1-Benzyl 4-tert-butyl 2-(((2-(ethoxycarbonyl)butyl)amino)-methyl)piperazine-1,4-dicarboxylate

A mixture of 1-benzyl 4-tert-butyl 2-formylpiperazine-1,4-dicarboxylate (intermediate S1-2) (1.10 g, 3.16 mmol), ethyl 2-(aminomethyl)butanoate hydrochloride (528 mg, 3.16 mmol) and triethylamine (638 mg, 6.32 mmol) in dichloromethane (20 mL) was stirred at room temperature for 30 minutes. Then sodium triacetoxyborohydride (2.01 g, 9.48 mmol) was added. After stirred at 40° C. overnight, the reaction mixture was diluted with dichloromethane (20 mL) and washed with water (10 mL), concentrated to give a residue, which was purified by C18 column (acetonitrile:water=30% to 90%) to give the title compound (800 mg, 60% yield) as yellow oil. ¹H NMR (400 MHz, DMSO-d₆) δ 7.37-7.29 (m, 5H), 5.12-5.04 (m, 2H), 4.08-3.76 (m, 6H), 3.31 (s, 1H), 2.91-2.66 (m, 6H), 2.37-2.31 (m, 1H), 1.45-1.42 (m, 11H), 1.17-1.14 (m, 3H), 0.80 (t, J=7.2 Hz, 3H).

### Intermediate S7-2: tert-Butyl 3-(((2-(ethoxycarbonyl)butyl)amino)methyl)-piperazine-1-carboxylate

To a solution of 1-benzyl 4-tert-butyl 2-(((2-(ethoxycarbonyl)butyl)amino)methyl)-piperazine-1,4-dicarboxylate (Intermediate S7-1) (800 mg, 1.68 mmol) in methanol (60 mL) was added palladium hydroxide (400 mg, 2.86 mmol). After stirred at 50° C. under hydrogen atmosphere (balloon) overnight, the reaction mixture was filtered. The filtrate was concentrated to give the title compound (330 mg, 57% yield) as colorless oil. ¹H NMR (400 MHz, DMSO-d₆) δ 4.09-4.04 (m, 2H), 3.79-3.70 (m, 2H), 2.82-2.80 (m, 1H), 2.69-2.64 (m, 2H), 2.59-2.55 (m, 1H), 2.47-2.33 (m, 6H), 2.03-1.97 (m, 1H), 1.83-1.67 (m, 1H), 1.51-1.44 (m, 2H), 1.40 (s, 9H), 1.18 (t, J=6.8 Hz, 3H), 0.83 (t, J=7.6 Hz, 3H).

### Intermediate S7-3: tert-Butyl 2-(2-(ethoxycarbonyl)butyl)-3-thioxohexahydro-imidazo[1,5-a]pyrazine-7(1H)-carboxylate

A solution of thiophosgene (150 mg, 1.30 mmol) in dichloromethane (2 mL) was dropwise added to a mixture of tert-butyl 3-(((2-(ethoxycarbonyl)butyl)amino)methyl)-piperazine-1-carboxylate (Intermediate S7-2) (300 mg, 0.87 mmol) and triethylamine (309 mg, 3.06 mmol) in dichloromethane (20 mL). After stirred at −10° C. under nitrogen atmosphere for 30 minutes, then at 35° C. overnight, the reaction mixture was diluted with ice water (20 mL) and extracted with dichloromethane (20 mL) twice. The combined extracts were washed with water (20 mL), dried over Na₂SO4₍ₛ₎, filtered and concentrated to give a residue, which was purified by C18 column (acetonitrile:water=30% to 70%) to give the title compound (140 mg, 42% yield) as yellow oil. LC-MS (ESI): R_{T}=1.778 min, mass calcd. for C₁₈H₃₁N₃O₄S 385.2, m/z found 386.2 [M+H]⁺.

### Intermediate S7-4:2-((7-(tert-Butoxycarbonyl)-3-thioxohexahydroimidazo[1,5-a]pyrazin-2(3H)-yl)methyl)butanoic acid

To a solution of tert-butyl 2-(2-(ethoxycarbonyl)butyl)-3-thioxohexahydroimidazo[1,5-a]pyrazine-7(1H)-carboxylate (Intermediate S7-3) (140 mg, 0.363 mmol) in tetrahydrofuran (10 mL) and water (10 mL) was added lithium hydroxide monohydrate (31 mg, 0.738 mmol). After stirred at room temperature overnight, the reaction mixture was concentrated to give a residue, which was purified by C18 column (acetonitrile:water=5% to 50%) to give the title compound (100 mg, 77% yield) as a yellow oil. LC-MS (ESI): R_{T}=1.233 min, mass calcd. for C₁₆H₂₇N₃O₄S 357.2, m/z found 358.2 [M+H]⁺.

### Intermediate S7: 2-((3-Thioxohexahydroimidazo[1,5-a]pyrazin-2(3H)-yl)methyl)butanoic acid hydrochloride

A solution of 2-((7-(tert-butoxycarbonyl)-3-thioxohexahydroimidazo[1,5-a]pyrazin-2(3H)-yl)methyl)butanoic acid (Intermediate S7-4) (100 mg, 0.280 mmol) in 4 N hydrochloride in ethyl acetate (10 mL) was stirred at room temperature for 1 hour. Then the mixture was concentrated under reduced pressure to give the title compound (80 mg, 100% yield) as white solids. LC-MS (ESI): R_{T}=0.439 min, mass calcd. for C₁₁H₂₀ClN₃O₂S 293.1, m/z found 258.2 [M−HCl+H]⁺.

### Compound 17: 2-((7-(((S)-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)methyl)butanoic acid (mixture of diastereomers)

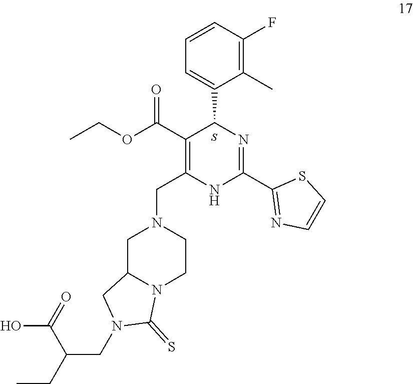

To a solution of 2-((3-thioxohexahydroimidazo[1,5-a]pyrazin-2(3H)-yl)methyl)butanoic acid hydrochloride (Intermediate S7) (80 mg, 0.280 mmol) in tetrahydrofuran (15 mL) was added triethylamine (126 mg, 1.25 mmol). After stirred for 5 minutes, (S)-ethyl 6-(bromomethyl)-4-(3-fluoro-2-methylphenyl)-2-(thiazol-2-yl)-1,4-dihydropyrimidine-5-carboxylate (112-A) (110 mg, 0.251 mmol) was added. After stirred at 40° C. overnight, the mixture was concentrated to give a residue, which was purified by C18 column (acetonitrile:water=20% to 70%) to give the title compound (12.1 mg, 95.1% purity, 8% yield) as yellow solids. LC-MS (ESI): R_{T}=3.652 min, mass calcd. for C₂₉H₃₅FN₆O₄S₂ 614.2, m/z found 615.2 [M+H]⁺. ¹H NMR (400 MHz, CD₃OD) δ 7.83-7.82 (m, 1H), 7.62 (d, J=3.2 Hz, 1H), 7.04-6.98 (m, 2H), 6.85-6.80 (m, 1H), 5.87 (d, J=2.0 Hz, 1H), 4.40-4.29 (m, 1H), 4.03-3.92 (m, 4H), 3.84-3.65 (m, 4H), 3.56-3.28 (m, 1H), 3.20-3.14 (m, 1H), 2.96-2.66 (m, 3H), 2.41 (s, 3H), 2.36-2.07 (m, 2H), 1.51-1.47 (m, 2H), 1.02 (t, J=7.2 Hz, 3H), 0.91-0.86 (m, 3H).

### Compound 18B: 1-((7-((6-(2-chloro-3-fluorophenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)methyl)cyclopropane-1-carboxylic acid single enantiomer)

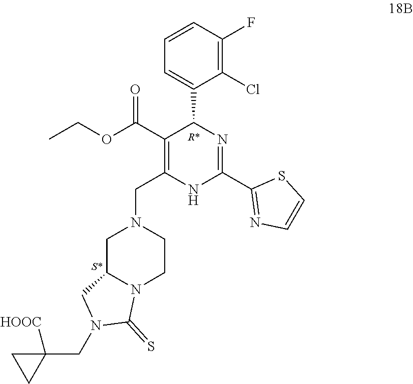

To a solution of ethyl 6-(bromomethyl)-4-(2-chloro-3-fluorophenyl)-2-(thiazol-2-yl)-1,4-dihydropyrimidine-5-carboxylate (H1-1A) (60 mg, 90% purity, 0.118 mmol) in tetrahydrofuran (4 mL) was added Intermediate S3-B (49 mg, 90% purity, 0.151 mmol) and triethylamine (87 mg, 0.583 mmol) under nitrogen atmosphere. After stirred at 40° C. under nitrogen atmosphere for 2.5 hours and then stirred at room temperature overnight, the reaction mixture was diluted with water (10 mL) and extracted with ethyl acetate (10 mL) twice. The combined organic layers were washed with brine (10 mL), dried over Na₂SO₄₍ₛ₎, filtered and concentrated to give a residue, which was purified by C18 column (acetonitrile:water=40% to 70%) to give the title compound (21 mg, 97.5% purity, 27.5% yield) as yellow solids. LC-MS (ESI): R_{T}=3.580 min, mass calcd. for C₂₈H₃₀ClFN₆O₄S₂ 632.1, m/z found 633.2 [M+H]⁺. ¹H NMR (400 MHz, CD₃OD) δ 7.84 (d, J=2.8 Hz, 1H), 7.64 (d, J=2.8 Hz, 1H), 7.22-7.13 (m, 2H), 7.06-7.01 (m, 1H), 6.12 (s, 1H), 4.40-4.37 (m, 1H), 4.01-3.92 (m, 4H), 3.90-3.71 (m, 4H), 3.30-3.25 (m, 1H), 3.20-3.16 (m, 1H), 2.86-2.80 (m, 2H), 2.36-2.29 (m, 1H), 2.10-2.05 (m, 1H), 1.15-1.09 (m, 2H), 1.03-0.96 (m, 5H).

### Compound 19A and 19B: (trans)-7-((6-(2-chloro-3-fluorophenyl)-5-(ethoxycarbonyl)-2-(thiazol-2-yl)-3,6-dihydropyrimidin-4-yl)methyl)-2-methyl-3-thioxooctahydro-imidazo[1,5-a]pyrazine-8-carboxylic acid (Single Isomers)

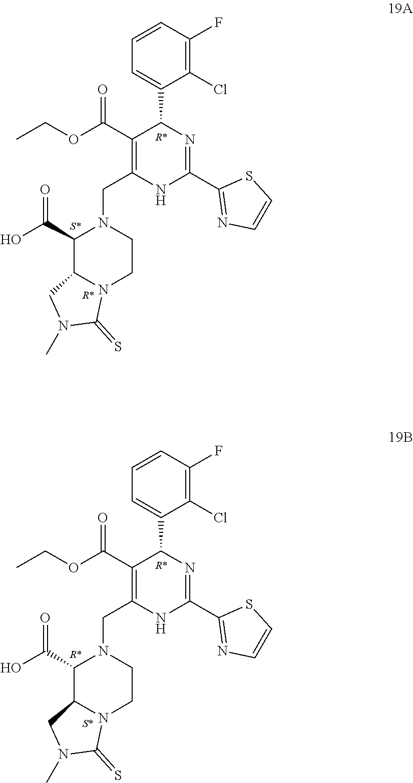

### Preparation of Intermediate S9

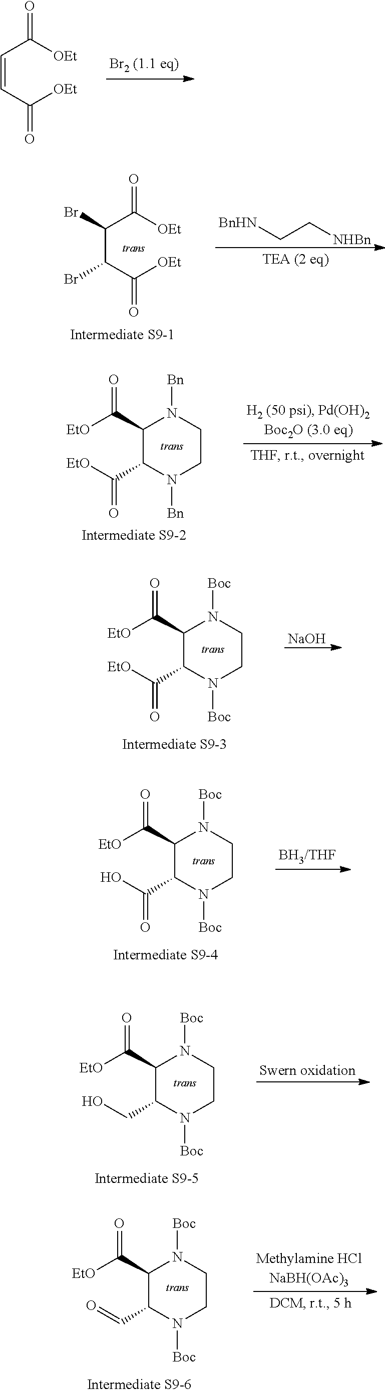

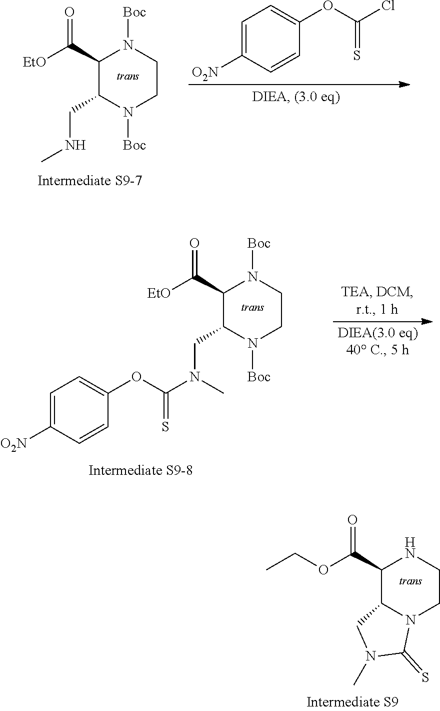 [SMILES: CCOC(=O)[C@@H]1[C@@H](CNC)N(C(=O)OC(C)(C)C)CCN1C(=O)OC(C)(C)C ; O=[N+]([O-])c1ccc(OC(=S)Cl)cc1 ; CCOC(=O)[C@@H]1[C@@H](CN(C)C(=S)Oc2ccc([N+](=O)[O-])cc2)N(C(=O)OC(C)(C)C)CCN1C(=O)OC(C)(C)C ; CCOC(=O)[C@H]1NCCN2C(=S)N(C)C[C@H]12]

### Intermediate S9-1: (trans)-Diethyl 2,3-dibromosuccinate

To a solution of diethyl maleate (10.8 g, 60 mmol) in perchloromethane (200 mL) was added bromine (10.66 g, 66.0 mmol) at 0° C. After stirred at room temperature overnight under nitrogen atmosphere, the reaction mixture was diluted with ice-water (100 mL), extracted with dichloromethane (100 mL) for three times. The combined organic layers were washed with saturated sodium sulfite aqueous solution (50 mL), dried over Na₂SO₄₍ₛ₎ and filtered. The filtrate was concentrated to give the crude compound (16.4 g, 82% yield) as colorless oil. ¹H NMR (400 MHz, DMSO-d₆) δ 6.75 (s, 2H), 4.23-4.18 (m, 4H), 1.27-1.23 (m, 6H).

### Intermediate S9-2: (trans)-Diethyl 1,4-dibenzylpiperazine-2,3-dicarboxylate

To a solution of (trans)-diethyl 2,3-dibromosuccinate (Intermediate S9-1) (20.0 g, 57.0 mmol) in toluene (60 mL) was added dropwise a solution of N¹,N²-dibenzylethane-1,2-diamine (14.0 g, 57.0 mmol) and triethylamine (11.7 g, 114 mmol) at 40° C. After stirred at 80° C. overnight, the mixture was allowed to cool down to room temperature, diluted with water (300 mL), extracted with ethyl acetate (100 mL) for three times. The combined organic layers were washed with brine (50 mL), dried over Na₂SO₄₍ₛ₎ and filtered. The filtrate was concentrated to give a residue, which was purified by silica gel column chromatography (petroleum ether:ethyl acetate=100:1 to 50:1) to afford the title compound (7.50 g, 32% yield) as yellow solids. ¹H NMR (400 MHz, CDCl₃) δ 7.30-7.20 (m, 10H), 4.25-4.13 (m, 4H), 3.92 (s, 4H), 3.89 (s, 2H), 3.29 (d, J=7.2 Hz, 2H), 2.53 (d, J=7.2 Hz, 2H), 1.23 (t, J=6.8 Hz, 6H).

### Intermediate S9-3: (trans)-1,4-Di-tert-butyl 2,3-diethyl piperazine-1,2,3,4-tetracarboxylate

To a solution of (trans)-diethyl 1,4-dibenzylpiperazine-2,3-dicarboxylate (Intermediate S9-2) (18.0 g, 41.0 mmol) in tetrahydrofuran (200 mL) was added di-tert-butyl dicarbonate (27.1 g, 123 mmol) and 20% palladium hydroxide on charcoal wt. (5.0 g). The reaction mixture was stirred at room temperature overnight under hydrogen atmosphere (50 psi). The mixture was filtered through a pad of celite. The filtrate was concentrated to give a residue, which was purified by silica gel column chromatography (petroleum ether:ethyl acetate=50:1 to 8:1) to afford the title compound (14.0 g, 80% yield) as yellow solids. ¹H NMR (400 MHz, DMSO-d₆) δ 5.24-5.19 (m, 2H), 4.27-4.14 (m, 4H), 3.78-3.67 (m, 2H), 3.05-2.94 (m, 1H), 2.87-2.80 (m, 1H), 1.40 (s, 9H), 1.38 (s, 9H), 1.23-1.16 (m, 6H).

### Intermediate S9-4: (trans)-1,4-Bis(tert-butoxycarbonyl)-3-(ethoxycarbonyl)-piperazine-2-carboxylic acid

To a solution of (trans)-1,4-di-tert-butyl 2,3-diethyl piperazine-1,2,3,4-tetracarboxylate (Intermediate S9-3) (5.66 g, 12.9 mmol) in ethanol (25 mL) was added dropwise a solution of sodium hydroxide (700 mg, 16.7 mmol) in water (8 mL) at 55° C. After stirred for 2 hours at 55° C., the mixture was allowed to cool down to room temperature, concentrated to remove ethanol and acidified with 2 M hydrochloride aqueous solution (about 20 mL) to pH 5. The mixture was extracted with ethyl acetate (100 mL) for three times. The combined organic layers were washed with brine (50 mL), dried over Na₂SO₄₍ₛ₎ and filtered. The filtrate was concentrated to give a residue, which was purified by silica gel column chromatography (petroleum ether:ethyl acetate=10:1 to 5:1) to afford the title compound (2.70 g, 52% yield) as white solids. ¹H NMR (400 MHz, DMSO-d₆) δ 13.49 (br s, 1H), 5.19-5.08 (m, 2H), 4.26-4.13 (m, 2H), 3.81-3.64 (m, 2H), 3.08-2.81 (m, 2H), 1.40 (s, 9H), 1.37 (s, 9H), 1.23-1.16 (m, 3H).

### Intermediate S9-5: (trans)-1,4-Di-tert-butyl 2-ethyl 3-(hydroxymethyl)piperazine-1,2,4-tricarboxylate

To a solution of (trans)-1,4-bis(tert-butoxycarbonyl)-3-(ethoxycarbonyl)piperazine-2-carboxylic acid (Intermediate S9-4) (2.37 g, 5.89 mmol) in tetrahydrofuran (20 mL) was added dropwise 1 M borane-tetrahydrofuran complex in tetrahydrofuran (14.1 mL, 14.1 mmol) at 0° C. After stirred at room temperature overnight under nitrogen atmosphere, the reaction mixture was acidified with 1 M hydrochloride aqueous solution (about 10 mL) to pH 3, extracted with dichloromethane (100 mL) for three times. The combined organic layers were washed with saturated sodium sulfite aqueous solution (50 mL), dried over Na₂SO₄₍ₛ₎ and filtered. The filtrate was concentrated to give a residue, which was purified by silica gel column chromatography (petroleum ether:ethyl acetate=10:1 to 2:1) to afford the title compound (1.60 g, 70% yield) as white solids. ¹H NMR (400 MHz, DMSO-d₆) δ 5.17-5.12 (m, 1H), 4.71 (d, J=13.6 Hz, 1H), 4.58-4.45 (m, 1H), 4.22-4.02 (m, 2H), 3.78-3.71 (m, 2H), 3.67-3.47 (m, 1H), 3.42-3.34 (m, 1H), 3.14-2.81 (m, 2H), 1.41-1.36 (m, 18H), 1.23-1.16 (m, 3H).

### Intermediate S9-6: (trans)-1,4-Di-tert-butyl 2-ethyl 3-formylpiperazine-1,2,4-tricarboxylate

To a solution of oxalyl chloride (0.38 mL) in dichloromethane (10 mL) was added dropwise a solution of dimethyl sulfoxide (695 mg, 8.80 mmol) in dichloromethane (2 mL) at −70° C. After stirred for 1 hour at this temperature, the reaction mixture was added dropwise a solution of (trans)-1,4-di-tert-butyl 2-ethyl 3-(hydroxymethyl)piperazine-1,2,4-tricarboxylate (Intermediate S9-5) (800 mg, 2.00 mmol) in dichloromethane (10 mL) at −70° C. After stirred for 2 hours at this temperature, the reaction mixture was quenched with triethylamine (1.64 g, 16.0 mmol) at −70° C. and then stirred for 1 hour at room temperature. The mixture was diluted with water (50 mL), extracted with dichloromethane (20 mL) for three times. The combined organic layers were washed with brine (10 mL), dried over Na₂SO₄₍ₛ₎ and filtered. The filtrate was concentrated to give the crude compound (550 mg, 71% yield) as yellow oil. ¹H NMR (400 MHz, DMSO-d₆) δ 9.61-9.57 (m, 1H), 5.38-5.28 (m, 1H), 5.19-5.09 (m, 1H), 4.25-4.14 (m, 2H), 3.83-3.57 (m, 2H), 3.01-2.73 (m, 2H), 1.41-1.37 (m, 18H), 1.27 -1.17 (m, 3H).

### Intermediate S9-7: (trans)-1,4-Di-tert-butyl 2-ethyl 3-((methylamino)methyl)-piperazine-1,2,4-tricarboxylate

To a solution of (trans)-1,4-di-tert-butyl 2-ethyl 3-formylpiperazine-1,2,4-tricarboxylate (Intermediate S9-6) (2.44 g, 6.00 mmol) in dichloromethane (30 mL) was added methylamine hydrochloride (620 mg, 9.00 mmol), NaBH(OAc)₃ (3.28 g, 15.0 mmol) and two drops of acetic acid. The reaction mixture was stirred for 5 hours at room temperature and quenched with saturated sodium bicarbonate aqueous solution (20 mL). The mixture was extracted with dichloromethane (30 mL) for three times. The combined organic layers were washed with brine (20 mL), dried over Na₂SO₄₍ₛ₎ and filtered. The filtrate was concentrated to give a residue, which was purified by silica gel column chromatography (dichloromethane:methanol=30:1) to give the title compound (450 mg, 19% yield) as yellow oil. H NMR (400 MHz, DMSO-d₆) δ 4.79-4.72 (m, 1H), 4.63-4.48 (m, 1H), 4.20-4.05 (m, 2H), 3.75-3.63 (m, 2H), 3.05-2.80 (m, 2H), 2.75-2.66 (m, 1H), 2.51 (s, 2H), 2.33-2.32 (m, 3H), 1.41-1.36 (m, 18H), 1.36-1.19 (m, 3H).

### Intermediate S9-8: (trans)-1,4-Di-tert-butyl 2-ethyl 3-((methyl((4-nitrophenoxy)carbonothioyl)amino)methyl)piperazine-1,2,4-tricarboxylate

To a solution of (trans)-1,4-di-tert-butyl 2-ethyl 3-((methylamino)methyl)piperazine-1,2,4-tricarboxylate (Intermediate S9-7) (1.00 g, 90% purity, 2.24 mmol) in dichloromethane (8 mL) was added O-(4-nitrophenyl) carbonochloridothioate (1.10 g, 90% purity, 4.55 mmol) and N,N-diisopropylethylamine (873 mg, 6.75 mmol). After stirred at 40° C. overnight, the reaction mixture was cooled down to room temperature and quenched with ice-water (15 mL) and extracted with dichloromethane (20 mL) for three times. The combined organic layers were washed with brine (15 mL), dried over Na₂SO₄₍ₛ₎ and filtered. The filtrate was concentrated to give a residue, which was purified by silica gel column chromatography (petroleum ether:ethyl acetate=10:1 to 4:1) to afford the title compound (1.45 g, 90% purity from ¹H NMR, 99% yield) as yellow solids. ¹H NMR (400 MHz, CDCl₃) δ 8.30-8.26 (m, 2H), 7.35-7.29 (m, 0.5H), 7.25-7.21 (m, 1.5H), 5.38-5.18 (m, 1H), 4.71-4.50 (m, 1H), 4.35-4.15 (m, 3H), 4.06-3.85 (m, 3H), 3.60-3.41 (m, 3H), 3.31-2.99 (m, 2H), 1.50-1.45 (m, 18H), 1.31-1.26 (m, 3H).

### Intermediate S9: (trans)-Ethyl 2-methyl-3-thioxooctahydroimidazo[1,5-a]pyrazine-8-carboxylate

To a solution of (trans)-1,4-di-tert-butyl 2-ethyl 3-((methyl((4-nitrophenoxy)carbonothioyl)amino)methyl)piperazine-1,2,4-tricarboxylate (Intermediate S9-8) (158 mg, 90% purity, 0.244 mmol) in dichloromethane (3 mL) was added trifluoroacetic acid (2 mL). The mixture was stirred for 1 hour at room temperature. And then the mixture was concentrated to remove trifluoroacetic acid to give a residue, which was dissolved in dichloromethane (5 mL). To the solution was added N,N-diisopropylethylamine (95 mg, 0.735 mmol). After stirred at 40° C. for 5 hours, the reaction mixture was cooled to room temperature, diluted with water (10 mL) and extracted with ethyl acetate (10 mL) twice. The combined organic layers were washed with brine (10 mL), dried over Na₂SO₄₍ₛ₎, filtered and concentrated to give a residue, which was purified by silica gel column chromatography (petroleum ether:ethyl acetate=2:1) to afford the title compound (68 mg, 85% purity from ¹H NMR, 97% yield) as yellow solids. LC-MS (ESI): R_{T}=1.080 min, mass calcd. for C₁₀H₁₇N₃O₂S 243.1, m/z found 244.1 [M+H]⁺. ¹H NMR (400 MHz, CDCl₃) δ 4.45-4.41 (m, 1H), 4.24 (q, J=7.2 Hz, 2H), 3.84-3.72 (m, 2H), 3.50-3.46 (m, 1H), 3.29 (d, J=9.2 Hz, 1H), 3.17 (s, 3H), 3.13-3.09 (m, 1H), 3.00 (td, J=12.4, 3.2 Hz, 1H), 2.80 (td, J=12.4, 3.2 Hz, 1H), 1.31 (t, J=7.2 Hz, 3H).

### Chiral Separation of Intermediate S9

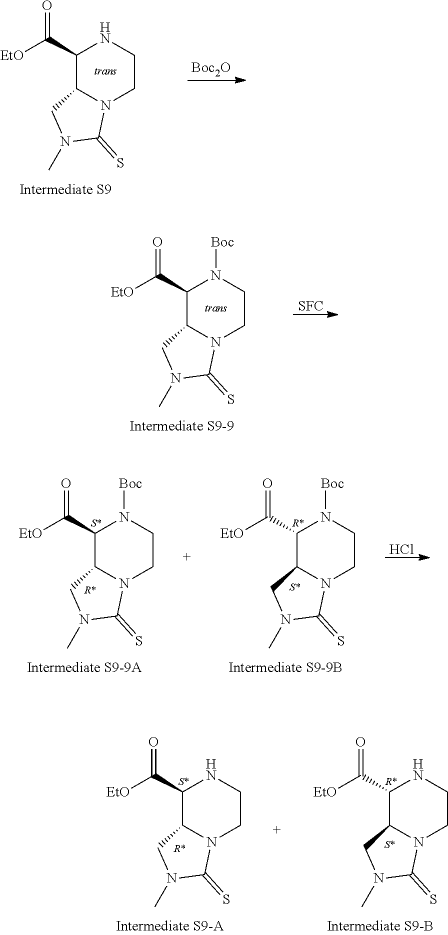

### Intermediate S9-9: (trans)-7-tert-Butyl 8-ethyl 2-methyl-3-thioxohexahydro-imidazo[1,5-a]pyrazine-7,8(1H)-dicarboxylate

LC-MS (ESI): R_{T}=1.58 min, mass calcd. for C₁₅H₂₅N₃O₄S 343.2, m/z found 288.0 [M+H−56]⁺. ¹H NMR (400 MHz, DMSO-d₆) δ 4.18-4.10 (m, 4H), 3.90-3.64 (m, 3H), 3.59-3.55 (m, 1H), 3.49-3.44 (m, 1H), 3.26-3.14 (m, 1H), 3.01 (s, 3H), 1.37 (s, 9H), 1.23-1.18 (m, 3H).

Racemic Intermediate S9-9 (1.30 g, 90% purity, 3.57 mmol) was separated by chiral Prep. HPLC (separation conditon: Column: Chiralpak IG 5 μm 20*250 mm; Mobile Phase: Hex:EtOH=80:20 at 18 mL/min; Temp: 30° C.; Wavelength: 230 nm) to give the title compounds Intermediate S9-9A (600 mg, 90% purity from ¹H NMR, 46% yield, 100% stereopure) and Intermediate S9-9B (610 mg, 90% purity from H NMR, 47% yield, 98.8% stereopure) as yellow solids.

Intermediate Intermediate S9-9A: LC-MS (ESI): R_{T}=1.57 min, mass calcd. for C₁₅H₂₅N₃O₄S 343.2, m/z found 288.0 [M+H−56]⁺. Chiral analysis (Column: Chiralpak IG 5 μm 4.6*250 mm; Mobile Phase: Hex:EtOH=70:30 at 1 mL/min; Temp: 30° C.; Wavelength: 254 nm, R_{T}=11.731 min). ¹H NMR (300 MHz, CDCl₃) δ 4.29-4.02 (m, 5H), 3.86-3.75 (m, 2H), 3.66-3.58 (m, 2H), 3.47-3.27 (m, 1H), 3.16 (s, 3H), 1.44 (s, 9H), 1.29 (t, J=7.2 Hz, 3H).

Intermediate Intermediate S9-9B: LC-MS (ESI): R_{T}=1.57 min, mass calcd. for C₁₅H₂₅N₃O₄S 343.2, m/z found 288.0 [M+H−56]⁺. Chiral analysis (Column: Chiralpak IG 5 μm 4.6*250 mm; Mobile Phase: Hex:EtOH=70:30 at 1 mL/min; Temp: 30° C.; Wavelength: 254 nm, R_{T}=14.006 min). ¹H NMR (300 MHz, CDCl₃) δ 4.28-4.03 (m, 5H), 3.92-3.69 (m, 2H), 3.66-3.50 (m, 2H), 3.42-3.28 (m, 1H), 3.15 (s, 3H), 1.44 (s, 9H), 1.29 (t, J=7.2 Hz, 3H).

Intermediate S9-A and S9-B were obtained from intermediate S9-9A and S9-9B by treating with 4M HCl solution in EA respectively. The volatiles were removed and the residue was used directly in next step reaction.

### Compound 19A-1 and 19B-1: (trans)-7-((6-(2-chloro-3-fluorophenyl)-5-(ethoxycarbonyl)-2-(thiazol-2-yl)-3,6-dihydropyrimidin-4-yl)methyl)-2-methyl-3-thioxooctahydro-imidazo[1,5-a]pyrazine-8-carboxylic acid

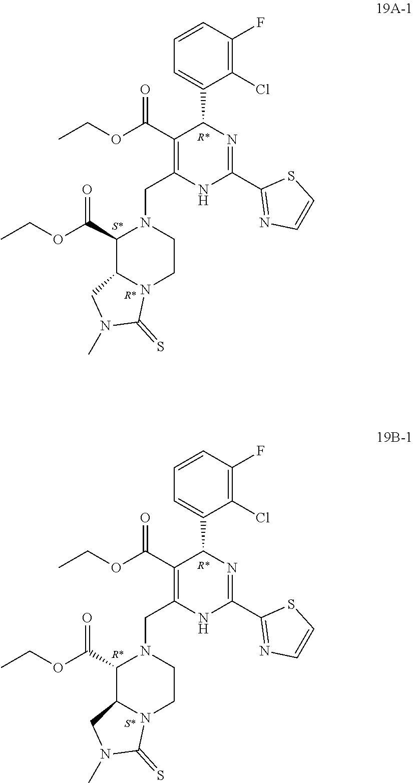

Compound 19A-1 and 19B-1 were prepared by coupling of H1-1A and intermediate S9-A and S9-B analogous to compound 18B.

Compound 19A-1: LC-MS (ESI): R_{T}=1.84 min, mass calcd. for C₂₇H₃₀ClFN₆O₄S₂ 620.1, m/z found 620.9 [M+H]⁺. ¹H NMR (400 MHz, CDCl₃) δ 9.77 (s, 1H), 7.84 (d, J=3.2 Hz, 1H), 7.44 (d, J=3.2 Hz, 1H), 7.19-7.13 (m, 2H), 7.05-7.01 (m, 1H), 6.29 (s, 1H), 4.45-4.41 (m, 1H), 4.35-4.25 (m, 3H), 4.06-3.98 (m, 3H), 3.69-3.64 (m, 2H), 3.56-3.51 (m, 1H), 3.30-3.23 (m, 2H), 3.19 (s, 3H), 2.82-2.79 (m, 1H), 2.53-2.46 (m, 1H), 1.31 (t, J=7.2 Hz, 3H), 1.11 (t, J=7.2 Hz, 3H).

Compound 19B-1: LC-MS (ESI): R_{T}=1.83 min, mass calcd. for C₂₇H₃₀ClFN₆O₄S₂ 620.1, m/z found 620.9 [M+H]⁺. ¹H NMR (400 MHz, CDCl₃) δ 9.77 (s, 1H), 7.86 (d, J=3.2 Hz, 1H), 7.43 (d, J=3.2 Hz, 1H), 7.19-7.11 (m, 2H), 7.07-7.00 (m, 1H), 6.25 (s, 1H), 4.54-4.51 (m, 1H), 4.25-4.13 (m, 3H), 4.06-3.97 (m, 3H), 3.77 (d, J=17.6 Hz, 1H), 3.65 (t, J=10.4 Hz, 1H), 3.54-3.50 (m, 1H), 3.35-3.28 (m, 1H), 3.25-3.22 (m, 1H), 3.19 (s, 3H), 2.93-2.91 (m, 1H), 2.64-2.57 (m, 1H), 1.20 (t, J=7.2 Hz, 3H), 1.10 (t, J=7.2 Hz, 3H).

Compound 19A and 19B were obtained by treating compound 19A-1 and 19B-1 with LiOH in THF/MeOH at rt.

Compound 19A: LC-MS (ESI): R_{T}=3.624 min, mass calcd. for C₂₅H₂₆ClFN₆O₄S₂ 592.1, m/z found 593.1. ¹H NMR (400 MHz, CD₃OD) δ 7.97 (d, J=3.2 Hz, 1H), 7.75 (d, J=3.2 Hz, 1H), 7.34-7.27 (m, 2H), 7.18-7.12 (m, 1H), 6.25 (s, 1H), 4.40-4.37 (m, 1H), 4.28 (d, J=16.8 Hz, 1H), 4.10-4.02 (m, 3H), 3.90 (d, J=16.8 Hz, 1H), 3.79-3.75 (m, 1H), 3.70-3.66 (m, 1H), 3.32-3.28 (m, 1H), 3.21-3.11 (m, 1H), 3.15 (s, 3H), 2.91-2.88 (m, 1H), 2.54-2.47 (m, 1H), 1.13 (t, J=6.8 Hz, 3H).

Compound 19B: LC-MS (ESI): R_{T}=3.620 min, mass calcd. for C₂₅H₂₆ClFN₆O₄S₂ 592.1, m/z found 593.2. ¹H NMR (400 MHz, CD₃OD) δ 7.95 (d, J=3.2 Hz, 1H), 7.73 (d, J=3.2 Hz, 1H), 7.35-7.29 (m, 2H), 7.18-7.13 (m, 1H), 6.21 (s, 1H), 4.45-4.41 (m, 1H), 4.22 (d, J=16.8 Hz, 1H), 4.08-4.02 (m, 3H), 3.92 (d, J=16.8 Hz, 1H), 3.76 (t, J=10.4 Hz, 1H), 3.68-3.64 (m, 1H), 3.32-3.28 (m, 1H), 3.19-3.18 (m, 1H), 3.16 (s, 3H), 3.06-3.03 (m, 1H), 2.64-2.57 (m, 1H), 1.12 (t, J=6.8 Hz, 3H).

### Compound 20A: 3-(7-((6-(2-chloro-3-fluorophenyl)-5-(methoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

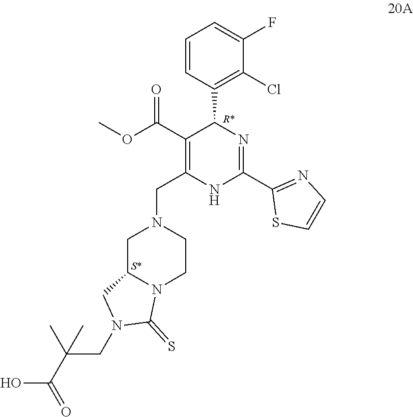

Compound 20A was prepared by intermediate H11-1A and S1-A analogous to compound 18B.

LC-MS (ESI): R_{T}=3.511 min, mass calcd. for C₂₇H₃₀ClFN₆O₄S₂ 620.1, m/z found 621.2 [M+H]⁺. ¹H NMR (400 MHz, DMSO-d₆) δ 8.02 (d, J=3.2 Hz, 1H), 7.95 (d, J=3.2 Hz, 1H), 7.39-7.29 (m, 2H), 7.24-7.22 (m, 1H), 6.09 (s, 1H), 4.35 (d, J=12.4 Hz, 1H), 3.99 (d, J=16.8 Hz, 1H), 3.94-3.87 (m, 2H), 3.79 (d, J=14.0 Hz, 1H), 3.74 (d, J=14.0 Hz, 1H), 3.64 (t, J=9.6 Hz, 1H), 3.52 (s, 3H), 3.18-3.12 (m, 2H), 2.97-2.88 (m, 2H), 2.35-2.24 (m, 1H), 2.09 (t, J=10.8 Hz, 1H), 1.12 (s, 6H).

### Compound 21A: 3-(7-((6-(2-chloro-4-fluorophenyl)-5-(ethoxycarbonyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

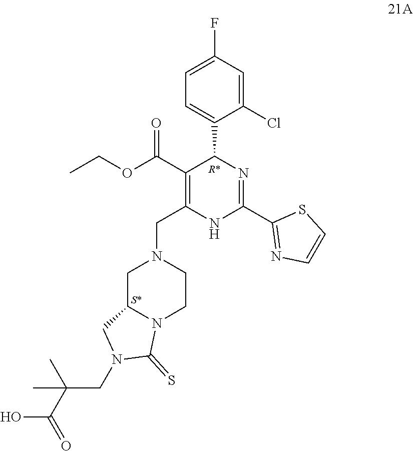

Compound 21A was prepared by intermediate H12-1A and S1-A analogous to compound 18B. LC-MS (ESI): R_{T}=3.522 min, mass calcd. for C₂₈H₃₂ClFN₆O₄S₂ 634.2, m/z found 635.2 [M+H]⁺. ¹H NMR (400 MHz, CD₃OD) δ 7.96 (d, J=2.8 Hz, 1H), 7.76 (d, J=3.2 Hz, 1H), 7.46-7.43 (m, 1H), 7.25-7.22 (m, 1H), 7.09-7.04 (m, 1H), 6.18 (s, 1H), 4.54-4.51 (m, 1H), 4.13 (s, 0.4H), 4.08-4.02 (m, 3.6H), 3.95 (s, 0.6H), 3.93-3.85 (m, 2.4H), 3.72-3.68 (m, 1H), 3.36-3.23 (m, 2H), 2.99-2.90 (m, 2H), 2.47-2.40 (m, 1H), 2.21-2.15 (m, 1H), 1.24 (s, 6H), 1.16 (t, J=6.8 Hz, 3H).

### Compound 22A: 3-(7-((2-(3,5-difluoropyridin-2-yl)-5-(ethoxycarbonyl)-6-(3-fluoro-2-methylphenyl)-3,6-dihydropyrimidin-4-yl)methyl)-3-thioxohexahydroimidazo-[1,5-a]pyrazin-2(3H)-yl)-2,2-dimethylpropanoic acid (Single Enantiomer)

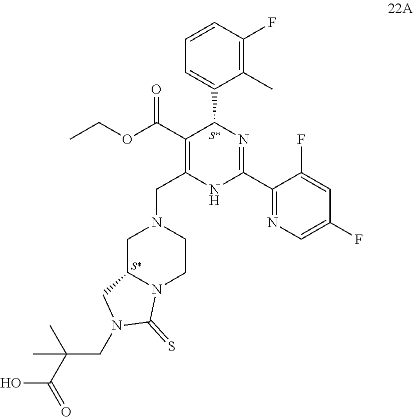

Compound 22A was prepared by intermediate H13-1A and S1-A analogous to compound 18B. LC-MS (ESI): R_{T}=3.642 min, mass calcd. for C₃₁H₃₅F₃N₆O₄S 644.2, m/z found 645.3 [M+H]⁺. ¹H NMR (400 MHz, CD₃OD) δ 8.52 (d, J=2.0 Hz, 1H), 7.75-7.70 (m, 1H), 7.17-7.08 (m, 2H), 6.97-6.92 (m, 1H), 6.06 (s, 1H), 4.52 (d, J=13.6 Hz, 1H), 4.19-4.06 (m, 4H), 3.96-3.86 (m, 3H), 3.72 (t, J=10.0 Hz, 1H), 3.28-3.24 (m, 2H), 2.98 (d, J=11.6 Hz, 1H), 2.89 (d, J=11.2 Hz, 1H), 2.54 (s, 3H), 2.46 (td, J=12.0, 3.2 Hz, 1H), 2.15 (t, J=11.2 Hz, 1H), 1.25 (s, 3H), 1.24 (s, 3H), 1.16 (t, J=7.2 Hz, 3H).

### Compound 23: 4-(7-((5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)benzoic acid (Mixture of Diasteromers)

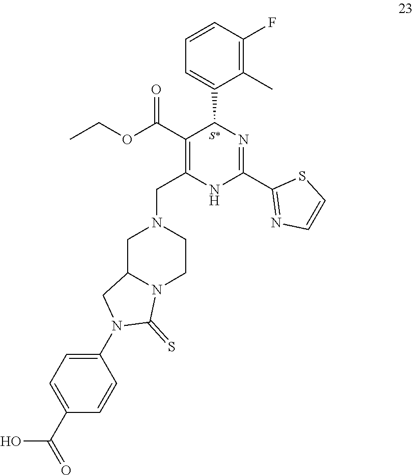

### Preparation of Intermediate S10

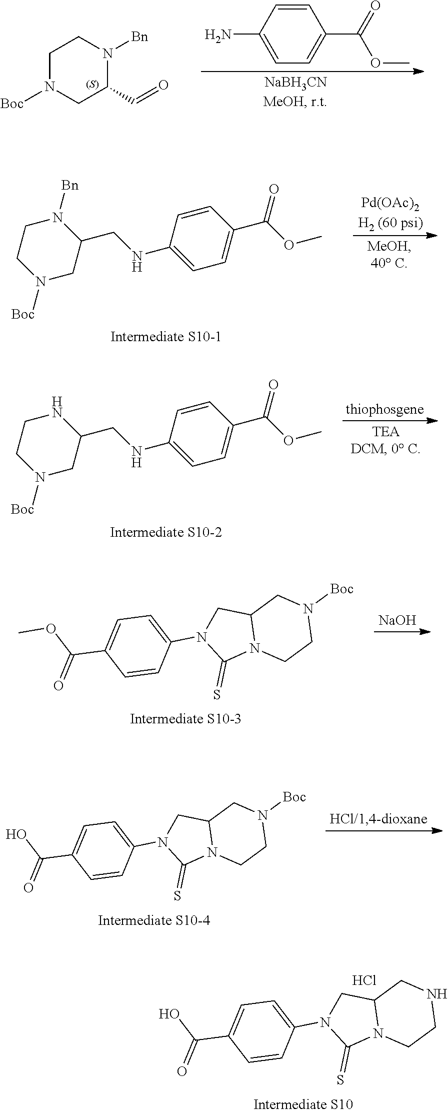

### Intermediate S10-1 Intermediate S10-2 Intermediate S10-3

### Intermediate S10-1: tert-butyl 4-benzyl-3-(((4-(methoxycarbonyl)phenyl)amino)-methyl)piperazine-1-carboxylate

The solution of (S)-tert-butyl 4-benzyl-3-formylpiperazine-1-carboxylate (2 g, 90% purity, 5.91 mmol) and methyl 4-aminobenzoate (900 mg, 5.95 mmol) in methanol (50 mL) was stirred at room temperature under nitrogen atmosphere for 16 hours. After sodium cyanoborohydride (700 mg, 11.1 mmol) was added at 0° C., the mixture was stirred at room temperature for 2 hours. Then the mixture was quenched with ice water (100 mL) and extracted with ethyl acetate (60 mL) for three times. The combined organic layers were dried over Na₂SO₄₍ₛ₎ and filtered. The filtrate was concentrated and purified by silica gel column chromatography (petroleum ether:ethyl acetate=8:1 to 2:1) to give the title compound (2 g, 78% purity, 60% yield) as light yellow oil. LC-MS (ESI): R_{T}=1.90 min, mass calcd. for C₂₅H₃₃N₃O₄ 439.2, m/z found 440.1 [M+H]⁺.

### Intermediate S10-2: tert-butyl 3-(((4-(methoxycarbonyl)phenyl)amino)methyl)-piperazine-1-carboxylate

To the solution of tert-butyl 4-benzyl-3-(((4-(methoxycarbonyl)phenyl)amino)methyl) piperazine-1-carboxylate (Intermediate S10-2) (2 g, 78% purity, 3.55 mmol) in methanol (40 mL) was added palladium (II) acetate (100 mg) and activated carbonate (500 mg) at room temperature. After stirred at 40° C. under 60 psi hydrogen atmosphere overnight, the mixture was filtered. The filtrate was concentrated to give the title compound (2 g, 56% purity, 90% yield) as light yellow oil which was directly used in next step without purification. LC-MS (ESI): R_{T}=1.51 min, mass calcd. for C₁₈H₂₇N₃O₄ 349.2, m/z found 350.0 [M+H]⁺.

### Intermediate S10-3: tert-butyl 2-(4-(methoxycarbonyl)phenyl)-3-thioxohexa-hydroimidazo[1,5-a]pyrazine-7(1H)-carboxylate

To a solution of tert-butyl 3-(((4-(methoxycarbonyl)phenyl)amino)methyl)piperazine-1-carboxylate (Intermediate S10-2) (2 g, 56% purity, 3.21 mmol) in dichloromethane (25 mL) was added triethylamine (1 g, 9.88 mmol) and thiophosgene (550 mg, 4.78 mmol) at 0° C. After stirred at 0° C. for 1 hour. The reaction mixture was quenched with water (80 mL) and extracted with dichloromethane (50 mL) twice. The combined organic layers were washed with brine (150 mL), dried over Na₂SO₄₍ₛ₎ and filtered. The filtrate was concentrated and purified by C18 column (acetonitrile:water=68% to 75%) to give the title compound (500 mg, 94% purity, 37% yield) as yellow solids. LC-MS (ESI): R_{T}=1.68 min, mass calcd. for C₁₉H₂₅N₃O₄S 391.2, m/z found 392.0 [M+H]⁺. ¹H NMR (400 MHz, CDCl₃) δ 8.08-8.04 (m, 2H), 7.77-7.73 (m, 2H), 4.63-4.59 (m, 1H), 4.42-4.31 (m, 1H), 4.17-4.14 (m, 1H), 4.03-3.98 (m, 1H), 3.91 (s, 3H), 3.77-3.71 (m, 2H), 3.12-3.05 (m, 1H), 2.97-2.89 (m, 1H), 2.82-2.71 (m, 1H), 1.50 (s, 9H).

### Intermediate S10-4:4-(7-(tert-butoxycarbonyl)-3-thioxohexahydroimidazo[1,5-a]pyrazin-2(3H)-yl)benzoic acid

To the solution of tert-butyl 2-(4-(methoxycarbonyl)phenyl)-3-thioxohexahydroimidazo[1,5-a]pyrazine-7(1H)-carboxylate (Intermediate S10-3) (100 mg, 94% purity, 0.24 mmol) in methanol (3 mL) and water (2 mL) was added sodium hydroxide (50 mg, 1.25 mmol) under nitrogen atmosphere at 0° C. After stirred at 35° C. for 16 hours, the mixture was cooled down and concentrated under reduced pressure to give a residue, which was diluted with water (20 mL). The resulting mixture was acidified with 2 M hydrochloride aqueous solution (1 mL) to pH ˜5 and extracted with ethyl acetate (30 mL) twice. The combined organic layers were washed with brine (50 mL), dried over Na₂SO₄₍ₛ₎ and filtered. The filtrate was concentrated to give the title compound (75 mg, 83% yield) as yellow solids which was directly used in next step without further purification. LC-MS (ESI): R_{T}=1.25 min, mass calcd. for C₁₈H₂₃N₃O₄S 377.1, m/z found 378.0 [M+H]⁺.

### Intermediate S10:4-(3-thioxohexahydroimidazo[1,5-a]pyrazin-2(3H)-yl)benzoic acid hydrochloride

A solution of 4-(7-(tert-butoxycarbonyl)-3-thioxohexahydroimidazo[1,5-a]pyrazin-2(3H)-yl) benzoic acid (Intermediate S10-4) (75 mg, 0.199 mmol) in 4 M hydrochloride in 1,4-dioxane (5 mL) was stirred at 25° C. for 3 hours. The reaction mixture was concentrated to give the title compound (50 mg, 90% purity from ¹H NMR, 72% yield) as white solids which was directly used in next step without purification. ¹H NMR (400 MHz, DMSO-d₆) δ 9.30 (s, 2H), 7.96 (d, J=8.8 Hz, 2H), 7.85 (d, J=8.8 Hz, 2H), 4.59-4.51 (m, 1H), 4.37-4.25 (m, 2H), 3.98-3.94 (m, 1H), 3.52-3.48 (m, 2H), 3.42-3.40 (m, 1H), 3.12-3.04 (m, 1H), 3.01-2.95 (m, 1H).

### Compound 23: 4-(7-((5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)benzoic acid (Mixture of Diasteromers)

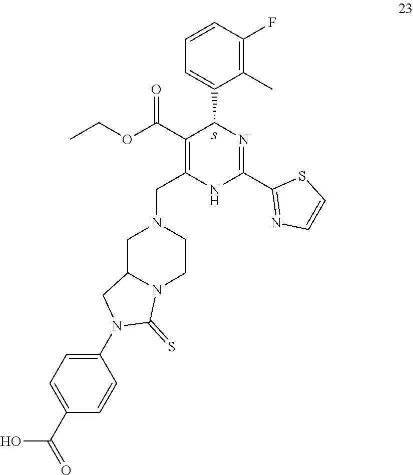

Compound 23 was prepared from intermediate H2-1A and S10 analogous to compound 18B.

LC-MS (ESI): R_{T}=3.457 min, mass calcd. for C₃₁H₃₁FN₆O₄S₂ 634.2, m/z found 635.2 [M+H]⁺. Chiral analysis (Column: Chiralpak IA 5 μm 4.6*250 mm; Mobile Phase: Hex: EtOH:TFA:DEA=50:50:0.1:0.1 at 1 mL/min; Temp: 30° C.; Wavelength: 254 nm, R_{T}=9.145 min (R, 9.7445%), 11.813 min (S, 90.2555%)). ¹H NMR (400 MHz, CD₃OD) δ 7.93 -7.90 (m, 2H), 7.84 (d, J=2.8 Hz, 1H), 7.72-7.68 (m, 2H), 7.64-7.63 (m, 1H), 7.09-7.00 (m, 2H), 6.86-6.81 (m, 1H), 5.88 (s, 1H), 4.57-4.53 (m, 1H), 4.16-4.05 (m, 3H), 3.96 (q, J=7.2 Hz, 2H), 3.88 (d, J=17.2 Hz, 1H), 3.73-3.69 (m, 1H), 3.31-3.25 (m, 1H), 2.98-2.95 (m, 2H), 2.49-2.39 (m, 4H), 2.25-2.20 (m, 1H), 1.03 (t, J=6.8 Hz, 3H).

### Compound 24A and 24B: (1S,3R)-3-(7-(((S)-5-(Ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)cyclopentanecarboxylic acid (single enantiomers)

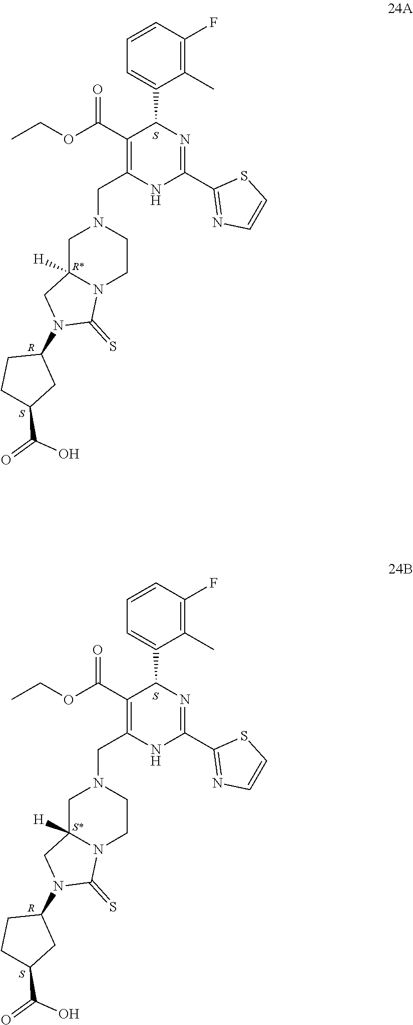

### Preparation of Intermediate S11

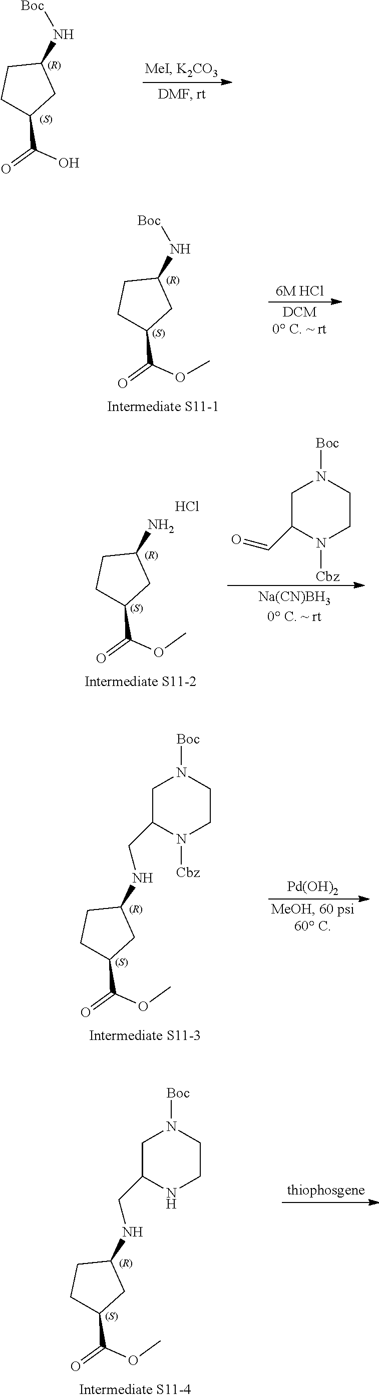

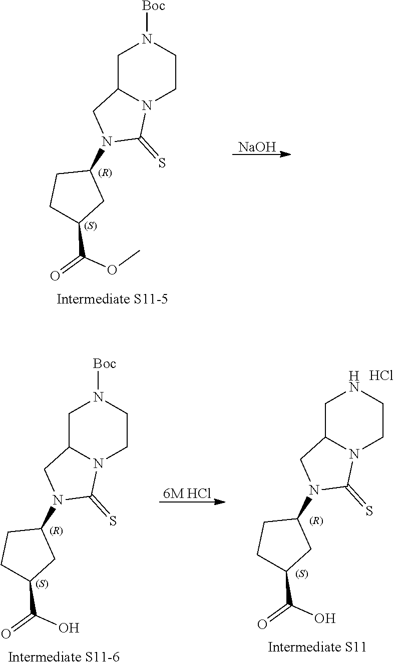

### Intermediate S11-1: (1S,3R)-Methyl 3-((tert-butoxycarbonyl)amino)cyclopentane-carboxylate

To a solution of (1S,3R)-3-((tert-butoxycarbonyl)amino)cyclopentanecarboxylic acid (700 mg, 95% purity from HNMR, 2.90 mmol) in N,N-dimethylformamide (10 mL) was added potassium carbonate (605 mg, 4.38 mmol) at room temperature, followed by addition iodomethane (500 mg, 3.52 mmol). After stirred overnight, the mixture was diluted with ethyl acetate (40 mL) and washed with water (30 mL) twice, brine (30 mL) twice, dried over Na₂SO₄₍ₛ₎, filtered and concentrated under reduced pressure to afford the desired product (700 mg, 95% purity from HNMR, 94% yield) as white solids. ¹H NMR (400 MHz, CDCl₃) δ 5.00-4.88 (m, 1H), 4.06-4.00 (m, 1H), 3.69 (s, 3H), 2.87-2.80 (m, 1H), 2.25-2.18 (m, 1H), 1.97-1.89 (m, 3H), 1.74-1.65 (m, 1.5H), 1.58-1.56 (m, 0.5H), 1.44 (s, 9H).

### Intermediate S11-2: (1S,3R)-Methyl 3-aminocyclopentanecarboxylate hydrochloride

To the solution of (1S,3R)-methyl 3-((tert-butoxycarbonyl)amino)cyclopentane-carboxylate (Intermediate S11-1) (700 mg, 95% purity, 2.73 mmol) in dichloromethane (10 mL) was added 6 M hydrochloride in diethyl ether (10 mL) at 0° C. under nitrogen atmosphere. After stirred at room temperature for 16 hours, the reaction mixture was concentrated under reduced pressure to give the title compound (540 mg, 90% purity from HNMR, 99% yield) as white solids. H NMR (400 MHz, DMSO-d₆) δ 8.31 (br s, 3H), 3.62 (s, 3H), 3.46-3.43 (m, 1H), 2.90-2.81 (m, 1H), 2.29-2.22 (m, 1H), 1.97-1.85 (m, 3H), 1.80-1.64 (m, 2H).

### Intermediate S11-3: 1-Benzyl 4-tert-butyl 2-((((1R,3S)-3-(methoxycarbonyl)cyclopentyl)amino)methyl)piperazine-1,4-dicarboxylate

To a solution of (1S,3R)-methyl 3-aminocyclopentanecarboxylate hydrochloride (Intermediate S11-2) (540 mg, 90% purity, 2.71 mmol) in methanol (10 mL) was added triethylamine (295 mg, 2.92 mmol) at room temperature. After stirred at room temperature under nitrogen atmosphere for 0.5 hour, a solution of 1-benzyl 4-tert-butyl 2-formylpiperazine-1,4-dicarboxylate (Intermediate S1-2) (750 mg, 1.94 mmol) in methanol (10 mL) was added and the mixture was stirred at room temperature for 1 hour. Then sodium cyanoborohydride (283 mg, 4.50 mmol) was added at 0° C. and the mixture was stirred at room temperature for 4 hours, quenched with ice water (20 mL), removed methanol under vacuo and extracted with ethyl acetate (30 mL) for three times. The combined organic layers were dried over Na₂SO₄₍ₛ₎ and filtered. The filtrate was concentrated under reduced pressure to give a residue, which was purified by C18 column (acetonitrile:water=60% to 70%) to give the title compound (640 mg, 95% purity from HNMR, 66% yield) as light yellow oil. ¹H NMR (400 MHz, CDCl₃) δ 7.36-7.34 (m, 5H), 5.19-5.10 (m, 2H), 4.10-3.92 (m, 4H), 3.66 (s, 3H), 3.05-2.68 (m, 8H), 2.15-1.81 (m, 5H), 1.46 (s, 9H).

### Intermediate S11-4: tert-Butyl 3-((((1R,3S)-3-(methoxycarbonyl)-cyclopentyl)amino)methyl)piperazine-1-carboxylate

To a solution of 1-benzyl 4-tert-butyl 2-((((1R,3S)-3-(methoxycarbonyl) cyclopentyl)amino)methyl)piperazine-1,4-dicarboxylate Intermediate S11-3 (640 mg, 95% purity, 1.28 mmol) in methanol (10 mL) was added 20% palladium hydroxide on carbon (370 mg, 2.64 mmol). After stirred at 60° C. under hydrogen atmosphere (60 psi) for 3 days, the mixture was filtered and the filtrate was concentrated under reduced pressure to give the title compound (380 mg, 90% purity from HNMR, 78% yield) as colorless oil. ¹H NMR (400 MHz, DMSO-d₆) δ 3.77-3.69 (m, 2H), 3.59 (s, 3H), 3.43-3.24 (m, 4H), 3.03-2.98 (m, 1H), 2.84-2.73 (m, 3H), 2.47-2.36 (m, 4H), 2.09-2.04 (m, 1H), 1.82-1.72 (m, 3H), 1.54-1.49 (m, 1H), 1.39 (s, 9H).

### Intermediate S11-5: tert-Butyl 2-((1R,3S)-3-(methoxycarbonyl)cyclopentyl)-3-thioxohexahydroimidazo[1,5-a]pyrazine-7(1H)-carboxylate

To a solution of tert-butyl 3-((((1R,3S)-3-(methoxycarbonyl)cyclopentyl)-amino)methyl)piperazine-1-carboxylate (Intermediate S11-4) (380 mg, 90% purity, 1.00 mmol) and triethylamine (320 mg, 3.16 mmol) in dichloromethane (5 mL) was added a solution of thiophosgene (180 mg, 1.57 mmol) in dichloromethane (5 mL) at 0° C. under nitrogen atmosphere. After stirred at room temperature overnight, the mixture was diluted with ice water (10 mL) and extracted with dichloromethane (20 mL) for three times. The combined organic layers were washed with brine (30 mL), dried over Na₂SO₄₍ₛ₎ and filtered. The filtrate was concentrated under reduced pressure to give a residue, which was purified by C18 column (acetonitrile:water=80% to 90%) to give the title compound (285 mg, 90% purity from HNMR, 67% yield) as brown solids. ¹HNMR (400 MHz, CDCl₃) δ 5.14-5.10 (m, 1H), 4.52-4.47 (m, 1H), 4.33-3.88 (m, 4H), 3.69 (s, 3H), 3.23-3.14 (m, 1H), 3.00-2.86 (m, 4H), 2.68-2.61 (m, 1H), 2.24-2.19 (m, 1H), 1.99-1.91 (m, 4H), 1.47 (s, 9H).

Racemic Intermediate S11-5 (405 mg, 90% purity, 0.950 mmol) was separated by chiral Prep. HPLC (separation condition: Column: Chiralpak IE 5 μm 20*250 mm; Mobile Phase: Hex:IPA=70:30 at 18 mL/min; Temp: 30° C.; Wavelength: 254 nm) to give the title compound Intermediate S11-5A (120 mg, 90% purity from HNMR, 30% yield, 100% stereopure) and Intermediate S11-5B (130 mg, 90% purity from HNMR, 32% yield, 98.3% stereopure) as yellow solids.

Intermediate S11-5A: LC-MS (ESI): R_{T}=1.63 min, mass calcd. for C₁₈H₂₉N₃O₄S 383.2, m/z found 384.1 [M+H]⁺. Chiral analysis (Column: Chiralpak Chiralpak IE 5 μm 4.6*250 mm; Mobile Phase: HEX:IPA=70:30 at 1 mL/min; Temp: 30° C.; Wavelength: 254 nm, R_{T}=14.668 min). ¹HNMR (400 MHz, CDCl₃) δ 5.15-5.09 (m, 1H), 4.51-4.47 (m, 1H), 4.27-4.05 (m, 2H), 3.77-3.73 (m, 1.3H), 3.70 (s, 3H), 3.66-3.64 (m, 0.7H), 3.24-3.19 (m, 1H), 3.03-2.86 (m, 3H), 2.69-2.59 (m, 1H), 2.26-2.19 (m, 1H), 1.99-1.91 (m, 3H), 1.85-1.77 (m, 1H), 1.69-1.65 (m, 1H), 1.47 (s, 9H).

Intermediate S11-5B: LC-MS (ESI): R_{T}=1.63 min, mass calcd. for C₁₈H₂₉N₃O₄S 383.2, m/z found 384.1 [M+H]⁺. Chiral analysis (Column: Chiralpak Chiralpak IE 5 μm 4.6*250 mm; Mobile Phase: HEX:IPA=70:30 at 1 mL/min; Temp: 30° C.; Wavelength: 254 nm, R_{T}=17.029 min). ¹HNMR (400 MHz, CDCl₃) δ 5.14-5.10 (m, 1H), 4.51-4.47 (m, 1H), 4.23-4.01 (m, 2H), 3.77-3.73 (m, 2H), 3.69 (s, 3H), 3.17-3.14 (m, 1H), 3.02-2.86 (m, 3H), 2.68-2.59 (m, 1H), 2.25-2.18 (m, 1H), 2.00-1.93 (m, 3H), 1.85-1.79 (m, 1H), 1.69-1.65 (m, 1H), 1.47 (s, 9H).

### Intermediate S11-6A: (1S,3R)-3-(7-(tert-Butoxycarbonyl)-3-thioxohexahydroimidazo[1,5-a]pyrazin-2(3H)-yl)cyclopentanecarboxylic acid

To a solution of tert-butyl 2-((1R,3S)-3-(methoxycarbonyl)cyclopentyl)-3-thioxohexahydroimidazo[1,5-a]pyrazine-7(1H)-carboxylate Intermediate S11-5A (120 mg, 90% purity, 0.282 mmol) in tetrahydrofuran (1 mL), methanol (1 mL) was added sodium hydroxide (30 mg, 0.75 mmol) in water (1 mL) at 0° C. After stirred at room temperature for 5 hours, the mixture was diluted with water (5 mL), acidified to pH 4-5 with 1M hydrochloride aqueous solution and extracted with ethyl acetate (10 mL) twice. The combined organic layers were dried over Na₂SO₄₍ₛ₎ and filtered. The filtrate was concentrated under reduced pressure to give title compound (110 mg, 90% purity from HNMR, 95% yield) as white solids. ¹HNMR (400 MHz, CDCl₃) δ 5.17-5.09 (m, 1H), 4.51-4.47 (m, 1H), 4.25-4.04 (m, 2H), 3.81-3.64 (m, 2H), 3.22-3.18 (m, 1H), 3.04-2.82 (m, 3H), 2.68-2.59 (m, 1H), 2.30-2.23 (m, 1H), 2.02-1.95 (m, 3H), 1.85-1.81 (m, 1H), 1.68-1.65 (m, 1H), 1.48 (s, 9H).

### Intermediate S11-6B was Prepared from Intermediate S11-5B Analogous to Intermediate S11-6A

¹HNMR (400 MHz, CDCl₃) δ 5.18-5.09 (m, 1H), 4.51-4.47 (m, 1H), 4.25-4.01 (m, 2H), 3.76-3.70 (m, 2H), 3.16-3.12 (m, 1H), 3.03-2.90 (m, 3H), 2.67-2.59 (m, 1H), 2.29-2.22 (m, 1H), 2.04-1.95 (m, 3H), 1.84-1.81 (m, 1H), 1.70-1.66 (m, 1H), 1.48 (s, 9H).

### Intermediate S11A: (1S,3R)-3-(3-Thioxohexahydroimidazo[1,5-a]pyrazin-2(3H)-yl)cyclopentanecarboxylic acid hydrochloride

To the solution of (1S,3R)-3-(7-(tert-butoxycarbonyl)-3-thioxohexahydroimidazo[1,5-a]pyrazin-2(3H)-yl)cyclopentanecarboxylic acid Intermediate S11-6A (110 mg, 90% purity, 0.268 mmol) in dichloromethane (3 mL) was added 6M hydrochloride in diethyl ether (3 mL) at 0° C. under nitrogen atmosphere. After stirred at room temperature for 16 hours, the reaction mixture was concentrated under reduced pressure to give the title compound (90 mg, 90% purity from HNMR, 99% yield) as white solids. HNMR (400 MHz, DMSO-d₆) δ 9.58-9.41 (m, 2H), 4.87-4.82 (m, 1H), 4.41-4.37 (m, 1H), 4.19-4.13 (m, 1H), 3.76-3.71 (m, 1H), 3.37-3.27 (m, 4H), 2.88-2.76 (m, 3H), 2.07-2.00 (m, 1H), 1.86-1.73 (m, 4H), 1.66-1.60 (m, 1H).

### Intermediate S11B was Prepared from Intermediate S11-6B Analogous to Intermediate S11A

¹HNMR (400 MHz, DMSO-d₆) δ 9.63-9.45 (m, 2H), 4.87-4.83 (m, 1H), 4.41-4.37 (m, 1H), 4.23-4.13 (m, 1H), 3.78-3.73 (m, 1H), 3.37-3.27 (m, 4H), 2.89-2.75 (m, 3H), 2.06-1.99 (m, 1H), 1.87-1.72 (m, 4H), 1.67-1.62 (m, 1H).

### Compound 24A and 24B were Prepared from Intermediate H2-1A and Intermediate S11-A and S11B, Respectively

24A: LC-MS (ESI): R_{T}=3.986 min, mass calcd. for C₃₀H₃₅FN₆O₄S₂ 626.2, m/z found 627.2 [M+H]⁺. Chiral analysis (Column: Chiralpak IA 5 μm 4.6*250 mm; Mobile Phase: HEX: EtOH:TFA=70:30:0.2 at 1 mL/min; Temp: 30° C.; Wavelength: 254 nm, R_{T}=8.161 min). ¹H NMR (400 MHz, CD₃OD) δ 7.83 (d, J=3.2 Hz, 1H), 7.62 (d, J=3.2 Hz, 1H), 7.06-6.98 (m, 2H), 6.85-6.81 (m, 1H), 5.87 (s, 1H), 4.99-4.90 (m, 1H), 4.37-4.33 (m, 1H), 4.04-3.92 (m, 4H), 3.84-3.80 (m, 1H), 3.69-3.64 (m, 1H), 3.27-3.24 (m, 1H), 3.18-3.13 (m, 1H), 3.00-2.96 (m, 1H), 2.80-2.67 (m, 2H), 2.41 (s, 3H), 2.25-2.16 (m, 2H), 2.12-2.05 (m, 1H), 1.90-1.75 (m, 4H), 1.67-1.62 (m, 1H), 1.04 (t, J=6.8 Hz, 3H).

24B: LC-MS (ESI): R_{T}=3.468 min, mass calcd. for C₃₀H₃₅FN₆O₄S₂ 626.2, m/z found 627.2 [M+H]⁺. Chiral analysis (Column: Chiralpak IA 5 μm 4.6*250 mm; Mobile Phase: HEX EtOH:TFA=70:30:0.2 at 1 mL/min; Temp: 30° C.; Wavelength: 254 nm, R_{T}=9.890 min). ¹H NMR (400 MHz, CD₃OD) δ 7.95 (d, J=3.2 Hz, 1H), 7.74 (d, J=2.8 Hz, 1H), 7.17-7.10 (m, 2H), 6.97-6.93 (m, 1H), 5.99 (s, 1H), 5.09-5.03 (m, 1H), 4.56-4.53 (m, 1H), 4.15-4.04 (m, 4H), 3.96-3.92 (m, 1H), 3.78-3.73 (m, 1H), 3.31-3.24 (m, 2H), 2.99-2.84 (m, 3H), 2.53 (s, 3H), 2.49-2.43 (m, 1H), 2.20-2.14 (m, 2H), 2.00-1.95 (m, 2H), 1.90-1.83 (m, 2H), 1.77-1.72 (m, 1H), 1.16 (t, J=7.2 Hz, 3H).

### Compound 24C and 24D: (1R,3S)-3-(7-(((S)-5-(Ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)cyclopentanecarboxylic acid (Single Enantiomers)

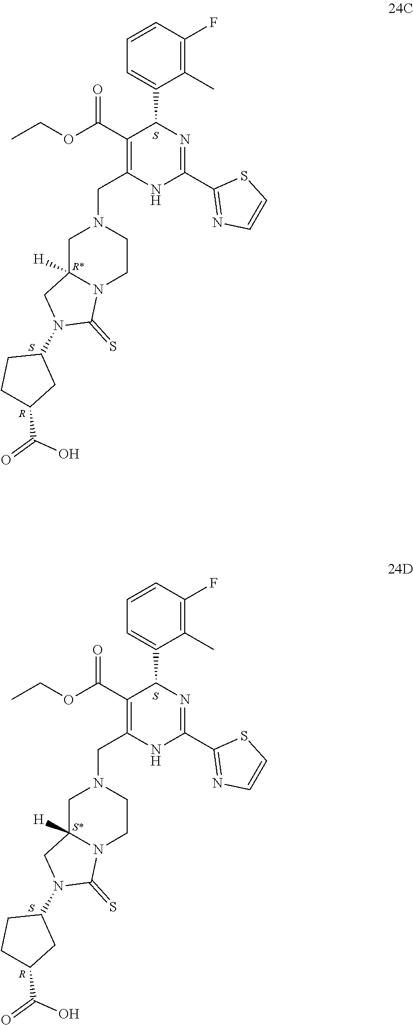

Compound 24C and 24D were prepared analogous to compound 24A and 24B.

Compound 24C: LC-MS (ESI): R_{T}=3.512 min, mass calcd. for C₃₀H₃₅FN₆O₄S₂ 626.2, m/z found 627.3 [M+H]⁺. Chiral analysis (Column: Chiralpak IE 5 μm 4.6*250 mm; Mobile Phase: Hex:EtOH:TFA=50:50:0.2 at 1.0 mL/min; Temp: 30° C.; Wavelength: 254 nm R_{T}=10.315 min). ¹H NMR (400 MHz, CD₃OD) δ 7.89 (d, J=3.2 Hz, 1H), 7.68 (d, J=3.2 Hz, 1H), 7.13-7.04 (m, 2H), 6.91-6.87 (m, 1H), 5.93 (s, 1H), 5.04-4.95 (m, 1H), 4.51-4.47 (m, 1H), 4.10-3.98 (m, 4H), 3.88 (d, J=16.8 Hz, 1H), 3.66 (t, J=10.0 Hz, 1H), 3.25-3.21 (m, 2H), 2.94-2.77 (m, 3H), 2.47 (s, 1.5H), 2.46 (s, 1.5H), 2.44-2.37 (m, 1H), 2.17-2.10 (m, 2H), 1.94-1.63 (m, 5H), 1.08 (t, J=7.2 Hz, 3H).

Compound 24D: LC-MS (ESI): R_{T}=3.735 min, mass calcd. for C₃₀H₃₅FN₆O₄S₂ 626.2, m/z found 627.3 [M+H]⁺. Chiral analysis (Column: Chiralpak IE 5 μm 4.6*250 mm; Mobile Phase: Hex:EtOH:TFA=50:50:0.2 at 1.0 mL/min; Temp: 30° C.; Wavelength: 254 nm, R_{T}=8.320 min). ¹H NMR (400 MHz, CD₃OD) δ 7.88 (d, J=3.2 Hz, 1H), 7.67 (d, J=3.2 Hz, 1H), 7.12-7.04 (m, 2H), 6.91-6.86 (m, 1H), 5.93 (s, 1H), 5.05-4.96 (m, 1H), 4.42-4.39 (m, 1H), 4.10-3.98 (m, 4H), 3.88 (d, J=17.2 Hz, 1H), 3.77 (t, J=10.0 Hz, 1H), 3.24-3.19 (m, 2H), 3.06-3.02 (m, 1H), 2.85-2.73 (m, 2H), 2.46 (s, 1.5H), 2.45 (s, 1.5H), 2.30-2.21 (m, 2H), 2.17-2.10 (m, 1H), 1.96-1.66 (m, 5H), 1.08 (t, J=7.2 Hz, 3H).

### Compound 25A: 3-(7-((5-(Ethoxycarbonyl)-6-(2-fluoro-4-methylphenyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

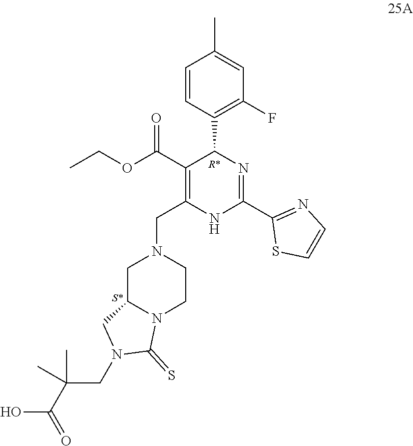

Compound 25A was prepared from intermediate H14-1A and S1-A analogous to compound 18B. LC-MS (ESI): R_{T}=3.825 min, mass calcd. for C₂₉H₃₅FN₆O₄S₂ 614.2, m/z found 615.2 [M+H]⁺. ¹H NMR (400 MHz, DMSO-d₆) δ9.59 (s, 1H), 8.01 (d, J=3.2 Hz, 1H), 7.93 (d, J=3.2 Hz, 1H), 7.19 (t, J=8.4 Hz, 1H), 6.99-6.94 (m, 2H), 5.88 (s, 1H), 4.37-4.34 (m, 1H), 3.99-3.93 (m, 3H), 3.86-3.82 (m, 2H), 3.75-3.72 (m, 1H), 3.66-3.59 (m, 2H), 3.24-3.12 (m, 2H), 2.92-2.90 (m, 1H), 2.79-2.76 (m, 1H), 2.27-2.21 (m, 4H), 2.03 (t, J=11.2 Hz, 1H), 1.07 (t, J=7.2 Hz, 3H), 0.99-0.90 (m, 6H).

### Compound 26A: 3-(7-((5-(Ethoxycarbonyl)-6-(3-fluoro-2-methylphenyl)-2-(5-methyloxazol-4-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

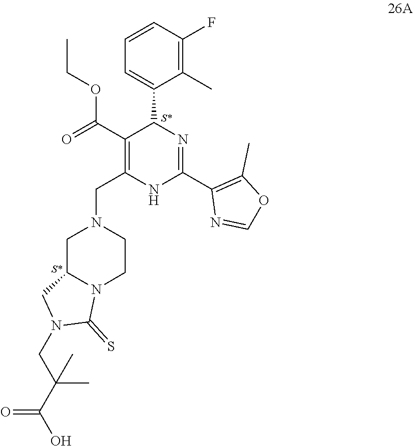

Compound 26A was prepared from intermediate H15-1A and S1-A analogous to compound 18B. LC-MS (ESI): R_{T}=3.732 min, mass calcd. for C₃₀H₃₇FN₆O₅S 612.2, m/z found 613.3 [M+H]⁺. ¹H NMR (400 MHz, DMSO-d₆) δ 12.35 (br s, 1H), 9.37 (s, 0.9H), 8.92 (s, 0.1H), 8.36 (s, 1H), 7.19-7.13 (m, 1H), 7.03-6.95 (m, 2H), 5.85 (s, 0.9H), 5.70 (s, 0.1H), 4.35 (d, J=11.6 Hz, 1H), 4.04-3.90 (m, 5H), 3.77 (d, J=1.6 Hz, 2H), 3.62 (t, J=10.4 Hz, 1H), 3.18-3.10 (m, 2H), 2.92 (d, J=10.4 Hz, 1H), 2.84 (d, J=13.6 Hz, 1H), 2.51 (s, 3H), 2.47 (d, J=2.8 Hz, 3H), 2.29 (td, J=17.2, 8.8 Hz, 1H), 2.06 (t, J=11.2 Hz, 1H), 1.13 (s, 3H), 1.12 (s, 3H), 1.06 (t, J=7.2 Hz, 3H).

### Compound 27A and 27B: 3-((S)-7-(((S)-5-(Ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)-2-methylpropanoic acid (single enantiomers)

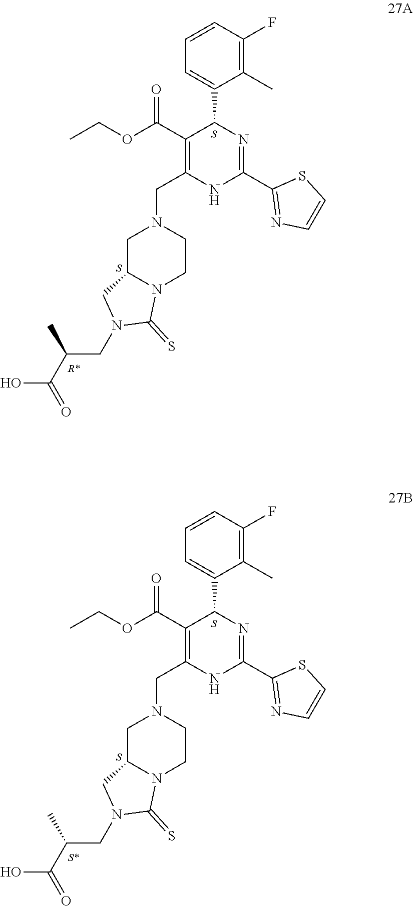

### Preparation of intermediate S12:2-Methyl-3-((S)-3-thioxohexahydroimidazo[1,5-a]pyrazin-2(3H)-yl)propanoic acid hydrochloride

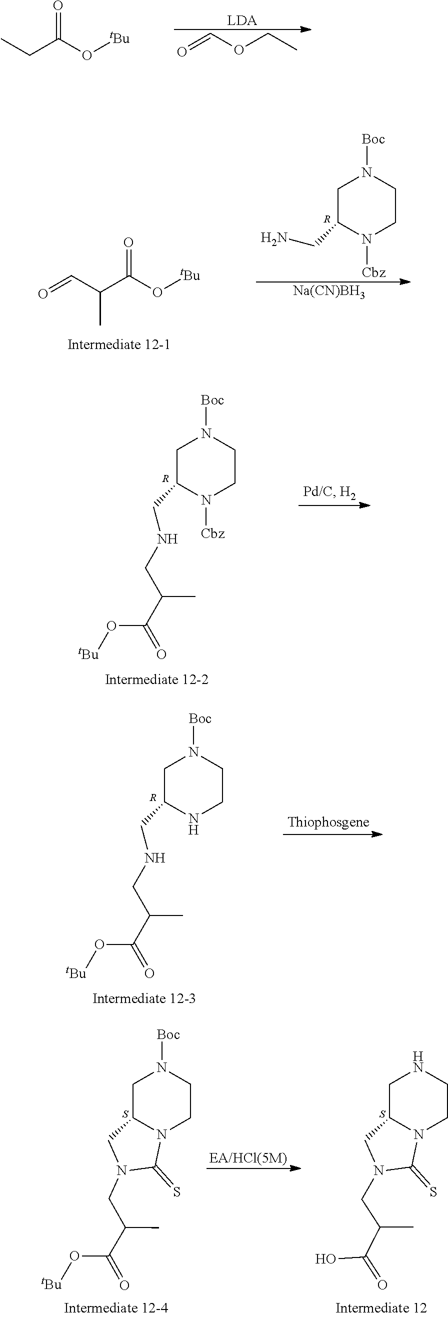

### Intermediate S12-1: tert-Butyl 2-methyl-3-oxopropanoate

To a stirred solution of tert-butyl propionate (14.1 g, 108 mmol) in tetrahydrofuran (500 mL) at −78° C. was added a solution of 2 M lithium diisopropylamide in tetrahydrofuran (65.5 mL, 130 mmol). After stirred at −78° C. for 30 minutes, ethyl formate (25.2 g, 340 mmol) was added. After stirred at −78° C. for another 30 minutes, the mixture was warmed to room temperature. After stirred overnight, the reaction mixture was quenched with cold water (200 mL) and extracted with ethyl acetate (300 mL) twice. Then the aqueous layer was acidified to pH 4-5 with 1 M hydrochloride aqueous solution, extracted with diethyl ether (500 mL) twice. The combined organic layers were dried over Na₂SO₄₍ₛ₎, filtered, the filtrate was concentrated under reduced pressure to give a residue, which was purified by silica gel column chromatography (petroleum ether:ethyl acetate=10:1 to 8:1) to give the title compound (12.2 g, 30% purity from ¹H NMR, 21% yield) as brown oil (mixture of aldehyde form and enol form). ¹H NMR (400 MHz, CDCl₃) δ 11.46 (d, J=12.4 Hz, 0.5H), 9.76 (d, J=1.6 Hz, 0.5H), 6.97-6.94 (m, 0.5H), 3.32-3.27 (m, 0.5H), 1.49 (s, 9H), 1.30 (d, J=7.2 Hz, 3H).

### Intermediate S12-2: (2R)-1-Benzyl 4-tert-butyl 2-(((3-(tert-butoxy)-2-methyl-3-oxopropyl)amino)methyl)piperazine-1,4-dicarboxylate

To a solution of (R)-tert-butyl 3-(aminomethyl)-4-benzylpiperazine-1-carboxylate (1.0 g, 95% purity, 2.72 mmol), a mixture of tert-butyl 2-methyl-3-oxopropanoate (Intermediate S12-2) (2.0 g, 40% purity, 6.15 mmol) and 4A molecular sieves (2 g) in methanol (30 mL) was added acetic acid (500 mg, 8.33 mmol) at room temperature. After stirred at room temperature under nitrogen atmosphere for 1 hour, sodium cyanoborohydride (2.0 g, 31.8 mmol) was added at 0° C. and the mixture was stirred at room temperature for 4 hours, quenched with ice water (30 mL), removed methanol under vacuum and extracted with ethyl acetate (50 mL) for three times. The combined organic layers were dried over Na₂SO₄₍ₛ₎ and filtered. The filtrate was concentrated under reduced pressure to give a residue, which was purified by C18 column (acetonitrile:water=60% to 70%) to give the title compound (1.2 g, 90% purity from ¹H NMR, 81% yield) as light yellow oil. ¹H NMR (400 MHz, CDCl₃) δ 7.38-7.29 (m, 5H), 5.15-5.11 (m, 2H), 4.34-3.79 (m, 4.4H), 3.07-2.39 (m, 8.6H), 1.46 (s, 9H), 1.43 (s, 9H), 1.07 (d, J=6.4 Hz, 3H).

### Intermediate S12-3: (3R)-tert-Butyl 3-(((3-(tert-butoxy)-2-methyl-3-oxopropyl)amino)methyl)piperazine-1-carboxylate

To the solution of (R)-1-benzyl 4-tert-butyl 2-(((3-(tert-butoxy)-2-methyl-3-oxopropyl)amino)methyl)piperazine-1,4-dicarboxylate (Intermediate S12-2) (1.2 g, 90% purity, 2.20 mmol) in methanol (20 mL) was added 10% palladium on activated carbon wt. (600 mg, 0.564 mmol). After stirred at 50° C. under hydrogen atmosphere (60 psi) for 16 hours, the reaction mixture was filtered. The filtrate was concentrated under reduced pressure to give the title compound (650 mg, 27% purity, 22% yield) as yellow oil. ¹H NMR (400 MHz, CDCl₃) δ 4.06-3.81 (m, 1.6H), 3.65-3.55 (m, 0.4H), 3.48-3.26 (m, 0.6H), 3.08-2.44 (m, 10.4H), 2.29-2.04 (m, 1H), 1.45 (s, 18H), 1.10 (dd, J=6.8, 2.0 Hz, 3H).

### Intermediate S12-4: (8aS)-tert-Butyl 2-(3-(tert-butoxy)-2-methyl-3-oxopropyl)-3-thioxohexahydroimidazo[1,5-a]pyrazine-7(1H)-carboxylate

To a solution of (3R)-tert-butyl 3-(3-(tert-butoxy)-2-methyl-3-oxopropyl)amino)methyl)piperazine-1-carboxylate (Intermediate S12-3) (650 mg, 27% purity, 0.491 mmol) and triethylamine (400 mg, 3.95 mmol) in dichloromethane (20 mL) was added a solution of thiophosgene (200 mg, 1.74 mmol) in dichloromethane (10 mL) at 0° C. under nitrogen atmosphere. After stirred at room temperature overnight, the mixture was diluted with ice water (20 mL) and extracted with dichloromethane (100 mL) for three times. The combined organic layers were washed with brine (100 mL), dried over Na₂SO₄₍ₛ₎ and filtered. The filtrate was concentrated under reduced pressure to give residue, which was purified by C18 column (acetonitrile:water=70% to 80%) to give the title compound (100 mg, 98% purity from ¹H NMR, 50% yield) as brown oil. ¹H NMR (400 MHz, CDCl₃) 4.46 (d, J=11.6 Hz, 1H), 4.21-4.02 (m, 2H), 3.79-3.65 (m, 4H), 3.29-3.16 (m, 1H), 3.04-2.97 (m, 1H), 2.91-2.77 (m, 2H), 2.67-2.52 (m, 1H), 1.47 (s, 9H), 1.45 (s, 9H), 1.17 (d, J=6.8, 3H).

Racemic Intermediate S12-4 (150 mg, 95% purity, 0.357 mmol) was separated by chiral Prep. HPLC (separation condition: Column: Chiralpak IC 5 μm 20*250 mm; Mobile Phase: Hex:EtOH=85:15 at 18 mL/min; Temp: 35° C.; Wavelength: 230 nm) to give the title compounds Intermediate S12-4A (35 mg, 98% purity from ¹H NMR, 24% yield, 100% stereopure) and Intermediate S12-4B (35 mg, 98% purity from H NMR, 24% yield, 99.6% stereopure) as yellow solids

Intermediate S12-4A: LC-MS (ESI): R_{T}=1.845 min, mass calcd. for C₁₉H₃₃N₃O₄S 399.2, m/z found 400.2 [M+H]⁺. Chiral analysis (Column: Chiralpak IC 5 μm 4.6*250 mm; Mobile Phase: Hex:EtOH=85:15 at 1 mL/min; Wavelength: 254 nm, R_{T}=8.929 min). H NMR (400 MHz, CDCl₃) δ 4.46 (d, J=11.2 Hz, 1H), 4.29-3.99 (m, 2H), 3.85-3.65 (m, 4H), 3.29-3.14 (m, 1H), 3.04-2.97 (m, 1H), 2.88-2.75 (m, 2H), 2.70-2.53 (m, 1H), 1.47 (s, 9H), 1.45 (s, 9H), 1.16 (d, J=6.8 Hz, 3H).

Intermediate S12-4B: LC-MS (ESI): R_{T}=1.833 min, mass calcd. for C₁₉H₃₃N₃O₄S 399.2, m/z found 400.2 [M+H]⁺. Chiral analysis (Column: Chiralpak IC 5 μm 4.6*250 mm; Mobile Phase: Hex:EtOH=85:15 at 1 mL/min; Wavelength: 254 nm, R_{T}=10.189 min). ¹H NMR (400 MHz, CDCl₃) δ 4.46 (d, J=13.2 Hz, 1H), 4.27-3.95 (m, 2H), 3.84-3.64 (m, 4H), 3.29-3.23 (m, 1H), 3.04-2.98 (m, 1H), 2.91-2.76 (m, 2H), 2.67-2.52 (m, 1H), 1.47 (s, 9H), 1.45 (s, 9H), 1.16 (d, J=7.2 Hz, 3H).

### Intermediate S12:2-Methyl-3-((S)-3-thioxohexahydroimidazo[1,5-a]pyrazin-2(3H)-yl)propanoic acid hydrochloride

To a solution of (S)-tert-butyl 2-(3-(tert-butoxy)-2-methyl-3-oxopropyl)-3-thioxohexahydroimidazo[1,5-a]pyrazine-7(1H)-carboxylate (Intermediate S12-4) (100 mg, 90% purity, 0.225 mmol) was added 5 M hydrochloride in ethylacetate (3.5 mL, 17.5 mmol) at 0° C. After stirred at room temperature under nitrogen atmosphere for 1 hour, the reaction mixture was concentrated under reduced pressure to give the title compound (65 mg, 90% purity from ¹H NMR, 90% yield) as yellow solids. LC-MS (ESI): R_{T}=0.22 min and 0.26 min, mass calcd. for C₁₀H₁₈ClN₃O₂S 279.1, m/z found 244.1 [M+H]⁺. ¹H NMR (400 MHz, DMSO-d₆) δ 9.30 (br s, 2H), 4.38-3.34 (m, 1H), 4.19-4.09 (m, 1H), 3.79-3.68 (m, 2H), 3.65-3.54 (m, 1H), 3.43-3.38 (m, 4H), 2.56-2.76 (m, 3H), 1.06 (d, J=6.8 Hz, 3H).

### Intermediate S12-A and S12-B were Prepared from Intermediate S12-4A and Intermediate S12-4B, Respectively

Intermediate S12-A: ¹H NMR (400 MHz, DMSO-d₆) δ 9.78 (br s, 2H), 4.39-4.30 (m, 1H), 4.27-4.17 (m, 1H), 3.80-3.70 (m, 2H), 3.62-3.56 (m, 1H), 3.44-3.31 (m, 2H), 3.30-3.23 (m, 2H), 2.86-2.73 (m, 3H), 1.06-1.02 (m, 3H).

Intermediate S12-B: ¹H NMR (400 MHz, DMSO-d₆) δ 9.78 (br s, 2H), 4.39-4.30 (m, 1H), 4.27-4.17 (m, 1H), 3.80-3.70 (m, 2H), 3.62-3.56 (m, 1H), 3.44-3.31 (m, 2H), 3.30-3.23 (m, 2H), 2.86-2.73 (m, 3H), 1.10-1.02 (m, 3H).

### Compound 27A and 27B were Prepared from Intermediate H2-1A and S12-A and S12-B, Respectively

27A: LC-MS (ESI): R_{T}=3.695 min, mass calcd. for C₂₈H₃₃FN₆O₄S₂ 600.2, m/z found 601.2 [M+H]⁺. ¹H NMR (400 MHz, CD₃OD) δ 7.83 (d, J=3.2 Hz, 1H), 7.62 (d, J=3.6 Hz, 1H), 7.08-6.98 (m, 2H), 6.83 (t, J=9.2 Hz, 1H), 5.87 (s, 1H), 4.38 (d, J=12.8 Hz, 1H), 4.03-3.92 (m, 4H), 3.82 (d, J=16.8 Hz, 1H), 3.75-3.66 (m, 2H), 3.59-3.54 (m, 1H), 3.18-3.11 (m, 2H), 2.88-2.72 (m, 3H), 2.41 (s, 3H), 2.34 (t, J=11.6 Hz, 1H), 2.08 (t, J=11.2 Hz, 1H), 1.06-1.00 (m, 6H).

27B: LC-MS (ESI): R_{T}=3.278 min, mass calcd. for C₂₈H₃₃FN₆O₄S₂ 600.2, m/z found 601.2 [M+H]⁺. ¹H NMR (400 MHz, CD₃OD) δ 7.83 (d, J=3.2 Hz, 1H), 7.61 (d, J=3.6 Hz, 1H), 7.08-6.96 (m, 2H), 6.85-6.80 (m, 1H), 5.87 (s, 1H), 4.38 (d, J=11.6 Hz, 1H), 4.01-3.92 (m, 4H), 3.88-3.78 (m, 2H), 3.54 (t, J=10.0 Hz, 1H), 3.38-3.31 (m, 2H), 3.16-3.15 (m, 1H), 2.84-2.81 (m, 1H), 2.74-2.72 (m, 1H), 2.64-2.54 (m, 1H), 2.41 (s, 3H), 2.37-2.31 (m, 1H), 2.11 (t, J=10.8 Hz, 1H), 1.06-1.00 (m, 6H).

### Compound 28: 4-(7-(((S)-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)-2,2-dimethylbutanoic acid (Mixture of Diasteromers)

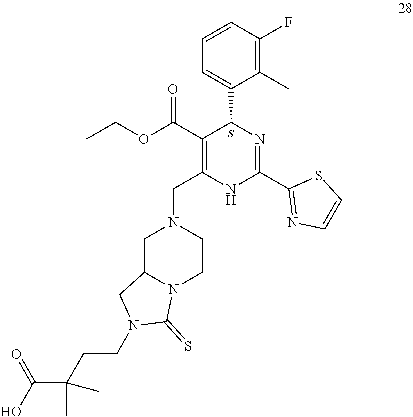

### Preparation of Intermediate S14:2,2-dimethyl-4-(3-thioxohexahydroimidazo[1,5-a]pyrazin-2(3H)-yl)butanoic acid

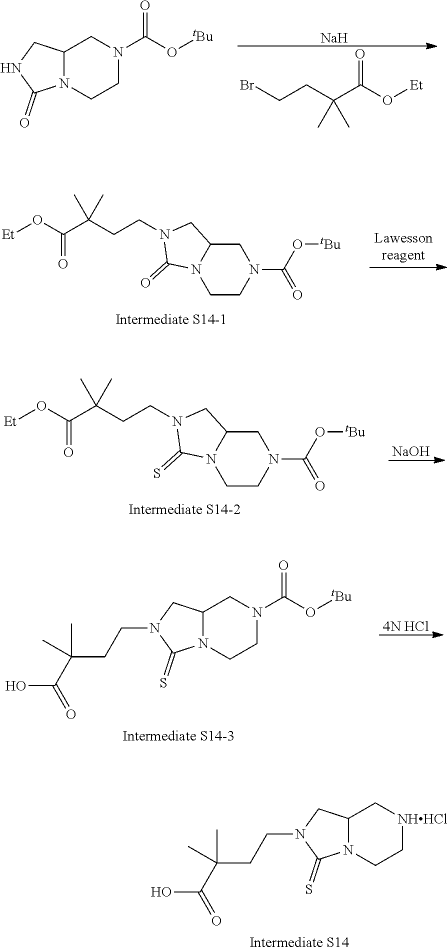

### Intermediate S14-1: tert-Butyl 2-(4-ethoxy-3,3-dimethyl-4-oxobutyl)-3-oxohexahydroimidazo[1,5-a]pyrazine-7(1H)-carboxylate

To a solution of tert-butyl 3-oxohexahydroimidazo[1,5-a]pyrazine-7(1H)-carboxylate (482 mg, 2.00 mmol) in tetrahydrofuran (15 mL) and N,N-dimethylformamide (15 mL) was added 60% wt. sodium hydride in mineral oil (160 mg, 4.00 mmol) at 0° C. under nitrogen atmosphere. After stirring at room temperature for 30 minutes, ethyl 4-bromo-2,2-dimethylbutanoate (540 mg, 2.42 mmol) was added. After stirred at room temperature for 2 days, the reaction mixture was concentrated under reduce pressure to give a residue, which was diluted with ethyl acetate (30 mL), washed with water (10 mL) for three times, brine (10 mL), dried over Na₂SO₄₍ₛ₎ and filtered. The filtrate was concentrated and purified by silica gel column chromatography (petroleum ether:ethyl acetate=20:1 to 2:1) to afford the title compound (160 mg, 21% yield) as colorless oil. LC-MS (ESI): R_{T}=1.60 min, mass calcd. for C₁₉H₃₃N₃O₅ 383.2, m/z found 384.4 [M+H]⁺. ¹H NMR (400 MHz, CDCl₃) δ 4.12 (q, J=7.2 Hz, 2H), 3.99 (br s, 1H), 3.81-3.78 (m, 1H), 3.54 (br s, 1H), 3.44-3.40 (m, 1H), 3.21-3.17 (m, 2H), 2.94-2.91 (m, 1H), 2.85-2.60 (m, 3H), 1.77-1.72 (m, 2H), 1.47 (s, 9H), 1.26 (t, J=7.2 Hz, 3H), 1.22 (s, 6H).

### Intermediate S14-2: tert-Butyl 2-(4-ethoxy-3,3-dimethyl-4-oxobutyl)-3-thioxohexahydroimidazo[1,5-a]pyrazine-7(1H)-carboxylate

To a solution of tert-butyl 2-(4-ethoxy-3,3-dimethyl-4-oxobutyl)-3-oxohexahydroimidazo[1,5-a]pyrazine-7(1H)-carboxylate (Intermediate S14-1) (160 mg, 0.418 mmol) in toluene (15 mL) was added Lawsson's reagent (101 mg, 0.250 mmol). After stirred at 110° C. for 4 hours, the reaction mixture was quenched by saturated sodium bicarbonate solution (10 mL), diluted with ethyl acetate (30 mL). The organic layer was separated and washed with water (10 mL), brine (10 mL), dried over Na₂SO₄₍ₛ₎ and filtered. The filtrate was concentrated under reduced pressure to give a residue, which was purified by Pre-TLC (petroleum ether:ethyl acetate=4:1) to give the desired compound (69 mg, 3% yield) as colorless oil. LC-MS (ESI): R_{T}=1.73 min, mass calcd. for C₁₉H₃₃N₃O₄S 399.2, m/z found 400.2 [M+H]⁺.

### Intermediate S14-3: 4-(7-(tert-Butoxycarbonyl)-3-thioxohexahydroimidazo[1,5-a]pyrazin-2(3H)-yl)-2,2-dimethylbutanoic acid

To a solution of tert-butyl 2-(4-ethoxy-3,3-dimethyl-4-oxobutyl)-3-thioxohexahydroimidazo[1,5-a]pyrazine-7(1H)-carboxylate (Intermediate S14-2) (50 mg, 0.125 mmol) in methanol (2.5 mL) and water (0.7 mL) was added sodium hydroxide (40 mg, 1.00 mmol). After stirred at 50° C. overnight, the reaction mixture was concentrated under reduced pressure. The obtained residue was acidified with 1 N hydrochloride aqueous solution to pH ˜4. Then it was diluted with ethyl acetate (20 mL), washed with water (10 mL) for three times, brine (10 mL) twice, dried over Na₂SO₄₍ₛ₎ and filtered. The filtrate was concentrated under reduced pressure to give the title compound (44 mg, 90% yield) as colorless oil. LC-MS (ESI): R_{T}=1.32 min, mass calcd. for C₁₇H29N₃O₄S 371.2, m/z found 372.1 [M+H]⁺. ¹H NMR (300 MHz, CDCl₃) δ 4.50-4.04 (m, 3.4H), 3.85-3.69 (m, 3.6H), 3.31-3.21 (m, 1H), 3.08-2.76 (m, 3H), 2.09-1.81 (m, 2H), 1.51 (s, 9H), 1.33 (s, 6H).

### Intermediate S14:2,2-Dimethyl-4-(3-thioxohexahydroimidazo[1,5-a]pyrazin-2(3H)-yl)butanoic acid hydrochloride

To a solution of 4-(7-(tert-butoxycarbonyl)-3-thioxohexahydroimidazo[1,5-a]pyrazin-2(3H)-yl)-2,2-dimethylbutanoic acid Intermediate S14-3 (44 mg, 0.118 mmol) in 1,4-dioxane (1 mL) was added 4 N hydrochloride in 1,4-dioxane (3 mL). After stirred at room temperature for 1.5 hours, the mixture was concentrated under reduced pressure to give the title compound (37 mg, 90% yield) as colorless oil. LC-MS (ESI): R_{T}=0.31 min, mass calcd. for C₁₂H₂₂ClN₃O₂S 271.2, m/z found 272.0 [M−HCl+H]⁺. ¹H NMR (400 MHz, CDCl₃) δ 4.56-4.51 (m, 1H), 4.11-4.04 (m, 1H), 3.39-3.25 (m, 4H), 3.22-3.21 (m, 4H), 2.96-2.84 (m, 2H), 1.79-1.67 (m, 2H), 1.15 (s, 6H).

### Compound 28 was Prepared from Intermediate H2-1A and S14 Analogous to Compound 18B

LC-MS (ESI): R_{T}=3.612 min, mass calcd. for C₃₀H₃₇FN₆O₄S₂ 628.3, m/z found 629.3 [M+H]⁺. ¹H NMR (400 MHz, CD₃OD) δ 7.83-7.82 (m, 1H), 7.62 (d, J=3.2 Hz, 1H), 7.06-6.98 (m, 2H), 6.85-6.80 (m, 1H), 5.88 (s, 1H), 4.38-4.28 (m, 1H), 4.06-3.79 (m, 5H), 3.69-3.51 (m, 3H), 3.21-3.13 (m, 1H), 2.98-2.66 (m, 2H), 2.41 (s, 3H), 2.36-1.68 (m, 5H), 1.20-1.13 (m, 5H), 1.02 (t, J=7.2 Hz, 3H).

### Compound 29B: 1-((7-(((S)-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)methyl)cyclobutane-1-carboxylic acid (Single Enantiomer)

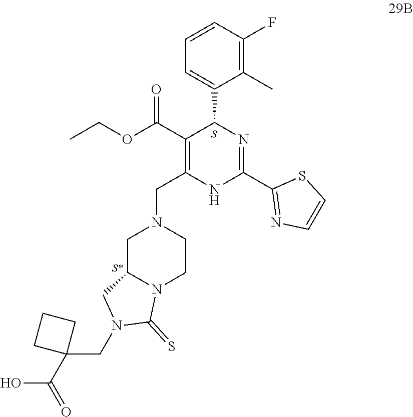

### Preparation of Intermediate S15:1-((3-thioxohexahydroimidazo[1,5-a]pyrazin-2(3H)-yl)methyl)cyclobutane-1-carboxylic acid

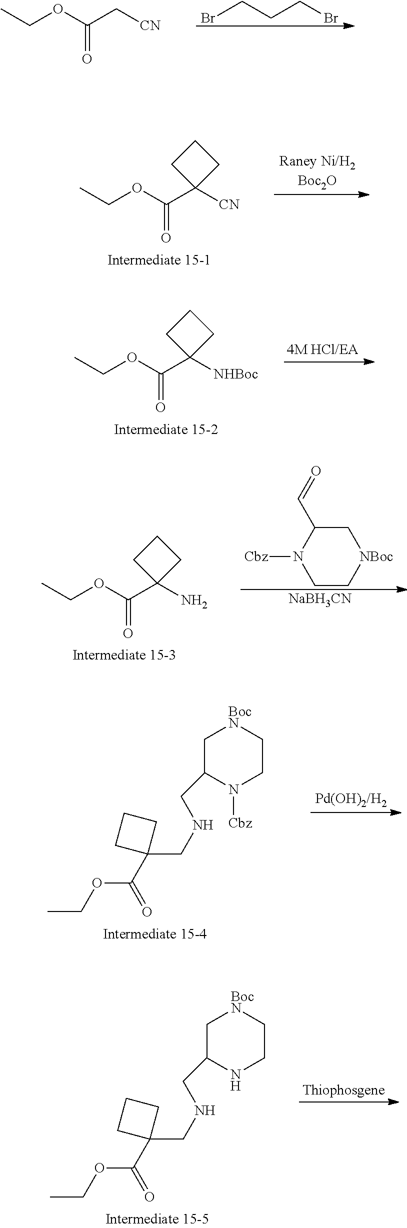

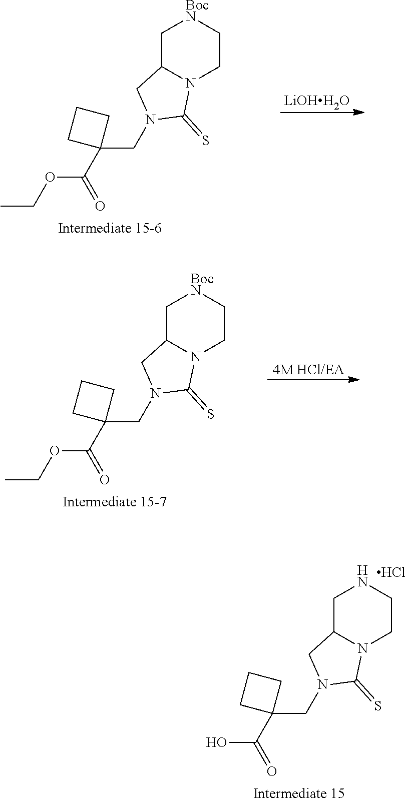 [SMILES: CCOC(=O)C1(CN2CC3CN(C(=O)OC(C)(C)C)CCN3C2=S)CCC1 ; "LiOH·H2O" ; CCOC(=O)C1(CN2CC3CN(C(=O)OC(C)(C)C)CCN3C2=S)CCC1 ; "4M HCl/EA" ; Cl ; O=C(O)C1(CN2CC3CNCCN3C2=S)CCC1]

### Intermediate S15-1: ethyl 1-cyanocyclobutane-1-carboxylate

To a solution of ethyl cyanoacetate (10.0 g, 88.4 mmol) in acetone (200 mL) was added 1,3-dibromopropane (17.0 g, 84.2 mmol) and potassium carbonate (30.0 g, 217 mmol) at 0° C. After stirred at 56° C. overnight, the mixture was filtered. The filtrate was concentrated and purified by silica gel column chromatography (petroleum ether:ethyl acetate=4:1) to give the title compound (7.00 g, 90% purity from H NMR, 47% yield) as white solids. H NMR (400 MHz, CDCl₃) δ 4.27 (q, J=7.2 Hz, 2H), 2.76-2.66 (m, 2H), 2.64-2.59 (m, 2H), 2.34-2.24 (m, 1H), 2.22-2.11 (m, 1H), 1.34 (t, J=7.2 Hz, 3H)

### Intermediate S15-2: Ethyl 1-(((tert-butoxycarbonyl)amino)methyl)cyclobutane-carboxylate

To a solution of ethyl 1-cyanocyclobutanecarboxylate (Intermediate S15-1) (2.00 g, 90% purity, 11.8 mmol) in ethanol (10 mL) was added di-tert-butyl dicarbonate (5.20 g, 23.8 mmol), triethylamine (3.60 g, 35.6 mmol) and Raney Nickel (2.00 g, 22.8 mmol) at room temperature. After stirred at 40° C. for 4 hours under hydrogen atmosphere (50 Psi), the reaction mixture was filtered and the filtrate was concentrated and purified by silica gel chromatography (petroleum ether:ethyl acetate=4:1) to give the title compound (2.30 g, 90% purity from H NMR, 68% yield) as yellow oil. H NMR (400 MHz, CDCl₃) δ 4.92 (s, 1H), 4.17 (q, J=7.2 Hz, 2H), 3.49 (d, J=6.4 Hz, 2H), 2.43-2.36 (m, 2H), 2.09-1.90 (m, 4H), 1.44 (s, 9H), 1.29 (t, J=7.2 Hz, 3H).

### Intermediate S15-3: Ethyl 1-(aminomethyl)cyclobutanecarboxylate hydrochloride

To a solution of ethyl 1-(((tert-butoxycarbonyl)amino)methyl)cyclobutanecarboxylate (Intermediate S15-2) (2.30 g, 90% purity, 8.04 mmol) in dichloromethane (10 mL) was added 4 M hydrochloride in ethyl acetate (10 mL, 40 mmol) under nitrogen atmosphere. After stirred at room temperature under nitrogen atmosphere for 1 hour, the reaction mixture was concentrated to give the title compound (1.50 g, 90% purity from H NMR, 87% yield) as white solids. ¹H NMR (400 MHz, DMSO-d₆) δ 8.33 (s, 3H), 4.13 (q, J=7.2 Hz, 2H), 3.16 (s, 2H), 2.36-2.28 (m, 2H), 2.16-2.09 (m, 2H), 2.04-1.83 (m, 2H), 1.23 (t, J=7.2 Hz, 3H).

### Intermediate S15-4:1-Benzyl 4-tert-butyl 2-((((1-(ethoxycarbonyl)cyclobutyl)-methyl)amino)methyl)piperazine-1,4-dicarboxylate

To a solution of ethyl 1-(aminomethyl)cyclobutanecarboxylate hydrochloride (Intermediate S15-3) (1.00 g, 90% purity, 4.65 mmol) in methanol (10 mL) was added triethylamine (306 mg, 3.02 mmol) at room temperature. The reaction mixture was stirred for 0.5 hour, and then 1-benzyl 4-tert-butyl 2-formylpiperazine-1,4-dicarboxylate (Intermediate S1-2) (1.30 g, 90% purity, 3.35 mmol) was added at room temperature. After stirred at room temperature under nitrogen atmosphere for 1 hour, sodium cyanoborohydride (528 mg, 8.40 mmol) was added at 0° C. Then the mixture was stirred at room temperature for 2 hours, quenched with ice water (20 mL), removed methanol under reduced pressure and extracted with ethyl acetate (20 mL) for three times. The combined organic layers were dried over Na₂SO₄₍ₛ₎ and filtered. The filtrate was concentrated and purified by C18 column (acetonitrile:water=05% to 95%) to give the title compound (1.00 g, 90% purity from ¹H NMR, 55% yield) as light yellow oil. LC-MS (ESI): R_{T}=1.88 min, mass calcd. for C₂₆H₃₉N₃O₆ 489.3, m/z found 490.2 [M+H]⁺. ¹H NMR (400 MHz, CDCl₃) δ 7.37-7.30 (m, 5H), 5.14 (d, J=12.4 Hz, 1H), 5.12 (d, J=12.4 Hz, 1H), 4.28-4.09 (m, 4H), 4.05-3.82 (m, 2H), 3.03-2.83 (m, 5H), 2.79-2.73 (m, 2H), 2.43-2.30 (m, 2H), 1.89 (br s, 4H), 1.45 (s, 9H), 1.28-1.22 (m, 3H).

### Intermediate S15-5: tert-Butyl 3-((((1-(ethoxycarbonyl)cyclopropyl)methyl)-amino)methyl)piperazine-1-carboxylate

To a solution of 1-benzyl 4-tert-butyl 2-((((1-(ethoxycarbonyl)cyclobutyl)methyl)-amino)methyl)piperazine-1,4-dicarboxylate (Intermediate S15-4) (1.00 g, 90% purity, 1.84 mmol) in ethanol (10 mL) was added 10% palladium hydroxide on charcoal wt. (600 mg, 0.427 mmol) at room temperature. After stirred at room temperature under hydrogen atmosphere (balloon) overnight, the reaction mixture was filtered and concentrated to afford the title compound (690 mg, 90% purity from H NMR, 95% yield) as light yellow oil. ¹H NMR (400 MHz, CDCl₃) δ 4.15 (q, J=7.2 Hz, 2H), 3.99-3.75 (m, 2H), 3.00-2.87 (m, 4H), 2.80-2.67 (m, 3H), 2.60-2.36 (m, 4H), 1.99-1.86 (m, 4H), 1.45 (s, 9H), 1.26 (t, J=7.2 Hz, 3H).

### Intermediate S15-6: tert-Butyl 2-((1-(ethoxycarbonyl)cyclobutyl)methyl)-3-thioxohexahydroimidazo[1,5-a]pyrazine-7(1H)-carboxylate

To a solution of tert-butyl 3-((((1-(ethoxycarbonyl)cyclopropyl)methyl)-amino)methyl)piperazine-1-carboxylate (Intermediate S15-5) (690 mg, 90% purity, 1.75 mmol) and triethylamine (530 mg, 5.24 mmol) in dichloromethane (10 mL) was added a solution of thiophosgene (301 mg, 2.62 mmol) in dichloromethane (3 mL) at 0° C. under nitrogen atmosphere. After stirred at room temperature overnight, the mixture was diluted with ice water (10 mL) and extracted with dichloromethane (10 mL) for three times. The combined organic layers were washed with brine (10 mL), dried over Na₂SO₄₍ₛ₎ and filtered. The filtrate was concentrated and purified by silica gel column chromatography (petroleum ether:ethyl acetate=8:1 to 2:1) to give the title compound (360 mg, 90% purity from ¹H NMR, 47% yield) as yellow solids. LC-MS (ESI): R_{T}=1.75 min, mass calcd. for C₁₉H₃₁N₃O₄S 397.2, m/z found 398.1 [M+H]⁺. ¹H NMR (400 MHz, CDCl₃) δ 4.48-4.43 (m, 1H), 4.21-4.05 (m, 6H), 3.74-3.68 (m, 1H), 3.60-3.53 (m, 1H), 3.07-2.97 (m, 2H), 2.87-2.84 (m, 1H), 2.74-2.49 (m, 1H), 2.45-2.38 (m, 2H), 2.17-2.08 (m, 3H), 1.98-1.86 (m, 1H), 1.47 (s, 9H), 1.31-1.22 (m, 3H).

Racemic Intermediate S15-6 (360 mg, 90% purity, 0.815 mmol) was separated by chiral Prep. HPLC (separation conditon: Column: Chiralpak IG 5 μm 20*250 mm; Mobile Phase: CO₂:MeOH=80:20 at 50 g/min; Temp: 30° C.; Wavelength: 230 nm, Back pressure: 100 bar) to give Intermediate S15-6A (118 mg, 90% purity from ¹H NMR, 33% yield, 99.6% stereopure) and Intermediate S15-6B (130 mg, 90% purity from H NMR, 36% yield, 97.7% stereopure) as yellow solids.

Intermediate S15-6A: LC-MS (ESI): R_{T}=1.75 min, mass calcd. for C₁₉H₃₁N₃O₄S 397.2, m/z found 398.1 [M+H]⁺. Chiral analysis (Column: Chiralpak IG 5 μm 4.6*250 mm; Mobile Phase: CO₂:MeOH=80:20 at 3 g/min; Temp: 40° C.; Wavelength: 230 nm, Back pressure: 100 bar, R_{T}=4.09 min). H NMR (400 MHz, CDCl₃) δ 4.49-4.45 (m, 1H), 4.23-3.98 (m, 6H), 3.77-3.68 (m, 1H), 3.56 (t, J=9.6 Hz, 1H), 3.07-2.99 (m, 2H), 2.87-2.76 (m, 1H), 2.64-2.53 (m, 1H), 2.47-2.38 (m, 2H), 2.17-2.06 (m, 3H), 1.98-1.89 (m, 1H), 1.47 (s, 9H), 1.29 (t, J=7.2 Hz, 3H).

Intermediate S15-6B: LC-MS (ESI): R_{T}=1.75 min, mass calcd. for C₁₉H₃₁N₃O₄S 397.2, m/z found 398.1 [M+H]⁺. Chiral analysis (Column: Chiralpak IG 5 μm 4.6*250 mm; Mobile Phase: CO₂:MeOH=80:20 at 3 g/min; Temp: 40° C.; Wavelength: 230 nm, Back pressure: 100 bar, R_{T}=5.35 min). ¹H NMR (400 MHz, CDCl₃) δ 4.48-4.45 (m, 1H), 4.23-4.04 (m, 6H), 3.78-3.68 (m, 1H), 3.56 (t, J=9.6 Hz, 1H), 3.07-2.98 (m, 2H), 2.92-2.76 (m, 1H), 2.65-2.51 (m, 1H), 2.46-2.38 (m, 2H), 2.16-2.06 (m, 3H), 1.98-1.91 (m, 1H), 1.47 (s, 9H), 1.31-1.26 (m, 3H).

### Intermediate S15-7: 1-((7-(tert-Butoxycarbonyl)-3-thioxohexahydroimidazo[1,5-a]pyrazin-2(3H)-yl)methyl)cyclobutanecarboxylic acid

To a solution of tert-butyl 2-((1-(ethoxycarbonyl)cyclobutyl)methyl)-3-thioxo-hexahydroimidazo[1,5-a]pyrazine-7(1H)-carboxylate (Intermediate S15-6) (110 mg, 90% purity, 0.249 mmol) in tetrahydrofuran (2 mL), methanol (3 mL) and water (2 mL) was added lithium hydroxide monohydrate (31 mg, 0.739 mmol) under nitrogen atmosphere. After stirred at room temperature overnight, the reaction mixture was acidified with 1 M hydrochloride aqueous solution (10 mL) till pH=5 and extracted with ethyl acetate (15 mL) for three times. The combined organic layers were dried over anhydrous Na₂SO₄₍ₛ₎, filtered and concentrated to give the desired compound (88 mg, 72% purity, 69% yield) as light yellow solids. LC-MS (ESI): R_{T}=1.26 min, mass calcd. for C₁₇H₂₇N₃O₄S 369.2, m/z found 370.1 [M+H]⁺.

### Intermediate S15-7 was Prepared from Intermediate Intermediate S15-6B

LC-MS (ESI): R_{T}=1.19 min, mass calcd. for C₁₇H₂₇N₃O₄S 369.2, m/z found 370.0 [M+H]⁺.

### Intermediate S15:1-((3-Thioxohexahydroimidazo[1,5-a]pyrazin-2(3H)-yl)methyl)cyclobutanecarboxylic acid hydrochloride

To a solution of 1-((7-(tert-butoxycarbonyl)-3-thioxohexahydroimidazo[1,5-a]pyrazin-2(3H)-yl)methyl)cyclobutanecarboxylic acid (Intermediate S15-7) (88 mg, 72% purity, 0.171 mmol) in dichloromethane (3 mL) was added 4 M hydrochloride in ethyl acetate (2 mL, 8 mmol) under nitrogen atmosphere. After stirred at room temperature under nitrogen atmosphere for 1 hour, the reaction mixture was concentrated to give the title compound (58 mg, 90% purity from ¹H NMR, 99% yield) as white solids. ¹H NMR (400 MHz, CD₃OD) δ 4.59-4.55 (m, 1H), 4.09-3.99 (m, 3H), 3.70-3.63 (m, 1H), 3.34-3.25 (m, 3H), 2.95-2.89 (m, 1H), 2.82 (d, J=16.0 Hz, 1H), 2.70 (d, J=16.0 Hz, 1H), 2.37-2.27 (m, 2H), 2.09-1.97 (m, 3H), 1.86-1.77 (m, 1H).

### Intermediate S15-B was Prepared from Intermediate S15-7B

¹H NMR (400 MHz, CD₃OD) δ 4.59-4.55 (m, 1H), 4.09-3.98 (m, 3H), 3.68-3.63 (m, 1H), 3.34-3.24 (m, 3H), 2.95-2.88 (m, 1H), 2.85-2.78 (m, 2H), 2.37-2.28 (m, 2H), 2.09-1.97 (m, 3H), 1.86-1.76 (m, 1H).

### Compound 29B was Prepared from Intermediate H2-1A and S15-B Analogous to Compound 18B

LC-MS (ESI): R_{T}=3.692 min, mass calcd. for C₃₀H₃₅FN₆O₄S₂ 626.2, m/z found 627.2 [M+H]⁺. ¹H NMR (400 MHz, CD₃OD) δ 7.94 (d, J=3.2 Hz, 1H), 7.74 (d, J=3.2 Hz, 1H), 7.18-7.10 (m, 2H), 6.97-6.92 (m, 1H), 5.98 (s, 1H), 4.54-4.51 (m, 1H), 4.16-4.01 (m, 6H), 3.93 (d, J=16.8 Hz, 1H), 3.68-3.63 (m, 1H), 3.32-3.28 (m, 1H), 3.23-3.19 (m, 1H), 2.99-2.96 (m, 1H), 2.89-2.85 (m, 1H), 2.52 (s, 3H), 2.48-2.35 (m, 3H), 2.19-2.05 (m, 4H), 1.94-1.82 (m, 1H), 1.14 (t, J=7.2 Hz, 3H).

### Compound 30A: 3-(7-(((S)-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)-3-methylbutanoic acid (Single Enantiomer)

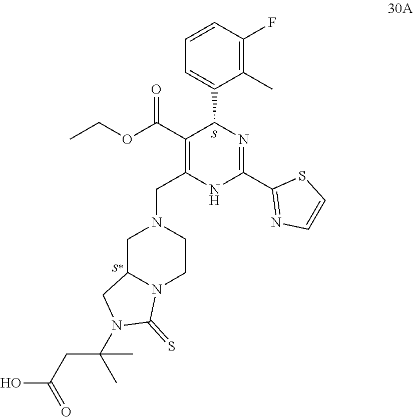

### Preparation of Intermediate S15: ethyl 3-methyl-3-(3-thioxohexahydroimidazo[1,5-a]pyrazin-2(3H)-yl)butanoate

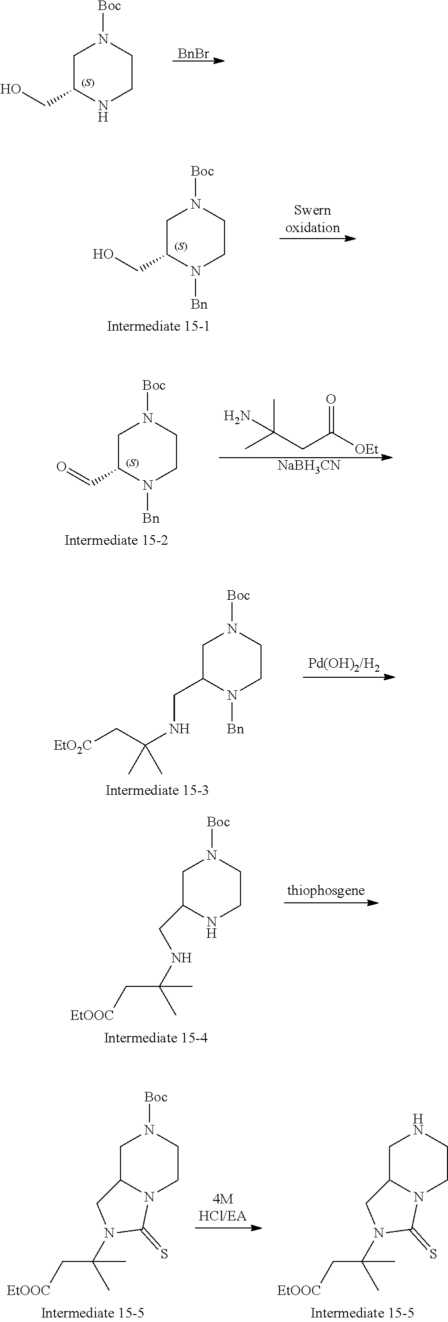

### Intermediate S15-1: (S)-tert-Butyl 4-benzyl-3-(hydroxymethyl)piperazine-1-carboxylate

To the solution of (S)-tert-butyl 3-(hydroxymethyl)piperazine-1-carboxylate (5.0 g, 23.1 mmol) in acetonitrile (50 mL) was added triethylamine (6.4 mL, 46.0 mmol) and benzyl bromide (3.3 mL, 27.8 mmol). After stirred at 80° C. under nitrogen atmosphere overnight, the mixture was concentrated and purified by silica gel chromatography (petroleum ether:ethyl acetate=4:1 to 2:1) to give the title compound (4.2 g, 95% purity from HNMR, 56% yield) as white solids. LC-MS (ESI): R_{T}=1.60 min, mass calcd. for C₁₇H₂₆N₂O₃ 306.2, m/z found 307.1 [M+H]⁺. ¹HNMR (400 MHz, CDCl₃) δ 7.35-7.28 (m, 5H), 4.02 (d, J=13.2 Hz, 1H), 3.87 (dd, J=11.6, 5.6 Hz, 1H), 3.69 (dd, J=13.6, 3.2 Hz, 1H), 3.60-3.51 (m, 2H), 3.42 (d, J=13.2 Hz, 1H), 3.38-3.32 (m, 1H), 3.22-3.11 (m, 1H), 2.80-2.74 (m, 1H), 2.63-2.54 (m, 1H), 2.31-2.25 (m, 1H), 1.46 (s, 9H).

### Intermediate S15-2: tert-Butyl 4-benzyl-3-formylpiperazine-1-carboxylate

To the solution of oxalyl dichloride (3.3 mL, 39.0 mmol) in dichloromethane (50 mL) was added dimethyl sulfoxide (3.7 mL, 52.1 mmol) at −78° C. dropwise. The mixture was stirred at −78° C. for 15 minutes and then a solution of (S)-tert-butyl 4-benzyl-3-(hydroxymethyl)piperazine-1-carboxylate (Intermediate S15-1) (4.2 g, 95% purity, 13.0 mmol) in dichloromethane (5 mL) was added dropwise. After stirred at −78° C. for 1.5 hours, a solution of triethylamine (11 mL, 79.1 mmol) in dichloromethane (5 mL) was added. The mixture was stirred at −78° C. for 30 minutes and warmed to room temperature for another 30 minutes. The mixture was quenched with water (30 mL) and extracted with dichloromethane (50 mL) twice. The combined organic layers were dried over Na₂SO₄₍ₛ₎ and filtered. The filtrate was concentrated to give the crude title compound (4.3 g, 90% purity from HNMR, 98% yield) as yellow oil which was directly used in next step without further purification. LC-MS (ESI): R_{T}=1.74 min, mass calcd. for C₁₇H₂₄N₂O₃ 304.2, m/z found 305.0 [M+H]⁺. ¹HNMR (400 MHz, CDCl₃) δ 9.67 (d, J=2.4 Hz, 1H), 7.34-7.28 (m, 5H), 3.90 (d, J=13.2 Hz, 1H), 3.65 (dd, J=13.2, 4.0 Hz, 1H), 3.58 (d, J=13.2 Hz, 1H), 3.54-3.46 (m, 2H), 3.32-3.28 (m, 1H), 3.11-3.00 (m, 1H), 2.96-2.91 (m, 1H), 2.34-2.27 (m, 1H), 1.45 (s, 9H).

### Intermediate S15-3: tert-Butyl 4-benzyl-3-(((4-ethoxy-2-methyl-4-oxobutan-2-yl)amino)methyl)piperazine-1-carboxylate

To a solution of ethyl 3-amino-3-methylbutyrate hydrochloride (1.0 g, 5.51 mmol) in methanol (25 mL) was added triethylamine (0.8 mL, 5.74 mmol) at room temperature. After stirred at room temperature for 0.5 hour, a solution of tert-butyl 4-benzyl-3-formylpiperazine-1-carboxylate (Intermediate S15-2) (1.5 g, 90% purity, 4.44 mmol) in methanol (5 mL) was added and stirred at 30° C. for 3 hours. Then sodium cyanoborohydride (0.5 g, 7.96 mmol) was added at 0° C. and the mixture was stirred at room temperature for 1 hour. Then the mixture was quenched with water (10 mL), removed methanol under vacuo and extracted with ethyl acetate (20 mL) twice. The combined organic layers were dried over Na₂SO₄₍ₛ₎ and filtered. The filtrate was concentrated to give a residue, which was purified by silica gel chromatography (petroleum ether:ethyl acetate=3:1 to 1:1) to give the title compound (1.5 g, 90% purity from HNMR, 70% yield) as light yellow oil. LC-MS (ESI): R_{T}=1.89 min, mass calcd. for C₂₄H₃₉N₃O₄ 433.3, m/z found 433.9 [M+H]⁺. ¹HNMR (400 MHz, CDCl₃) δ 7.35-7.29 (m, 4H), 7.26-7.22 (m, 1H), 4.10 (q, J=7.2 Hz, 2H), 4.03-3.95 (m, 1H), 3.74-3.57 (m, 1H), 3.51-3.35 (m, 3H), 3.25-3.12 (m, 1H), 2.80-2.65 (m, 3H), 2.46-2.37 (m, 3H), 2.25-2.11 (m, 1H), 1.45 (s, 9H), 1.24 (t, J=7.2 Hz, 3H), 1.15 (s, 6H).

### Intermediate S15-4: tert-Butyl 3-(((4-ethoxy-2-methyl-4-oxobutan-2-yl)amino)methyl)piperazine-1-carboxylate

To the solution of tert-butyl 4-benzyl-3-(((4-ethoxy-2-methyl-4-oxobutan-2-yl)amino)methyl)piperazine-1-carboxylate (Intermediate S15-3) (1.5 g, 90% purity, 3.11 mmol) in methanol (30 mL) was added 20% palladium hydroxide on activated carbon (0.5 g). After stirred at 60° C. under hydrogen atmosphere (60 psi) overnight, the mixture was filtered and the filtrate was concentrated to give the title compound (1 g, 90% purity from HNMR, 84% yield) as colorless oil. ¹H NMR (400 MHz, CDCl₃) δ 4.13 (q, J=7.2 Hz, 2H), 3.76-3.61 (m, 3H), 3.03-2.94 (m, 2H), 2.73-2.62 (m, 3H), 2.52-2.45 (m, 1H), 2.41-2.37 (m, 1H), 2.02 (s, 1H), 1.46 (s, 9H), 1.26 (t, J=7.2 Hz, 3H), 1.16 (s, 6H).

### Intermediate S15-5: tert-Butyl 2-(4-ethoxy-2-methyl-4-oxobutan-2-yl)-3-thioxohexahydroimidazo[1,5-a]pyrazine-7(1H)-carboxylate

To the solution of tert-butyl 3-(((4-ethoxy-2-methyl-4-oxobutan-2-yl)amino)methyl)piperazine-1-carboxylate (Intermediate S15-4) (1.0 g, 90% purity, 2.62 mmol) and triethylamine (1 mL, 7.19 mmol) in dichloromethane (20 mL) was added and thiophosgene (0.3 mL, 3.91 mmol) at 0° C. After stirred at 0° C. for 1 hour, the mixture was quenched with water (10 mL). The mixture was extracted with dichloromethan (20 mL) twice. The combined organic layers were washed with brine (30 mL), dried over Na₂SO₄₍ₛ₎ and filtered. The filtrate was concentrated to give a residue, which was purified by C18 column (acetonitrile:water=70% to 85%) to give the title compound (400 mg, 95% purity from HNMR, 38% yield) as light yellow oil. LC-MS (ESI): R_{T}=1.72 min, mass calcd. for C₁₈H₃₁N₃O₄S 385.2, m/z found 386.0 [M+H]⁺. ¹HNMR (400 MHz, CDCl₃) δ 4.25 (d, J=10.0 Hz, 1H), 4.22-4.07 (m, 4H), 3.89 (t, J=9.6H, 1H), 3.77 (d, J=12.4 Hz, 1H), 3.68-3.61 (m, 1H), 3.43 (dd, J=9.6, 7.2 Hz, 1H), 3.34 (d, J=12.4 Hz, 1H), 2.91-2.85 (m, 2H), 2.73-2.61 (m, 1H), 1.65 (s, 3H), 1.61 (s, 3H), 1.47 (s, 9H), 1.24 (t, J=7.2 Hz, 3H).

Partial racemic Intermediate S15-5 (160 mg, 95% purity, 0.394 mmol, the ratio: 3.7:1) was separated by chiral prep. HPLC (Column: Chiralpak IG 5 μm 20*250 mm; Mobile Phase: Hex:EtOH=70:30 at 18 mL/min; Temp: 30° C.; Wavelength: 254 nm) to give Intermediate S15-5A (110 mg, 95% purity from HNMR, 69% yield, 100% ee) as colorless oil. LC-MS (ESI): R_{T}=1.73 min, mass calcd. for C₁₈H₃₁N₃O₄S 385.2, m/z found 386.0 [M+H]⁺. Chiral analysis (Column: Chiralpak IG 5 μm 4.6*250 mm; Mobile Phase: Hex:EtOH=70:30 at 1.0 mL/min; Temp: 30° C.; Wavelength: 254 nm, R_{T}=7.995 min). ¹HNMR (400 MHz, CDCl₃) δ 4.52 (d, J=10.4 Hz, 1H), 4.27-4.01 (m, 4H), 3.89 (t, J=9.6 Hz, 1H), 3.77 (d, J=18.8 Hz, 1H), 3.68-3.60 (m, 1H), 3.43 (dd, J=9.6, 7.2 Hz, 1H), 3.34 (d, J=16.0 Hz, 1H), 2.91-2.85 (m, 2H), 2.74-2.62 (m, 1H), 1.65 (s, 3H), 1.61 (s, 3H), 1.47 (s, 9H), 1.24 (t, J=7.2 Hz, 3H).

### Intermediate S15: Ethyl 3-methyl-3-(3-thioxohexahydroimidazo[1,5-a]pyrazin-2(3H)-yl)butanoate hydrochloride

A solution of tert-butyl 2-(4-ethoxy-2-methyl-4-oxobutan-2-yl)-3-thioxohexahydroimidazo[1,5-a]pyrazine-7(1H)-carboxylate (Intermediate S15-5) (80 mg, 95% purity, 0.197 mmol) in 4M hydrochloride in ethyl acetate (5 mL) was stirred at room temperature for 1 hour. The mixture was concentrated to give the title compound (60 mg, 98% purity, 93% yield) as white solids. LC-MS (ESI): R_{T}=1.295 min, mass calcd. for C₁₃H₂₃N₃O₂S 285.2, m/z found 286.2 [M+H]⁺.

### Intermediate S15-A was Prepared from Intermediate S15-5A

LC-MS (ESI): R_{T}=1.38 min, mass calcd. for C₁₃H₂₃N₃O₂S 285.2, m/z found 286.0 [M+H]⁺.

### Compound 30A-1: ethyl (4S)-6-((2-(4-ethoxy-2-methyl-4-oxobutan-2-yl)-3-thioxohexahydroimidazo[1,5-a]pyrazin-7(1H)-yl)methyl)-4-(3-fluoro-2-methylphenyl)-2-(thiazol-2-yl)-1,4-dihydropyrimidine-5-carboxylate

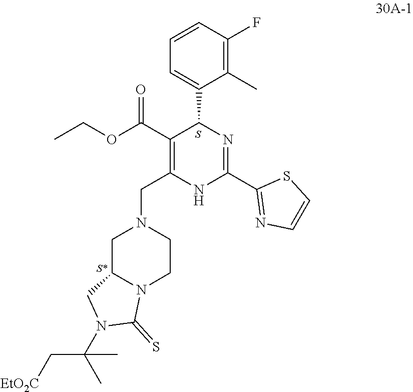

Compound 30A-1 was prepared from intermediate H2-1A and S15-A analogous to compound 18B. LC-MS (ESI): R_{T}=1.99 min, mass calcd. for C₃₁H₃₉FN₆O₄S₂ 642.3, m/z found 643.0 [M+H]⁺. ¹H NMR (400 MHz, CDCl₃) δ 9.55 (s, 1H), 7.82 (d, J=3.2 Hz, 1H), 7.42 (d, J=2.8 Hz, 1H), 7.10-7.05 (m, 1H), 6.99-6.97 (m, 1H), 6.93-6.88 (m, 1H), 6.01 (s, 1H), 4.63 (d, J=14.2 Hz, 1H), 4.14-3.99 (m, 5H), 3.92-3.85 (m, 3H), 3.70 (d, J=16.4 Hz, 1H), 3.47-3.39 (m, 2H), 3.23-3.16 (m, 1H), 2.86 (d, J=14.4 Hz, 1H), 2.75 (d, J=11.2 Hz, 1H), 2.55 (s, 1.5H), 2.54 (s, 1.5H), 2.51-2.47 (m, 1H), 2.24 (t, J=10.4 Hz, 1H), 1.64 (s, 3H), 1.61 (s, 3H), 1.26 (t, J=7.2 Hz, 3H), 1.12 (t, J=7.2 Hz, 3H).

### Compound 30A: 3-(7-(((S)-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)-3-methylbutanoic acid (Single Enantiomer)

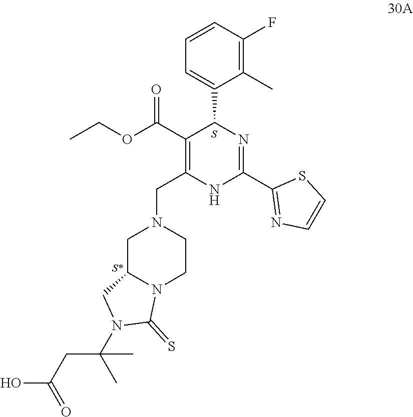

To the solution of compound 30A-1 (70 mg, 95% purity, 0.103 mmol) in tetrahydrofuran (0.4 mL), methanol (0.4 mL) and water (0.2 mL) was added lithium hydroxide monohydrate (15 mg, 0.357 mmol) at 0° C. After stirred at room temperature for 3 hours, the mixture was acidified with 1 M hydrochloride aqueous solution to pH 5˜6 and purified by C18 column (acetonitrile:water=40% to 65%) to give the title compound (25 mg, 99.1% purity, 39% yield) as yellow solids. LC-MS (ESI): R_{T}=3.484 min, mass calcd. for C₂₉H₃₅FN₆O₄S₂ 614.2, m/z found 615.3 [M+H]⁺. ¹H NMR (400 MHz, CD₃OD) δ 7.9 (d, J=3.2 Hz, 1H), 7.74 (d, J=3.2 Hz, 1H), 7.19-7.10 (m, 2H), 6.97-6.93 (m, 1H), 5.99 (s, 1H), 4.58 (d, J=14.4 Hz, 1H), 4.14-4.04 (m, 3H), 3.95-3.91 (m, 3H), 3.71 (d, J=16.4 Hz, 1H), 3.53-3.49 (m, 1H), 3.40-3.36 (m, 1H), 3.17 (td, J=12.0, 3.2 Hz, 1H), 2.94 (d, J=11.4 Hz, 1H), 2.84 (d, J=10.4 Hz, 1H), 2.53 (s, 1.5H), 2.52 (s, 1.5H), 2.44 (td, J=12.0, 3.2 Hz, 1H), 2.25-2.18 (m, 1H), 1.66 (s, 3H), 1.63 (s, 3H), 1.14 (t, J=7.2 Hz, 3H).

### Compound 31A: 3-(7-((6-(3-Fluoro-2-methylphenyl)-5-(methoxycarbonyl)-2-(5-methyloxazol-4-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

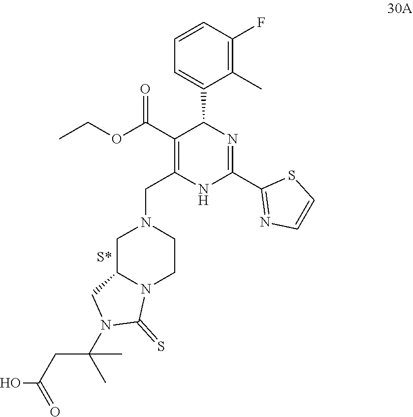 [SMILES: CCOC(=O)C1=C(CN2CCN3C(=S)N(C(C)(C)CC(=O)O)C[C@@H]3C2)NC(c2nccs2)=N[C@H]1c1cccc(F)c1C]

This compound was prepared from intermediate H16-1A and S1-A analogous to compound 18B. LC-MS (ESI): R_{T}=8.057 min, mass calcd. for C₂₉H₃₅FN₆O₅S 598.2, m/z found 599.2 [M+H]⁺. ¹H NMR (400 MHz, CD₃OD) δ 8.04 (s, 1H), 7.17-7.10 (m, 1H), 7.01 (d, J=7.6 Hz, 1H), 6.93 (t, J=9.0 Hz, 1H), 5.97 (s, 1H), 4.50 (d, J=13.2 Hz, 1H), 4.15-4.08 (m, 2H), 3.95-3.85 (m, 3H), 3.70 (t, J=9.6 Hz, 1H), 3.62 (s, 3H), 3.30-3.24 (m, 2H), 2.97-2.94 (m, 1H), 2.88-2.85 (m, 1H), 2.53 (s, 3H), 2.51 (s, 3H), 2.50-2.42 (m, 1H), 2.17 (t, J=10.8 Hz, 1H), 1.24 (s, 3H), 1.23 (s, 3H).

### Compound 32A and 32B: 3-(7-((5-(Ethoxycarbonyl)-6-(3-fluoro-2-methylphenyl)-2-(5-methyloxazol-4-yl)-3,6-dihydropyrimidin-4-yl)methyl)-3-thioxohexahydroimidazo[1,5-a]pyrazin-2(3H)-yl)-2-methylpropanoic acid (single enantiomers)

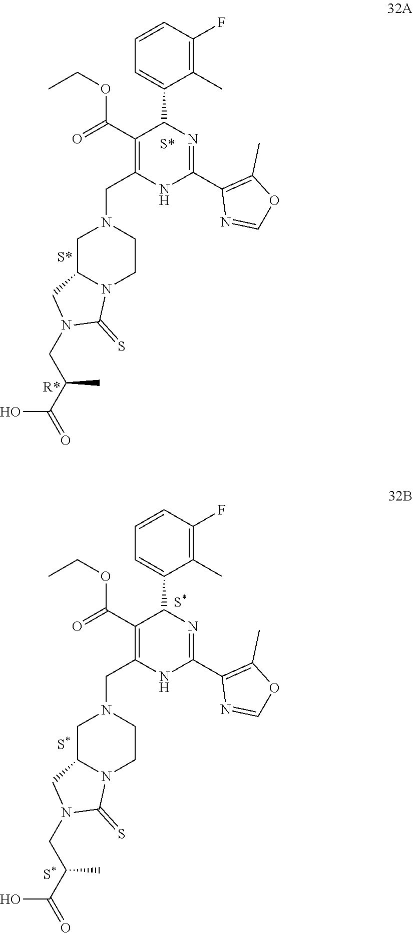

Compound 32A and 32B were prepared from intermediate H15-1A and S12-A and S12-B respectively analogous to compound 27A.

Compound 32A: LC-MS (ESI): R_{T}=3.546 min, mass calcd. for C₂₉H₃₅FN₆O₅S 598.7, m/z found 599.3 [M+H]⁺. Chiral analysis (Column: Chiralpak IE 5 μm 4.6*250 mm; Mobile Phase: Hex:EtOH:TFA=70:30:0.2 at 1 mL/min; Temp: 30° C.; Wavelength: 254 nm, R_{T}=15.665 min). ¹H NMR (400 MHz, CDCl₃) δ 9.28 (br s, 1H), 7.69 (s, 1H), 7.09-7.03 (m, 1H), 6.93-6.88 (m, 2H), 6.00 (s, 1H), 4.51 (d, J=12.8 Hz, 1H), 4.16 (d, J=17.2 Hz, 1H), 4.08-3.99 (m, 3H), 3.91-3.84 (m, 2H), 3.79-3.66 (m, 2H), 3.31-3.25 (m, 1H), 3.20-3.15 (m, 1H), 3.08-3.03 (m, 1H), 2.92-2.89 (m, 1H), 2.84-2.81 (m, 1H), 2.54 (s, 6H), 2.52-2.49 (m, 1H), 2.19 (t, J=11.2 Hz, 1H), 1.26 (s, 1.5H), 1.24 (s, 1.5H), 1.11 (t, J=7.2 Hz, 3H).

Compound 32B: LC-MS (ESI): R_{T}=3.699 min, mass calcd. for C₂₉H₃₅FN₆O₅S 598.7, m/z found 599.3 [M+H]⁺. Chiral analysis (Column: Chiralpak IE 5 μm 4.6*250 mm; Mobile Phase: Hex:EtOH:TFA=70:30:0.2 at 1 mL/min; Temp: 30° C.; Wavelength: 254 nm, R_{T}=21.466 min). ¹H NMR (400 MHz, CDCl₃) δ 9.28 (br s, 1H), 7.68 (s, 1H), 7.09-7.03 (m, 1H), 6.93-6.87 (m, 2H), 5.99 (s, 1H), 4.53 (d, J=12.8 Hz, 1H), 4.15 (d, J=17.2 Hz, 1H), 4.07-3.99 (m, 3H), 3.90-3.76 (m, 3H), 3.67 (t, J=9.6 Hz, 1H), 3.34-3.25 (m, 2H), 3.09-3.04 (m, 1H), 2.89 (d, J=10.4 Hz, 1H), 2.78 (d, J=10.8 Hz, 1H), 2.54 (s, 6H), 2.53-2.49 (m, 1H), 2.17 (t, J=12.0 Hz, 1H), 1.25 (d, J=6.8 Hz, 3H), 1.11 (t, J=7.2 Hz, 3H).

### Compound 33A: 3-(7-((6-(2-Chloro-4-fluorophenyl)-5-(ethoxycarbonyl)-2-(5-methyloxazol-4-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

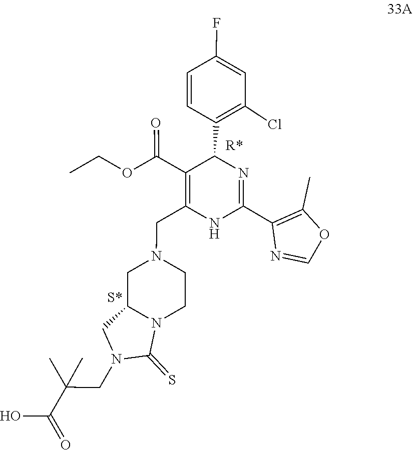 [SMILES: CCOC(=O)C1=C(CN2CCN3C(=S)N(CC(C)(C)C(=O)O)C[C@@H]3C2)NC(c2ncoc2C)=N[C@H]1c1ccc(F)cc1Cl]

This compound was prepared from intermediate H17-1A and S1-A analogous to compound 18B. LC-MS (ESI): R_{T}=8.716 min, mass calcd. for C₂₉H₃₄ClFN₆O₅S 632.2, m/z found 633.2 [M+H]⁺. ¹H NMR (400 MHz, DMSO-d₆) δ 12.28 (br s, 1H), 9.42 (s, 1H), 8.38 (s, 1H), 7.41 (dd, J=8.8, 2.8 Hz, 1H), 7.31 (dd, J=8.8, 6.4 Hz, 1H), 7.16 (td, J=8.4, 2.4 Hz, 1H), 6.00 (s, 0.96H), 5.89 (s, 0.04H), 4.35 (d, J=11.6 Hz, 1H), 4.02-3.89 (m, 5H), 3.76 (s, 2H), 3.63 (t, J=9.6 Hz, 1H), 3.18-3.10 (m, 2H), 2.93-2.68 (m, 2H), 2.51 (s, 3H), 2.28 (td, J=11.2, 2.8 Hz, 1H), 2.07 (t, J=10.8 Hz, 1H), 1.12 (s, 6H), 1.05 (t, J=7.6 Hz, 3H).

### Compound 34A: 3-(7-((6-(2-Chloro-4-fluorophenyl)-5-(methoxycarbonyl)-2-(5-methyloxazol-4-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

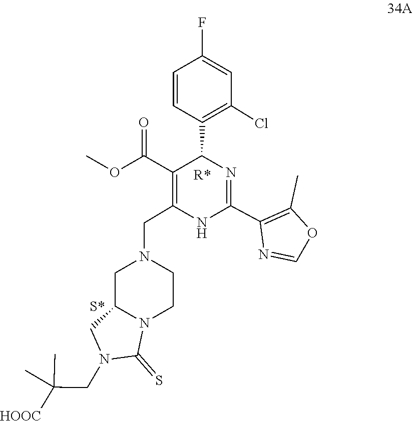 [SMILES: COC(=O)C1=C(CN2CCN3C(=S)N(CC(C)(C)C(=O)O)C[C@@H]3C2)NC(c2ncoc2C)=N[C@H]1c1ccc(F)cc1Cl]

This compound was prepared from intermediate H18-1A and S1-A analogous to analogous to compound 18B. LC-MS (ESI): R_{T}=9.350 min, mass calcd. for C₂₈H₃₂ClFN₆O₅S 618.2, m/z found 619.2 [M+H]⁺. ¹H NMR (400 MHz, DMSO-d₆) δ 9.46 (s, 1H), 8.37 (s, 1H), 7.41 (dd, J=8.8, 2.4 Hz, 1H), 7.29 (dd, J=8.8, 6.4 Hz, 1H), 7.15 (td, J=8.4, 2.4 Hz, 1H), 5.99 (s, 1H), 4.35 (d, J=12.0 Hz, 1H), 4.03-3.90 (m, 3H), 3.80-3.73 (m, 2H), 3.63 (t, J=10.0 Hz, 1H), 3.52 (s, 3H), 3.18-3.10 (m, 2H), 2.93-2.84 (m, 2H), 2.52 (s, 3H), 2.32-2.26 (m, 1H), 2.08 (t, J=10.8 Hz, 1H), 1.12 (s, 6H).

### Compound 35A and 35B: 7-(((S)-5-(Ethoxycarbonyl)-6-(3-fluoro-2-methylphenyl)-2-(thiazol-2-yl)-3,6-dihydropyrimidin-4-yl)methyl)-2-methyl-3-thioxooctahydroimidazo[1,5-a]pyrazine-8-carboxylic acid (Single Enantiomers)

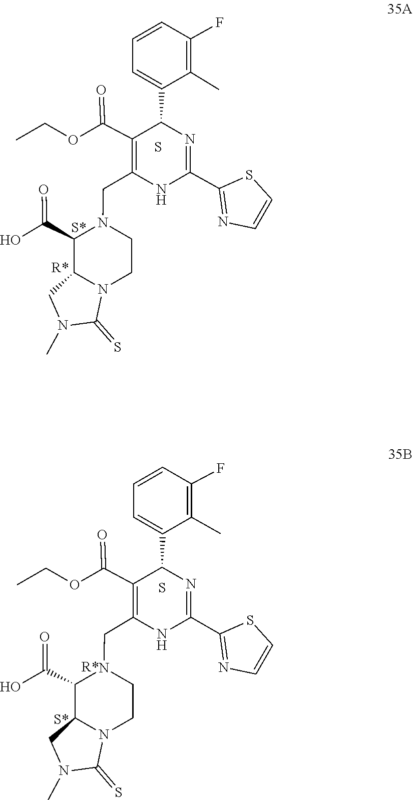

Compound 35A and 35B were prepared from intermediate H2-1A and S9-A and S9-B respectively analogous to compound 19A.

Compound 35A: LC-MS (ESI): R_{T}=3.340 min, mass calcd. for C₂₆H₂₉FN₆O₄S₂ 572.2, m/z found 573.2 [M+H]⁺. ¹H NMR (400 MHz, CD₃OD) δ 7.88 (d, J=3.2 Hz, 1H), 7.67 (d, J=3.2 Hz, 1H), 7.12-7.05 (m, 2H), 6.90-6.84 (m, 1H), 5.91 (s, 1H), 4.32-4.28 (m, 1H), 4.21 (d, J=17.2 Hz, 1H), 4.04-3.94 (m, 3H), 3.81 (d, J=16.8 Hz, 1H), 3.73-3.66 (m, 1H), 3.63 -3.57 (m, 1H), 3.21-3.19 (m, 1H), 3.12-3.10 (m, 1H), 3.07 (s, 3H), 2.82-2.80 (m, 1H), 2.44 (s, 3H), 2.41-2.35 (m, 1H), 1.06 (t, J=7.2 Hz, 3H).

Compound 35B: LC-MS (ESI): R_{T}=3.321 min, mass calcd. for C₂₆H₂₉FN₆O₄S₂ 572.2, m/z found 573.2 [M+H]⁺. ¹H NMR (400 MHz, CD₃OD) δ 7.94 (d, J=3.2 Hz, 1H), 7.73 (d, J=3.2 Hz, 1H), 7.21-7.13 (m, 2H), 6.97-6.93 (m, 1H), 5.96 (s, 1H), 4.45-4.42 (m, 1H), 4.23 (d, J=16.4 Hz, 1H), 4.12-4.03 (m, 3H), 3.92 (d, J=16.4 Hz, 1H), 3.79-3.74 (m, 1H), 3.69-3.65 (m, 1H), 3.30-3.26 (m, 1H), 3.19-3.16 (m, 1H), 3.15 (s, 3H), 3.08-3.05 (m, 1H), 2.67-2.58 (m, 1H), 2.52 (s, 3H), 1.13 (t, J=7.2 Hz, 3H).

### Compound 36B: 3-(7-((6-Deutero-5-(ethoxycarbonyl)-6-(3-fluoro-2-methylphenyl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

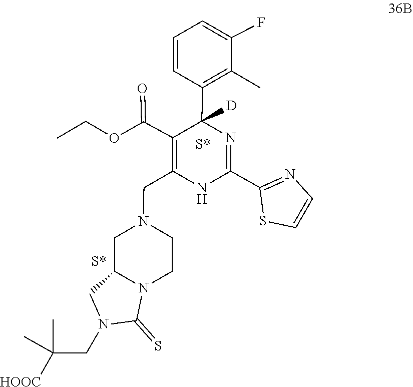 [SMILES: [2H][C@@]1(c2cccc(F)c2C)N=C(c2nccs2)NC(CN2CCN3C(=S)N(CC(C)(C)C(=O)O)C[C@@H]3C2)=C1C(=O)OCC]

This compound was prepared from intermediate H19-1B and S1-A analogous to compound 18B. LC-MS (ESI): R_{T}=3.311 min, mass calcd. for C₂₉H₃₄DFN₆O₄S₂ 611.2, m/z found 616.2 [M+H]⁺. ¹H NMR (400 MHz, CD₃OD) δ 7.95 (d, J=3.2 Hz, 1H), 7.74 (d, J=3.2 Hz, 1H), 7.18-7.11 (m, 2H), 6.97-6.93 (m, 1H), 4.53 (dd, J=13.2, 1.6 Hz, 1H), 4.14-4.04 (m, 4H), 3.97-3.85 (m, 3H), 3.70 (t, J=10.4 Hz, 1H), 3.37-3.30 (m, 1H), 3.28-3.24 (m, 1H), 2.98 (d, J=11.2 Hz, 1H), 2.90 (dd, J=11.2, 2.8 Hz, 1H), 2.53 (s, 3H), 2.45 (td, J=12.0, 3.6 Hz, 1H), 2.8 (t, J=10.8 Hz, 1H), 1.24 (s, 3H), 1.23 (s, 3H), 1.14 (t, J=7.2 Hz, 3H).

### Compound 37: 3-(7-((5-(ethoxycarbonyl)-6-(6-fluoro-2-methylpyridin-3-yl)-2-(thiazol-2-yl)-3,6-dihydropyrimidin-4-yl)methyl)-3-thioxohexahydroimidazo[1,5-a]pyrazin-2(3H)-yl)-2,2-dimethylpropanoic acid (Single Enantiomer)

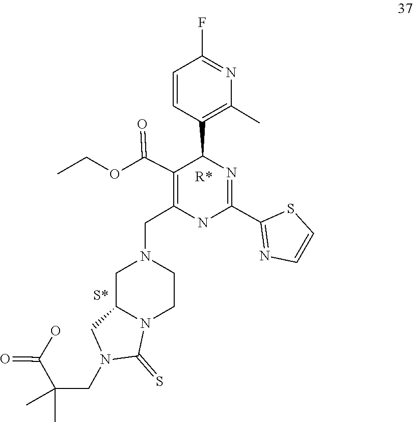

This compound was prepared from intermediate H20-1A and S1-A analogous to compound 18B. LC-MS (ESI): R_{T}=3.409 min, mass calcd. for C₂₈H₃₄FN₇O₄S₂ 615.2, m/z found 616.2 [M+H]⁺. ¹H NMR (400 MHz, CD₃OD) δ 7.94 (d, J=3.2 Hz, 1H), 7.80-7.74 (m, 2H), 6.85 (dd, J=8.0, 2.4 Hz, 1H), 5.95 (s, 1H), 4.53-4.49 (m, 1H), 4.13-4.04 (m, 4H), 3.95-3.83 (m, 3H), 3.71-3.66 (m, 1H), 3.35-3.34 (m, 0.6H), 3.28-3.22 (m, 1.4H), 2.97-2.87 (m, 2H), 2.75 (s, 3H), 2.47-2.40 (m, 1H), 2.20-2.14 (m, 1H), 1.23 (s, 3H), 1.22 (s, 3H), 1.14 (t, J=7.2 Hz, 3H).

GLS4 (WO 2008154817, example 5; Bioorganic & Medicinal Chemistry, 2017, 25, 1042-1056, compound 8n) was selected as reference 1; another compound (WO2015132276, example 76) was selected as reference 2. Chemical structure of both reference compounds was shown below.

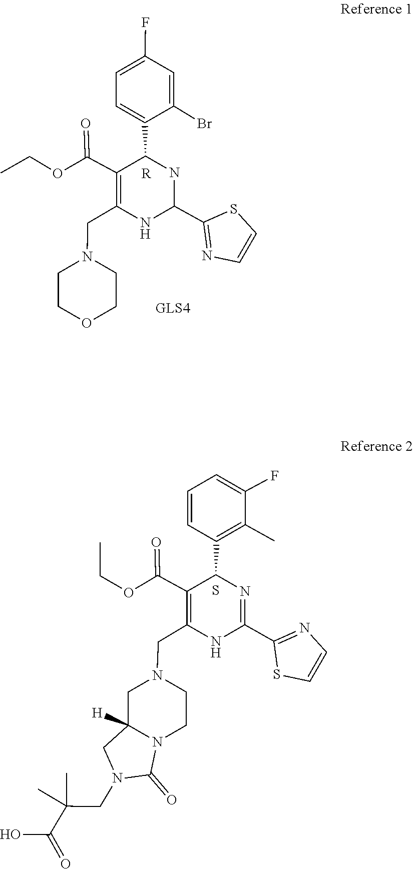

### Example 1: Anti-Viral Assay in HepG2.2.15 Cells

### Materials and Equipments

1) Cell Line

HepG2.2.15 (the HepG2.2.15 cell line can be produced by transfection of the HepG2 cell line as described in Sells, Chen, and Acs 1987 (Proc. Natl. Acad. Sci. USA 84: 1005-1009), and the HepG2 cell line is available from ATCC® under number HB-8065™)

2) Reagents

### DMEM/F12 (INVITROGEN-11330032)

### FBS (GIBCO-10099-141)

Dimethyl sulfoxide(DMSO) (SIGMA-D2650)
Penicillin-streptomycin solution (HYCLONE-SV30010)

### NEAA (INVITROGEN-1114050)

### L-Glutamine (INVITROGEN-25030081)

Geneticin Selective Antibiotic (G418, 500 mg/ml) (INVITROGEN-10131027)
Trypsinase digestion solution (INVITROGEN-25300062)

### CCK8 (BIOLOTE-35004)

### QIAamp 96 DNA Blood Kit (12) (QIAGEN-51162)

### FastStart Universal Probe Mast Mix (ROCHE-04914058001)

3) Consumables

96-well cell culture plate (COSTAR-3599)
Micro Amp Optical 96-well reaction plate (APPLIED BIOSYSTEMS-4306737)
Micro Amp Optical 384-well reaction plate (APPLIED BIOSYSTEMS)

4) Equipment

Plate reader (MOLECULAR DEVICES, SPECTRAMAX M2e)

### Centrifuge (BECKMAN, ALLEGRA-X15R)

Real Time PCR system (APPLIED BIOSYSTEMS, QUANTSTUDIO 6)
Real Time PCR system (APPLIED BIOSYSTEMS, 7900HT)

### Methods

1) Anti-HBV Activity and Cytotoxicity Determination

HepG2.2.15 cells were plated into 96-well plate in 2% FBS culture medium at the density of 40,000 cells/well and 5,000cells/well for HBV inhibitory activity and cytotoxicity determination, respectively. After incubation at 37° C., 5% CO2 overnight, cells were treated with medium containing compounds for 6 days with medium and compounds refreshed after 3 days of treatment. Each compound was tested in a 1:3 serial dilutions at 8 different concentrations in triplicate. The highest concentration of the compounds was 10 uM or 1 uM for anti-HBV activity assay and 100 uM for cytotoxicity determination.

Cell viability was determined by CCK-8 assay. After 6 days of compounds treatment, 20 μl CCK-8 reagents were added to each well of cytotoxicity assay plates. Cell plates were incubated at 37° C., 5% CO2 for 2.5 h. The absorbance at 450 nm wavelength and the absorbance at 630 nm wavelength as reference was measured.

The change of HBV DNA level induced by the compounds was assessed by quantitative real-time polymerase chain reaction (qPCR). Briefly, the HBV DNA in the culture medium was extracted using QIAamp 96 DNA Blood Kit according to the manual and then quantified by real-time PCR assay using the primers and probe in the table 1 below.

TABLE 1

| Primers | | SEQ |
|---|---|---|
| or Probe | Sequence | ID NO: |
| HBV-Fw | GTGTCTGCGGCGTTTTATCA | 1 |
| HBV-Rev | GACAAACGGGCAACATACCTT | 2 |
| HBV-Probe | CCTCTKCATCCTGCTGCTATGCCTCATC | 3 |
| With FAM | | |
| reporter | | |
| and TAMRA | | |
| quencher | | |

2) DATA Analysis

EC50 and CC50 values are calculated by the GRAPHPAD PRISM software. If the CV % of DMSO controls is below 15% and the reference compounds shows expected activity or cytotoxicity, the data of this batch of experiment is considered qualified.

RESULTS: See Table 2 below.

TABLE 2

| Compound | EC₅₀ | CC₅₀ |
|---|---|---|
| ID | (uM) | (uM) |
| 1A | 0.0063 | 22.5 |
| 1B | 0.0430 | 22.4 |
| 2 | 0.0490 | 55.4 |
| 3A | 0.0190 | 25.2 |
| 3B | 0.0017 | 17.8 |
| 4A | 0.0490 | 52.8 |
| 4B | 0.0019 | 23.1 |
| 5 | 0.0017 | 11.8 |
| 6 | 0.0670 | 20.5 |
| 6A | 0.0194 | 11.4 |
| 7 | 0.7200 | 68.7 |
| 8 | 0.7900 | >100 |
| 9A | 0.0640 | 14.9 |
| 9B | 0.0007 | 14.6 |
| 10B | 0.0029 | 23.2 |
| 11A | 0.0019 | 16.2 |
| 12B | 0.0020 | 14.5 |
| 13A | 0.0010 | 14.7 |
| 14 | 0.0059 | 10.6 |
| 14A | 0.0012 | 9.6 |
| 15A | 0.0046 | 25.6 |
| 15B | 0.3000 | 45.0 |
| 16A | 0.0045 | 15.3 |
| 16B | 0.8083 | 29.5 |
| 17 | 0.0056 | 13.2 |
| 18B | 0.0015 | 13.0 |
| 19A | 0.0405 | 89.9 |
| 19B | 0.0018 | 51.2 |
| 20A | 0.0035 | 16.4 |
| 21A | 0.0060 | 15.2 |
| 22A | 0.0089 | 12.8 |
| 23 | 0.0046 | 2.6 |
| 24A | 0.0391 | 15.5 |
| 24B | 0.0010 | 9.6 |
| 24C | 0.0014 | 11.6 |
| 24D | 0.0350 | 9.4 |
| 25A | 0.0916 | 31.0 |
| 26A | 0.0132 | 26.2 |
| 27A | 0.0048 | 23.6 |
| 27B | 0.0026 | 25.3 |
| 28 | 0.0058 | 10.9 |
| 29B | 0.0015 | 11.2 |
| 30A | 0.0050 | 8.6 |
| 31A | 0.0150 | 43.7 |
| 32A | 0.0249 | 41.0 |
| 32B | 0.0274 | 42.5 |
| 33A | 0.0120 | 37.8 |
| 34A | 0.0079 | 47.4 |
| 35A | 0.0479 | 77.0 |
| 35B | 0.0011 | 46.6 |
| 36B | 0.0026 | 23.0 |
| 37 | 0.1 | 100 |

As the potency data shown in table 2, all these compounds demonstrated highly potent in vitro activities against HBV HepG2.2.15 cell.

### Example 2: Metabolic Stability of Test Compound in Human Hepatocyte Cell

Materials and reagents: see table 3 below.

TABLE 3

| Items | Supplier |
|---|---|
| Cryopreserved human hepatocytes | Bioreclamation IVT |
| Verapamil | Sigma Chemical Co |
| L-15 Medium | Life Technologies |
| Williams' Medium E | Life Technologies |
| Human recombinant insulin | Life Technologies |
| GlutaMAX | Life Technologies |
| Isotonic Percoll | General Electric |
| Fetal bovine serum | Corning |
| HEPES | Life Technologies |

### Study Design

1. The cryopreserved human hepatocytes cells were thawed in 37° C. water bath and diluted with pre-warmed incubation medium to a working cell density of 1×10\hat{ }6 viable cells/mL.
2. The 198 μL pre-warmed hepatocyte suspensions were spiked with 2 μL of 100 μM compound or reference compound(Verapamil) at a final concentration of 1.0 μM in a 96-well plate. The plate was incubated at 37° C., 900 rpm. All incubations will be performed in singlet.
3. 25 μL aliquots of well contents were collected at time points of 0, 15, 30, 60, 90 and 120 minutes. The reactions were stopped by the addition of 6-fold volumes of cold acetonitrile with internal standards.
4. After centrifugation for 25 minutes at 3,220 g. Aliquot of 100 μL of the supernatant was mixed with 100 μL of ultra-pure H₂O and then used for LC-MS/MS analysis.

### Data Analysis

All calculations were carried out using Microsoft Excel. Peak areas were determined from extracted ion chromatograms. Determine the in vitro half-life (t_{1/2}) of parent compound by regression analysis of the percent parent disappearance vs. time curve.

The in vitro half-life (in vitro t_{1/2}) is determined from the slope value k:

in vitro t_{1/2}=0.693/k

Conversion of the in vitro t_{1/2}(in min) into the in vitro intrinsic clearance (in vitro CLᵢₙₜ, in μL/min/10\hat{ }6 cells) is done using the following equation:

in vitro CLᵢₙₜ=kV/N

V=incubation volume (0.2 mL);

N=number of hepatocytes per well (0.2×10\hat{ }6 cells).

Conversion of the in vitro t_{1/2}(in min) into the scale-up intrinsic clearance (CLᵢₙₜ(liver), in mL/min/kg) was done using the following equation:

CLᵢₙₜ(liver)=kV/N×scaling factor

TABLE 4
Scaling factors for in vivo intrinsic
clearance prediction are listed below:

| | Liver Weight | Hepatocyte | | Liver |
|---|---|---|---|---|
| | (g liver/kg | Concentration | Scaling | blood flow |
| Species | body weight) | (10⁶ cells/g liver) | Factor | (Q, mL/min/kg) |
| Human | 25.7 | 99 | 2544.3 | 20.7 |

Control compound verapamil will be included in the assay. Any value of the compound that is not within the specified limits will be rejected and the experiment would be repeated.

Result

TABLE 5
Results Summary of Metabolic Stability
of Compounds in Human Hepatocytes

| | | Clᵢₙₜ |
|---|---|---|
| Compound | T_{½} | (liver) |
| ID | (min) | (mL/min/kg) |
| Reference 1 | 15.3 | 115.3 |
| Reference 2 | 147.1 | 12.0 |
| 1A | 229.5 | 7.7 |
| 3B | 174.4 | 10.1 |
| 4B | >289.1 | <6.1 |

Hepatocyte's metabolic stability test has become the “gold standard” for evaluating hepatic metabolism and toxicity of drugs and other xenobiotics in vitro. As the human hepatocyte stability data shown in table 5, compounds 1A, 3B, and 4B showed improved metabolic stability in human hepatocyte cells when comparing with reference 1 and reference 2.

### Example 3: In Vitro Assessment of Cytochrome P450 (Cyp450) Induction in Cryopreserved Human Hepatocytes

Materials: See table 6 below.

TABLE 6

| Items | Supplier |
|---|---|
| Cryopreserved human hepatocytes | Bioreclamation IVT |
| Williams' Medium E | Sigma |
| (without phenol red) | |
| Williams' Medium E | Life Technologies |
| Human recombinant insulin | Life Technologies |
| GlutaMAX | Life Technologies |
| Isotonic Percoll | General Electric |
| Fetal bovine serum | Corning |
| HEPES | Life Technologies |
| Dexamethasone | Local suppliers |
| CellTiter-Fluor ™ | Promega |
| Cell Viability Assay kit | |
| Matrigel ® and collagen I | Corning |
| coated 96-well plates | |
| TaqMan Gene Expression | Life Technologies |
| Cells-to-Ct Kit | |
| TaqMan Gene Exression assay | Applied Biosystems |
| probe (Catalog# 4351370) for | |
| CYP3A4 (Hs00604506_m1), and | |
| (Catalog# 4448490) for | |
| ACTB (Hs01060665_g1) | |

Equipment:

Infinite 200 PRO microplate reader, Tecan

7500 QPCR system, Applied Biosystems.

Study Design

Preparation and Plating of Human Hepatocytes
1. The cryopreserved human hepatocytes were thawed in 37° C. water bath and diluted by plating medium to a seeding density of 0.55×10\hat{ }6 cells/mL.
2. Transfer 100 μL to each well of collagen I coated 96-well plate. Place plate(s) in incubator and incubate at 37° C. for 4-6 hours.
3. After incubation, observe cell morphology, agitate plate(s) to loosen debris, and replace medium. Place plate in incubator and incubate for 18 hours.

Incubation with Test Compound(s)
1. Prepare dilute test compound and positive control inducers with 37° C. prepared incubation medium to respective working concentrations (Table 11). Final concentration of DMSO in the treatment group will be 0.1%. Prepare 25 mM chlorpromazine in DMSO and dilute 1000-fold with incubation medium as a cytotoxicity control.

TABLE 11
Test compound and positive control inducer concentrations

| | | Stock | Working |
|---|---|---|---|
| | | concentration | concentration |
| Enzyme | Treatment | (mM) | (μM) |
| CYP3A4 | Rifampicin | 10, 20 | 10, 20 |
| CYP3A4 | Test compound | 10 | 10 |

2. Remove the Hepatocyte plate from the incubator. Observe cell morphology. Replace the medium in the appropriate wells with 125 μL of the toxicity controls, DMSO controls, inducers, or test article solutions, each in triplicate.
3. After 24 hours and 48 hours, remove the Hepatocyte plate from the incubator and observe cell morphology. Renew the medium with test articles that freshly diluted from DMSO stocks. Return plate to the incubator.

3. Cell Viability Assessment

After 72 hours of treatment, warm the incubation medium to 37° C. Remove the induction plate(s) from the incubator. Observe cell morphology. Cell viability was assessed by CellTiter-Fluor™ Cell Viability Assay kit.

4. mRNA Preparation and RT-PCR
1. mRNA was prepared and measured using the Cells-to-Ct kit. Add DNase to Lysis solution.
2. 15 μL of sample lysate was added to 35 L of Reverse Transcription Master Mix (containing 2×RT Buffer, 20×RT Enzyme Mix and Nuclease-free Water) for a final 50 μL reaction volume.
3. Separate PCR cocktails were prepared for CYP3A4; containing the CYP specific probe set and that of ACTB as the endogenous control gene. A typical PCR cocktail contained TaqMan Universal Master Mix (2×), Taqman Gene Expression Assay probe (20×, CYP, FAM labeled), Taqman Gene Expression Assay probe (20×, ACTB, VIC labeled) and RNase-free water.
4. 4 μL cDNA samples or RT mix without cell lysate (negative control) were added to PCR cocktail to make the final volume of 20 μL. Templates for standard curve are prepared from a 3-fold serial dilution of the cDNA sample mixture of respective Rifampicin induced samples at highest concentration.
5. Reactions were analyzed on an Applied Biosystems Real Time PCR system (AB 7500). Each PCR was performed in triplicate.

Data Analysis

All calculations are carried out using Microsoft Excel.

1) Cell viability

Percent cell viability (%)=(I₍ₛₐₘₚₗₑ₎−I_{(background)})/(I_{(vehicle)}−I_{(background)})×100

Where “I” means fluorescence intensity.

2) mRNA quantification

For mRNA level determination, the mRNA content in each well is expressed as 2^{Ct(ACTB)-Ct(CYP)}.

Fold of induction=mRNA_{(induced)}/mRNA_{(vehicle)}

3) The percent adjusted positive control is determined by:

% of positive control=[(fold induction of test article)/(fold induction of positive control)]*100

Result

TABLE 7
Induction potential of CYP3A4 by test compound based on mRNA level
determination

| | | | | | mRNA | |
|---|---|---|---|---|---|---|
| | | | | | Fold | Percentage |
| | | | | Cell | induction | of positive |
| Compound | | CYP | Concentration | viability | (Mean ± | control at 10 |
| ID | Donor | isoform | (μM) | (%) | SD) | uM (%) |
| Reference 2 | 1 | CYP3A4 | 10 | 117 | 5.05 ± 0.61 | 20.50 |
| 1A | 1 | CYP3A4 | 10 | 95 | 0.37 ± 0.12 | 1.91 |

Induction of cytochrome P450 (CYP450) enzymes is associate with an increase prevalence of clinical drug-drug interactions and may result in therapeutic failure. CYP3A4 is by far the most abundant isoform and is responsible for the majority of CYP450-related metabolism of all marketed drugs. The CYP induction activity of compound 1A is far less than two-folds against vehicle control and far less than 20% against the positive control on CYP3A4 isoform. Compound 1A demonstrated no CYP induction effect when comparing with compound reference 2, thus devoid of CYP induction liability.

### Example 4: A Pharmacokinetic and Tissue Distribution Study of Compound Via Intravenous and Oral Administration in Male C57BL/6 Mice

Materials and Methods

Male C57BL/6 mice with a weight range of 20-25 g (Hua Fu Kang, China) were used. Animals were fasted overnight and free access to food 4 hours after dosing.

Test compound (correction factor: 1.00) was dissolved in a 20% hydroxypropyl-β-cyclodextrin (HP-β-CD) at a final concentration of 1 mg/ml for the intravenous (IV) formulation and at final concentrations of 0.5 mg/ml for the oral (PO) formulation. The intravenous formulation was dosed at 2 ml/kg to obtain a dose of 2 mg/kg. The oral formulations were dosed at 10 ml/kg to obtain final doses of 5 mg/kg.

Blood samples were taken at 7 and 20 min, 1, 2, 4, 8 and 24 h after intravenous dose administration. Blood and liver samples were taken at 30 min, 1, 2, 4, 8, 12 and 24 h after oral dose administration.

Approximately 0.020 mL blood will be collected into BD blood collection tubes containing K₃-EDTA at each time point. Samples were placed immediately on melting ice and plasma was obtained following centrifugation at 4° C. for 5 minutes at approximately 4000×g. Plasma samples were adjusted to pH 3-4 by phosphoric acid and stored at −75±15° C. prior to analysis. The whole process was completed within 1 hour.

Liver samples were collected at adopted time point, and the vial containing the tissues sample was snap-frozen in liquid nitrogen right away and kept at −75±15° C. prior to analysis. All liver samples were weighed and homogenized with phosphoric acid solution (pH to 3-4) by liver weight (g) to phosphoric acid solution volume (mL) ratio 1:4 before analysis.

Plasma and liver samples were analyzed using LC-MS/MS methods. The lower limit of quantification (LLOQ) for plasma was 1.0 ng/ml and for liver was 2.5 ng/g. A non-compartmental analysis using the “Linear up log down” rule was used for all data. A limited pharmacokinetic analysis was performed using Phoenix™ Professional (Version 6.1).

Results: See table 8 below for plasma PK results, and table 9 for PO liver PK results.

TABLE 8
Summary of IV/PO Pharmacokinetic results in mice plasma.

| | IV | | | PO | | | |
|---|---|---|---|---|---|---|---|
| Compound | Dose | Cl | AUC_{inf}/Dose | Dose | Cₘₐₓ | AUC_{inf}/Dose | |
| ID | (mg/kg) | (mL/min/kg) | (h*ng*kg/mg*mL) | (mg/kg) | (ng/mL) | (h*ng*kg/mg*mL) | F (%) |
| Reference 1 | 2.46 | 73.9 | 232.5 | 9.95 | 14.5 | 17.3 | 7.5 |
| Reference 2 | 2.08 | 49.1 | 345.2 | 5.34 | 733 | 181.5 | 52.7 |
| 1A | 2.12 | 16.6 | 1078.3 | 5.28 | 2353 | 784.8 | 72.9 |
| 3B | 1.91 | 20.2 | 830.9 | 5.4 | 2337 | 1049.3 | 126 |

TABLE 9
Summary of PO liver Pharmacokinetic results in mice.

| | Liver | | | | |
|---|---|---|---|---|---|
| Compound | Dose | Cₘₐₓ/Dose | AUC_{inf}/Dose | Cₘₐₓ Ratio | AUC_{Inf} Ratio |
| ID | (mg/kg) | (kg*ng/mg*mL) | (h*ng*kg/g*mg) | (Liver/Plasma) | (Liver/Plasma) |
| Reference 1 | 9.95 | 5.1 | 69.2 | 3.4 | 4.0 |
| Reference 2 | 5.34 | 1676 | 2894 | 12.2 | 15.9 |
| 1A | 5.28 | 6225 | 13733 | 14.0 | 17.5 |
| 3B | 5.4 | 6624 | 20884 | 15.3 | 19.9 |

Mouse in-vivo PK studies are critical to ensure drug candidates have appropriate PK properties that can be evaluated in preclinical pharmacology and safety studies. When comparing with compounds reference 1 and reference 2, compounds 1A and 3B showed a far slower clearance, over 3-folds higher dose-normalized AUC and increased bioavailability in plasma, and far increased dose-normalized Cₘₐₓ, and dose-normalized AUC_{inf} in liver.

### Example 5: A Pharmacokinetic Study of Test Compound after Intravenous and Oral Administration in Male SD Rats

Materials and Methods

Male SD rats with a weight range of 250-300 g (Si Bei Fu Laboratory Animal Technology Co. Ltd, China) were used. Animals were fasted overnight and free access to food 4 hours after dosing.

Test compound (correction factor: 1.00) was dissolved in a 20% hydroxypropyl-β-cyclodextrin (HP-β-CD) at a final concentration of 1 mg/ml for the intravenous (IV) formulation and at final concentrations of 0.5 mg/ml for the oral (PO) formulation.

The intravenous formulation was dosed at 2 ml/kg to obtain a dose of 2 mg/kg. The oral formulations were dosed at 10 ml/kg to obtain final doses of 5 mg/kg.

Blood samples were taken at 5, 15 and 30 min, 1, 2, 4, 8 and 24 h after intravenous dose administration. Blood samples were taken at 15 and 30 min, 1, 2, 4, 8, 12 and 24 h after oral dose administration.

Approximately 0.20 mL blood will be collected into BD blood collection tubes containing Sodium Fluoride (NaF), Potassium Oxalate (KoX) and K₃-EDTA at each time point. Samples were placed immediately on melting ice and plasma was obtained following centrifugation at 4° C. for 5 minutes at approximately 4000×g. Plasma samples were adjusted to pH 3-4 by phosphoric acid and stored at −75±15° C. prior to analysis. The whole process was completed within 1 hour.

Plasma samples were analyzed using LC-MS/MS methods. The lower limit of quantification (LLOQ) for plasma was 1.0 ng/ml.

A non-compartmental analysis using the “Linear up log down” rule was used for all data. A limited pharmacokinetic analysis was performed using Phoenix™ Professional (Version 6.1). Results: See table 10 below for plasma PK results.

TABLE 10
Summary of IV/PO Pharmacokinetic results in rat plasma.

| | IV | | | PO | | | |
|---|---|---|---|---|---|---|---|
| Compound | Dose | Cl | AUC_{inf}/Dose | Dose | Cₘₐₓ | AUC_{inf}/Dose | |
| ID | (mg/kg) | (mL/min/kg) | (h*ng*kg/mg*mL) | (mg/kg) | (ng/mL) | (h*ng*kg/mg*mL) | F (%) |
| Reference 2 | 1.93 | 65.6 | 254.9 | 5.05 | 332 | 129.7 | 50.8 |
| 1A | 1.83 | 11.6 | 1578.1 | 4.98 | 4059 | 1780.7 | 113 |
| 3B | 1.76 | 23.4 | 771.6 | 5.28 | 1181 | 389.2 | 50.3 |
| 4B | 2.09 | 28.8 | 594.3 | 6.84 | 962 | 336.8 | 56.5 |

Rat in-vivo PK studies are critical to ensure drug candidates have appropriate PK properties that can be evaluated in preclinical pharmacology and safety studies. Compounds 1A, 3B, and 4B showed a far slower clearance, over two-folds higher dose-normalized AUC (AUC_{inf/Dose}) and an increased (or equal) bioavailability (F (%)) when comparing with reference 2 compound.

1. A compound of Formula (I) 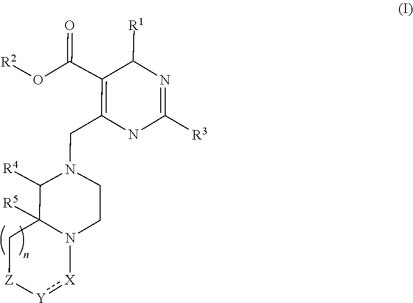
including the deuterated isomers, stereoisomers or tautomeric forms thereof, or a pharmaceutically acceptable salt thereof, wherein:
R¹ is selected from the group consisting of phenyl, thiophenyl, pyridyl, and pyridonyl, optionally substituted with one or more substituents selected from the group consisting of C₁₋₄ alkyl, halogen and CN;
R² is C₁₋₄alkyl;
R³ is selected from the group consisting of thiazolyl, pyridyl, and oxazolyl, optionally substituted with one or more substituents selected from fluorine and C₁₋₆alkyl;
n is an integer of 0 or 1;
R⁴ and R⁵ are independently selected from H and —COOH; [symbol]
is a single bond or a double bond;
when X and Y are linked by a single bond, X is selected from the group consisting of C(═S), C(═NR⁶), C(═CHR⁷) and CHR⁸, and Y is NR⁹;
when X and Y are linked by a double bond, X is C—SR⁹ or C—OR⁹, and Y is N atom;
Z is selected from the group consisting of CH₂, and C(═O);
R⁶ is selected from the group consisting of CN, C(═O)CH₃ and SO₂CH₃;
R⁷ is CN;
R⁸ is CF₃;
R⁹ is selected from the group consisting of H, —C₁₋₆alkyl, —C₁₋₆alkyl-R¹⁰, —C₁₋₆alkoxy-C₁₋₆alkyl-R¹⁰, —(CH₂)ₚ—C(R¹¹R¹²)—R¹⁰ and —(CH₂)ₚ-Q-R¹⁰;
p is an integer of 0, 1, 2, or 3;
R¹¹ and R¹² together with carbon atom to which they are attached form a 3- to 7-saturated membered ring, optionally containing a heteroatom, the heteroatom being an oxygen or a nitrogen, the nitrogen being substituted with H, —C₁₋₆alkyl, —C₁₋₆alkoxy-C₁₋₆alkyl and —C₁₋₆alkylcarbonyl;
Q is selected from the group consisting of aryl, heteroaryl, and a 3- to 7-saturated membered ring, optionally containing a heteroatom, the heteroatom being an oxygen or a nitrogen, the nitrogen being substituted with H, —C₁₋₆alkyl, —C₁₋₆alkoxy-C₁₋₆alkyl and —C₁₋₆alkylcarbonyl;
R¹⁰ is selected from —COOH, —C(═O)NHS(═O)₂—C₁₋₆alkyl, tetrazolyl, and carboxylic acid bioisosteres.

2. The compound of claim 1, wherein the carboxylic acid bioisosters are —S(═O)₂(OH), —P(═O)(OH)₂, —C(═O)NHOH, —C(═O)NHCN, 1,2,4-oxadiazol-5(4H)-one, and 3-hydroxy-4-methylcyclobut-3-ene-1,2-dione.

3. The compound of claim 1, wherein R¹ is phenyl substituted with one or more substituents selected from halogens and C₁₋₆alkyl.

4. The compound of claim 1, wherein R² is methyl or ethyl.

5. The compound of claim 1, wherein R³ is thiazolyl.

6. The compound of claim 1, wherein R⁴ and R⁵ are H.

7. The compound of claim 1, wherein X is C(═S).

8. The compounds of claim 1, wherein Z is CH₂.

9. The compound of claim 1, wherein R⁹ is —C₁₋₆alkyl-CO₂H, —(CH₂)ₚ—C(R¹¹R¹)—R¹⁰ or —(CH₂)ₚ-Q-R¹⁰.

10. The compound of claim 1, wherein Q is phenyl.

11. The compound of claim 1, wherein Q is a C₃₋₆cycloalkyl, or R¹¹ and R¹² together with carbon atom to which they are attached form a C₃₋₆cycloalkyl.

12. The compound of claim 1, wherein Q is a 3- to 6-saturated membered ring containing an oxygen, or R¹¹ and R¹² together with carbon atom to which they are attached form a 3- to 6-saturated membered ring containing an oxygen.

13. The compound according to claim 1, selected from the group consisting of the compounds having the following formulae:

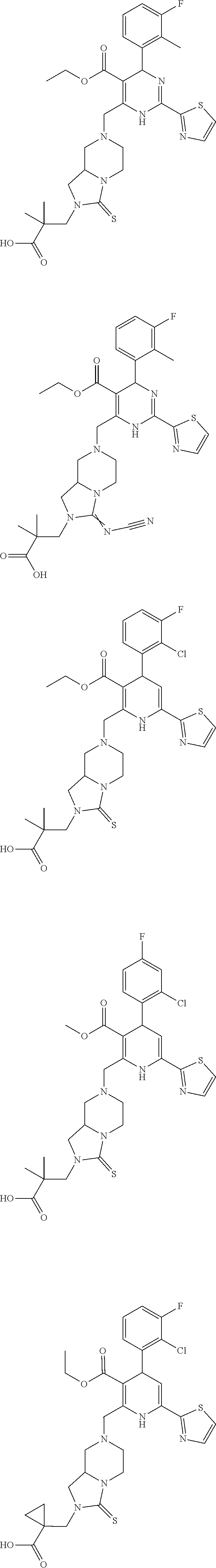 [SMILES: CCOC(=O)C1=C(CN2CCN3C(=S)N(CC(C)(C)C(=O)O)CC3C2)NC(c2nccs2)=NC1c1cccc(F)c1C ; CCOC(=O)C1=C(CN2CCN3C(=NC#N)N(CC(C)(C)C(=O)O)CC3C2)NC(c2nccs2)=NC1c1cccc(F)c1C ; CCOC(=O)C1=C(CN2CCN3C(=S)N(CC(C)(C)C(=O)O)CC3C2)NC(c2nccs2)=CC1c1cccc(F)c1Cl ; COC(=O)C1=C(CN2CCN3C(=S)N(CC(C)(C)C(=O)O)CC3C2)NC(c2nccs2)=CC1c1ccc(F)cc1Cl ; CCOC(=O)C1=C(CN2CCN3C(=S)N(CC4(C(=O)O)CC4)CC3C2)NC(c2nccs2)=CC1c1cccc(F)c1Cl]

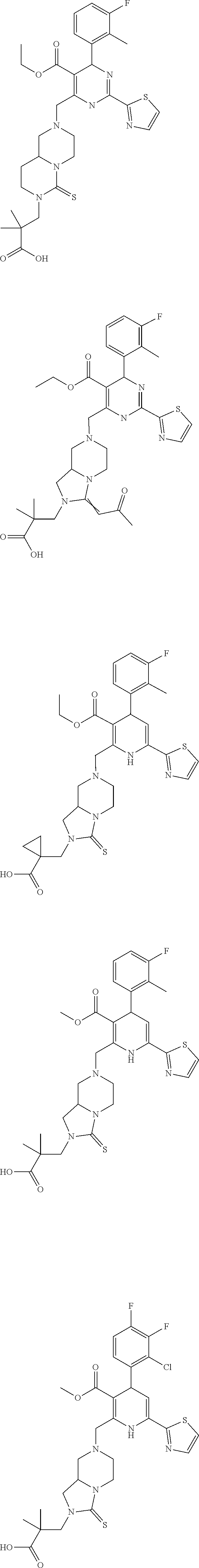

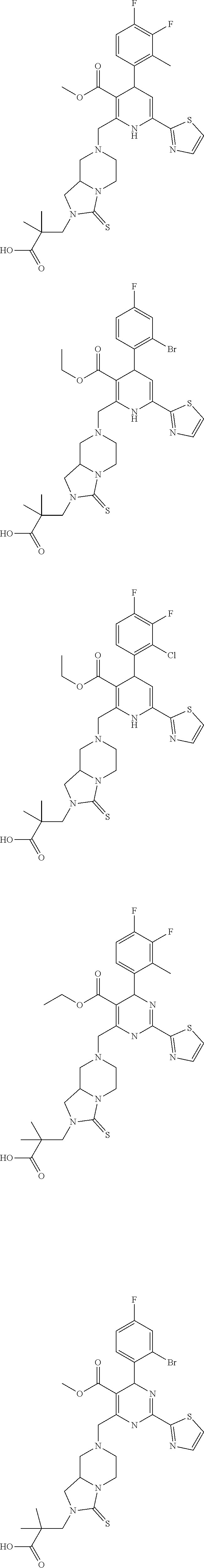

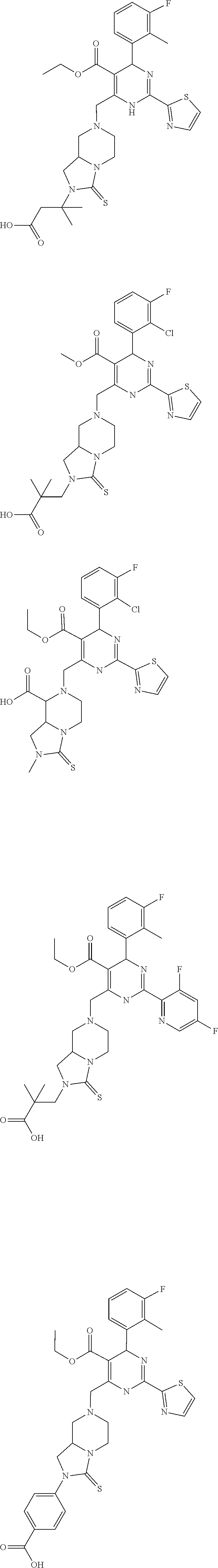

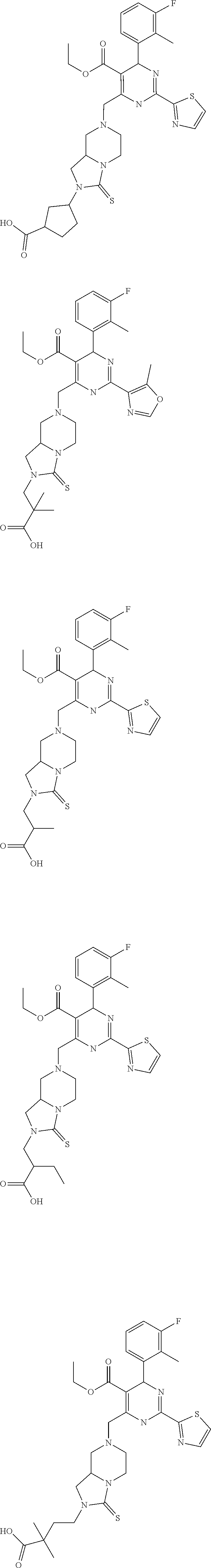

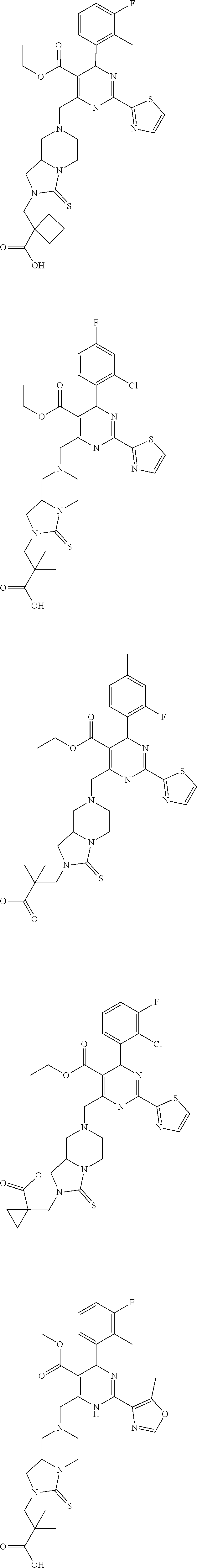

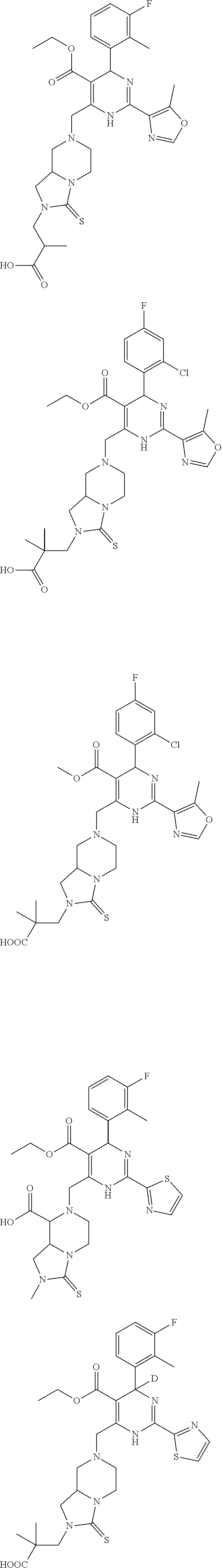 [SMILES: CCOC(=O)C1=C(CN2CCN3C(=S)N(CC(C)C(=O)O)CC3C2)NC(c2ncoc2C)=NC1c1cccc(F)c1C ; CCOC(=O)C1=C(CN2CCN3C(=S)N(CC(C)(C)C(=O)O)CC3C2)NC(c2ncoc2C)=NC1c1ccc(F)cc1Cl ; COC(=O)C1=C(CN2CCN3C(=S)N(CC(C)(C)C(=O)O)CC3C2)NC(c2ncoc2C)=NC1c1ccc(F)cc1Cl ; CCOC(=O)C1=C(CN2CCN3C(=S)N(C)CC3C2C(=O)O)NC(c2nccs2)=NC1c1cccc(F)c1C ; [2H]C1(c2cccc(F)c2C)N=C(c2nccs2)NC(CN2CCN3C(=S)N(CC(C)(C)C(=O)O)CC3C2)=C1C(=O)OCC]

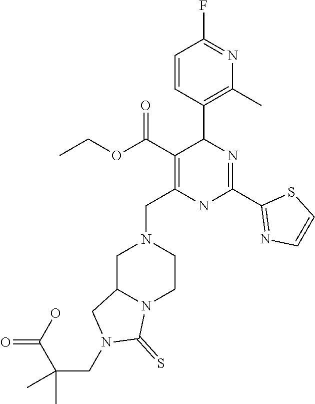

14. A pharmaceutical composition, which comprises the compound of claim 1 and which further comprises at least one pharmaceutically acceptable carrier.

15. The compound or pharmaceutically acceptable salt of any one of claim 1, for use as a medicament.

16. The compound or pharmaceutically acceptable salt of any one of claim 1, for use in the prevention or treatment of an HBV infection or of an HBV-induced disease in mammal in need thereof.

17. The compound or pharmaceutically acceptable salt of claim 1, for use in the prevention or treatment of chronic Hepatitis B.

18. A product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt of claim 1, and wherein said second compound is another HBV inhibitor which is selected from the group consisting of HBV combination drugs, HBV DNA polymerase inhibitors, immunomodulators, toll-like (TLR) receptor modulators, interferon alpha receptor ligands, hyaluronidase inhibitors, hepatitis b surface antigen (HbsAg) inhibitors, cytotoxic T-lymphocyte-associated protein 4 (ipi4) inhibitors, cyclohilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, farnsoid X receptor agonists, HBV antibodies, CCR2 chemokine antagonists, thymosin agonists, cytokines, nuceloprotein modulators, retinoic acid-inducible gene 1 stimulators, NOD2 stimulators, phosphatidylinositol 3-kinase (P13K) inhibitors, indoleamine 2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and anti-HBV drugs.

19. A process for the preparation of a compound according to claim 1, comprising the steps of:
a. The condensation of aldehyde of Formula (II), wherein Formula (II) is 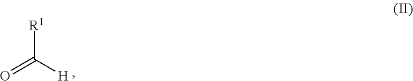
acetoacetate of Formula (III), wherein Formula (III) is 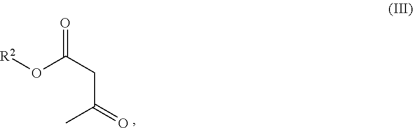
and amidine of Formula (IV), wherein Formula (IV) is 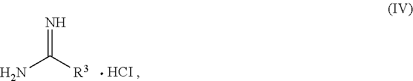
in the presence of a base, the base being preferably NaOAc, to form a compound according to Formula (I-1): 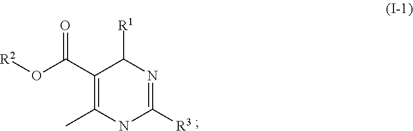
b. The bromination of compound of Formula (I-1), the brominating agent being preferably N-Bromosuccinimide, to form a compound according to Formula (I-2),
wherein Formula (I-2) is 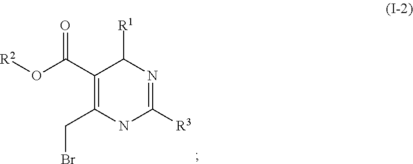
c. The coupling of compound of Formula (I-2) with a compound of Formula (V),
wherein Formula (V) is 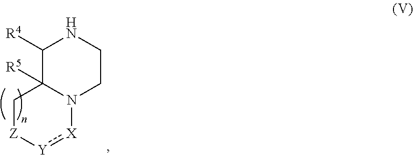
in the presence of a base, the base being preferably triethylamine, to form a compound according to Formula (I).

20. A compound, selected from the group consisting of the compounds having the following formulae, including any salts thereof: 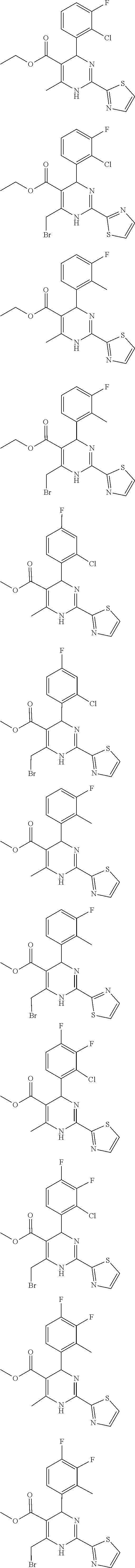 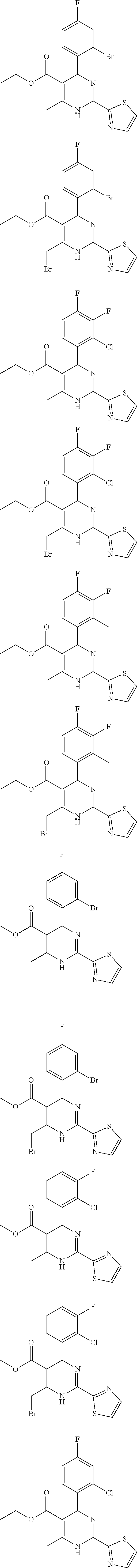 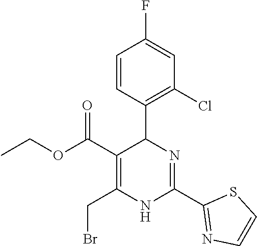 [SMILES: CCOC(=O)C1=C(CBr)NC(c2nccs2)=NC1c1ccc(F)cc1Cl] 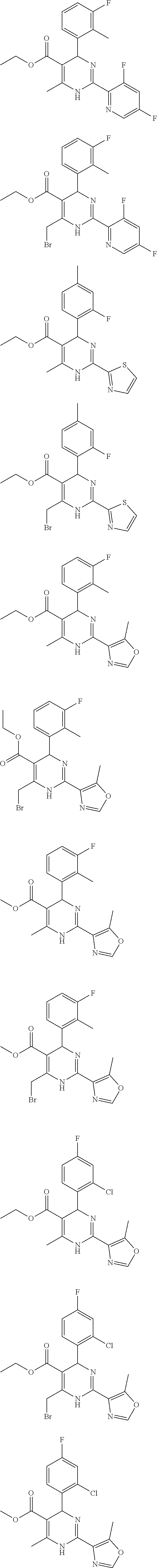 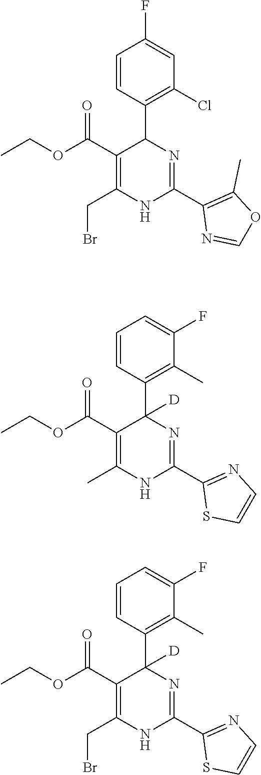 [SMILES: CCOC(=O)C1=C(CBr)NC(c2ncoc2C)=NC1c1ccc(F)cc1Cl ; [2H]C1(c2cccc(F)c2C)N=C(c2nccs2)NC(C)=C1C(=O)OCC ; [2H]C1(c2cccc(F)c2C)N=C(c2nccs2)NC(CBr)=C1C(=O)OCC] 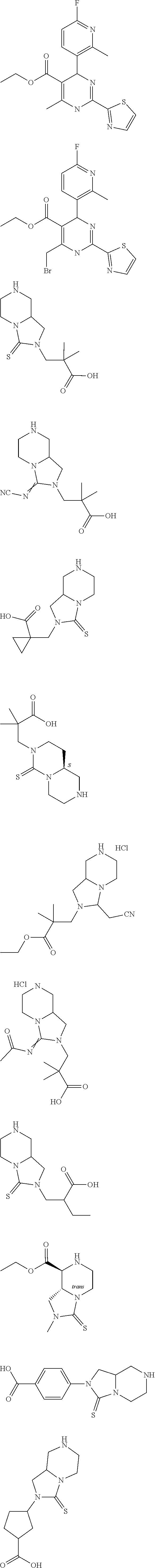 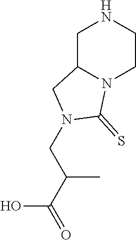 [SMILES: CC(CN1CC2CNCCN2C1=S)C(=O)O]
